(19) United States
(12) Patent Application Publication
(10) Pub. No.: US 2011/0319392 A1
(43) Pub. Date: Dec. 29, 2011

(54) Thiazole Sulfonamide And Oxazole Sulfonamide Kinase Inhibitors

(76) Inventors: George Adjabeng, Durham, NC (US); Erich Baum, Durham, NC (US); Neil Bifulco, JR., Durham, NC (US); Ronda G. Davis-Ward, Durham, NC (US); Scott Howard Dickerson, Durham, NC (US); Kelly Horne Donaldson, Durham, NC (US); Keith Hornberger, Durham, NC (US); Kimberly Petrov, Durham, NC (US); Tara Renae Reheault, Durham, NC (US); Douglas McCord Sammond, Durham, NC (US); Gregory M. Schaaf, Durham, NC (US)

(21) Appl. No.: 13/254,463

(22) PCT Filed: Mar. 10, 2010
(86) PCT No.: PCT/US10/26738

§ 371 Date: Sep. 2, 2011

Related U.S. Application Data
(60) Provisional application No. 61/159,513, filed on Mar. 12, 2009.

(51) Int. Cl. A61K 31/506; C07D 419/04; C07D 413/14; A61P 35/00; A61K 31/5377; A61K 31/5383; A61K 31/551; C07D 417/14; C07D 498/04

(52) U.S. Cl. 514/218; 544/331; 544/122; 544/105; 544/295; 544/123; 540/575; 544/82; 544/230; 514/275; 514/252.18; 514/235.8; 514/252.19; 514/233.2; 514/232.2

(57) ABSTRACT

The present invention provides thiazole sulfonamide and oxazole sulfonamide compounds, compositions containing the same, as well as processes for the preparation and methods for their use as pharmaceutical agents.

### FIELD OF THE INVENTION

The present invention relates to thiazole and oxazole compounds, compositions containing the same, as well as processes for the preparation and methods of using such compounds and compositions.

### BACKGROUND OF THE INVENTION

Both receptor tyrosine kinases and serine/threonine kinases have been implicated in cellular signaling pathways that control cell function, division, growth, differentiation, and death (apoptosis) through reversible phosphorylation of the hydroxyl groups of tyrosine or serine and threonine residues, respectively, in proteins. In signal transduction, for example, extracellular signals are transduced via membrane receptor activation, with amplification and propagation using a complex choreography of cascades of protein phosphorylation, and protein dephosphorylation events to avoid uncontrolled signaling. These signaling pathways are highly regulated, often by complex and intermeshed kinase pathways where each kinase may itself be regulated by one or more other kinases and protein phosphatases. The biological importance of these finely tuned systems is such that a variety of cell proliferative disorders have been linked to defects in one or more of the various cell signaling pathways mediated by tyrosine or serine/threonine kinases.

Receptor tyrosine kinases (RTKs) catalyze phosphorylation of certain tyrosyl amino acid residues in various proteins, including themselves, which govern cell growth, proliferation and differentiation. The ErbB family of RTKs includes EGFR, ErbB2, ErbB3, and ErbB4. Aberrant activity in the ErbB family kinases has been implicated in a range of hyperproliferative disorders including psoriasis, rheumatoid arthritis, bronchitis, and several cancers. The biological role of ErbB family RTKs and their implication in different disease states is discussed, for instance, in Ullrich, A., et al., Cell (Apr. 20, 1990) 61: 203-212; Aaronson, S., Science (1991) 254:1146-1153; Salomon, D., et al., Crit. Rev. Oncol./Hematol. (1995) 19:183-232; Woodburn, J. R., Pharmacol. Ther. (1999) 82: 2-3, 241-250; Normanno, N., et al., Curr. Drug Targets (2005) 6:243-257; and Hynes, N. et al., Nat. Rev. Cancer (2005) 5:341-345. In particular, elevated EGFR activity has been implicated in non-small cell lung, squamous cell lung, breast, bladder, head and neck squamous cell, esophageal, gastric, colorectal, pancreatic, thyroid, glial, cervical, and ovarian cancers (Salomon (1995) supra; Woodburn (1999) supra; Normanno (2005) supra; Hynes (2005) supra) and hepatocellular cancer Thomas, M. B., et al., Cancer (2007) 110(5):1059-1067 and Huether A., et al., J. Hepatol. (2005) 43(4):661-669. Furthermore, overexpression and/or mutation of ErbB2 has been implicated in the following:
Barret's adenocarcinoma (Brian et al., Hum. Pathol. (2000) 31(1) 35-39);
bladder cancer (Simon et al., Int. J. Cancer (2003) 107(5) 764-772);
breast cancer (Slamon et al., Science (1987) 235(4785) 177-182);
central nervous system tumors including primary CNS tumors such as glioblastomas, astrocytomas, ependymomas and glioblastoma multiforme (Potti et al., Cancer Invest. (2004) 22(4) 537-544; Koka et al., Am. J. Clin. Oncol. (2003) 26(4) 332-335; and Haynik et al., Appl. Immunohistochem. Mol. Morphol. (2007) 15(1) 56-68) and secondary CNS tumors (i.e., metastases to the central nervous system of tumors originating outside of the central nervous system),
gastric cancer including esophageal cancer, gastro-esophageal cancer, and salivary gland mucoepidermoid carcinoma (Tanner et al., Ann. Oncol. (2005) 16(2) 273-278; Yano et al., Oncol. Rep. (2006) 15(1) 65-71; Mimura et al. Br. J. Cancer (2005) 92(7) 1253-1260; and Nguyen et al. J. Otolaryngol. (2003) 32(5) 328-331);
carcinoma of the head and neck including squamous cell carcinoma of the head and neck (Weed et al. Head Neck (2004) 26(4) 353-364);
lung cancer (Stephens et al. Nature (2004) 431 525-526), including small cell lung cancer (Canoz et al. Lung (2006) 184(5) 267-272) and non-small cell lung cancer (Hirsch et al. Br. J. Cancer (2002) 86(9) 1449-1456);
melanoma (Nyormoi & Bar-Eli Clin. Exp. Metastasis (2003) 20(3) 251-263);
ovarian cancer (Slamon et al. Science (1989) 244(4905) 707-712) and endometrial cancer (Morrison et al. J. Clin. Oncol. (2006) 24(15) 2376-2385 and Liu Taiwan. J. Obstet. Gynecol. (2007) 46(1) 26-32);
pancreatic endocrine tumors (Goebel et al. Cancer Res. (2002) 62(13) 3702-3710); renal cancer (Latif et al. BJU Int. (2002) 89(1)5-9) and Wilm's tumor (Menard et al. Cell. Mol. Life. Sci. (2004) 61(23) 2965-2978); and
uterine cancer including uterine sarcoma (Slomovitz et al. J. Clin. Oncol. (2004) 22(15) 3126-3132; and Amant et al. Gynecol. Oncol. (2004) 95(3) 583-587).

A timeline of events pertaining to the role of the ErbB family kinases in cancer may be found in Gschwind, A., et al., Nat. Rev. Cancer (2004) 4:361-370. By virtue of the role played by the ErbB family kinases in these cancers and the relative success of inhibitors of these kinases in the clinic, it is widely acknowledged that inhibitors of one or more ErbB family kinases will be useful for the treatment of such cancers. Downstream of the several RTKs, including EGFR and ErbB2, lie several signaling pathways, among them being the Ras-Raf-MEK-ERK kinase pathway. It is currently understood that activation of Ras GTPase proteins or other upstream kinases in response to growth factors, hormones, cytokines, etc. stimulates phosphorylation and activation of Raf kinases. These kinases then phosphorylate and activate the intracellular protein kinases MEK1 and MEK2, which in turn phosphorylate and activate other protein kinases, ERK1 and ERK2. This signaling pathway, also known as the mitogen-activated protein kinase (MAPK) pathway or cytoplasmic cascade, mediates cellular responses to growth signals. The ultimate function of this is to link receptor activity at the cell membrane with modification of other membrane-associated, cytoplasmic, or nuclear targets that govern cell proliferation, differentiation, and survival. Mutations in various Ras GTPases and the B-Raf kinase have been identified that can lead to sustained and constitutive activation of the MAPK pathway, ultimately resulting in increased cell division and survival. As a consequence of this, these mutations have been strongly linked with the establishment, development, and progression of a wide range of human cancers. The biological role of the Raf kinases, and specifically that of B-Raf, in signal transduction is described in Davies, H., et al., Nature (2002) 9:1-6; Garnett, M. J. & Marais, R., Cancer Cell (2004) 6:313-319; Zebisch, A. & Troppmair, J., Cell. Mol. Life. Sci. (2006) 63:1314-1330; Midgley, R. S. & Kerr, D. J., Crit. Rev. One/Hematol. (2002) 44:109-120; Smith, R. A., et al., Curr. Top. Med. Chem. (2006) 6:1071-1089; and Downward, J., Nat. Rev. Cancer (2003) 3:11-22.

Naturally occurring mutations of the B-Raf kinase that activate MAPK pathway signaling have been found in a large percentage of human melanomas (Davies (2002) supra) and thyroid cancers (Cohen et al. J. Nat. Cancer Inst. (2003) 95(8) 625-627 and Kimura et al. Cancer Res. (2003) 63(7) 1454-1457), as well as at lower, but still significant, frequencies in the following:
Barret's adenocarcinoma (Garnett et al., Cancer Cell (2004) δ 313-319 and Sommerer et al. Oncogene (2004) 23(2) 554-558),
billiary tract carcinomas (Zebisch et al., Cell. Mol. Life. Sci. (2006) 63 1314-1330), breast cancer (Davies (2002) supra),
cervical cancer (Moreno-Bueno et al. Clin. Cancer Res. (2006) 12(12) 3865-3866), cholangiocarcinoma (Tannapfel et al. Gut (2003) 52(5) 706-712),
central nervous system tumors including primary CNS tumors such as glioblastomas, astrocytomas and ependymomas (Knobbe et al. Acta Neuropathol. (Berl.) (2004) 108(6) 467-470, Davies (2002) supra, and Garnett et al., Cancer Cell (2004) supra) and secondary CNS tumors (i.e., metastases to the central nervous system of tumors originating outside of the central nervous system),
colorectal cancer, including large intestinal colon carcinoma (Yuen et al. Cancer Res. (2002) 62(22) 6451-6455, Davies (2002) supra and Zebisch et al., Cell. Mol. Life Sci. (2006),
gastric cancer (Lee et al. Oncogene (2003) 22(44) 6942-6945),
carcinoma of the head and neck including squamous cell carcinoma of the head and neck (Cohen et al. J. Nat. Cancer Inst. (2003) 95(8) 625-627 and Weber et al. Oncogene (2003) 22(30) 4757-4759),
hematologic cancers including leukemias (Garnett et al., Cancer Cell (2004) supra, particularly acute lymphoblastic leukemia (Garnett et al., Cancer Cell (2004) supra and Gustafsson et al. Leukemia (2005) 19(2) 310-312), acute myelogenous leukemia (AML) (Lee et al. Leukemia (2004) 18(1) 170-172, and Christiansen et al. Leukemia (2005) 19(12) 2232-2240), myelodysplastic syndromes (Christiansen et al. Leukemia (2005) supra) and chronic myelogenous leukemia (Mizuchi et al. Biochem. Biophys. Res. Commun. (2005) 326(3) 645-651); Hodgkin's lymphoma (Figl et al. Arch. Dermatol. (2007) 143(4) 495-499), non-Hodgkin's lymphoma (Lee et al. Br. J. Cancer (2003) 89(10) 1958-1960), megakaryoblastic leukemia (Eychene et al. Oncogene (1995) 10(6) 1159-1165) and multiple myeloma (Ng et al. Br. J. Haematol. (2003) 123(4) 637-645),
hepatocellular carcinoma (Garnett et al., Cancer Cell (2004),
lung cancer (Brose et al. Cancer Res. (2002) 62(23) 6997-7000, Cohen et al. J. Nat. Cancer Inst. (2003) supra and Davies (2002) supra), including small cell lung cancer (Pardo et al. EMBO J. (2006) 25(13) 3078-3088) and non-small cell lung cancer (Davies (2002) supra),
ovarian cancer (Russell & McCluggage J. Pathol. (2004) 203(2) 617-619 and Davies (2002) supra), endometrial cancer (Garnett et al., Cancer Cell (2004) supra, and Moreno-Bueno et al. Clin. Cancer Res. (2006) supra),
pancreatic cancer (Ishimura et al. Cancer Lett. (2003) 199(2) 169-173),
pituitary adenoma (De Martino et al. J. Endocrinol. Invest. (2007) 30(1) RC1-3),
prostate cancer (Cho et al. Int. J. Cancer (2006) 119(8) 1858-1862),
renal cancer (Nagy et al. Int. J. Cancer (2003) 106(6) 980-981),
sarcoma (Davies (2002) supra), and
skin cancers (Rodriguez-Viciana et al. Science (2006) 311(5765) 1287-1290 and Davies (2002) supra).
   Overexpression of c-Raf has been linked to AML (Zebisch et al., Cancer Res. (2006) 66(7) 3401-3408, and Zebisch (Cell. Mol. Life. Sci. (2006)) and erythroleukemia (Zebisch et la., Cell. Mol. Life. Sci. (2006).

By virtue of the role played by the Raf family kinases in these cancers and exploratory studies with a range of preclinical and therapeutic agents, including one specifically targeted to inhibition of B-Raf kinase activity (King A. J., et al., (2006) Cancer Res. 66:11100-11105), it is generally accepted that inhibitors of one or more Raf family kinases will be useful for the treatment of such cancers.

PCT publication No. WO02/043467, published 27 May 2004 to Cyclacel Ltd., recites antiviral compounds of the formula

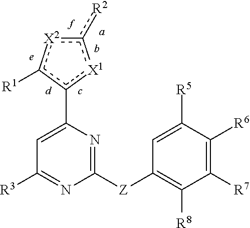

wherein
(A) one of X¹ and X² is S, and the other of X¹ and X² is N;
   “a” is a single bond; and
   “b”, “c”, “d”, “e” and “f” are single or double bonds so as to form a thiazolyl ring;
   R² is independently as defined below for R¹ and R³; or
(B) one of X¹ and X² is S, and the other of X¹ and X² is NR⁹;
   “a” and “d” are each double bonds; and
   “b”, “c”, “e” and “f” are each single bonds;
   R² is oxo;
   R⁹ is H or alkyl;
      where:
Z is NH, NHCO, NHSO₂, NHCH₂, CH₂, CH₂CH₂, or CH═CH,
R¹ and R³ are independently H, alkyl, aryl, arylalkyl, heterocycle, halogen, NO₂, CN, OH, alkoxy, aryloxy, NH₂, NH-alkyl, N—(R′)(R″), NH-aryl, N-(aryl)₂, NHCOR′, COOH, COO-alkyl, COO-aryl, CONH₂, CONH—R′, CON—(R′)(R″), CONH-aryl, CON-(aryl)₂, SO₃H, SO₂NH₂, CF₃, CO—R′, or CO-aryl, wherein said alkyl, NH-aryl, COO-alkyl, NH-alkyl, aryl, arylalkyl and heterocycle groups may be further substituted with one or more groups selected from halogen, NO₂, CN, OH,
   O-methyl, NH₂, COOH, N—(R′)(R″), CONH₂ and CF₃;
R⁴, R⁵, R⁶, R⁷ and R⁸ are independently from each other H, substituted or unsubstituted lower alkyl, halogen, NO₂, CN, OH, substituted or unsubstituted alkoxy, NH₂, NH—R′, alkyl-aryl, alkyl-heteroaryl, NH(C═NH)NH₂, N(R)₃⁺, N—(R′)(R″), COOH, COO—R′, CONH₂, CONH—R′, CON—(R′)(R″), SO₃H, SO₂NH₂, CF₃, or (CH₂)ₙ—O—(CH₂)ₘNR′R″, (CH₂)ₙCO₂(CH₂)ₘOR′″ wherein n is 0, 1, 2 or 3 and m is 1, 2 or 3;
wherein R′ and R″ are each independently substituted or unsubstituted alkyl or alkenyl groups that may be the same or different;
   and pharmaceutically acceptable salts thereof.

See also, PCT Publication Nos. WO2004/043953, published 27 May 2004 and WO2005/116025, published 8 Dec. 2005, also to Cyclacel Lmtd.

PCT Publication No. 2004/056368, published 8 Jul. 2004 to Cyclacel Lmtd., recites compounds for treating diabetes and CNS disorders, alopecia, CV disorders and stroke, having the formula:

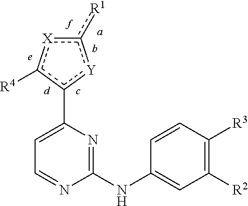

wherein
(A) one of X and Y is S, and the other is N; or
   one of X and Y is NH or N—R⁵, and the other is C—R⁶;
   “a” is a single bond; and
   “b”, “c”, “d”, “e” and “f” are single or double bonds so as to form a heteroaryl ring;
   R¹ is R⁷ with the proviso that R¹ is other than H or Me; or
(B) one of X and Y is S, and the other is NH or N—R⁵;
   “a” and “d” are each double bonds; and
   “b”, “c”, “e” and “f” are each single bonds;
   R¹ is oxo; and
R², R³, R⁴, R⁵, and R⁶ are each independently H or R⁷;
R⁷ is a group (CH₂)ₙ—R⁸, wherein n is 0, 1, 2, 3 or 4 and wherein R⁸ is selected from alkyl, aryl, heteroaryl, heterocycloalkyl, F, Cl, Br, I, CF₃, NO₂, CN, OH, O-alkyl, O-aryl, O-heteroaryl, O-heterocycloalkyl, CO-alkyl, CO-aryl, CO-heteroaryl,
   CO-heterocycloalkyl, COO-alkyl, NH₂, NH-alkyl, NH-aryl, N(alkyl)₂, NH-heteroaryl, NH-heterocycloalkyl, COOH, CONH₂, CONH-alkyl, CON(alkyl)₂, CONH-aryl, CONH-heteroaryl, CONH-heterocycloalkyl, SO₃H, SO₂-alkyl,
   SO₂-aryl, SO₂-heteroaryl, SO₂-heterocycloalkyl, SO₂NH₂, SO₂NH-alkyl, SO₂N(alkyl)₂, SO₂NH-aryl, SO₂NH-heteroaryl, SO₂NH-heterocycloalkyl, wherein said alkyl, aryl, heteroaryl, and heterocycloalkyl groups are optionally substituted with one or more groups selected from halogen, NO₂, OH, O-methyl, NH₂, COOH, CONH₂ and CF₃.

### SUMMARY OF THE INVENTION

In a first aspect of the present invention, there is provided compounds of formula (I):

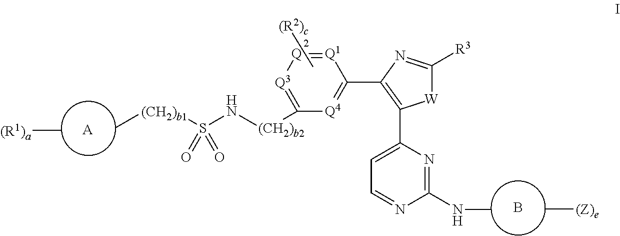

wherein:
a is 0, 1, 2 or 3;
each R¹ is the same or different and is independently selected from halo, alkyl, haloalkyl, —OR⁶, —CO₂H, —CO₂R⁶, and —CN;
Ring A is C₃₋₆cycloalkyl, phenyl, and 5-6 membered heterocycle or heteroaryl having 1 or 2 heteroatoms selected from N, O and S;
one of b1 and b2 is 0 and the other is 0 or 1;
c is 0, 1 or 2;
each R² is the same or different and is independently selected from halo, alkyl, haloalkyl, —OR⁶, and —CN;
each of Q¹, Q², Q³, and Q⁴ is CH or C—R² or one of Q¹, Q², Q³, and Q⁴ is N and the others are CH or C—R²;
W is —O— or —S—;
R³ is selected from H, alkyl, haloalkyl, alkenyl, C₃₋₆cycloalkyl, phenyl, Het, —CH₂-Het, —NR⁶R⁷, —N(R⁶)—C₃₋₆cycloalkyl, —N(R⁶)Het, —N(R⁶)R⁵—Het, —N(R⁶)—R⁶—OR⁷, N(R⁶)—R⁵—NR⁶R⁷, —N(H)C(O)R⁶, —N(R⁶)—C(O)—NR⁶R⁷, —N(H)SO₂R⁶, —N(R⁶)—R⁵—S(O)₂R⁷, and —N(R⁶)—S(O)₂—NR⁶R⁷,
   wherein each of said cycloalkyl is optionally substituted with 1 or 2 substituents which are the same or different and are independently selected from halo, C₁₋₃alkyl, haloC₁₋₃alkyl, OH, O—C₁₋₃alkyl, oxo, S(C₁₋₃alkyl), SO₂, NH₂, N(H)C₁₋₃alkyl and N(C₁₋₃alkyl)₂, and
   wherein said phenyl is optionally substituted with 1, 2 or 3 substituents which are the same or different and are each independently selected from halo,
      C₁₋₃alkylene-O—C₁₋₃alkyl, OH, C₁₋₃alkylene-OH, NH₂, N(H)Cl_{—}3alkyl, N(C₁₋₃alkyl)₂, CN and NO₂;
   each Het is the same or different and is independently a 4-6 membered heterocycle having 1 or 2 heteroatoms selected from N, O and S and optionally substituted with 1 or 2 substituents which are the same or different and are each independently selected from halo, C₁₋₃alkylene-O—C₁₋₃alkyl, OH, C₁₋₃alkylene-OH, oxo, SO₂(C₁₋₃alkyl), C₁₋₃alkylene-SO₂(C₁₋₃alkyl), NH₂, N(H)C₁₋₃alkyl, N(C₁₋₃alkyl)₂, CN, and —CH₂CN;
each R⁵ is the same or different and is independently C₁₋₄alkylene;
Ring B is selected from phenyl, 9-10 membered aryl, 5-6 membered heteroaryl and 9-10 membered heteroaryl, each heteroaryl having 1, 2, or 3 heteroatoms selected from N, O and S;
   wherein when Ring B is selected from phenyl and 5-6 membered heteroaryl, then
   e is 0, 1, 2 or 3; and
   each Z is the same or different and is independently selected from:
      halo, alkyl, haloalkyl, alkenyl,
      Het², —R⁵Het², Het³-Het²,
      oxo, —OR⁶, —R⁵—OR⁶, —O—R⁵—OR⁶, —OHet², —O—R⁵—Het², —O—R⁵—NR⁶R⁷,
      —O—R⁵—S(O)₂R⁶, —C(O)R⁶, —C(O)CH₂NR⁶R⁷, —CO₂R⁶, —R⁵—CO₂R⁶, —S(O)_{f} R⁶,
      —R⁵—S(O)₂R⁶, —S(O)₂NR⁶R⁷, —R⁵—S(O)₂NR⁶R⁷, —S(O)₂—R⁵—NR⁶R⁷, —NR⁶R⁷, —R⁵—NR⁶R⁷, —N(R⁶)Het², —N(R⁶)—R⁵—OR⁷, —N(R⁶)—R⁵—S(O)_{f}R⁷, —N(R⁶)—R⁵—CN, —N(R⁶)—R⁵—NR⁶R⁷, —N(H)S(O)₂R⁶, —N(R⁶)—C(O)—NR⁶R⁷, —N(R⁶)—S(O)₂—NR⁶R⁷, —CN, and —R⁵—CN; and
   wherein when Ring B is selected from 9-10 membered aryl and 9-10 membered heteroaryl, then
   e is 0, 1 or 2; and
   each Z is the same or different and is independently selected from: halo, alkyl, oxo, —C(O)R⁶, —C(O)CH₂NR⁶R⁷, —OR⁶, and —NR⁶R⁷;
   each Het² is the same or different and is independently a heterocycle optionally substituted with 1 or 2 substituents which are the same or different and are each independently selected from halo, C₁₋₃alkyl, haloC₁₋₃alkyl, O—C₁₋₃alkyl, C₁₋₃alkylene-O—C₁₋₃alkyl, OH, C₁₋₃alkylene-OH, oxo, C(O)(C₁₋₃alkyl), SO₂(C₁₋₃alkyl), C₁₋₃alkylene-SO₂(C₁₋₃alkyl), NH₂, N(H)C₁₋₃alkyl, N(C₁₋₃alkyl)₂, CN and C₁₋₃alkylene-CN;
   Het³ is a 5-6 membered heterocycle having 1 or 2 heteroatoms selected from N, O and S and optionally substituted with 1 or 2 substituents which are the same or different and are each independently selected from halo,
      haloC₁₋₃alkyl, and O—C₁₋₃alkyl;
   f is 0, 1 or 2; and
      each R⁶ and each R⁷ is the same or different and is independently H, alkyl or haloalkyl;
      and pharmaceutically acceptable salts thereof.

In a second aspect of the present invention, there is provided compounds of formula (I-i)

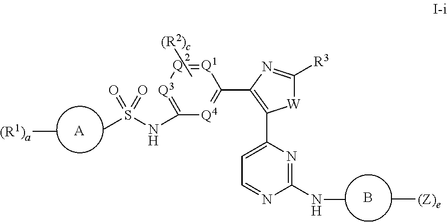

wherein all variables are as defined above, and pharmaceutically acceptable salts thereof.

In a third aspect of the present invention, there is provided compounds of formula (I-i-a)

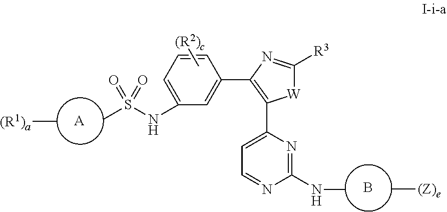

wherein all variables are as defined above, and pharmaceutically acceptable salts thereof.

In a fourth aspect of the present invention, there is provided compounds of formula (I-i-b1)

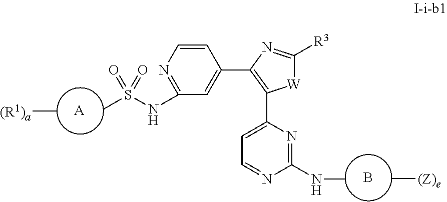

wherein all variables are as defined above, and pharmaceutically acceptable salts thereof.

In a fifth aspect of the present invention, there is provided compounds of formula (I-ii)

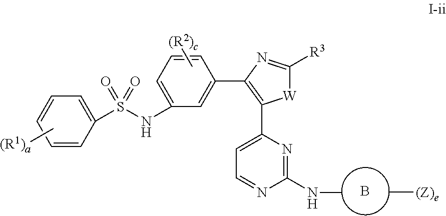

wherein all variables are as defined above, and pharmaceutically acceptable salts thereof.

In a sixth aspect of the present invention, there is provided compounds of formula (I-i-as)

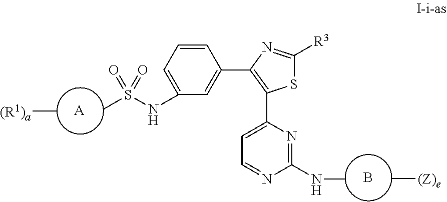

wherein all variables are as defined above, and pharmaceutically acceptable salts thereof.

In a seventh aspect, the present invention provides compounds of formula (I-iv-a), (I-iv-b), (I-iv-c), (I-iv-d), (I-iv-e) and (I-iv-f):

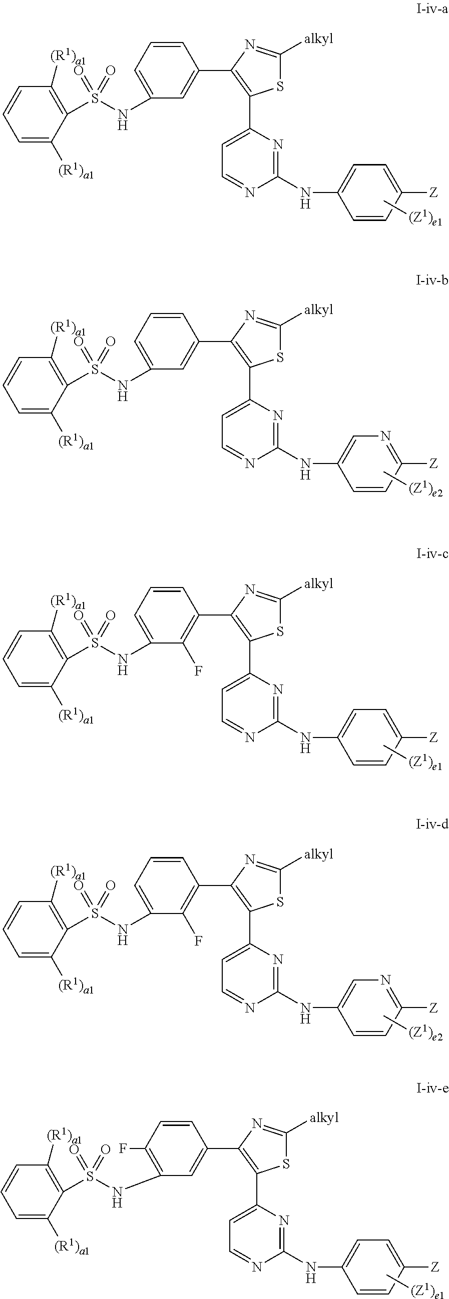

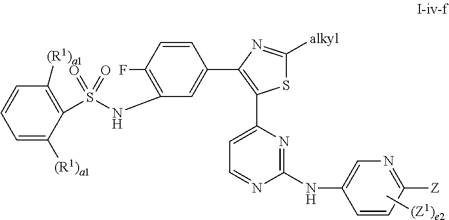

wherein a1 is 0 or 1;
R¹ is halo;
e1 is 0, 1 or 2;
e2 is 0 or 1;
Z is as defined in claim 1; and
Z¹ is halo, alkyl, haloalkyl, O-alkyl, CO₂H
or a pharmaceutically acceptable salt thereof.
In an eighth aspect of the present invention, there is provided a compound selected from:
N-[3-(2-(1-Methylethyl)-5-{2-[(6-{4-[2-(methyloxy)ethyl]-1-piperazinyl}-3-pyridinyl)amino]-4-pyrimidinyl}-1,3-thiazol-4-yl)phenyl]benzenesulfonamide;
N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-4-fluorobenzenesulfonamide;
N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-2-fluorobenzenesulfonamide;
2,6-Difluoro-N-{3-[5-(2-{p-fluoro-4-({1-[2-(methylsulfonyl)ethyl]-4-piperidinyl}oxy)phenyl]amino}-4-pyrimidinyl)-2-(1-methylethyl)-1,3-thiazol-4-yl]phenyl}benzenesulfonamide hydrochloride;
5-({4-[4-(3-{[(2,6-Difluorophenyl)sulfonyl]amino}phenyl)-2-(1-methylethyl)-1,3-thiazol-5-yl]-2-pyrimidinyl}amino)-2-(4-morpholinyl)benzoic acid;
2,6-Difluoro-N-{3-[5-[2-({6-[(9aR)-hexahydropyrazino[2,1-c][1,4]oxazin-8(1H)-yl]-3-pyridinyl}amino)-4-pyrimidinyl]-2-(1-methylethyl)-1,3-thiazol-4-yl]phenyl}benzenesulfonamide;
2,6-Difluoro-N-{3-[5-{2-[(3-fluoro-4-{4-[2-(methylsulfonyl)ethyl]-1-piperazinyl}phenyl)amino]-4-pyrimidinyl}-2-(1-methylethyl)-1,3-thiazol-4-yl]phenyl}benzenesulfonamide hydrochloride;
2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide;
N-{3-[5-(2-{[3-(Dimethylamino)-4-(methyloxy)phenyl]amino}-4-pyrimidinyl)-2-(1-methylethyl)-1,3-thiazol-4-yl]phenyl}-2,6-difluorobenzenesulfonamide trifluoroacetate;
2,6-Difluoro-N-[3-(2-(1-methylethyl)-5-{2-[(5-methyl-2-(methyloxy)-4-(4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-{3-[2-(1-methylethyl)-5-(2-{[2-(methyloxy)-4-(4-{-4-[2-(methylsulfonyl)ethyl]-1-piperazinyl}-1-piperidinyl)phenyl]amino}-4-pyrimidinyl)-1,3-thiazol-4-yl]phenyl}benzenesulfonamide;
2,6-Difluoro-N-{3-[5-[2-({3-fluoro-4-[4-(2-fluoroethyl)-1-piperazinyl]phenyl}amino)-4-pyrimidinyl]-2-(1-pyrrolidinyl)-1,3-thiazol-4-yl]phenyl}benzenesulfonamide;
2-Fluoro-N-{2-fluoro-5-[5-{2-[(3-fluoro-4-{4-[2-(methylsulfonyl)ethyl]-1-piperazinyl}phenyl)amino]-4-pyrimidinyl}-2-(1-methylethyl)-1,3-thiazol-4-yl]phenyl}benzenesulfonamide hydrochloride;
2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide;
N-{5-[5-(2-{[6-(4-Cyano-1-piperidinyl)-3-pyridinyl]amino}-4-pyrimidinyl)-2-(1-methylethyl)-1,3-thiazol-4-yl]-2-fluorophenyl}-2,6-difluorobenzenesulfonamide;
2,6-Difluoro-N-{2-fluoro-5-[5-[2-({3-fluoro-4,4-(2-fluoroethyl)-1-piperazinyl]phenyl}amino)-4-pyrimidinyl]-2-(1-methylethyl)-1,3-thiazol-4-yl]phenyl}benzenesulfonamide;
2,6-Difluoro-N-{2-fluoro-5-[5-(2-{[6-(4-morpholinyl)-3-pyridinyl]amino}-4-pyrimidinyl)-2-(1-pyrrolidinyl)-1,3-thiazol-4-yl]phenyl}benzenesulfonamide;
N-{5-[2-(1,1-Dimethylethyl)-5-(2-{[6-(4-morpholinyl)-3-pyridinyl]amino}-4-pyrimidinyl)-1,3-thiazol-4-yl]-2-fluorophenyl}-2,6-difluorobenzenesulfonamide hydrochloride;
2,6-Difluoro-N-{2-fluoro-3-[5-(2-{[5-fluoro-6-(4-morpholinyl)-3-pyridinyl]amino}-4-pyrimidinyl)-2-(1-methylethyl)-1,3-thiazol-4-yl]phenyl}benzenesulfonamide; and
2,6-Difluoro-N-{4-[2-(1-methylethyl)-5-(2-{[6-(4-morpholinyl)-3-pyridinyl]amino}-4-pyrimidinyl)-1,3-thiazol-4-yl]-2-pyridinyl}benzenesulfonamide; and free base or pharmaceutically acceptable salts thereof.

In a ninth aspect of the present invention, there is provided a compound selected from
2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide; and
2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide;
   and pharmaceutically acceptable salts thereof.

In a tenth aspect of the present invention, there is provided a compound selected from
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide hydrochloride;
2,6-Difluoro-N-[3-(2-(1-methylethyl)-5-{2-[(5-methyl-2-(methyloxy)-4-{-[2-(methylsulfonyl)ethyl]-1-piperazinyl}phenyl)amino]-4-pyrimidinyl}-1,3-thiazol-4-yl)phenyl]benzenesulfonamide dihydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide 4-methylbenzenesulfonate;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide methanesulfonate hydrate; and
Mono sodium salt of 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide.

In an eleventh aspect of the present invention, there is provided a pharmaceutical composition comprising a compound of formula (I) (including any particular sub-generic formula described herein) or a pharmaceutically acceptable salt thereof. In one embodiment, the pharmaceutical composition further comprises one or more of pharmaceutically acceptable carriers, diluents or excipients.

In another aspect of the present invention, there is provided a method of treating a susceptible neoplasm in a mammal in need thereof, comprising administering to the mammal a therapeutically effective amount of a compound of formula (I) (including any particular sub-generic formula described herein) or a pharmaceutically acceptable salt thereof. Susceptible neoplasms include Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer.

In another aspect of the present invention, there is provided a method of treating breast cancer, colorectal cancer, melanoma, non-small cell lung cancer, ovarian cancer, or thyroid cancer, in a mammal in need thereof, comprising administering to the mammal a therapeutically effective amount of a compound of formula (I) or a pharmaceutically acceptable salt thereof.

In another aspect of the present invention, there is provided a method of treating a susceptible neoplasm in a mammal in need thereof, comprising administering to the mammal a therapeutically effective amount of a compound selected from
2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide;
   and pharmaceutically acceptable salts thereof.

In another aspect of the present invention, there is provided a method of treating breast cancer, colorectal cancer, melanoma, non-small cell lung cancer, ovarian cancer, or thyroid cancer, in a mammal in need thereof, comprising administering to the mammal a therapeutically effective amount of a compound selected from
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide hydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide dihydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide 4-methylbenzenesulfonate;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide methanesulfonate hydrate; and
Mono sodium salt of 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide.

In another aspect of the present invention, there is provided a process for preparing a compound of formula (I) or a pharmaceutically acceptable salt thereof. The process comprises reacting a (VIII¹):

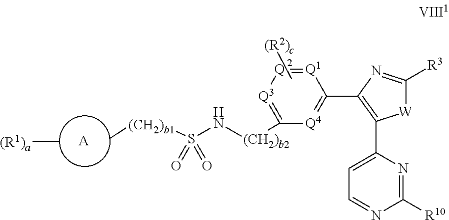

wherein R¹⁰ is halo or thiomethyl;
   with an aniline of formula (IX):

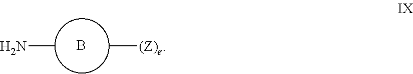

In another aspect of the present invention, there is provided a process for preparing a compound of formula (I), wherein b1 and b2 are 0, or a pharmaceutically acceptable salt thereof. The process comprises reacting a compound of formula (XIV):

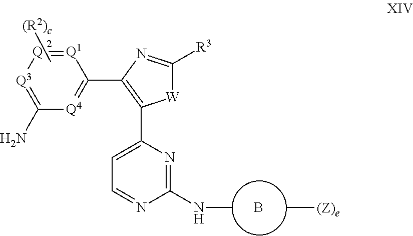

with a compound of formula (VII):

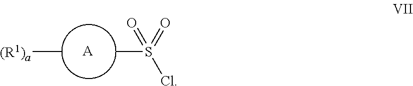

In another aspect, the present invention provides a process for preparing a compound of formula (I) or a pharmaceutically acceptable salt thereof. The process comprising reacting a compound of formula (XXXI):

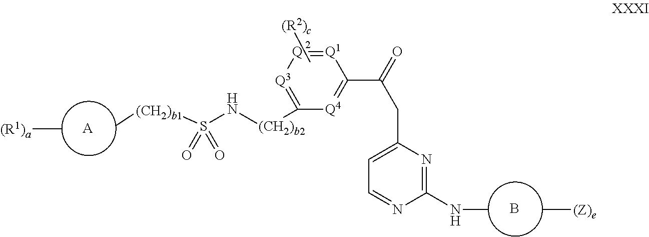

with a suitable brominating agent followed by reaction with one of:
i) a thiourea,
ii) a formamide,
iii) an amide,
iv) a thioamide, or
v) a urea.

In another aspect of the present invention, there is provided a compound of formula (I), (including any particular sub-generic formula described herein) or a pharmaceutically acceptable salt thereof for use in therapy.

In another aspect, there is provided a compound of formula (I) (including any particular sub-generic formula described herein) or a pharmaceutically acceptable salt thereof for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In another aspect, there is provided a compound of formula (I) (including any particular sub-generic formula described herein) or a pharmaceutically acceptable salt thereof for use in the treatment of breast cancer, colorectal cancer, melanoma, non-small cell lung cancer, ovarian cancer, or thyroid cancer in a mammal (e.g., human) in need thereof.

In another aspect, there is provided a compound selected from
2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide;
   and pharmaceutically acceptable salts thereof;
   for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In another aspect, there is provided a compound selected from
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide hydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide dihydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide 4-methylbenzenesulfonate;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide methanesulfonate hydrate; and
Mono sodium salt of 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
   for use in the treatment of breast cancer, colorectal cancer, melanoma, non-small cell lung cancer, ovarian cancer, or thyroid cancer in a mammal (e.g., human) in need thereof.

In a another aspect of the present invention, there is provided the use of a compound of formula (I) (including any particular sub-generic formula described herein) or a pharmaceutically acceptable salt thereof, in the preparation of a medicament for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In a another aspect of the present invention, there is provided the use of a compound of formula (I) (including any particular sub-generic formula described herein) or a pharmaceutically acceptable salt thereof, in the preparation of a medicament for use in the treatment of breast cancer, colorectal cancer, melanoma, non-small cell lung cancer, ovarian cancer, or thyroid cancer in a mammal (e.g., human) in need thereof.

In a another aspect of the present invention, there is provided the use of a compound selected from
2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide;
   and pharmaceutically acceptable salts thereof;
   for the preparation of a medicament for the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In a another aspect of the present invention, there is provided the use of a compound selected from
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide hydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide dihydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide 4-methylbenzenesulfonate;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide methanesulfonate hydrate; and
Mono sodium salt of 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
   for the preparation of a medicament for the treatment of breast cancer, colorectal cancer, melanoma, non-small cell lung cancer, ovarian cancer, or thyroid cancer in a mammal (e.g., human) in need thereof.

In another aspect of the present invention, there is provided a pharmaceutical composition comprising a compound of formula (I) (including any particular sub-generic formula described herein) or a pharmaceutically acceptable salt thereof for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In another aspect of the present invention, there is provided a pharmaceutical composition comprising a compound of formula (I) (including any particular sub-generic formula described herein) or a pharmaceutically acceptable salt thereof for use in the treatment of breast cancer, colorectal cancer, melanoma, non-small cell lung cancer, ovarian cancer, or thyroid cancer in a mammal (e.g., human) in need thereof.

In another aspect of the present invention, there is provided a pharmaceutical composition comprising a compound selected from
2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide;
   and pharmaceutically acceptable salts thereof;
   for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In another aspect of the present invention, there is provided a pharmaceutical composition comprising a compound selected from
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide hydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide dihydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide 4-methylbenzenesulfonate;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide methanesulfonate hydrate; and
Mono sodium salt of 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
   for use in the treatment of breast cancer, colorectal cancer, melanoma, non-small cell lung cancer, ovarian cancer, or thyroid cancer in a mammal (e.g., human) in need thereof.

These and other aspects of the invention are described further in the Detailed Description and Examples which follow.

### BRIEF DESCRIPTION OF THE DRAWINGS

FIG. 1: TGA analysis of compound of Example 19d, 2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide methanesulfonate hydrate.

### DETAILED DESCRIPTION OF THE INVENTION

As used herein the term “ErbB family kinase” refers to ErbB kinases including EGFR (also known as ErbB1), ErbB2, and ErbB4.

As used herein, the term “Raf family kinase” refers to Raf kinases including A-Raf, B-Raf and c-Raf (also known as Raf-1).

As used herein, “compound(s) of formula (I)” means any compound having the structural formula (I) as defined by the variable definitions provided, solvates, including hydrates thereof, and amorphous and crystal forms, including one or more polymorphic forms and mixtures thereof. In the case of compounds of formula (I) which possess one or more chiral centers, the compounds may be in the form of a racemic mixture, or one or more isomerically enriched or pure stereoisomers, including enantiomers and diastereomers thereof. In such embodiments, “compound(s) of formula (I)” includes the racemic form as well as the enriched or pure enantiomers and diastereomers. Enantiomerically enriched or pure compounds will be designated using conventional nomenclature, including the designations +, −, R, S, d, I, D and L, according to the predominant isomer present. Where a compound of the invention contains an alkenyl or alkenylene group, cis (E) and trans (Z) isomerism may also occur. In such embodiments, “compound(s) of formula (I)” includes the individual stereoisomers of the compound of the invention, which will be indicated using conventional, cis/trans nomenclature. It should also be understood that compounds of formula (I) may exist in tautomeric forms other than that shown in the formula and alternative tautomeric forms are also included within “compound(s) of formula (I).”

As used herein, “compound(s) of the invention” means a compound of formula (I) (as defined above) in any version, i.e., as the free base or as a pharmaceutically acceptable salt thereof. The compound as any version may be in any form, including amorphous or crystalline forms, specific polymorphic forms, solvates, including hydrates (e.g., mono-, di- and hemi-hydrates), and mixtures of various forms.

Intermediates may also be present as salts. Thus, in reference to intermediates, the phrase “compound(s) of formula (number)” means a compound having that structural formula or a pharmaceutically acceptable salt thereof.

The term “alkyl” as used herein refers to linear or branched hydrocarbon chains having from 1 to 8 carbon atoms, unless a different number of atoms is specified. Examples of “alkyl” as used herein include, but are not limited to, methyl, ethyl, n-propyl, n-butyl, n-pentyl, isobutyl, isopropyl, and tert-butyl. Similarly, the term “alkylene” refers to linear or branched divalent hydrocarbon chains containing from 1 to 8 carbon atoms, unless a different number of atoms is specified. Examples of “alkylene” as used herein include, but are not limited to, methylene, ethylene, propylene, butylene, and isobutylene.

As used herein, the term “alkenyl” refers to linear or branched hydrocarbon chains having from 2 to 8 carbon atoms, unless a different number of atoms is specified, and at least one and up to three carbon-carbon double bonds. Examples of “alkenyl” as used herein include, but are not limited to ethenyl and propenyl.

As used herein, the term “cycloalkyl” refers to a saturated monocyclic carbocyclic ring or a saturated bicyclic carbocyclic ring, including fused and spiro systems, having from 3 to 8 carbon atoms, unless a different number of atoms is specified. In one embodiment, “cycloalkyl” refers to a saturated monocyclic carbocyclic ring having from 3 to 8 carbon atoms, unless a different number is specified. “Cycloalkyl” includes by way of example cyclopropyl, cyclobutyl, cyclopentyl, cyclohexyl, cycloheptyl and cyclooctyl. Preferred cycloalkyl groups include substituted and unsubstituted C₃₋₆cycloalkyl.

The terms “halo” and “halogen” are synonymous and refer to fluoro, chloro, bromo and iodo.

As used herein, “haloalkyl” refers to an alkyl, as defined above, substituted by one or more halogen atoms, fluoro, chloro, bromo or iodo. Where the haloalkyl group has less than 8 carbon atoms, the number of carbon atoms in the group is indicated as, for example, “haloC₁₋₃alkyl”, which indicates that the haloalkyl group has 1, 2 or 3 carbon atoms. Examples of haloalkyl as used herein include, but are not limited to fluoromethyl, difluoromethyl, trifluoromethyl, fluoroethyl, trifluoroethyl and the like.

The term “oxo” as used herein refers to the group ═O attached directly to a carbon atom of a hydrocarbon ring (e.g., cycloalkyl or cycloalkenyl) or a C, N or S of a heterocyclic or heteroaryl ring to result in oxides, N-oxides, sulfones and sulfoxides.

As used herein, the term “aryl” refers to aromatic monocyclic carbocyclic groups, aromatic fused bicyclic carbocyclic groups, and bicyclic fused carbocyclic groups which have both aromatic and non-aromatic rings, each having the specified number of carbon atoms. In all embodiments wherein the compound of formula (I) includes two or more aryl groups, the aryl groups may be the same or different and are independently selected. Examples of particular aryl groups include but are not limited to phenyl, indenyl and naphthyl.

As used herein, the terms “heterocycle” and “heterocyclic” are synonymous and refer to monocyclic saturated or unsaturated non-aromatic groups, and fused bicyclic saturated or unsaturated non-aromatic groups, each having from 4 to 10 members (unless a different number of members is specified), and spiro systems having from 7 to 12 members. The monocyclic, bicyclic and spiro systems, include 1, 2, or 3 heteroatoms selected from N, O and S, unless a different number of heteroatoms is specified. In one embodiment, “heterocycle” and “heterocyclic” refer to monocyclic saturated or unsaturated non-aromatic groups and fused bicyclic saturated or unsaturated non-aromatic groups, each having from 4 to 10 members (unless a different number of members is specified) including 1, 2, or 3 heteroatoms selected from N, O and S, unless a different number of heteroatoms is specified. In embodiments wherein the heterocycle has 6 or fewer members, it should be clear that such embodiments do not include 7-12 membered spiro systems. In all embodiments wherein the heterocycle includes 2 or more heteroatoms, the heteroatoms may be the same or different and are independently selected from N, O and S. In all embodiments wherein the compound of formula (I) includes two or more heterocyclic groups, the heterocyclic groups may be the same or different and are independently selected. Examples of particular heterocyclic groups include but are not limited to tetrahydrofuran, dihydropyran, tetrahydropyran, pyran, thietane, 1,4-dioxane, 1,3-dioxane, 1,3-dioxolane, piperidine, piperazine, pyrrolidine, morpholine, thiomorpholine, thiazolidine, oxazolidine, tetrahydrothiopyran, tetrahydrothiophene and the like.

As used herein, the term “N-heterocycle” refers to monocyclic saturated or unsaturated non-aromatic groups, fused bicyclic saturated or unsaturated non-aromatic groups, each having from 4 to 10 members (unless a different number of members is specified), and spiro systems having from 7 to 12 members. The monocyclic, bicyclic and spiro systems, include at least one N and optionally 1 or 2 additional heteroatoms selected from N, O and S, unless a different number of additional heteroatoms is specified. In one embodiment, “N-heterocycle” refers to monocyclic saturated or unsaturated non-aromatic groups and fused bicyclic saturated or unsaturated non-aromatic groups, each having from 4 to 10 members (unless a different number of members is specified) including at least one N and optionally 1 or 2 additional heteroatoms selected from N, O and S, unless a different number of additional heteroatoms is specified. In embodiments wherein the N-heterocycle has 6 or fewer members, it should be clear that such embodiments do not include 7-12 membered spiro systems. By “additional heteroatoms” is meant 1 or 2 heteroatoms in addition to the N already specified in the N-heterocycle ring. In all embodiments wherein the heterocycle includes 1 or more additional heteroatoms, the heteroatoms may be the same or different and are independently selected from N, O and S. In all embodiments wherein the compound of formula (I) includes two or more N-heterocyclic groups, the N-heterocyclic groups may be the same or different and are independently selected. Examples of N-heterocycles include piperidine, piperazine, pyrrolidine, morpholine, thiomorpholine and the like.

As used herein, the term “heteroaryl” refers to aromatic, monocyclic groups, aromatic fused bicyclic groups and fused bicyclic groups which have both aromatic and non-aromatic rings, each having from 5 to 10 members (unless a different number of members is specified) including 1, 2, 3, or 4 (particularly 1, 2 or 3) heteroatoms selected from N, O and S, unless a different number of heteroatoms is specified. In all embodiments wherein the heteroaryl includes 2 or more heteroatoms, the heteroatoms may be the same or different and are independently selected from N, O and S. In all embodiments wherein the compound of formula (I) includes two or more heteroaryl groups, the heteroaryl groups may be the same or different and are independently selected. Examples of particular heteroaryl groups include but are not limited to furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, triazine, quinoline, tetrahydroquinoline, isoquinoline, tetrahydroisoquinoline, benzofuran, benzothiophene, indole, indoline, indazole, benzodioxane, benzodioxin, benzodithiane, benzoxazine, benzopiperidine and benzopiperazine.

As used herein, the term “N-heteroaryl” refers to aromatic, monocyclic groups, aromatic fused bicyclic groups and fused bicyclic groups which have both aromatic and non-aromatic rings, each having from 5 to 10 members (unless a different number of members is specified) including at least one N and optionally 1, 2 or 3 (particularly 1 or 2) additional heteroatoms selected from N, O and S, unless a different number of heteroatoms is specified. By “additional heteroatoms” is meant 1, 2 or 3 heteroatoms in addition to the N already specified in the N-heteroaryl ring. In all embodiments wherein the heteroaryl includes 1 or more additional heteroatoms, the heteroatoms may be the same or different and are independently selected from N, O and S. In all embodiments wherein the compound of formula (I) includes two or more N-heteroaryl groups, the N-heteroaryl groups may be the same or different and are independently selected. Examples of N-heteroaryls include pyrrole, imidazole, pyrazole, thiazole, isoxazole, pyridine, pyridazine, pyrazine, pyrimidine, triazine, quinoline, isoquinoline, indole, indoline, benzopiperidine and benzopiperzine.

As used herein, the term “members” (and variants thereof e.g., “membered”) in the context of heterocyclic and heteroaryl groups refers to the total number of ring atoms, including carbon and heteroatoms N, O and/or S. Thus, an example of a 6-membered heterocyclic ring is piperidine and an example of a 6-membered heteroaryl ring is pyridine.

As used herein, the term “optionally substituted” means unsubstituted groups or rings (e.g., cycloalkyl, heterocycle, and heteroaryl rings) and rings substituted with one or more specified substituents.

The present invention provides compounds of formula (I):

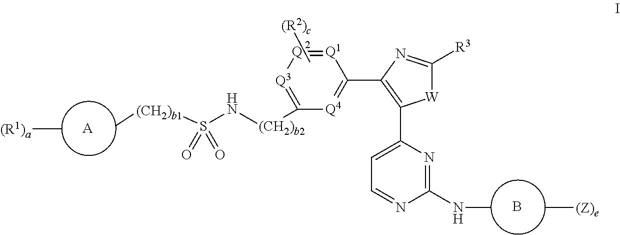

wherein:
a is 0, 1, 2 or 3;
each R¹ is the same or different and is independently selected from halo, alkyl, haloalkyl, —OR⁶, —CO₂H, —CO₂R⁶, and —CN;
Ring A is C₃₋₆cycloalkyl, phenyl, and 5-6 membered heterocycle or heteroaryl having 1 or 2 heteroatoms selected from N, O and S;
one of b1 and b2 is 0 and the other is 0 or 1;
c is 0, 1 or 2;
each R² is the same or different and is independently selected from halo, alkyl, haloalkyl, —OR⁶, and —CN;
each of Q¹, Q², Q³, and Q⁴ is CH or C—R² or one of Q¹, Q², Q³, and Q⁴ is N and the others are CH or C—R²;
W is —O— or —S—;
R³ is selected from H, alkyl, haloalkyl, alkenyl, C₃₋₆cycloalkyl, phenyl, Het, —CH₂-Het, —NR⁶R⁷, —N(R⁶)—C₃₋₆cycloalkyl, —N(R⁶)Het, —N(R⁶)R⁵—Het, —N(R⁶)—R⁵—OR⁷, —N(R⁶)—R⁵—NR⁶R⁷, —N(H)C(O)R⁶, —N(R⁶)—C(O)—NR⁶R⁷, —N(H)SO₂R⁶, —N(R⁶)—R⁵—S(O)₂R⁷, and —N(R⁶)—S(O)₂—NR⁶R⁷,
   wherein each of said cycloalkyl is optionally substituted with 1 or 2 substituents which are the same or different and are independently selected from halo, C₁₋₃alkyl, haloC_{f-3}alkyl, OH, O—C₁₋₃alkyl, oxo, S(C₁₋₃alkyl), SO₂, NH₂,
      N(H)C₁₋₃alkyl and N(C₁₋₃alkyl)₂, and
   wherein said phenyl is optionally substituted with 1, 2 or 3 substituents which are the same or different and are each independently selected from halo,
      C₁₋₃alkyl, haloC₁₋₃alkyl, O—C₁₋₃alkyl, C₁₋₃alkylene-O—C₁₋₃alkyl, OH, C₁₋₃alkylene-OH, NH₂, N(H)C₁₋₃alkyl, N(C₁₋₃alkyl)₂, CN and NO₂;
   each Het is the same or different and is independently a 4-6 membered heterocycle having 1 or 2 heteroatoms selected from N, O and S and optionally substituted with 1 or 2 substituents which are the same or different and are each independently selected from halo,
      C₁₋₃alkyl, haloC₁₋₃alkyl, O—C₁₋₃alkyl, C₁₋₃alkylene-O—C₁₋₃alkyl, OH, C₁₋₃alkylene-OH, oxo, SO₂(C₁₋₃alkyl), C₁₋₃alkylene-SO₂(C₁₋₃alkyl), NH₂, N(H)C₁₋₃alkyl, N(C₁₋₃alkyl)₂, CN, and —CH₂CN;
each R⁵ is the same or different and is independently C₁₋₄alkylene;
Ring B is selected from phenyl, 9-10 membered aryl, 5-6 membered heteroaryl and 9-10 membered heteroaryl, each heteroaryl having 1, 2, or 3 heteroatoms selected from N, O and S;
   wherein when Ring B is selected from phenyl and 5-6 membered heteroaryl, then
   e is 0, 1, 2 or 3; and
   each Z is the same or different and is independently selected from:
      halo, alkyl, haloalkyl, alkenyl,
      Het², —R⁵Het², Het³-Het²,
      oxo, —OR⁶, —R⁵—OR⁶, —O—R⁵—OR⁶, —OHet², —O—R⁵—Het², —O—R⁵—NR⁶R⁷,
      —O—R⁵—S(O)₂R⁶, —C(O)R⁶, —C(O)CH₂NR⁶R⁷, —CO₂R⁶, —R⁵—CO₂R⁶, —S(O)_{f} R⁶,
      —R⁵—S(O)₂R⁶, —S(O)₂NR⁶R⁷, —R⁵—S(O)₂NR⁶R⁷, —S(O)₂—R⁵—NR⁶R⁷, —NR⁶R⁷, —R⁵—NR⁶R⁷, —N(R⁶)Het², —N(R⁶)—R⁵—OR⁷, —N(R⁶)—R⁵—S(O)_{f}R⁷, —N(R⁶)—R⁵—CN, —N(R⁶)—R⁵—NR⁶R⁷, —N(H)S(O)₂R⁶, —N(R⁶)—C(O)—NR⁶R⁷, —N(R⁶)—S(O)₂—NR⁶R⁷, —CN, and —R⁵—CN; and
   wherein when Ring B is selected from 9-10 membered aryl and 9-10 membered heteroaryl, then:
   e is 0, 1 or 2; and
   each Z is the same or different and is independently selected from: halo, alkyl, oxo, —C(O)R⁶, —C(O)CH₂NR⁶R⁷, —OR⁶, and —NR⁶R⁷;
   each Het² is the same or different and is independently a heterocycle optionally substituted with 1 or 2 substituents which are the same or different and are each independently selected from halo, C₁₋₃alkyl, haloC₁₋₃alkyl,
      O—C₁₋₃alkyl, C₁₋₃alkylene-O—C₁₋₃alkyl, OH, C₁₋₃alkylene-OH, oxo, C(O)(C₁₋₃alkyl), SO₂(C₁₋₃alkyl), C₁₋₃alkylene-SO₂(C₁₋₃alkyl), NH₂, N(H)C₁₋₃alkyl, N(C₁₋₃alkyl)₂, CN and C₁₋₃alkylene-CN;
   Het³ is a 5-6 membered heterocycle having 1 or 2 heteroatoms selected from N, O and S and optionally substituted with 1 or 2 substituents which are the same or different and are each independently selected from halo,
      C₁₋₃alkyl, and O—C₁₋₃alkyl;
   f is 0, 1 or 2; and
      each R⁶ and each R⁷ is the same or different and is independently H, alkyl or haloalkyl;
      and pharmaceutically acceptable salts thereof.

The compounds of the invention are described in the conventional manner employing variables to represent a number of possible substituents or groups. The original, particular and preferred definitions of variables described herein apply equally to compounds of formula (I) and compounds of the invention. For brevity, the following description will generally refer to “compounds of the invention” rather than to both, as compounds of the invention encompasses all compounds of formula (I). It should be understood that the definition of variables utilized to describe the compounds of the invention will be selected in light of the knowledge possessed by the ordinarily skilled organic chemist such that embodiments which such chemist would consider to be obviously inoperative or unstable are avoided. For example, the organic chemist of ordinary skill in the art would appreciate that moieties such as —N(H)CH₂F, —N(H)CH₂NH₂, —OCH₂NH₂, and the like, result in potentially unstable acetals, aminals or iminium ions. As such, the present invention should be understood such that the variables are defined in a manner which avoids such embodiments.

For ease of reference, portions of formula (I) may sometimes be referred to herein as a “head” portion or a “tail” portion. The “head” and “tail” portions are indicated in the following illustration by the oval and the box, respectively.

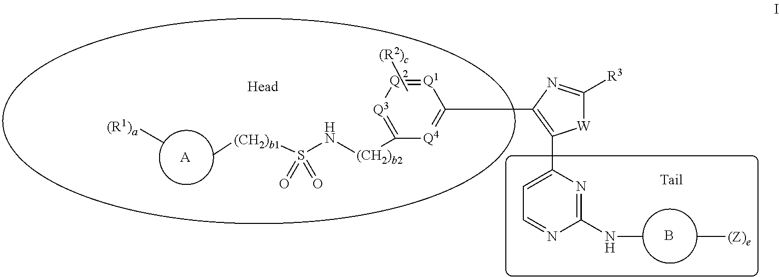

Referring first to the head portion of the compounds of the invention, a particular embodiment is defined wherein a is 0, 1 or 2. In another particular embodiment, a is 1 or 2. In those embodiments wherein Ring A is phenyl, particular embodiments are defined wherein a is 1 or 2, more particularly 2. In embodiments wherein Ring A is 5-6 membered heterocycle or heteroaryl, particular embodiments are defined wherein a is 0 or 1, more particularly 0. In those embodiments wherein Ring A is cycloalkyl, particular embodiments are defined wherein a is 0.

In those embodiments of the compounds of the invention wherein a is 1, 2 or 3, each R¹ may be bound to Ring A through any suitable carbon or heteroatom of Ring A (to provide, for example, N-methyl or N-oxides). In certain embodiments, wherein a is any of 1, 2 or 3, each R¹ is the same or different and is independently selected from halo (particularly F or Cl), alkyl, haloalkyl, and —OR⁶ or any subset thereof. In those embodiments wherein R¹ is —OR⁶, where R⁶ is H, it will be understood that when Ring A is a heterocycle or heteroaryl, the compounds of the invention include the tautomeric form wherein the heterocycle or heteroaryl Ring A is substituted by oxo. Specific examples of groups defining R¹ include but are not limited to F, Cl, Br, CH₃, CF₃, CH₂CH₃, CH(CH₃)₂, OCH₃, OCF₃, OCH₂CH₃, and CN. In one example of a preferred embodiment, each R¹ is the same or different and is independently selected from F, Cl, CH₃, CF₃, OCH₃, and OCF₃ or any subset thereof. In one preferred embodiment, each R¹ is F.

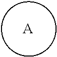

in formula (I) is referred to herein as “Ring A.” Ring A is selected from cycloalkyl, phenyl, 5-6 membered heterocycle and 5-6 membered heteroaryl said heterocycle or heteroaryl having 1 or 2 heteroatoms selected from N, O and S. Ring A may be bonded to the methylene (when b1 is 1) or the sulfonyl through any suitable carbon or heteroatom of Ring A. In one embodiment, Ring A is selected from cycloalkyl, phenyl, and 5-6 heteroaryl having 1 or 2 heteroatoms selected from N, O and S. In one particular embodiment, Ring A is selected from C₃₋₆cycloalkyl, phenyl, and 5-6 membered heteroaryl having 1 or 2 heteroatoms selected from N, O and S. In one embodiment, Ring A is selected from phenyl and 5-6 membered heteroaryl having 1 or 2 heteroatoms selected from N, O and S. The 5-6 membered heteroaryl may be a 5-6 membered N-heteroaryl optionally having 1 additional heteroatom selected from N, O and S. In one preferred embodiment, Ring A is phenyl. In another particular embodiment, Ring A is a 5-6 membered N-heteroaryl optionally having 1 additional heteroatom selected from N, O and S. In one particular embodiment, Ring A is furan.

Specific examples of groups defining Ring A include but are not limited to cyclopropyl, cyclobutyl, cyclopentyl, cyclohexyl, furanyl, tetrahydrofuranyl, thiophenyl, pyrrolyl, pyrrolinyl, pyrrolidinyl, oxazolyl, isoxazolyl, thiazolyl, isothiazolyl, imidazolyl, imidazolidinyl, pyrazolyl, pyrazolinyl, pyrazolidinyl, phenyl, pyranyl, tetrahydropyranyl, pyridinyl, piperidinyl, isoxanyl, morpholinyl, thiomorpholinyl, pyridazinyl, pyrimidinyl, pyrazinyl, and piperazinyl, or any subset thereof. In one embodiment, Ring A is selected from cyclohexyl, furanyl, thiophenyl, pyrrolyl, oxazolyl, isoxazolyl, thiazolyl, isothiazolyl, imidazolyl, pyrazolyl, phenyl, pyridinyl, pyrimidinyl, and pyridazinyl, or any subset thereof. More particularly, Ring A is selected from phenyl, pyridinyl, furanyl, imidazolyl and thiazolyl. In one example of a preferred embodiment, Ring A is phenyl. In another particular embodiment, Ring A is pyridinyl. In one particular embodiment, Ring A is furanyl.

In the compounds of the invention, one of b1 and b2 is 0 and the other is 0 or 1. When b1 is 0, the sulfonyl is bound directly to Ring A. Similarly, when b2 is 0, the amine is bound directly to the ring defined as:

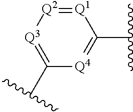

In one particular embodiment, b1 is 0. In one particular embodiment b2 is 0. In one preferred embodiment both b1 and b2 are 0. This embodiment of the compounds of formula (I) is illustrated by formula (I-i)

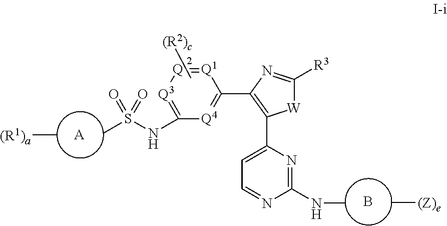

wherein all variables are as defined herein.

The ring defined as

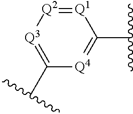

is a phenyl or pyridinyl ring wherein when the ring is a pyridinyl, the N of the pyridinyl ring may be at any of positions indicated by Q¹, Q², Q³, and Q⁴. In one preferred embodiment, each of Q¹, Q², Q³, and Q⁴ is CH or C—R² and thus the ring is a phenyl ring or substituted phenyl ring. It is to be understood the definition of Q¹, Q², Q³, and Q⁴ is consistent with the definition of c and thus not more than two of Q¹, Q², Q³, and Q⁴ is C—R². In another embodiment, at least one of Q¹, Q², Q³, and Q⁴ is N. In one particular embodiment, Q³ is N, and Q¹, Q², and Q⁴ are CH or
C—R², more particularly CH. In another particular embodiment, Q⁴ is N, and Q¹, Q², and Q³ are CH or C—R², more particularly CH. Specific embodiments of the compounds of formula (I) are illustrated by formulas (I-i-a) and (I-i-b):

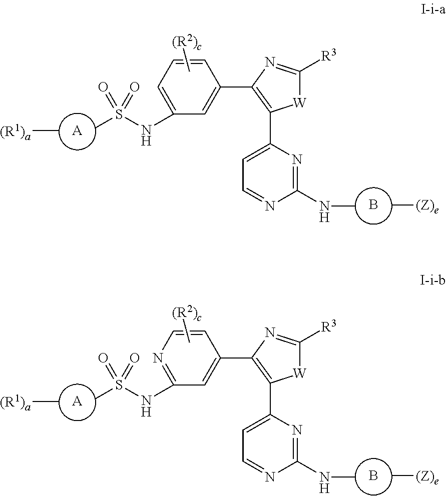

wherein all variables are as defined herein.

The optional substituents on the phenyl or pyridinyl ring are indicated by —(R²)_{c}. The phenyl or pyridinyl ring may have up to two substituents defined by R². In one particular embodiment, c is 0 or 1. In those embodiments wherein at least one of Q¹, Q², Q³, and Q⁴ is N, c is typically 0. This embodiment of the invention is illustrated by formula (I-i-b1):

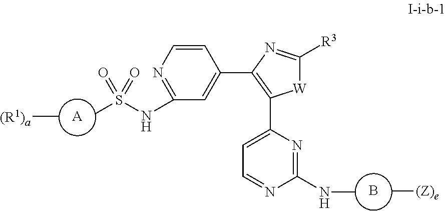

wherein all variables are as defined herein.

In those embodiments wherein each of Q¹, Q², Q³, and Q⁴ is CH or C—R², c is typically 0 or 1. In one preferred embodiment, c is 0 and thus each of Q¹, Q², Q³, and Q⁴ is CH. This embodiment is illustrated by formula (I-i-a1):

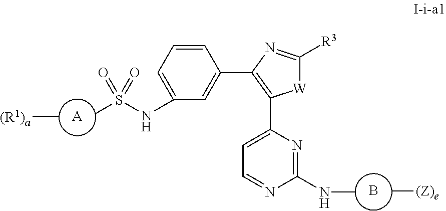

wherein all variables are as defined herein.

In one preferred embodiment, c is 0.

In certain embodiments wherein c is 1 or 2, each R² is the same or different and is independently selected from halo, alkyl, —OR⁶, and —CN, or any subset thereof. In one particular embodiment, when c is 1 or 2, each R² is the same or different and is independently selected from halo and alkyl; more preferably halo, particularly F or Cl. In one example of a preferred embodiment, each R² is the same or different and is independently selected from halo (particularly F or Cl), alkyl (particularly methyl), and —O-alkyl (particularly —OCH₃), or any subset thereof. In one preferred embodiment, R² is F; more preferably, c is 1 and R² is F.

In one embodiment, compounds of the invention are defined wherein W is O. In one preferred embodiment the compounds of the invention are defined wherein W is S. In one particular preferred embodiment, W is S, c is 0 and each of Q¹, Q², Q³, and Q⁴ is CH. This embodiment is illustrated by formula (I-i-as):

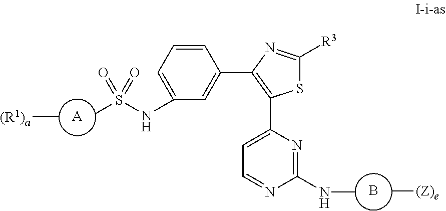

wherein all variables are as defined herein.

In one embodiment, R³ is selected from H, alkyl, haloalkyl, Het, —NR⁶R⁷, —N(R⁶)—C₃₋₆cycloalkyl, —N(R⁶)Het, —N(R⁶)R⁵—Het, —N(R⁶)—R⁵—OR⁷, —N(R⁶)—R⁵NR⁶R⁷, —N(H)C(O)R⁶, —N(H)SO₂R⁶, and —N(R⁶)—R⁵—S(O)₂R⁷, or any subset thereof. In one particular embodiment, R³ is selected from alkyl, haloalkyl, Het, and —NR⁶R⁷, or any subset thereof. In one particular embodiment, R³ is Het. In one particular embodiment, R³ is selected from alkyl, haloalkyl, optionally substituted pyranyl, optionally substituted 4-6 membered N-heterocycle, and —NR⁶R⁷, or any subset thereof. One more particular embodiment is defined wherein R³ is selected from alkyl, optionally substituted pyranyl, optionally substituted 5-6 membered N-heterocycle (wherein the optional substituents are defined in “Het”), and —NR⁶R⁷, or any subset thereof. In one preferred embodiment, R³ is alkyl or —NR⁶R⁷. In another example of a preferred embodiment, R³ is alkyl, and particularly branched alkyl (particularly methyl, ethyl, isopropyl, or tert-butyl, or any subset thereof). In one specific preferred embodiment, R³ is isopropyl. In another specific embodiment, R₃ is pyranyl (e.g., tetrahydropyranyl) or pyrazolyl.

Het (as employed in the definition of R³ and in groups defining R³, e.g., —R⁵let, —N(R⁶)Het, etc.) refers to a 4-6 membered heterocycle having 1 or 2 heteroatoms selected from N, O and S and optionally substituted with 1 or 2 substituents which are the same or different and are each independently selected from halo, C₁₋₃alkyl, haloC₁₋₃alkyl, O—C₁₋₃alkyl, C₁₋₃alkylene-O—C₁₋₃alkyl, OH, C₁₋₃alkylene-OH, oxo, SO₂(C₁₋₃alkyl), C₁₋₃alkylene-SO₂(C₁₋₃alkyl), NH₂, N(H)C₁₋₃alkyl, N(C₁₋₃alkyl)₂, CN, and CH₂CN or any subset thereof. In one embodiment, Het in the definition of R³ and in groups defining R³ is a 4-6 membered N-heterocycle optionally having 1 additional heteroatom selected from N, O and S and optionally substituted as described above. In one particular embodiment, Het is a 4-6 membered N-heterocycle having no additional heteroatoms and optionally 1 substituent as described above. In one embodiment, Het is selected from optionally substituted morpholinyl, pyrrolidinyl, piperidinyl, azetidinyl, piperizinyl, thiomorpholinyl, or any subset thereof, wherein the optional substituents are as recited above. In one particular embodiment, Het in the definition of R³ and in groups defining R³, is pyrrolidinyl or morpholinyl. In one particular embodiment, R³ is pyrrolidinyl.

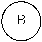

in formula (I) is referred to herein as “Ring B.” Ring B is selected from phenyl, 9-10 membered aryl, 5-6 membered heteroaryl and 9-10 membered heteroaryl, each heteroaryl having 1, 2 or 3 heteroatoms selected from N, O and S. Ring B may be bonded to —N(H)— through any suitable carbon or heteroatom of Ring B. Specific groups defining Ring B include but are not limited to furanyl, thiophenyl, pyrrolyl, oxazolyl, isoxazolyl, thiazolyl, isothiazolyl, imidazolyl, pyrazolyl, triazolyl, phenyl, pyridinyl, pyridazinyl, pyrimidinyl, pyrazinyl, naphthyl, dihydro-naphthyl, indenyl, dihydroindenyl, benzofuranyl, benzothiophenyl, indolyl, isoindolyl, indolinyl, indazolyl, benzimidazolyl, quinolinyl, isoquinolinyl, tetrahydroisoquinolinyl, quinazolinyl, quinoxalinyl, benzopiperidinyl, benzopiperazinyl, benzomorpholinyl, benzotriazolyl, benzopyranyl, chroman, isochroman, benzodioxanyl, and benzodioxolanyl, or any subset thereof.

In one embodiment, Ring B is phenyl or 5-6 membered heteroaryl having 1, 2 or 3 heteroatoms selected from N, O and S, more particularly 1 or 2 heteroatoms selected from N, O and S.

In one example of a preferred embodiment, Ring B is phenyl.

In another embodiment, Ring B is a 5-6 membered heteroaryl having 1 or 2 heteroatoms selected from N, O and S. In one preferred embodiment, Ring B is a 5-6 membered N-heteroaryl optionally having 1 additional heteroatom which is selected from N, O and S. Specific examples wherein Ring B is a 5-6 membered heteroaryl include furanyl, pyrrolyl, pyrazolyl, thiophenyl, isoxazolyl, pyridinyl, pyrimidinyl and pyrazinyl. In one specific example of a preferred embodiment, Ring B is pyridinyl.

In another embodiment, Ring B is 9-10 membered aryl, particularly indenyl or naphthyl.

In another embodiment, Ring B is a 9-10 membered bicyclic fused heteroaryl having 1, 2 or 3 heteroatoms selected from N, O and S, more particularly 1 or 2 heteroatoms selected from N, O and S. In one particular embodiment, Ring B is a 9-10 membered bicyclic fused N-heteroaryl optionally having 1 additional heteroatom which is selected from N, O and S. Specific examples wherein Ring B is a 9-10 membered bicyclic fused heteroaryl include benzopiperidinyl, benzomorpholinyl, benzofuranyl and benzodioxanyl. In one specific example of a particular embodiment, Ring B is benzopiperidinyl or benzomorpholinyl.

The substituents Z may be bound to Ring B through any available carbon or heteroatom of Ring B. However, the moiety Z should be understood to be defined in view of the definition of Ring B so as to avoid embodiments which the organic chemist of ordinary skill would consider to be obviously unstable or inoperative. For example, in those embodiments wherein Ring B is a heteroaryl, e is not 0, and Z is bound to a heteroatom of Ring B; then Z is defined as a moiety bound to Ring B through either carbon or a heteroatom suitable for binding to the heteroatom of Ring B. Thus for example, when Ring B is a heteroaryl and Z is bound to a N of Ring B, then Z is defined as a moiety capable of binding to the N of Ring B; accordingly in such embodiment, Z may not for example, be halo. Z moieties suitable for binding to a N of Ring B will be apparent to those skilled in the art and include but are not limited to H (where e is 0), alkyl (e.g., N-methyl) and oxo (e.g., N-oxide).

When Ring B is selected from phenyl and 5-6 membered heteroaryl, e is 0, 1, 2 or 3. When e is 0, Ring B is unsubstituted. In one embodiment wherein Ring B is phenyl or 5-6 membered heteroaryl, e is 1, 2 or 3. In the embodiment, wherein Ring B is phenyl, e is preferably 1, 2 or 3, more particularly, 2 or 3. In embodiments wherein Ring B is a 5-6 membered heteroaryl such as pyridinyl, e is particularly 1 or 2.

In the embodiments wherein Ring B is phenyl or 5-6 membered heteroaryl and e is 1, 2 or 3, each Z is the same or different and is independently selected from:
halo, alkyl, haloalkyl, alkenyl,
Het², —R⁵Het², Het³—Het²,
oxo, —OR⁶, —R⁵—OR⁶, —O—R⁵—OR⁶, —OHet², —O—R⁵—Het², —O—R⁵—NR⁶R⁷, —O—R⁵—S(O)₂R⁶, —C(O)R⁶, —C(O)CH₂NR⁶R⁷, —CO₂R⁶, —R⁵—CO₂R⁶, —S(O)_{f}R⁶ (e.g., SO₂R⁶), —R⁵—S(O)₂R⁶, —S(O)₂NR⁶R⁷, —R⁵—S(O)₂NR⁶R⁷, —S(O)₂—R⁵—NR⁶R⁷, —NR⁶R⁷, —R⁵—NR⁶R⁷, —N(R⁶)Het², —N(R⁶)—R⁵—OR⁷, —N(R⁶)—R⁵—S(O)_{f}R⁷, —N(R⁶)—R⁵—CN, —N(R⁶)—R⁵—NR⁶R⁷, —N(H)S(O)₂R⁶, —N(R⁶)—C(O)—NR⁶R⁷, —N(R⁶)—S(O)₂—NR⁶R⁷, —CN, and —R⁵—CN, or any subset thereof.

Het² and Het³ in this particular embodiment may each be optionally substituted. The following particular and preferred definitions of Z are applicable to those embodiments wherein Ring B is phenyl or 5-6 membered heteroaryl and e is 1, 2 or 3. In one embodiment, each Z is the same or different and is independently selected from:
halo, alkyl, haloalkyl, alkenyl,
Het², —R⁵Het², Het³-Het²,
oxo, —OR⁶, —R⁵—OR⁶, —O—R⁵—OR⁶, —OHet², —O—R⁵—Het², —O—R⁵—NR⁶R⁷,
—O—R⁵—S(O)₂R⁶, —CO₂R⁶, —R⁵—CO₂R⁶, —S(O)_{f}R⁶, —R⁵—S(O)₂R⁶, —S(O)₂NR⁶R⁷,
—R⁵—S(O)₂NR⁶R⁷, —S(O)₂—R⁵—NR⁶R⁷,
—NR⁶R⁷, —R⁵—NR⁶R⁷, —N(R⁶)Het², —N(R⁶)—R⁵—OR⁷, —N(R⁶)—S(O)_{f}R⁷, —N(R⁶)—R⁵—CN, —N(R⁶)—R⁵—NR⁶R⁷, —N(H)S(O)₂R⁶, —N(R⁶)—C(O)—NR⁶R⁷, —N(R⁶)—S(O)₂—NR⁶R⁷, —CN, and —R⁵—CN, or any subset thereof.
   Het² and Het³ in this particular embodiment are each optionally substituted.

In one particular embodiment, each Z is the same or different and is independently selected from:
halo, alkyl, haloalkyl,
Het², —R⁵Het², Het³-Het²,
oxo, —OR⁶, —O—R⁵—OR⁶, —OHet², —O—R⁵-Het², —O—R⁵—NR⁶R⁷,
—S(O)_{f}R⁶ (e.g., SO₂R⁶), —R⁵—S(O)₂R⁶, —R⁵—S(O)₂NR⁶R⁷,
—NR⁶R⁷, —R⁵—NR⁶R⁷, —N(R⁶)—R⁵—NR⁶R⁷, and —CN, or any subset thereof.
   Het² and Het³ in this particular embodiment are each optionally substituted.

In one particular embodiment wherein e is 1, 2 or 3, at least one Z is at the ortho position and is O—R⁶, particularly O-alkyl, more particularly O-methyl. For illustration, the following example shows one Z group bound at the ortho position wherein Ring B is phenyl, e′ is 0, 1 or 2 and the remaining Z group(s) and all other variables are as defined herein.

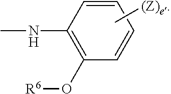

The embodiment may be further illustrated when Ring B is pyridinyl as follows:

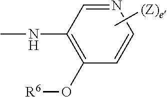

wherein e′ is 0, 1 or 2 and the remaining Z group(s) and all other variables are as defined herein.

In one particular embodiment, each Z is the same or different and is independently selected from halo, alkyl, Het², Het³-Het², oxo, —OR⁶, —OHet², —O—R⁵—Het², —S(O)_{f}R⁶ (e.g., SO₂R⁶), and —O—R⁵—NR⁶R⁷, or any subset thereof. Het² and Het³ in this particular embodiment are each optionally substituted.

In one particular embodiment, e is 1, 2 or 3 and one Z is selected from Het², Het³-Het², and —OHet², or any subset thereof, wherein Het² and Het³ are each optionally substituted. In one particular embodiment, one substituent Z is located in the para position and is selected from Het², Het³-Het², and —OHet², or any subset thereof, wherein Het² and Het³ are each optionally substituted.

In one embodiment wherein Ring B is phenyl, e is 3, one Z is alkyl, one Z is O—R⁶, particularly O-alkyl (e.g., O-methyl) and the third Z is Het². Het² in this embodiment may be optionally substituted. One specific example of this embodiment may be illustrated as follows:

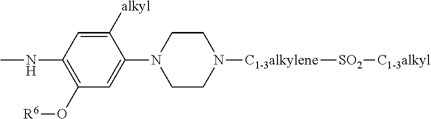

wherein Het² in this example is substituted piperazinyl and all variables are as defined herein.

In one embodiment wherein Ring B is 5-6 membered heteroaryl (e.g., pyridinyl), e is 2, one Z is H or O—R⁶, particularly O-alkyl (e.g., O-methyl) and the other Z is Het². Het² in this embodiment may be optionally substituted. An example of these embodiments may be illustrated as follows:

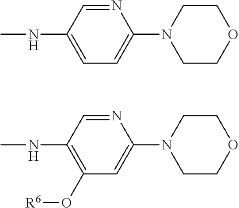

wherein Het² in this example is unsubstituted morpholine and all variables are as defined herein.

In one embodiment wherein Ring B is 5-6 membered heteroaryl (e.g., pyridinyl), e is 1 and Z is selected from Het², —O—R⁵—OR⁶, —OHet², —O—R⁵—Het², —O—R⁵—NR⁶R⁷. Het² in this embodiment is optionally substituted.

When Ring B is a 9-10 membered aryl or heteroaryl, e is 0, 1 or 2 and each Z is the same or different and is independently selected from halo, alkyl, oxo, —C(O)R⁶, —C(O)CH₂NR⁶R⁷, —OR⁶ and —NR⁶R⁷. In one embodiment, wherein Ring B is bicyclic fused 9-10 membered aryl or heteroaryl, e is 0 or 1. In one particular embodiment, e is 0 and hence, Ring B is unsubstituted. In one embodiment, Ring B is a 9-10 membered bicyclic fused aryl or heteroaryl, e is 1 and Z is halo, C₁₋₃alkyl, oxo or —OC₁₋₃alkyl. In one embodiment wherein Ring B is 9-10 membered bicyclic fused aryl or heteroaryl, e is 1 and Z is selected from alkyl and oxo, such as methyl, ethyl, isopropyl, N-methyl, N-ethyl, oxo and N-oxide.

Following is a list of specific examples of groups defining Z. In the following specific examples, R⁵ refers to C₁₋₄alkylene, however in certain examples below, it will be appreciated that R⁵ should be understood to be C₂ alkylene in order to avoid unstable species. Specific examples of groups defining Z include but are not limited to:
halo (e.g., F or Cl);
alkyl (e.g., CH₃),
haloalkyl (e.g., CF₃),
Het² (e.g., 5-6 membered heterocycle or 5-6 membered heteroaryl having 1 or 2 heteroatoms selected from N, O and S and substituted variants thereof)
R⁵—Het² (e.g., alkyl-morpholine, alkyl-piperidine, alkyl-pyrrolidine, and substituted variants thereof),
Het³-Het² (e.g., piperidinyl-piperidine and substituted variants thereof),
oxo (e.g., N-oxide, carbonyl or sulfonyl),
OR⁶ (e.g., OH, OCH₃),
R⁵—OR⁶ (e.g., CH₂CH₂—OCH₃),
O—R⁵—OR⁶ (e.g., O—CH₂CH₂—OCH₃),
OHet² (e.g., O-piperidine, O-pyrrolidine and substituted variants thereof)
O—R⁵—Het² (e.g., O—(CH₂)₂-morpholine, O—(CH₂)₂-pyrrolidine, O—(CH₂)₂-piperidine and substituted variants thereof),
O—R⁵—NR⁶R⁷ (e.g., O—(CH₂)₂—NH₂, O—(CH₂)₂—N(H)CH₃,
   O—(CH₂)₂—N(CH₃)₂, O—(CH₂)₂—N(CH₂CH₃)₂, O—(CH₂)₃—NH₂,
   O—(CH₂)₃—N(H)CH₃, O—(CH₂)₃—N(CH₃)₂, O—(CH₂)₃—N(CH₂CH₃)₂,
   O—CH₂CH(CH₃)—NH₂, O—CH₂CH(CH₃)—N(H)CH₃, O—CH₂CH(CH₃)—N(CH₃)₂,
   O—CH₂CH(CH₃)—N(CH₂CH₃)₂,
S(O)_{f}R⁶, particularly SO₂R⁶ (e.g., SO₂CH₃, SO₂CH₂CH₃, SO₂(CH₂)₂OH),
R⁵—SO₂R⁶(CH₂—SO₂CH₃, CH₂—SO₂CH₂CH₃, (CH₂)₂—SO₂CH₃ and (CH₂)₂—SO₂CH₂CH₃),
SO₂NR⁶R⁷ (e.g., SO₂NH₂, SO₂N(H)CH₃,
R⁵—SO₂NR⁶R⁷ (e.g., (CH₂)₂—SO₂NH₂),
R⁵—NR⁶R⁷ (e.g., —(CH₂)₃—N(CH₃)₂),
N(H)Het² (e.g., N(H)-piperidine, N(H)-piperazine and substituted variants thereof),
N(CH₃)—R⁵—OR⁷ (e.g., N(CH₃)—(CH₂)₃—OCH₃),
N(H)—R⁵—NR⁶R⁷ (e.g., NH—(CH₂)₃—N(CH₃)₂),
N(CH₃)—R⁵—NR⁶R⁷ (e.g., N(CH₃)—(CH₂)₃—N(CH₃)₂),
CN, and R⁵—CN (e.g., (CH₂)₂CN),
or any subset thereof.

In the definition of Z, each Het² is the same or different and is independently a heterocycle optionally substituted with 1 or 2 substituents selected from:
halo, C₁₋₃alkyl, haloC₁₋₃alkyl, O—C₁₋₃alkyl, C₁₋₃alkylene-O—C₁₋₃alkyl, OH,
C₁₋₃alkylene-OH, oxo, C(O)(C₁₋₃alkyl), SO₂(C₁₋₃alkyl), C₁₋₃alkylene-SO₂(C₁₋₃alkyl),
NH₂, N(H)C₁₋₃alkyl, N(C₁₋₃alkyl)₂, CN and C₁₋₃alkylene-CN, or any subset thereof.

Het² includes 4-10 membered heterocycles and spiro systems having from 7 to 12 membered spiro systems, wherein the heterocycles and spirosystems include 1, 2, or 3 heteroatoms selected from N, O and S. In one embodiment, Het² is not a 7-12 membered spiro system. In all embodiments wherein Het² is substituted with 2 substituents, the substituents may be the same or different and are each independently selected from the foregoing list.

In one particular embodiment, Het² is independently a heterocycle having 1 or 2 heteroatoms selected from N, O and S and optionally substituted with 1 or 2 substituents selected from the foregoing. In one more particular embodiment, Het² is a 5-6 membered heterocycle having 1 or 2 heteroatoms selected from N, O and S and optionally substituted with 1 or 2 substituents as defined above. In one particular embodiment, Het² is selected from 5-6 membered N-heterocycles optionally having 1 additional heteroatom selected from N, O and S and optionally substituted with 1 or 2 substituents as defined above.

In certain embodiments of the invention, the group Het² is unsubstituted. In those embodiments wherein Het² is substituted, a particular embodiment is defined wherein the substituent(s) is/are selected from
C₁₋₃alkyl, haloC₁₋₃alkyl, O—C₁₋₃alkyl, C₁₋₃alkylene-OH, oxo, C(O)(C₁₋₃alkyl),
SO₂(C₁₋₃alkyl), C₁₋₃alkylene-SO₂(C₁₋₃alkyl), NH₂, N(H)C₁₋₃alkyl, N(C₁₋₃alkyl)₂, and
C₁₋₃alkylene-CN, or any subset thereof.

In a more particular embodiment, the optional substituent(s) on Het² is/are selected from
C₁₋₃alkyl, haloC₁₋₃alkyl, O—C₁₋₃alkyl, C₁₋₃alkylene-OH, oxo, C(O)(C₁₋₃alkyl), SO₂(C₁₋₃alkyl), C₁₋₃alkylene-SO₂(C₁₋₃alkyl) and C₁₋₃alkylene-CN, or any subset thereof.

Specific examples of groups defining Het² within the definition of Z include but are not limited to:

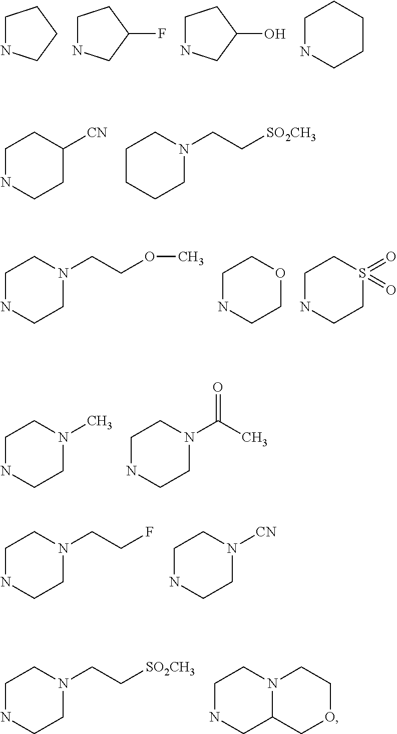

or any subset thereof. The point of attachment to Ring B being variable.

Het³ in the definition of Z is employed in the group Het³-Het². Het³ is a 5-6 membered heterocycle having 1 or 2 heteroatoms selected from N, O and S and optionally substituted with 1 or 2 substituents selected from halo, C₁₋₃alkyl, haloC₁₋₃alkyl and

O—C₁₋₃alkyl. It should be understood that reference to substituents on Het³ refers to optional substituents in addition to Het². In all embodiments wherein Het³ is substituted with 2 substituents, the substituents may be the same or different and are each independently selected from the foregoing list. In one embodiment of the invention, Het³ in the definition of Z is unsubstituted, except by Het².

In one embodiment, the compounds of the invention are defined wherein R⁶ and R⁷ are the same or different and are each independently selected from H, C₁₋₃alkyl and haloC₁₋₃alkyl, or any subset thereof.

One preferred set of compounds of the invention is defined by the formula (I-ii):

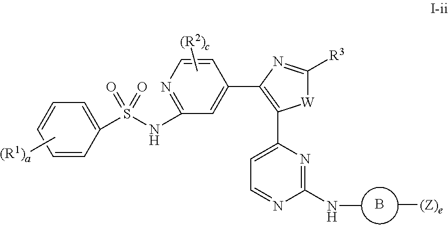

wherein all variables are as defined herein.

Within the compounds of formula (I-ii) a preferred set of compounds is defined by formulas (I-iii-a) (I-iii-b) and (I-iii-c):

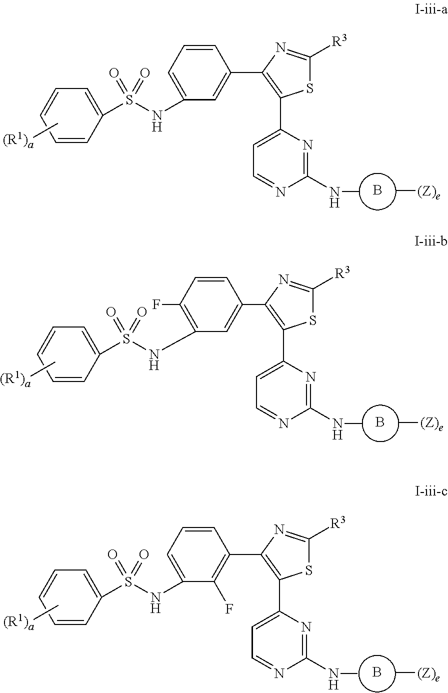

wherein all variables are as defined herein.

Other preferred compounds of the invention are defined by formulas (I-iv-a), (I-iv-b), (I-iv-c), (I-iv-d), (I-iv-e) and (I-iv-f):

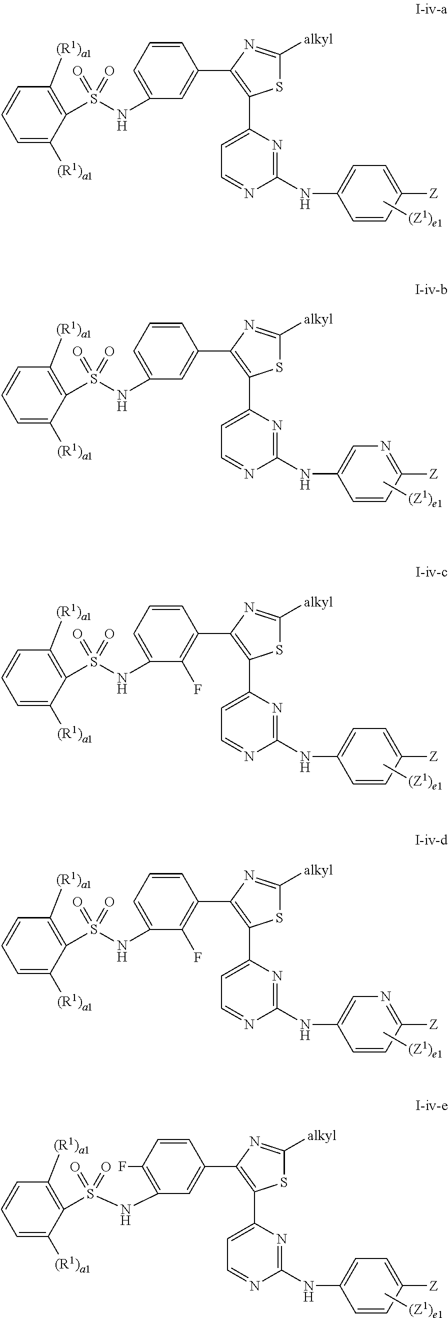

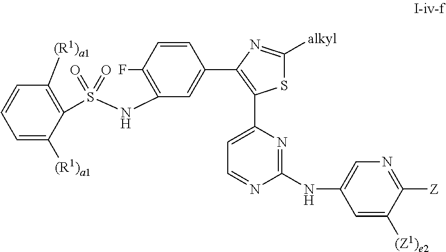

wherein all variables are as defined herein.

More particularly, the compounds of formula (I-iv-a), (I-iv-b), (I-iv-c), (I-iv-d), (I-iv-e) and (I-iv-f) are defined wherein
a1 is 0 or 1;
R¹ is halo (particularly F);
e1 is 1 or 2;
e2 is 0 or 1;
Z is as defined above; and
Z¹ is halo, alkyl, haloalkyl, O-alkyl, or CO₂H, more particularly halo (e.g., F), alkyl (e.g., methyl) or O-alkyl (e.g., OMe).

It is to be understood that the present invention includes all combinations and subsets of the particular groups defined hereinabove.

Specific examples of compounds of the present invention include those recited in the Examples which follow as well as pharmaceutically acceptable salts of compounds exemplified as the free base and free base versions and other pharmaceutically acceptable salts of those compounds exemplified as salts.

Preferred compounds of formula (I) are selected from:
N-[3-(2-(1-Methylethyl)-5-{2-[(6-{4-[2-(methyloxy)ethyl]-1-piperazinyl}-3-pyridinyl)amino]-4-pyrimidinyl}-1,3-thiazol-4-yl)phenyl]benzenesulfonamide;
N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-4-fluorobenzenesulfonamide;
N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-2-fluorobenzenesulfonamide;
2,6-Difluoro-N-{3-[5-(2-{p-fluoro-4-({1-[2-(methylsulfonyl)ethyl]-4-piperidinyl}oxy)phenyl]amino}-4-pyrimidinyl)-2-(1-methylethyl)-1,3-thiazol-4-yl]phenyl}benzenesulfonamide hydrochloride;
5-({4-[4-(3-{[(2,6-Difluorophenyl)sulfonyl]amino}phenyl)-2-(1-methylethyl)-1,3-thiazol-5-yl]-2-pyrimidinyl}amino)-2-(4-morpholinyl)benzoic acid;
2,6-Difluoro-N-{3-[5-[2-({6-[(9aR)-hexahydropyrazino[2,1-c][1,4]oxazin-8(1H)-yl]-3-pyridinyl}amino)-4-pyrimidinyl]-2-(1-methylethyl)-1,3-thiazol-4-yl]phenyl}benzenesulfonamide;
2,6-Difluoro-N-{3-[5-{2-[(3-fluoro-4-{4-[2-(methylsulfonyl)ethyl]-1-piperazinyl}phenyl)amino]-4-pyrimidinyl}-2-(1-methylethyl)-1,3-thiazol-4-yl]phenyl}benzenesulfonamide hydrochloride;
2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide;
N-{3-[5-(2-{[3-(Dimethylamino)-4-(methyloxy)phenyl]amino}-4-pyrimidinyl)-2-(1-methylethyl)-1,3-thiazol-4-yl]phenyl}-2,6-difluorobenzenesulfonamide trifluoroacetate;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-{3-[2-(1-methylethyl)-5-(2-{[2-(methyloxy)-4-(4-{4-[2-(methylsulfonyl)ethyl]-1-piperazinyl}-1-piperidinyl)phenyl]amino}-4-pyrimidinyl)-1,3-thiazol-4-yl]phenyl}benzenesulfonamide;
2,6-Difluoro-N-{3-[5-[2-({3-fluoro-4-[4-(2-fluoroethyl)-1-piperazinyl]phenyl}amino)-4-pyrimidinyl]-2-(1-pyrrolidinyl)-1,3-thiazol-4-yl]phenyl}benzenesulfonamide;
2-Fluoro-N-{2-fluoro-5-[5-{2-[(3-fluoro-4-{4-[2-(methylsulfonyl)ethyl]-1-piperazinyl}phenyl)amino]-4-pyrimidinyl}-2-(1-methylethyl)-1,3-thiazol-4-yl]phenyl}benzenesulfonamide hydrochloride;
2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide;
N-{5-[5-(2-{[6-(4-Cyano-1-piperidinyl)-3-pyridinyl]amino}-4-pyrimidinyl)-2-(1-methylethyl)-1,3-thiazol-4-yl]-2-fluorophenyl}-2,6-difluorobenzenesulfonamide;
2,6-Difluoro-N-{2-fluoro-5-[5-[2-({3-fluoro-4-[4-(2-fluoroethyl)-1-piperazinyl]phenyl}amino)-4-pyrimidinyl]-2-(1-methylethyl)-1,3-thiazol-4-yl]phenyl}benzenesulfonamide;
2,6-Difluoro-N-{2-fluoro-5-[5-(2-{[6-(4-morpholinyl)-3-pyridinyl]amino}-4-pyrimidinyl)-2-(1-pyrrolidinyl)-1,3-thiazol-4-yl]phenyl}benzenesulfonamide;
N-{5-[2-(1,1-Dimethylethyl)-5-(2-{[6-(4-morpholinyl)-3-pyridinyl]amino}-4-pyrimidinyl)-1,3-thiazol-4-yl]-2-fluorophenyl}-2,6-difluorobenzenesulfonamide hydrochloride;
2,6-Difluoro-N-{2-fluoro-3-[5-(2-{[5-fluoro-6-(4-morpholinyl)-3-pyridinyl]amino}-4-pyrimidinyl)-2-(1-methylethyl)-1,3-thiazol-4-yl]phenyl}benzenesulfonamide; and
2,6-Difluoro-N-{4-[2-(1-methylethyl)-5-(2-{[6-(4-morpholinyl)-3-pyridinyl]amino}-4-pyrimidinyl)-1,3-thiazol-4-yl]-2-pyridinyl}benzenesulfonamide;
   including both free base and pharmaceutically acceptable salt versions thereof.

In particular, preferred compounds of formula (I) include but are not limited to:
2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-{3-[2-(1-methylethyl)-5-(2-{[2-(methyloxy)-4-(4-{4-[2-(methylsulfonyl)ethyl]-1-piperazinyl}-1-piperidinyl)phenyl]amino}-4-pyrimidinyl)-1,3-thiazol-4-yl]phenyl}benzenesulfonamide;
2,6-Difluoro-N-{3-[5-[2-({3-fluoro-4-[4-(2-fluoroethyl)-1-piperazinyl]phenyl}amino)-4-pyrimidinyl]-2-(1-pyrrolidinyl)-1,3-thiazol-4-yl]phenyl}benzenesulfonamide;
2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide;
N-{5-[5-(2-{[6-(4-Cyano-1-piperidinyl)-3-pyridinyl]amino}-4-pyrimidinyl)-2-(1-methylethyl)-1,3-thiazol-4-yl]-2-fluorophenyl}-2,6-difluorobenzenesulfonamide;
2,6-Difluoro-N-{2-fluoro-5-[5-(2-{[6-(4-morpholinyl)-3-pyridinyl]amino}-4-pyrimidinyl)-2-(1-pyrrolidinyl)-1,3-thiazol-4-yl]phenyl}benzenesulfonamide; and
N-{5-[2-(1,1-Dimethylethyl)-5-(2-{[6-(4-morpholinyl)-3-pyridinyl]amino}-4-pyrimidinyl)-1,3-thiazol-4-yl]-2-fluorophenyl}-2,6-difluorobenzenesulfonamide hydrochloride;
   including both free base and pharmaceutically acceptable salt versions thereof.

One example of a more preferred compound of formula (I) is 2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide and pharmaceutically acceptable salts thereof.

Another example of a more preferred compound of formula (I) is 2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide.

The following pharmaceutically acceptable salt forms of the compound are also examples of preferred compounds of the invention:
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide hydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide dihydrochloride;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide 4-methylbenzenesulfonate;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide methanesulfonate hydrate; and
Mono sodium salt of 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide.

Another example of a more preferred compound of formula (I) is 2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide and pharmaceutically acceptable salts thereof.

It will be appreciated by those skilled in the art that the compounds of formula (I) may be utilized as a pharmaceutically acceptable salt version thereof. The pharmaceutically acceptable salts of the compounds of formula (I) include conventional salts formed from pharmaceutically acceptable (i.e., non-toxic) inorganic or organic acids or bases as well as quaternary ammonium salts. Representative salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate (methanesulfonate), methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate (methylbenzenesulfonate), triethiodide, trimethylammonium and valerate. Other salts, such as oxalic, which are not themselves pharmaceutically acceptable, may be useful in the preparation of salts useful as intermediates in obtaining compounds of this invention and these form a further aspect of the invention. In one embodiment, the compound of formula (I) is in the form of the hydrochloride salt, including mono- and di-hydrochloride salts. In one embodiment, the compound of formula (I) is in the form of the methanesulfonate salt. In one embodiment, the compound of formula (I) is in the form of the methylbenzenesulfonate salt. In one embodiment, the compound of formula (I) is in the form of the sodium salt. Certain salt versions of the compounds may be solvates, particularly hydrates. In one embodiment, the compound of formula (I) is in the form of a mono-, di- or hemi-hydrate.

Processes for preparing pharmaceutically acceptable salts of compounds such as the compounds of formula (I) are conventional in the art. See, e.g., Burger's Medicinal Chemistry And Drug Discovery 5th Edition, Vol 1: Principles And Practice. As will be apparent to those skilled in the art, in the processes described below for the preparation of compounds of formula (I), certain intermediates, may be in the form of pharmaceutically acceptable salts of the compound. Processes for preparing pharmaceutically acceptable salts of intermediates are known in the art and are analogous to the processes for preparing pharmaceutically acceptable salts of other compounds such as the compounds of formula (I).

Compounds of the invention are believed to inhibit of one or more kinases and in particular one or more Raf family kinases (“Raf inhibitor”) and/or ErbB family kinases (i.e., EGFR, ErbB2 and ErbB4 (“ErbB inhibitor”). Compounds of the invention may also inhibit one or more other kinases, and particularly other tyrosine kinases. Certain compounds of the invention may inhibit B-Raf (“B-Raf inhibitor”). Certain compounds of the invention may inhibit ErbB2 (“ErbB2 inhibitor”). Compounds of the invention may be inhibitors of either Raf family kinases or ErbB family kinases, or in some instances may inhibit both. It is well documented that Raf inhibitors, including B-Raf inhibitors, and ErbB inhibitors, including ErbB2 inhibitors, are believed to be useful as anticancer and antitumor agents. See, e.g., Davies (2002) supra, Garnett (2004) supra, Zebisch (2006) supra, Normanno (2005) supra and Hynes (2005) supra. The anticancer and antitumor effects of these kinase inhibitors is currently believed to result from inhibition of one or more Raf family kinases and/or ErbB family kinases, and the effect of such inhibition on cell lines whose growth and/or viability is dependent on the kinase activity of Raf and/or ErbB family kinases.

Some compounds of the invention may be selective inhibitors of Raf family kinases (“selective Raf inhibitor”), meaning that preferential inhibition of one or more Raf family kinases is significantly greater than that of any number of other kinases, for example by a factor of 5-fold or more. Certain compounds of the invention may be selective inhibitors of ErbB family kinases (“selective ErbB inhibitor”), meaning that preferential inhibition of one or more ErbB family kinases is significantly greater than that of any number of other kinases, for example by a factor of 5-fold or more. Still other compounds of the invention may selectively inhibit Raf family kinases and ErbB family kinases over other kinases (“selective dual Raf/ErbB inhibitor”), meaning that inhibition of one or more Raf family kinases and one or more ErbB family kinases is each significantly greater than that of any number of other kinases, for example by a factor of 5-fold or more.

However, the present invention is not limited to compounds which are selective inhibitors of one or both of Raf family kinases and/or ErbB family kinases; rather, the present invention expressly contemplates that certain compounds of the invention may possess activity against multiple kinases, including kinases other than Raf family kinases and ErbB family kinases. For example, particular compounds of the invention may possess activity against multiple other kinases, including but not limited to IGFR, IR, IRR, Src, VEGFR, PDGFR, Met, Lyn, Lck, AIkS, Aurora A and B, JNK, Syk, p38, BTK, FAK, Abl, Ack1, Arg, BLK, CAMK1δ, CDK6, CK1, cKit, CSK, DDR2, Ephrin receptors, FGFR, Flt3, Fms, Fyn, Hck, HIPK2, Itk, MINK, Mnk2, PAK3, PKCμ, PKD2, PTK5, Ret, Ron, SIK, Tie2, TrkB, Yes, as well. Particular compounds of the invention may be deemed to be unselective or non-selective, meaning that they are not deemed to be selective for any particular kinase over others.

As used herein, a Raf inhibitor is a compound that exhibits a pIC₅₀ of greater than about 6 against at least one Raf family kinase in one or more of the Raf inhibition enzyme assays described below and/or an IC₅₀ of not greater than about 5 μM potency against one cell line that expresses mutated B-Raf kinase (e.g., A375P F11s, Colo205, HT-29, SK-MEL-3, SK-MEL-28) in the cellular proliferation assay described below. In a particular embodiment, a Raf inhibitor refers to a compound of the invention that exhibits a pIC₅₀ of greater than about 6.5 against at least one Raf family kinase in one or more of the Raf inhibition enzyme assays described below and an IC₅₀ of not greater than about 500 nM potency against at least one cell line that expresses mutated B-Raf kinase in the cellular proliferation assay described below.

An ErbB inhibitor is a compound which exhibits a pIC₅₀ of greater than about 6 against at least one ErbB family kinase in one or more of the ErbB inhibition enzyme assays described below and/or an IC₅₀ of not greater than about 5 μM potency against at least one cell line (e.g., BT474 or HN5) that overexpresses at least one ErbB family kinase in the cellular proliferation assay described below. Similarly, in one particular embodiment, a ErbB inhibitor refers to a compound of the invention which exhibits a pIC₅₀ of greater than about 6.5 against at least one ErbB family kinase in one or more of the ErbB inhibition enzyme assays described below and/or an IC₅₀ of not greater than about 500 nM potency against at least one cell line that overexpresses at least one ErbB family kinase in the cellular proliferation assay described below.

A “dual Raf/ErbB inhibitor” refers to a compound of the invention which exhibits a pIC₅₀ of greater than about 6 against at least one ErbB family kinase and against at least one Raf family kinase in one or more of the enzyme inhibition assays described below and an IC₅₀ of not greater than about 5 μM potency against at least one cell line that overexpresses at least one ErbB family kinase in the cellular proliferation assay described below and an IC₅₀ of not greater than about 5 μM potency against at least one cell line that expresses mutated B-Raf kinase in the cellular proliferation assay described below. In one embodiment, a “dual Raf/ErbB inhibitor” refers to a compound of the invention which exhibits a pIC₅₀ of greater than about 6.5 against at least one ErbB family kinase and against at least one Raf family kinase in one or more of the enzyme inhibition assays described below and an IC₅₀ of not greater than about 500 nM potency against at least one cell line that overexpresses at least one ErbB family kinase in the cellular proliferation assay described below and an IC₅₀ of not greater than about 500 nM potency against at least one cell line that expresses mutated B-Raf kinase in the cellular proliferation assay described below.

A “B-Raf inhibitor” refers to a compound of the invention that exhibits a pIC₅₀ of greater than about 6.5 against B-Raf in one or more of the Raf inhibition enzyme assays described below and an IC₅₀ of not greater than 500 nM potency against at least one cell line that expresses mutated B-Raf kinase in the cellular proliferation assay described below. An “ErbB2 inhibitor” refers to a compound of the invention which exhibits a pIC₅₀ of greater than about 6.5 against at least one ErbB family kinase in one or more of the ErbB inhibition enzyme assays described below and/or an IC₅₀ of not greater than about 500 nM potency against at least one cell line that overexpresses at least one ErbB family kinase (e.g., ErbB2) in the cellular proliferation assay described below. A “dual B-Raf/ErbB2 inhibitor” refers to a compound of the invention which exhibits a pIC₅₀ of greater than about 6.5 against at least one ErbB family kinase and against B-Raf in one or more of the enzyme inhibition assays described below and an IC₅₀ of not greater than about 500 nM potency against at least one cell line that overexpresses at least one ErbB family kinase (e.g., ErbB2) in the cellular proliferation assay described below and an IC₅₀ of not greater than about 500 nM potency against at least one cell line that expresses mutated B-Raf kinase in the cellular proliferation assay described below.

The present invention provides compounds for use in medical therapy in a mammal, e.g., a human, in need thereof. The present invention provides methods for the treatment of several conditions in a mammal, in need thereof, all of which comprise the step of administering a therapeutically effective amount of a compound of the invention. All methods described herein are applicable to mammals, and particularly to humans. As used herein, the term “treatment” or “treating” in the context of therapeutic methods, refers to alleviating the specified condition, eliminating or reducing the symptoms of the condition, slowing or eliminating the progression, invasion, or metastatic spread of the condition and preventing or delaying the reoccurrence of the condition in a previously afflicted subject. The present invention further provides use of the compounds of the invention for the preparation of a medicament for the treatment of several conditions in a mammal (e.g., human) in need thereof.

In particular, the present invention provides compounds for use in the treatment of a condition mediated by at least one Raf family kinase (e.g., B-Raf) or at least one ErbB family kinase in a mammal in need thereof. The present invention provides a method for treating a condition mediated by at least one Raf family kinase (e.g., B-Raf) or at least one ErbB family kinase in a mammal (e.g., a human) in need thereof, which method comprises administering to the mammal a therapeutically effective amount of the compound of the invention.

In another embodiment, the invention provides compounds for use in regulating, modulating, binding or inhibiting one or more Raf family kinases (e.g., B-Raf) in a mammal and compounds for use in regulating, modulating, binding or inhibiting one or more ErbB family kinases (e.g., ErbB2) in a mammal. The invention also provides methods for regulating, modulating, binding, or inhibiting at least one Raf family kinase (e.g., B-Raf) in a mammal and methods for regulating, modulating, binding, or inhibiting at least one ErbB family kinases (e.g., ErbB2) in a mammal, each method comprising administering a therapeutically effective amount of a compound of the invention. “Regulating, modulating, binding or inhibiting at least one Raf family kinase” refers to regulating, modulating, binding or inhibiting the activity of at least one Raf family kinase, as well as regulating, modulating, binding or inhibiting overexpression of an upstream regulator of at least one Raf family kinase in order to inhibit the cellular potency of its signaling ability. “Regulating, modulating, binding or inhibiting at least one ErbB family kinase” refers to regulating, modulating, binding or inhibiting the activity of at least one ErbB family kinase, as well as regulating, modulating, binding or inhibiting overexpression of an upstream regulator of at least one ErbB family kinase in order to inhibit the cellular potency of its signaling ability.

In a particular embodiment, the invention provides compounds for use in the treatment of a condition mediated by inappropriate activity of one or more Raf family kinases (e.g., B-Raf), or an upstream activator of one or more Raf family kinases, or inappropriate activity of one or more ErbB family kinases or an upstream activator of one or more ErbB family kinases in a mammal. The invention further provides methods for the treatment of a condition mediated by inappropriate activity of one or more Raf family kinases (particularly B-Raf) or ErbB family kinases (particularly ErbB2), in a mammal in need thereof, comprising administering to the mammal, a therapeutically effective amount of a compound of the invention. In an additional aspect, the present invention provides the use of a compound of the invention for the preparation of a medicament for the treatment of a condition mediated by inappropriate activity of one or more Raf family kinases (particularly B-Raf) or ErbB family kinases (particularly ErbB2), in a mammal. One example of a condition mediated by inappropriate activity of one or more Raf family kinases or ErbB family kinases includes neoplasms.

By “inappropriate activity” is meant Raf family kinase or ErbB family kinase activity that deviates from the expected activity for that kinase or for an upstream activator of that kinase in a particular mammal. The inappropriate activity of a Raf family kinase may arise from one or more of A-Raf, B-Raf or c-Raf or an upstream activator of a Raf family kinase. The inappropriate activity of an ErbB family kinase may arise from one or more of EGFR, ErbB2 or ErbB4 or an upstream activator of an ErbB family kinase. Inappropriate Raf family kinase or ErbB family kinase activity may take the form of, for instance, an abnormal increase in activity, or an aberration in the timing and/or control of Raf or ErbB family kinase activity, respectively. Such inappropriate activity may result, for example, from overexpression or mutation of the protein kinase, upstream activator, receptor or ligand leading to inappropriate or uncontrolled activation of the corresponding kinase or receptor. Furthermore, it is also contemplated that unwanted Raf family kinase or ErbB family kinase activity may reside in an abnormal source, such as a neoplasm. Thus, the level of Raf family kinase or ErbB family kinase activity does not need to be abnormal to be considered inappropriate in the case where the activity derives from an abnormal source including, but not limited to, upstream activators (e.g., activated mutant Ras GTPases) or neoplasm. In one example of inappropriate Raf family activity not resulting from mutation or overexpression of a Raf family kinase, inappropriate activity of a Ras GTPase may result from mutation or overexpression of said Ras GTPase, for example the G13D mutation in KRas2, and may lead to overactivation of the MAPK pathway mediated by Raf family activity.

Thus, in one embodiment, the present invention provides compounds for use in the treatment of a condition which directly or indirectly results from a mutation of one or more Raf or ErbB family kinases (particularly B-Raf or ErbB2) or overexpression of one or more Raf or ErbB family kinases (particularly B-Raf or ErbB2), or a mutation of an upstream activator of one or more Raf or ErbB family kinases, or overexpression of an upstream activator of one or more Raf or ErbB family kinases in a mammal in need thereof. The present invention provides methods for the treatment of a condition which directly or indirectly results from mutation of a Raf or ErbB family kinase or overexpression of a Raf or ErbB family kinase, or a mutation of an upstream activator of a Raf or ErbB family kinase or overexpression of an upstream activator of a Raf or ErbB family kinase in a mammal in need thereof, comprising administering to the mammal a therapeutically effective amount of a compound of the invention. In an additional aspect, the present invention provides the use of a compound of the invention for the preparation of a medicament for the treatment of a condition which directly or indirectly results from mutation of a Raf or ErbB family kinase or overexpression of a Raf or ErbB family kinase, or a mutation of an upstream activator of a Raf or ErbB family kinase or overexpression of an upstream activator of a Raf or ErbB family kinase in a mammal. Conditions which are mediated by at least one Raf family kinase and conditions which are mediated by at least one ErbB family kinase, and particularly conditions mediated by inappropriate activity of one or Raf or ErbB family kinases, including those which directly or indirectly result from mutation of a Raf or ErbB family kinase, overexpression of a Raf or ErbB family kinase, or mutation of an upstream activator of a Raf or ErbB family kinase or overexpression of an upstream activator of a Raf or ErbB family kinase are known in the art and include but are not limited to neoplasms.

Compounds of the invention may also be used in the treatment of conditions attenuated by inhibition of a Raf family kinase (particularly B-Raf) or inhibition of an ErbB family kinase (particularly ErbB2). Further provided are methods for treating a condition attenuated by inhibition of a Raf family kinase (particularly B-Raf) or inhibition of an ErbB family kinase (particularly ErbB2) in a mammal in need thereof, comprising administering to the mammal a therapeutically effective amount of a compound of the invention. Also provided is the use of a compound of the invention for the preparation of a medicament for the treatment of a condition attenuated by inhibition of a Raf family kinase (particularly B-Raf) or inhibition of an ErbB family kinase (particularly ErbB2) in a mammal. Conditions attenuated by inhibition of a Raf family kinase (including B-Raf) or inhibition of an ErbB family kinase (particularly ErbB2) include but are not limited to neoplasms.

Accordingly, compounds of the invention may be used in the treatment of a neoplasm, particularly a susceptible neoplasm (a cancer or tumor) in a mammal. The present invention also provides a method for treating a neoplasm, particularly a susceptible neoplasm in a mammal in need thereof, which method comprises administering to the mammal a therapeutically effective amount of the compound of the invention. The invention also provides the use of a compound of the invention for the preparation of a medicament for the treatment of neoplasm, particularly a susceptible neoplasm, in a mammal. “Susceptible neoplasm” as used herein refers to neoplasms which are susceptible to treatment by a kinase inhibitor and particularly neoplasms that are susceptible to treatment by either a Raf inhibitor or an ErbB inhibitor. Neoplasms which have been associated with inappropriate activity of one or more Raf family kinases and particularly neoplasms which exhibit mutation of a Raf family kinase, overexpression of a Raf family kinase, or mutation of an upstream activator of a Raf family kinase or overexpression of an upstream activator of a Raf family kinase, and are therefore susceptible to treatment with an Raf inhibitor are known in the art, and include both primary and metastatic tumors and cancers. See, Catalogue of Somatic Mutations in Cancer (COSMIC), the Welcome Trust Sanger Institute, http://www.sanger.ac.uk/genetics/CGP/cosmic/and those references cited in the background. Neoplasms which have been associated with inappropriate activity of one or more ErbB family kinases and particularly neoplasms which are exhibit mutation of an ErbB family kinase, overexpression of an ErbB family kinase, or mutation of an upstream activator of an ErbB family kinase or overexpression of an upstream activator of an ErbB family kinase, and are therefore susceptible to treatment with an ErbB inhibitor are known in the art, and include both primary and metastatic tumors and cancers. See, references cited in the background.

Specific examples of susceptible neoplasms within the scope of the invention include, but are not limited to:
Barret's adenocarcinoma;
billiary tract carcinomas;
bladder cancer;
breast cancer;
cervical cancer;
cholangiocarcinoma;
central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system),
colorectal cancer, including large intestinal colon carcinoma;
esophageal cancer
gastric cancer;
carcinoma of the head and neck including squamous cell carcinoma of the head and neck;
hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia;
hepatocellular carcinoma;
lung cancer including small cell lung cancer, non-small cell lung cancer and squamous cell lung cancer;
ovarian cancer, endometrial cancer, cervical cancer;
pancreatic cancer;
pituitary adenoma;
prostate cancer;
renal cancer;
sarcoma;
skin cancers including melanomas;
thyroid cancers; and
uterine cancer.

Accordingly, in one embodiment, the present invention provides a method for the treatment of Barret's adenocarcinoma; billiary tract carcinomas; bladder cancer; breast cancer; cervical cancer; cholangiocarcinoma; central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system); colorectal cancer, including large intestinal colon carcinoma; esophageal cancer; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer, non-small cell lung cancer and squamous cell lung cancer; ovarian cancer; endometrial cancer; cervical cancer; pancreatic cancer; pituitary adenoma; prostate cancer; renal cancer; sarcoma; skin cancers including melanomas; thyroid cancers; and uterine cancer, or any subset thereof, in a mammal in need thereof, the method comprising administering a therapeutically effective amount of a compound of the invention to the mammal.

The present invention also provides the a compound of formula (I) for use in the treatment of Barret's adenocarcinoma; billiary tract carcinomas; bladder cancer; breast cancer; cervical cancer; cholangiocarcinoma; central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system); colorectal cancer, including large intestinal colon carcinoma; esophageal cancer; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer, non-small cell lung cancer and squamous cell lung cancer; ovarian cancer; endometrial cancer; cervical cancer; pancreatic cancer; pituitary adenoma; prostate cancer; renal cancer; sarcoma; skin cancers including melanomas; thyroid cancers; and uterine cancer, or any subset thereof, in a mammal in need thereof.

The present invention further provides the use of a compound of formula (I) for the preparation of a medicament for the treatment of Barret's adenocarcinoma; billiary tract carcinomas; bladder cancer; breast cancer; cervical cancer; cholangiocarcinoma; central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system); colorectal cancer, including large intestinal colon carcinoma; esophageal cancer; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer, non-small cell lung cancer and squamous cell lung cancer; ovarian cancer; endometrial cancer; cervical cancer; pancreatic cancer; pituitary adenoma; prostate cancer; renal cancer; sarcoma; skin cancers including melanomas; thyroid cancers; and uterine cancer, or any subset thereof, in a mammal in need thereof.

As is well known in the art, tumors may metastasize from a first or primary locus of tumor to one or more other body tissues or sites. In particular, metastases to the central nervous system (i.e., secondary CNS tumors), and particularly the brain (i.e., brain metastases), are well documented for tumors and cancers, such as breast, lung, melanoma, renal and colorectal. As used herein, reference to uses or methods for treatment or treatments for a “neoplasm,” “tumor” or “cancer” in a subject includes use for and treatment of the primary neoplasm, tumor or cancer, and where appropriate, also the use for and treatment of metastases (i.e., metastatic tumor growth) as well.

In one particular embodiment, the invention provides a method for treating breast cancer in a mammal in need thereof, comprising administering to the mammal a therapeutically effective amount of a compound of the invention. In one particular embodiment, the invention provides a method for treating colorectal cancer in a mammal in need thereof, comprising administering to the mammal a therapeutically effective amount of a compound of the invention. In one particular embodiment, the invention provides a method for treating melanoma in a mammal in need thereof, comprising administering to the mammal a therapeutically effective amount of a compound of the invention. In one particular embodiment, the invention provides a method for treating non-small cell lung cancer in a mammal in need thereof, comprising administering to the mammal a therapeutically effective amount of a compound of the invention. In one particular embodiment, the invention provides a method for treating ovarian cancer in a mammal in need thereof, comprising administering to the mammal a therapeutically effective amount of a compound of the invention. In one particular embodiment, the invention provides a method for treating thyroid cancer in a mammal in need thereof, comprising administering to the mammal a therapeutically effective amount of a compound of the invention.

In one particular embodiment, the susceptible neoplasm is breast cancer and the invention provides compounds for use in the treatment of breast cancer in a mammal and the use of such compounds for the preparation of a medicament for the treatment of breast cancer in a mammal. In another embodiment, the susceptible neoplasm is colorectal cancer and the invention provides compounds for use in the treatment of colorectal cancer in a mammal and the use of such compounds for the preparation of a medicament for the treatment of colorectal cancer in a mammal. In another embodiment, the susceptible neoplasm is melanoma, and the invention provides compounds for use in the treatment of melanoma in a mammal and the use of such compounds for the preparation of a medicament for the treatment of melanoma in a mammal. In another embodiment, the susceptible neoplasm is non-small cell lung cancer, and the invention provides compounds for use in the treatment of non-small cell lung cancer in a mammal and the use of such compounds for the preparation of a medicament for the treatment of non-small cell lung cancer in a mammal. In another embodiment, the susceptible neoplasm is ovarian cancer and the invention provides compounds for use in the treatment of ovarian cancer in a mammal and the use of such compounds for the preparation of a medicament for the treatment of ovarian cancer in a mammal. In another embodiment, the susceptible neoplasm is thyroid cancer, and the invention provides compounds for use in the treatment of thyroid cancer in a mammal and the use of such compounds for the preparation of a medicament for the treatment of thyroid cancer in a mammal.

The compounds of the invention can be used alone in the treatment of each of the foregoing conditions or can be used to provide additive or potentially synergistic effects with certain existing chemotherapies, radiation, biological or immunotherapeutics (including monoclonal antibodies) and vaccines. The compounds of the invention may be useful for restoring effectiveness of certain existing chemotherapies and radiation and or increasing sensitivity to certain existing chemotherapies and/or radiation.

As used herein, the term “therapeutically effective amount” means an amount of a compound of the invention which is sufficient, in the subject to which it is administered, to elicit the biological or medical response of a cell culture, tissue, system, mammal (including human) that is being sought, for instance, by a researcher or clinician. The term also includes within its scope amounts effective to enhance normal physiological function. For example, a therapeutically effective amount of a compound of the invention for the treatment of a condition mediated by at least one Raf family kinase or at least one ErbB family kinase is an amount sufficient to treat the condition in the subject. Similarly, a therapeutically effective amount of a compound of the invention for the treatment of a susceptible neoplasm is an amount sufficient to treat the particular susceptible neoplasm in the subject. In one embodiment of the present invention, a therapeutically effective amount of a compound of the invention is an amount sufficient to regulate, modulate, bind or inhibit at least one Raf family kinase or at least one ErbB family kinase. More particularly, in such embodiment, the therapeutically effective amount of a compound of the invention is an amount sufficient to regulate, modulate, bind or inhibit B-Raf and/or ErbB2 kinases.

The precise therapeutically effective amount of the compounds of the invention will depend on a number of factors. There are variables inherent to the compounds including, but not limited to, the following: molecular weight, inhibitory activity at the target kinase, absorption, bioavailability, distribution in the body, tissue penetration, half-life, metabolism, protein binding, and excretion. These variables determine what dose of compound needs to be administered in order to inhibit the target kinase by a sufficient percentage and for a sufficient amount of time to have the desired effect on the tumor. In general, the goal will be to inhibit the target kinase by 50% or more for as long as possible. The duration of drug exposure will be limited only by the compound half-life, and side effects from treatment requiring cessation of dosing. The amount of compound administered will also depend on factors related to patients and disease including, but not limited to, the following: the age, weight, concomitant medications and medical condition of the subject being treated, the precise condition requiring treatment and its severity, the nature of the formulation, and the route of administration. Ultimately the dose will be at the discretion of the attendant physician or veterinarian. Typically, the compound of the invention will be given for treatment in the range of 0.01 to 30 mg/kg body weight of recipient (mammal) per day and more usually in the range of 0.1 to 10 mg/kg body weight per day. Thus, for a 70 kg adult human being treated for a condition mediated by at least one Raf family kinase or at least one ErbB family kinase, the actual amount per day would usually be from 1 to 2000 mg and this amount may be given in a single or multiple doses per day. Dosing regimens may vary significantly and will be determined and altered based on clinical experience with the compound. The full spectrum of dosing regimens may be employed ranging from continuous dosing (with daily doses) to intermittent dosing. A therapeutically effective amount of a pharmaceutically acceptable salt of a compound of formula (I) may be determined as a proportion of the therapeutically effective amount of the compound of formula (I) as the free base. It is envisaged that similar dosages would be appropriate for treatment of the susceptible neoplasms described above.

While it is possible that, for use in therapy, a therapeutically effective amount of a compound of the invention may be administered as the raw chemical, it is typically presented as the active ingredient of a pharmaceutical composition or formulation. Accordingly, the invention further provides a pharmaceutical composition comprising a compound of the invention. The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the invention with one or more pharmaceutically acceptable carriers, diluents and/or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the invention (as a free-base, solvate (including hydrate) or salt, in any form), depending on the condition being treated, the route of administration, and the age, weight and condition of the patient. Preferred unit dosage formulations are those containing a daily dose, weekly dose, monthly dose, a sub-dose or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including capsules, tablets, liquid-filled capsules, disintegrating tablets, immediate, delayed and controlled release tablets, oral strips, solutions, syrups, buccal and sublingual), rectal, nasal, inhalation, topical (including transdermal), vaginal or parenteral (including subcutaneous, intramuscular, intravenous or intradermal) route. Such formulations may be prepared by any method known in the art of pharmacy, for example by bringing into association the active ingredient with the carrier(s), excipient(s) or diluent. Generally, the carrier, excipient or diluent employed in the pharmaceutical formulation is “non-toxic,” meaning that it/they is/are deemed safe for consumption in the amount delivered in the pharmaceutical composition, and “inert” meaning that it/they does/do not appreciably react with or result in an undesired effect on the therapeutic activity of the active ingredient.

Pharmaceutical formulations adapted for oral administration may be presented as discrete units such as liquid-filled or solid capsules; immediate, delayed or controlled release tablets; powders or granules; solutions or suspensions in aqueous or non-aqueous liquids; edible foams or whips; oil-in-water liquid emulsions, water-in-oil liquid emulsions or oral strips, such as impregnated gel strips.

For instance, for oral administration in the form of a tablet or capsule, the active drug component can be combined with an oral pharmaceutically acceptable carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Solid capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solutions, syrups and elixirs can be prepared in dosage unit form so that a given quantity contains a predetermined amount of the compound. Solutions and syrups can be prepared by dissolving the compound in a suitably flavored aqueous solution, while elixirs are prepared through the use of a pharmaceutically acceptable alcoholic vehicle. Suspensions can be formulated by dispersing the compound in a pharmaceutically acceptable vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, unit dosage formulations for oral administration can be microencapsulated. The formulation can also be prepared to prolong or sustain the release as for example by coating or embedding particulate material in polymers, wax or the like.

The compounds of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropyl-methacrylamidephenol, polyhydroxyethylaspartamidephenol, or polyethyleneoxide-polylysine substituted with palmitoyl residues. Furthermore, the compounds may be coupled to a class of biodegradable polymers useful in achieving controlled release of a drug, for example, polycentric acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research (1986) 3(6):318.

Pharmaceutical formulations adapted for topical administration may be formulated as ointments, creams, suspensions, lotions, powders, solutions, pastes, gels, sprays, aerosols or oils. For treatments of external tissues, such as skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical formulations adapted for topical administrations to the eye include eye drops wherein the active ingredient is dissolved or suspended in a suitable carrier, especially an aqueous solvent. Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, metered dose inhalers, dry powder inhalers, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation of pharmaceutically acceptable tonicity with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

In the above-described methods of treatment and uses, a compound of the invention may be employed alone, in combination with one or more other compounds of the invention or in combination with other therapeutic methods or agents. In particular, in methods of treating a condition attenuated by inhibition of at least one Raf family kinase and/or at least one ErbB family kinase and in methods of treating susceptible neoplasms, combination with other chemotherapeutic, biologic, hormonal, antibody and supportive care agents is envisaged as well as combination with surgical therapy and radiotherapy. Supportive care agents include analgesics, anti-emetics and agents used to treat hematologic side effects such as neutropenia. Analgesics are well known in the art. Anti-emetics include but are not limited to 5HT₃ antagonists such as ondansetron, granisetron, dolasetron, palonosetron and the like; proclorperazine; metaclopromide; diphenhydramine; promethazine; dexamethasone; lorazepam; haloperidol; dronabinol; olanzapine; and neurokinin-1 antagonists such as aprepitant, fosaprepitant and casopitant administered alone or in various combinations.

The term “chemotherapeutic” as used herein refers to any chemical agent having a therapeutic effect on the subject to which it is administered. “Chemotherapeutic” agents include but are not limited to anti-neoplastic agents. As used herein, “anti-neoplastic agents” include both cytotoxic and cytostatic agents including biological, immunological and vaccine therapies. Combination therapies according to the invention thus comprise the administration of at least one compound of the invention and the use of at least one other treatment method. In one embodiment, combination therapies according to the invention comprise the administration of at least one compound of the invention and surgical therapy. In one embodiment, combination therapies according to the invention comprise the administration of at least one compound of the invention and radiotherapy. In one embodiment, combination therapies according to the invention comprise the administration of at least one compound of the invention and at least one supportive care agent (e.g., at least one anti-emetic agent). In one embodiment, combination therapies according to the present invention comprise the administration of at least one compound of the invention and at least one other chemotherapeutic agent. In one particular embodiment, the invention comprises the administration of at least one compound of the invention and at least one anti-neoplastic agent.

As an additional aspect, the present invention provides the methods of treatment and uses as described above, which comprise administering a compound of the invention together with at least one chemotherapeutic agent. In one particular embodiment, the chemotherapeutic agent is an anti-neoplastic agent. In another embodiment, the invention provides a pharmaceutical composition as described above further comprising at least one other chemotherapeutic agent, more particularly, the chemotherapeutic agent is an anti-neoplastic agent. The invention also provides methods of treatment and uses as described above, which comprise administering a compound of the invention together with at least one supportive care agent (e.g., anti-emetic agent).

The compounds of the invention and at least one additional anti-neoplastic or supportive care therapy may be employed in combination concomitantly or sequentially in any therapeutically appropriate combination. The administration of a compound of the invention with one or more other anti-neoplastic agents may be in combination in accordance with the invention by administration concomitantly in (1) a unitary pharmaceutical composition including both or all compounds or (2) separate pharmaceutical compositions each including one or more of the compounds. The components of the combination may be administered separately in a sequential manner wherein one active ingredient is administered first and the other(s) second or vice versa. Such sequential administration may be close in time or remote in time.

When a compound of the invention is used in combination with an anti-neoplastic and/or supportive care agent, the dose of each compound may differ from that when the compound is used alone. Appropriate doses will be readily appreciated by those skilled in the art. The appropriate dose of the compound(s) of the invention and the other therapeutically active agent(s) and the relative timings of administration will be selected in order to achieve the desired combined therapeutic effect, and are within the expertise and discretion of the attendant clinician.

Typically, any chemotherapeutic agent that has activity against a susceptible neoplasm being treated may be utilized in combination with the compounds the invention, provided that the particular agent is clinically compatible with therapy employing a compound of the invention. Typical anti-neoplastic agents useful in the present invention include, but are not limited to: alkylating agents, anti-metabolites, antitumor antibiotics, antimitotic agents, topoisomerase I and II inhibitors, hormones and hormonal analogues; signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

Alkylating agents are non-phase specific anti-neoplastic agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Alkylating agents may be employed in combination with the compounds of the invention in the compositions and methods described above. Examples of alkylating agents include but are not limited to nitrogen mustards such as cyclophosphamides, temozolomide, melphalan, and chlorambucil; Oxazaphosphorines; alkyl sulfonates such as busulfan; nitrosoureas such as carmustine; triazenes such as dacarbazine; and platinum coordination complexes such as cisplatin, oxaliplatin and carboplatin.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. The end result of discontinuing S phase is cell death. Antimetabolite neoplastic agents may be employed in combination with the compounds of the invention in the compositions and methods described above. Examples of antimetabolite anti-neoplastic agents include but are not limited to purine and pyrimidine analogues and anti-folate compounds, and more specifically, hydroxyurea, cytosine, arabinoside, ralitrexed, tegafur, fluorouracil (e.g., 5FU), methotrexate, cytarabine, mercaptopurine and thioguanine.

Antitumor antibiotic agents are non-phase specific agents, which bind to or intercalate with DNA. Typically, such action disrupts ordinary function of the nucleic acids, leading to cell death. Antitumor antibiotics may be employed in combination with the compounds of the invention in the compositions and methods described above. Examples of antitumor antibiotic agents include, but are not limited to, actinomycins such as dactinomycin; anthracyclines such as daunorubicin, doxorubicin, idarubicin, epirubicin and mitoxantrone; mitomycin C and bleomycins.

Antimicrotubule or antimitotic agents are phase specific agents active against the microtubules of tumor cells during M or the mitosis phase of the cell cycle. Antimitotic agents may be employed in combination with the compounds of the invention in the compositions and methods described above. Examples of antimitotic agents include, but are not limited to, diterpenoids, vinca alkaloids, polo-like kinase (Plk) inhibitors and CenpE inhibitors. Examples of diterpenoids include, but are not limited to, paclitaxel and its analog docetaxel. Examples of vinca alkaloids include, but are not limited to, vinblastine, vincristine, vindesine and vinorelbine. Plk inhibitors are discussed further below.

Topoisomerase inhibitors include inhibitors of Topoisomerase II and inhibitors of Topoisomerase I. Topoisomerase II inhibitors, such as epipodophyllotoxins, are anti-neoplastic agents derived from the mandrake plant, that typically affect cells in the S and G2 phases of the cell cycle by forming a ternary complex with topoisomerase II and DNA, causing DNA strand breaks. The strand breaks accumulate and cell death follows. Examples of epipodophyllotoxins include, but are not limited to, etoposide and teniposide. Camptothecins, including camptothecin and camptothecin derivatives, are available or under development as Topoisomerase I inhibitors. Examples of camptothecins include, but are not limited to amsacrine, irinotecan, topotecan, and the various optical forms of 7-(4-methylpiperazino-methylene)-10,11-ethylenedioxy-20-camptothecin. Topoisomerase inhibitors may be employed in combination with the compounds of the invention in the compositions and methods described above.

Hormones and hormonal analogues are useful compounds for treating cancers in which there is a relationship between the hormone(s) and growth and/or lack of growth of the cancer. Antitumor hormones and hormonal analogues may be employed in combination with the compounds of the invention in the compositions and methods described above. Examples of hormones and hormonal analogues believed to be useful in the treatment of neoplasms include, but are not limited to antiestrogens, such as tamoxifen, toremifene, raloxifene, fulvestrant, iodoxyfene and droloxifene; anti-androgens; such as flutamide, nilutamide, bicalutamide and cyproterone acetate; adrenocorticosteroids such as prednisone and prednisolone; aminoglutethimide and other aromatase inhibitors such as anastrozole, letrazole, vorazole, and exemestane; progestins such as megestrol acetate; 5α-reductase inhibitors such as finasteride and dutasteride; and gonadotropin-releasing hormones (GnRH) and analogues thereof, such as Leutinizing Hormone-releasing Hormone (LHRH) agonists and antagonists such as goserelin luprolide, leuprorelin and buserelin.

Signal transduction pathway inhibitors are those inhibitors which block or inhibit a chemical process which evokes an intracellular change. As used herein this change is cell proliferation or differentiation or survival. Signal transduction pathway inhibitors useful in the present invention include, but are not limited to, inhibitors of receptor tyrosine kinases, non-receptor tyrosine kinases, SH2/SH3 domain blockers, serine/threonine kinases, phosphatidyl inositol-3-OH kinases, myoinositol signaling, and Ras oncogenes. Signal transduction pathway inhibitors may be employed in combination with the compounds of the invention in the compositions and methods described above.

Several protein tyrosine kinases catalyze the phosphorylation of specific tyrosine residues in various proteins involved in the regulation of cell growth. Such protein tyrosine kinases can be broadly classified as receptor or non-receptor kinases.

Receptor tyrosine kinase inhibitors which may be combined with the compounds of the invention include those involved in the regulation of cell growth, which receptor tyrosine kinases are sometimes referred to as “growth factor receptors.” Examples of growth factor receptor inhibitors, include but are not limited to inhibitors of: insulin growth factor receptors (IGF-1R, IR and IRR); epidermal growth factor family receptors (EGFR, ErbB2, and ErbB4); platelet-derived growth factor receptors (PDGFRs), vascular endothelial growth factor receptors (VEGFRs), tyrosine kinase with immunoglobulin-like and epidermal growth factor homology domains (TIE-2), macrophage colony stimulating factor (c-fms), c-kit, c-met, fibroblast growth factor receptors (FGFRs), hepatocyte growth factor receptors (HGFRs), Trk receptors (TrkA, TrkB, and TrkC), ephrin (Eph) receptors and the RET protooncogene.

Several inhibitors of growth factor receptors are under development and include ligand antagonists, antibodies, tyrosine kinase inhibitors, anti-sense oligonucleotides and aptamers. Any of these growth factor receptor inhibitors may be employed in combination with the compounds of the invention in any of the compositions and methods/uses described herein. Trastuzumab (Herceptin®) is an example of an anti-erbB2 antibody inhibitor of growth factor function. One example of an anti-erbB1 antibody inhibitor of growth factor function is cetuximab (Erbitux™, C225). Bevacizumab (Avastin®) is an example of a monoclonal antibody directed against VEGFR. Examples of small molecule inhibitors of epidermal growth factor receptors include but are not limited to lapatinib (Tykerb™) and erlotinib (TARCEVA®). lmatinib (GLEEVEC®) is one example of a PDGFR inhibitor. Examples of VEGFR inhibitors include pazopanib, ZD6474, AZD2171, PTK787, sunitinib and sorafenib.

In one embodiment, the invention provides methods of treatment of any of the various conditions enumerated above comprising administering a compound of the invention in combination with an EGFR or ErbB inhibitor. In one particular embodiment, the methods of the present invention comprise administering a compound of the invention in combination with lapatinib. In one particular embodiment, the methods of the present invention comprise administering a compound of the invention in combination with trastuzumab. In one particular embodiment, the methods of the present invention comprise administering a compound of the invention in combination with erlotinib. In one particular embodiment, the methods of the present invention comprise administering a compound of the invention in combination with gefitinib.

In another embodiment, the present invention provides methods of treatment of any of the various conditions enumerated above comprising administering a compound of the invention in combination with a VEGFR inhibitor. In one particular embodiment, the methods of the present invention comprise administering a compound of the invention in combination with pazopanib.

Tyrosine kinases that are not transmembrane growth factor receptor kinases are termed non-receptor, or intracellular tyrosine kinases. Inhibitors of non-receptor tyrosine kinases are sometimes referred to as “anti-metastatic agents” and are useful in the present invention. Targets or potential targets of anti-metastatic agents, include, but are not limited to, c-Src, Lck, Fyn, Yes, Jak, Abl kinase (c-Abl and Bcr-Abl), FAK (focal adhesion kinase) and Bruton's tyrosine kinase (BTK). Non-receptor kinases and agents, which inhibit non-receptor tyrosine kinase function, are described in Sinha, S, and Corey, S. J., (1999) J. Hematother. Stem Cell Res. 8:465-80; and Bolen, J. B. and Brugge, J. S., (1997) Annu. Rev. of Immunol. 15:371-404.

SH2/SH3 domain blockers are agents that disrupt SH2 or SH3 domain binding in a variety of enzymes or adaptor proteins including, but not limited to, PI3-K p85 subunit, Src family kinases, adaptor molecules (Shc, Crk, Nck, Grb2) and Ras-GAP. Examples of Src inhibitors include, but are not limited to, dasatinib and BMS-354825 (J. Med. Chem. (2004) 47:6658-6661).

Inhibitors of serine/threonine kinases may also be used in combination with the compounds of the invention in any of the compositions and methods described above. Examples of serine/threonine kinase inhibitors that may also be used in combination with a compound of the present invention include, but are not limited to, polo-like kinase inhibitors (Plk family e.g., Plk1, Plk2, and Plk3), which play critical roles in regulating processes in the cell cycle including the entry into and the exit from mitosis; MAP kinase cascade blockers, which include other Ras/Raf kinase inhibitors, mitogen or extracellular regulated kinases (MEKs), and extracellular regulated kinases (ERKs); Aurora kinase inhibitors (including inhibitors of Aurora A and Aurora B); protein kinase C (PKC) family member blockers, including inhibitors of PKC subtypes (alpha, beta, gamma, epsilon, mu, lambda, iota, zeta); inhibitors of kappa-B (IkB) kinase family (IKK-alpha, IKK-beta); PKB/Akt kinase family inhibitors; and inhibitors of TGF-beta receptor kinases. Examples of Plk inhibitors are described in PCT Publication No. WO04/014899 and WO07/03036 both to GlaxoSmithKline. Other examples of serine/threonine kinase inhibitors are known in the art. In another embodiment, the present invention provides methods of treatment of any of the various conditions enumerated above comprising administering a compound of the invention in combination with a Plk inhibitor. In one particular embodiment, the methods of the present invention comprise administering a compound of the invention in combination with 5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide.

Urokinase, also referred to as urokinase-type Plasminogen Activator (uPA), is a serine protease. Activation of the serine protease plasmin triggers a proteolysis cascade which is involved in thrombolysis or extracellular matrix degradation. Elevated expression of urokinase and several other components of the plasminogen activation system have been correlated with tumor malignancy including several aspects of cancer biology such as cell adhesion, migration and cellular mitotic pathways as well. Inhibitors of urokinase expression may be used in combination with the compounds of the invention in the compositions and methods described above.

Inhibitors of Ras oncogene may also be useful in combination with the compounds of the present invention. Such inhibitors include, but are not limited to, inhibitors of farnesyltransferase, geranyl-geranyl transferase, and CAAX proteases as well as anti-sense oligonucleotides, ribozymes and immunotherapy. Such inhibitors have been shown to block Ras activation in cells containing mutant Ras, thereby acting as antiproliferative agents.

Inhibitors of kinases involved in the IGF-1 R signaling axis may also be useful in combination with the compounds of the present invention. Such inhibitors include but are not limited to inhibitors of JNK1/2/3, PI3K, AKT and MEK, and 14.3.3 signaling inhibitors. Examples of AKT inhibitors are described in PCT Publication No. WO 2007/058850, published 24 May 2007 which corresponds to PCT Application No. PCT/US2006/043513, filed 9 Nov. 2006, to GlaxoSmithKline. One particular AKT inhibitor disclosed therein is 4-(2-(4-amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol.

Cell cycle signaling inhibitors, including inhibitors of cyclin dependent kinases (CDKs) are also useful in combination with the compounds of the invention in the compositions and methods described above. Examples of cyclin dependent kinases, including CDK2, CDK4, and CDK6 and inhibitors for the same are described in, for instance, Rosania G. R., et al., Exp. Opin. Ther. Patents (2000) 10:215-230.

Receptor kinase angiogenesis inhibitors may also find use in the present invention. Inhibitors of angiogenesis related to VEGFR and TIE-2 are discussed above in regard to signal transduction inhibitors (both are receptor tyrosine kinases). Other inhibitors may be used in combination with the compounds of the invention. For example, anti-VEGF antibodies, which do not recognize VEGFR (the receptor tyrosine kinase), but bind to the ligand; small molecule inhibitors of integrin (alphaᵥ beta₃) that inhibit angiogenesis; endostatin and angiostatin (non-RTK) may also prove useful in combination with the compounds of the invention. One example of a VEGFR antibody is bevacizumab (AVASTIN®).

Inhibitors of phosphatidyl inositol-3-OH kinase family members including blockers of PI3-kinase, ATM, DNA-PK, and Ku may also be useful in combination with the present invention.

Also of potential use in combination with the compounds of the invention are myo-inositol signaling inhibitors such as phospholipase C blockers and myoinositol analogues.

Antisense therapies may also be used in combination with the compounds of the invention. Examples of such antisense therapies include those directed towards the targets described above such as ISIS 2503 and gene therapy approaches such as those using thymidine kinase or cytosine deaminase.

Agents used in immunotherapeutic regimens may also be useful in combination with the compounds of the invention. Immunotherapeutic regimens include ex-vivo and in-vivo approaches to increasing immunogenicity of patient tumor cells such as transfection with cytokines (IL-2, IL-4, GMCFS and MCFS), approaches to increase T-cell activity, approaches with transfected immune cells and approaches with anti-idiotypic antibodies. Another potentially useful immunotherapeutic regimen is monoclonal antibodies with wild-type Fc receptors that may illicit an immune response in the host (e.g., IGF-1R monoclonal antibodies).

Agents used in proapoptotic regimens (e.g., Bcl-2 antisense oligonucleotides) may also be used in combination with the compounds of the invention. Members of the Bcl-2 family of proteins block apoptosis. Upregulation of Bcl-2 has therefore been linked to chemoresistance. Studies have shown that the epidermal growth factor (EGF) stimulates anti-apoptotic members of the Bcl-2 family (i.e., mcl-1). Therefore, strategies designed to downregulate the expression of Bcl-2 in tumors have demonstrated clinical benefit and are now in Phase II/III trials, namely Genta's G3139 bcl-2 antisense oligonucleotide. Such proapoptotic strategies using the antisense oligonucleotide strategy for Bcl-2 are discussed in Water, J. S., et al., J. Clin. Oncol. (2000) 18:1812-1823; and Kitada, S., et al., Antisense Res. Dev. (1994) 4:71-79.

Compounds of the invention may be prepared using the processes described below. In all of the schemes described below, it is understood that protecting groups may be employed where necessary in accordance with general principles known to those of skill in the art, for example, see Green, T. W. and Wuts, P. G. M. (1991) Protecting Groups in Organic Synthesis, John Wiley & Sons. The selection of a particular protecting group and processes for installation and removal of protecting groups is within the skill of those in the art. The selection of processes for installation and removal of protecting groups as well as the reaction conditions and order of their execution shall be consistent with the preparation of compounds of the invention.

Compounds of the invention wherein b2 is 0, may be conveniently prepared by the methods outlined in Scheme 1 below.

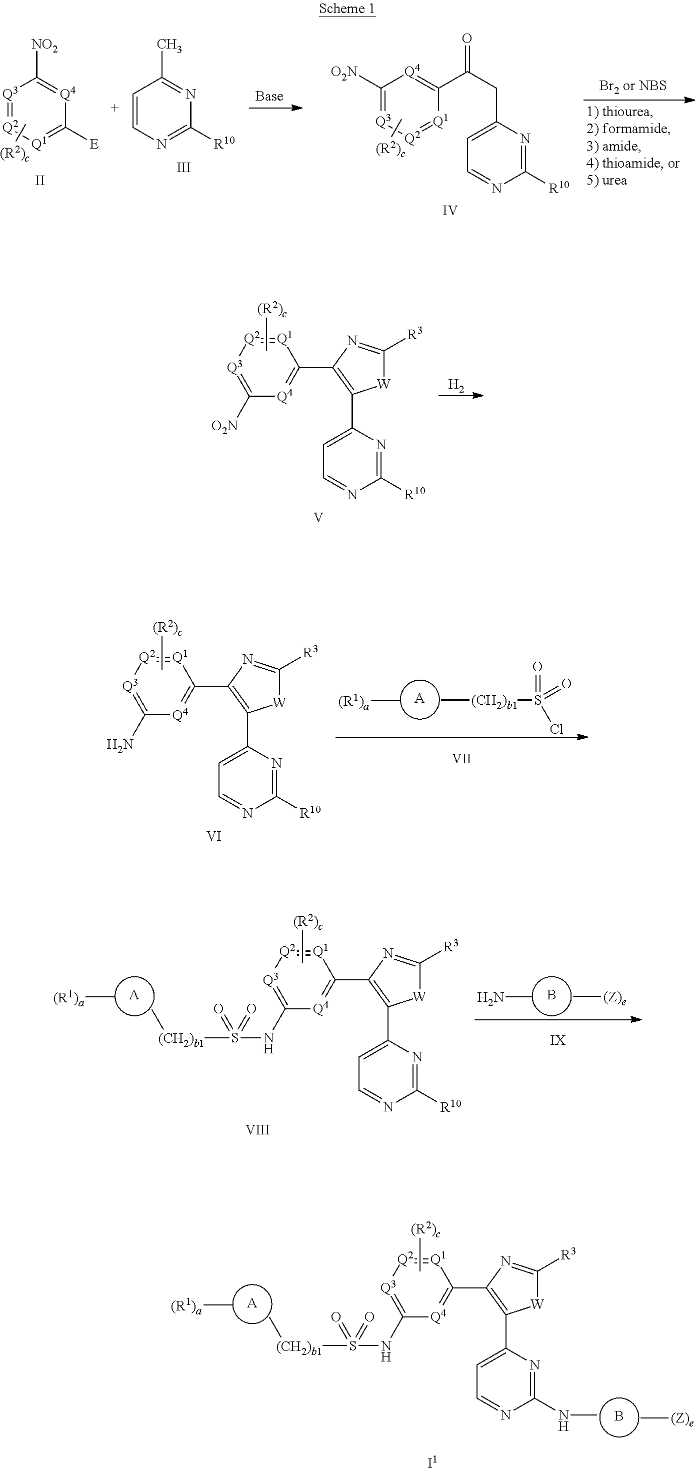

wherein:
R¹⁰ is halo (preferably chloro) or thiomethyl;
E is a suitable carboxylic ester or ester equivalent, particularly a methyl ester, ethyl ester, or Weinreb's amide; and
   all other variables are as defined above.

In this and subsequent synthetic routes, NBS is N-bromosuccinamide.

Generally, the process for preparing the compounds of formula (I) (all formulas and all variables having been defined above) comprises the step of: reacting a compound of formula (VIII) with an aniline of formula (IX) to prepare a compound of formula (I), wherein b2 is 0.

More specifically, the process for preparing compounds of the invention, comprises the steps of:
a) condensing the compound of formula (II) with a substituted pyrimidine compound of formula (III) to prepare a compound of formula (IV);
b) reacting the compound of formula (IV) with a suitable brominating agent followed by one of:
   i) a thiourea,
   ii) a formamide,
   iii) an amide,
   iv) a thioamide, or
   v) a urea;
   to prepare a compound of formula (V);
c) reducing the compound of formula (V) to prepare a compound of formula (VI);
d) reacting a compound of formula (VI) with a compound of formula (VII) to prepare a compound of formula (VIII);
e) reacting the compound of formula (VIII) with an aniline of formula (IX) to prepare a compound of formula (I¹);
f) optionally converting the compound of formula (I¹) to a pharmaceutically acceptable salt thereof; and
g) optionally converting the compound of formula (I¹) or a pharmaceutically acceptable salt thereof to a different compound of formula (I) or a pharmaceutically acceptable salt thereof.

As will be apparent to those skilled in the art, the order of the foregoing steps is not critical to the process of the present invention, and the process may be carried out using any suitable order of steps.

Compounds of formula (I) wherein b2 is 0 (i.e., compounds of formula (I¹) are prepared by reacting a compound of formula (VIII) with an aniline of formula (IX).

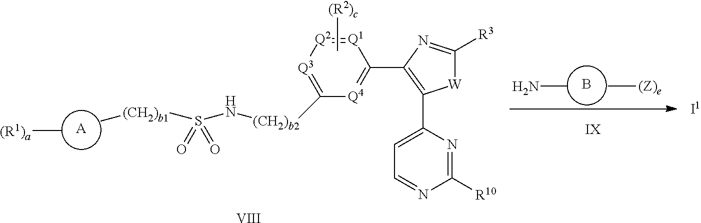

wherein all variables are as defined above.

Those skilled in the art will recognize that the conditions required for the above reaction will differ depending upon the definition of R¹⁰. When R¹⁰ is halo (preferably chloro), the reaction is generally performed in a solvent. Suitable solvents include but are not limited to isopropanol, 1,4-dioxane, ethanol, dimethylacetamide, trifluoroethanol, and N,N-dimethylformamide. The reaction is typically carried out under reflux conditions or in a microwave apparatus at a temperature of from about 90 to about 220° C., preferably from about 160 to about 190° C. As will be apparent to those skilled in the art of organic chemistry, it may be desirable to catalyze this reaction for the preparation of certain compounds of formula (I). For example, for compounds of formula (IX) and (I), wherein e is 0 and Ring B is aryl, it may be desirable to carry out the reaction in the presence of a catalytic amount of an acid such as hydrochloric acid, hydrobromic acid or para toluenesulfonic acid. As will further be apparent to those skilled in the art, it may also be desirable to install appropriate protecting groups prior to reacting the compound of formula (VIII) with the compound of formula (IX). For example, in the embodiment, wherein Z is a group containing a primary or secondary amine, the addition is preferably carried out when the amine is protected as, for example, its corresponding trifluoroacetamide. The choice, installation and removal of appropriate protecting groups for reactions such as this is conventional in the art. Compounds of formula (IX) are commercially available or may be synthesized using techniques conventional in the art.

When R¹⁰ is thiomethyl, the thiomethyl may first be converted to a more suitable leaving group, for example sulfoxide, sulfone, or chloride. The thiomethyl can be converted into a sulfoxide or sulfone by oxidation with an appropriate oxidizing agent, for example oxone, sodium periodate, or meta-chloroperbenzoic acid, in an appropriate solvent, for example dichloromethane, methanol, or water. Those skilled in the art will recognize that this will produce an analogue of the compound of formula (VIII) in which R¹⁰ is a sulfoxide or sulfone. The oxidized product can then be reacted with an aniline of formula (IX) to generate a compound of formula (I).

These reactions are generally performed in a suitable solvent, for example 2-propanol, dimethylacetamide, or dioxane, optionally with the addition of acid, for example hydrochloric acid, and at a temperature of 25-110° C., preferably 70-90° C., or in a microwave reactor at a temperature of 90-220° C., preferably 160-190° C.

Alternately, the pyrimidinyl sulfoxide or sulfone can be converted to the corresponding hydroxyl pyrimidine by reaction with an appropriate aqueous acid, for example hydrochloric acid or acetic acid, at a temperature of 25-110° C., preferably 70-90° C. The hydroxyl pyrimidine can then be converted to a chloride using an appropriate chlorinating reagent, for example phosphorous oxychloride or thionyl chloride, optionally in a solvent, for example dichloromethane, at a temperature of 25-120° C., preferably 60-80° C. Those skilled in the art will recognize that this process will produce a compound of formula (VIII) wherein R¹⁰ is chloro, which can be reacted with an aniline of formula (IX) as described above.

Compounds of formula (VIII) may be prepared by reacting a compound of formula (VI) with any of a compound of formula (VII).

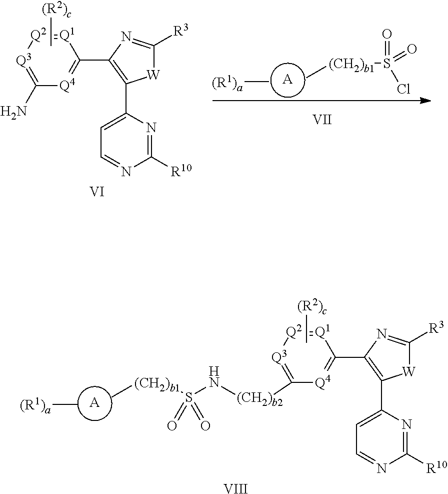

wherein all variables are as defined above in Scheme 1.

Compounds of formula (VIII) may be prepared by reacting a compound of formula (VI) with a compound of formula (VII). This reaction may be carried out using conditions conventional in the art for such coupling reactions, including the use of a solvent such as tetrahydrofuran, 1,4-dioxane or dichloromethane at ambient temperature or with heating from about 40° C. to about 100° C. Those skilled in the art will recognize that it may be desirable to carry out this reaction in the presence of a suitable base, for example triethylamine. Compounds of formula (VII) are commercially available or may be synthesized using techniques conventional in the art.

Compounds of formula (VI) may be prepared by reducing a compound of formula (V).

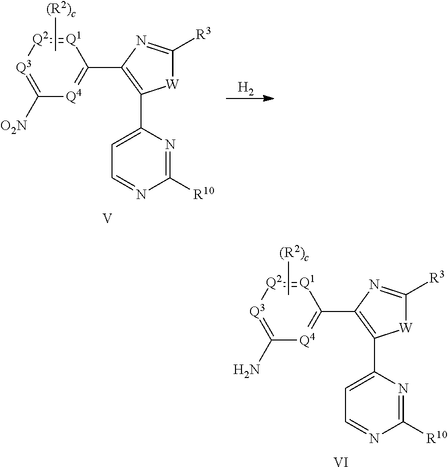

wherein all variables are as defined above in Scheme 1.

Those skilled in the art will recognize that this reaction may be accomplished in several ways. One process for the reduction of a compound of formula (V) involves the treatment of a compound of formula (V) with an atmosphere of hydrogen gas at about 14-100 psi, preferably about 30-50 psi in a suitable solvent, such as ethanol or methanol, and in the presence of a suitable catalyst, for example, platinum on carbon, palladium on carbon, or sulfided platinum on carbon. Another process for reducing a compound of formula (V) involves treating a compound of formula (V) with a suitable reducing agent such as sodium sulfide or tin tetrachloride, in a suitable solvent, for example, ethanol or tetrahydrofuran, optionally with the addition of a suitable acid, for example hydrochloric acid at a temperature of 25-100° C., particularly 50-70° C. Those skilled in the art will recognize that some reduction conditions include reagents, for example ethanol, that may also react with R¹⁰, for example when R¹⁰ is chloro. Those skilled in the art will also recognize that other reduction conditions described above or known to those of skill can be selected in such instances.

Compounds of formula (V) may be prepared by reacting a compound of formula (IV) with a suitable brominating reagent, particularly bromine or NBS, followed by reacting with one of: 1) a thiourea, 2) a formamide 3) an amide 4) a thioamide or 5) a urea depending upon whether the thiazole or oxazole and which particular substituents R³, are desired.

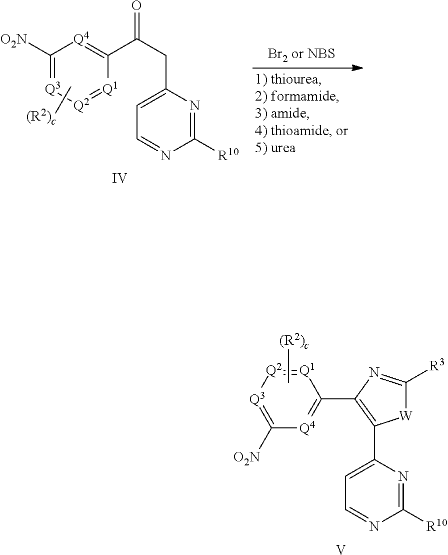

wherein all variables are as defined above.

In this and subsequent Schemes, reference to thiourea, formamide, amide, thioamide or urea in connection with this type of reaction refers to unsubstituted thiourea, formamide, amide, thioamide or urea and substituted analogs thereof. In particular, the thiourea, formamide, amide, thioamide or urea may be substituted with the desired group R³. Suitably substituted analogs of thiourea, formamide, amide, thioamide or urea are commercially available or may be prepared using conventional techniques.

When an aminothiazole (i.e., the compound of formula (V) wherein W is S and R³ is selected from —NR⁶R⁷, —N(R⁶)—C₃₋₆cycloalkyl, —N(R⁶)Het, —N(R⁶)R⁵Het, —N(R⁶)—R⁵—OR⁷, —N(R⁶)—R⁵NR⁶R⁷, —N(H)C(O)R⁶, —N(R⁶)—C(O)—NR⁶R⁷, —N(H)SO₂R⁶, —N(R⁶)—R⁵—S(O)₂R⁷, and —N(R⁶)—S(O)₂—NR⁶R⁷, is desired, the reaction can be accomplished by the initial bromination of a compound of formula (IV) using an appropriate brominating reagent, for example bromine in solvent such as acetic acid or NBS.

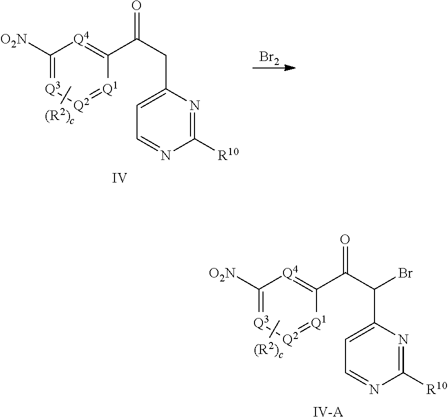

The reaction is typically carried out in an appropriate solvent, for example dichloromethane, N,N-dimethylformamide, or N,N-dimethylacetamide, and at a temperature of 25-50° C., particularly 25° C. The brominated analog (i.e., a compound of formula (IV-A)) is then reacted with an appropriately substituted thiourea.

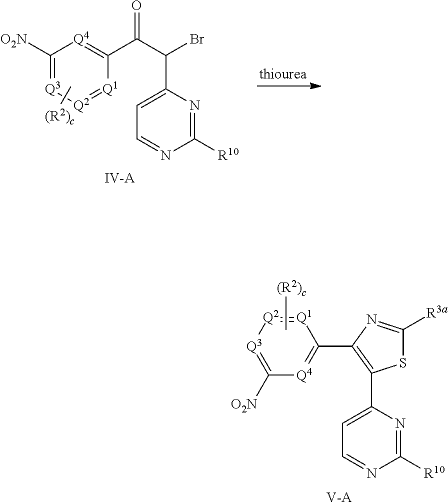

wherein R^{3a} is selected from —NR⁶R⁷, —N(R⁶)—C₃₋₆cycloalkyl, —N(R⁶)Het, —N(R⁶)R⁵Het,
N(R⁶)—R⁵—OR⁷, —N(R⁶)—R⁵NR⁶R⁷, —N(H)C(O)R⁶, —N(R⁶)—C(O)—NR⁶R⁷, —N(H)SO₂R⁶,
—N(R⁶)—R⁵—S(O)₂R⁷, and —N(R⁶)—S(O)₂—NR⁶R⁷;
   and all other variables are as defined above.

The reaction is typically carried out in an appropriate solvent, for example, dichloromethane, THF, dioxane, or acetonitrile, optionally in the presence of a suitable base, for example magnesium carbonate or sodium bicarbonate, and at a temperature of 25-90° C., particularly 25-50° C. Those skilled in the art will recognize that the thiourea can be unsubstituted, thus resulting in a compound of formula (V-A) wherein R³ is NH₂, or the thiourea may bear one or more additional substituents on one of the nitrogen atoms, for example as in N-[2-(4-morpholinyl)ethyl]thiourea.

In this and subsequent reactions, a compound, such as a compound of formula (V), wherein R³ is an amino group (or substituted amino), may be further converted to a corresponding compound wherein R³ is other than amino (or substituted amino) using the techniques described herein and those conventional in the art.

For example, the aminothiazole compound of formula (V-A) prepared according to the preceding description may be converted to an unsubstituted thiazole (i.e., a compound of formula (V) wherein R³ is H) using methods familiar to those of skill in the art. For example, the thiazole may be prepared by reacting the aminothiazole with an appropriate reagent, for example t-butyl nitrite, in an appropriate solvent, for example tetrahydrofuran, and at a temperature of 35-75° C., particularly 40-60° C.

When a substituted thiazole is desired, an aminothiazole of formula (V-A) may be modified according to methods that will be familiar to those skilled in the art. For example, the aminothiazole compound of formula (V-A) may be converted to a compound of formula (V-B) by reaction with reagents capable of replacing the amino group with a halide, preferably a bromide.

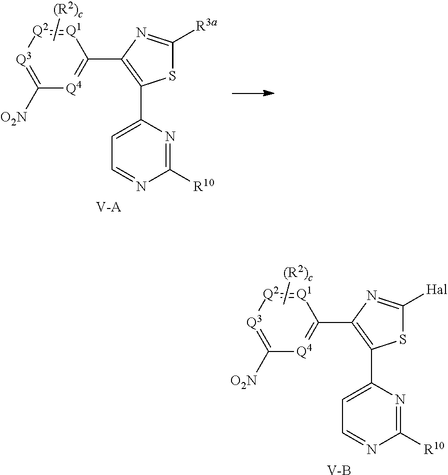

wherein Hal is halo, preferably Br; and all other variables are as defined above.

The conversion to a halo-thiazole of formula (V-B) may be carried out by reaction with for example, t-butyl nitrite and copper (II) bromide in a suitable solvent, such as tetrahydrofuran or acetonitrile, and at a temperature from −10° C. to 50° C., preferably 0° C. to 25° C. The halo-thiazole of formula (V-B), may then be reacted under a variety of conditions known to those in the art to produce different thiazole compounds of formula (V) wherein R³ can be a variety of substituents consistent with the definition of R³ in reference to compounds of Formula (I).

One example of such a reaction is similar to the method of J. Tsuji “Palladium Reagents and Catalysts: Innovations in Organic Synthesis”, Wiley, Chichester, UK, 1995, involving reaction of the halo-thiazole of formula (V-B) with a reagent capable of undergoing palladium-based coupling to prepare compounds of formula (V-C) wherein R^{3c} is alkyl, haloalkyl, or alkenyl.

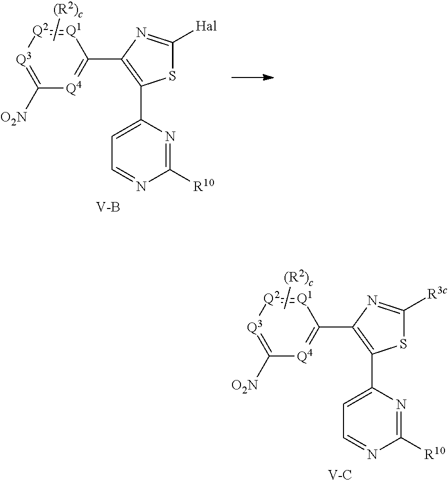

wherein Hal is halogen;
R^{3c} is alkyl, haloalkyl or alkenyl; and
all other variables are as defined above.

For example the halo-thiazole of formula (V-B) may be reacted with a boronic acid, boronate ester, alkyl tin, alkyl zinc or Grignard reagent, in an appropriate solvent, for example tetrahydrofuran, dioxane, or dimethylformamide, in the presence of a catalyst capable of inducing such a transformation, particularly a palladium catalyst, for example palladiumdicholorobistriphenylphosphine, and at a temperature of 25-150° C., preferably 25-60° C. Those skilled in the art will recognize that these coupling reactions will often require the addition of a suitable base, such as aqueous sodium carbonate, cesium carbonate, or triethylamine and/or the addition of a suitable ligand for the palladium species, for example a trialkylphosphine or a triarylphosphine, for example triphenylphosphine.

Another example of such a reaction involves the reaction of the halo-thiazole of formula (V-B) with a reagent capable of displacing the bromide, for example an amine, such as piperidine, methylamine, or methyl piperazine.

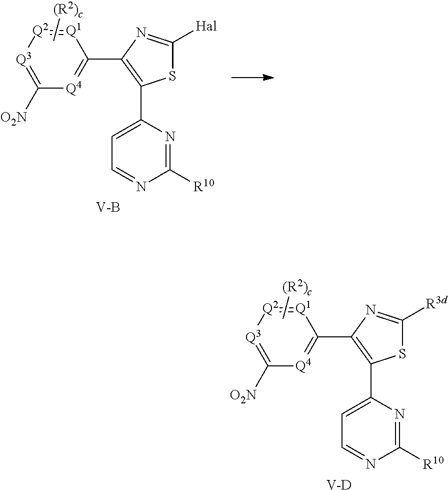

wherein Hal is halogen;
R^{3d} is selected from, —NR⁶R⁷, —N(R⁶)—C₃₋₆cycloalkyl, —N(R⁶)—R⁵—OR⁷, —N(R⁶)—R⁵—NR⁶R⁷ and —N(R⁶)—R⁵—S(O)₂R⁷;
all other variables are as defined above.

In the case of reacting a halo-thiazole of formula (V-B) with an amine, substituted amine (e.g., dimethylamine) or N-heterocycle (e.g., morpholine or N-methyl piperidine, the reaction is generally performed by reacting the compound of formula (V-B) with the amine, substituted amine or N-heterocycle, optionally in a suitable solvent, such as 2-propanol, dioxane, or dimethylformamide, at a temperature of 25° C. to 150° C., preferably 50-90° C., optionally in the presence of a suitable acid, for example hydrochloric acid.

According to another process of producing a substituted thiazole of formula (V), a compound of formula (IV-A) is reacted with a thioamide, for example thioacetamide, to prepare a compound of formula (V-E) wherein R^{3e} is alkyl.

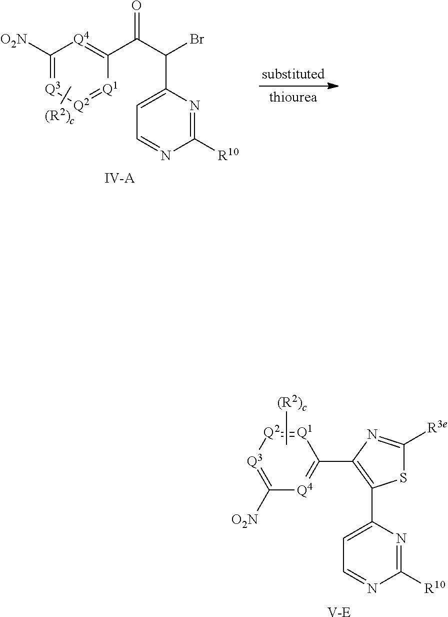

wherein all variables are as defined above.

Alkyl substituted thioamides for use in this process are commercially available or may be prepared using conventional techniques. Typically, the reaction is carried out in an appropriate solvent, for example, dichloromethane, tetrahydrofuran, dimethylformamide, N,N-dimethylacetamide, or acetonitrile, particularly dimethylformamide or N,N-dimethylacetamide, optionally in the presence of a suitable base, for example magnesium carbonate or sodium bicarbonate, and at a temperature of 35-100° C., preferably 50-70° C.

In the embodiment wherein an oxazole of formula (V) is desired wherein R³ is H, the reaction can be accomplished by reacting the compound of formula (IV-A) with formamide in the presence of an acid, such as sulfuric acid, and at a temperature of 60-150° C., preferably 100-130° C.

A substituted oxazole of formula (V-F) may be prepared from the compound of formula (IV-A).

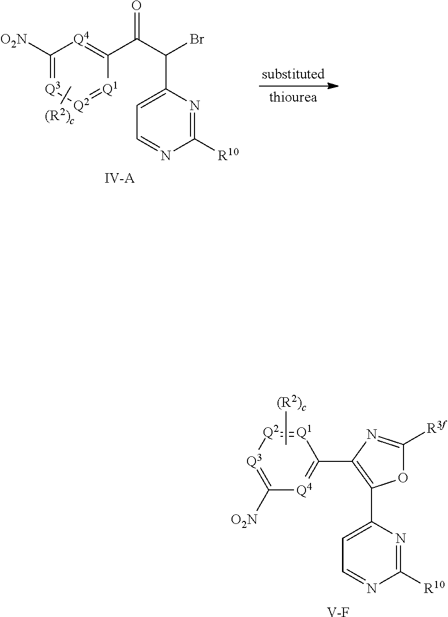

wherein R^{3f} is selected from —NR⁶R⁷, —N(R⁶)—C₃₋₆cycloalkyl, —N(R⁶)Het, —N(R⁶)R⁵—Het, —N(R⁶)—R⁵—OR⁷, —N(R⁶)—R⁵—NR⁶R⁷, —N(H)C(O)R⁶, —N(R⁶)—C(O)—NR⁶R⁷, —N(H)SO₂R⁶, —N(R⁶)—R⁵—S(O)₂R⁷, and —N(R⁶)—S(O)₂—NR⁶R⁷; and
   all other variables are as defined above.

The reaction may be carried out by reacting the compound of formula (IV-A) with a urea or substituted urea in an appropriate solvent, for example, dichloromethane, tetrahydrofuran, dioxane, or acetonitrile, optionally in the presence of a suitable base, for example magnesium carbonate or sodium bicarbonate, and at a temperature of 25-170° C., particularly 60-150° C. or in a microwave reactor at a temperature of 100-190° C., particularly 120-160° C. Those skilled in the art will envision substituted ureas that may be employed in the foregoing method to prepare compounds of formula (V-F) wherein R^{3f} is as defined above. One example of a substituted urea for use in this method is N-[2-(4-morpholinyl)ethyl]urea. Suitable substituted ureas are commercially available or can be made using techniques known to those skilled in the art.

A substituted oxazole of formula (V-G), may also be prepared from a compound of formula (IV-A).

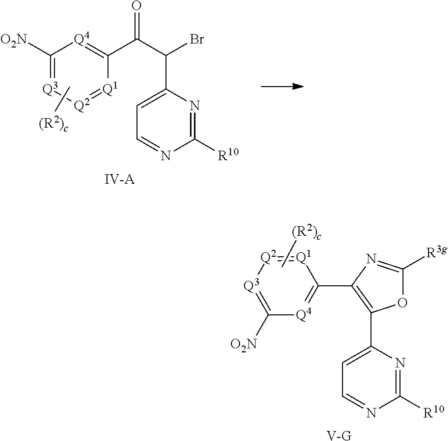

wherein R^{3g} is alkyl or haloalkyl and all other variables are as defined above.

Typically, the reaction may be carried out by reacting the compound of formula (IV-A) with an amide (i.e., a compound of formula R^{3g}—C(O)NH₂), for example acetamide, in an appropriate solvent, for example, dichloromethane, tetrahydrofuran, dimethylformamide, or acetonitrile, particularly dimethylformamide, optionally in the presence of a suitable base, for example magnesium carbonate or sodium bicarbonate, and at a temperature of 35-170° C., preferably 60-150° C. or in a microwave reactor at a temperature of 100-190° C., particularly 130-170° C. Suitable amides for use in this reaction will be apparent to those skilled in the art and are commercially available or may be prepared using conventional techniques.

As will be appreciated by those skilled in the art a bromo-substituted oxazole of formula (V-H),

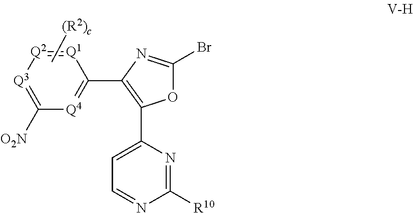

wherein all other variables are as defined above;
   may also be prepared by conversion of an oxazole of formula (V-F) (wherein R³ is an amine or substituted amino group) to the bromo analog using techniques known to those of skill in the art, including those described above.

Those of skill in the art will recognize that some of the reactions described above may be incompatible with compounds of formula (V) in which R¹³ is chloride. In such embodiments, the foregoing reactions may be performed using compounds of formula (V) wherein R¹³ is thiomethyl, and subsequently converting the thiomethyl to a more suitable leaving group, such as a sulfoxide, sulfone or chloride using techniques conventional in the art, including those described above.

Compounds of formula (IV) may be prepared by reacting a compound of formula (II) with a substituted pyrimidine of formula (III).

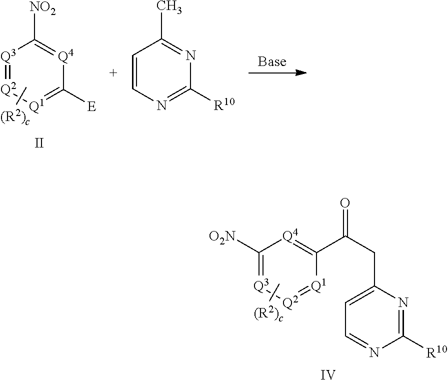

wherein all variables are as defined above.

These reactions are generally performed by reacting a compound of formula (II) and a compound of formula (III) in the presence of a suitable base capable of deprotonating a compound of formula (III), for example lithium hexamethyldisilazide (LHMDS), sodium hexamethyldisilazide, or lithium diisopropylamide, particularly LHMDS, in an appropriate solvent, such as THF, and at a temperature of from about −78° C. to about 25° C., particularly about 0° C. to about 25° C.

As noted above, the order of the foregoing steps is not critical to the practice of the present invention. In another embodiment, compounds of the invention may also be prepared according to Scheme 2.

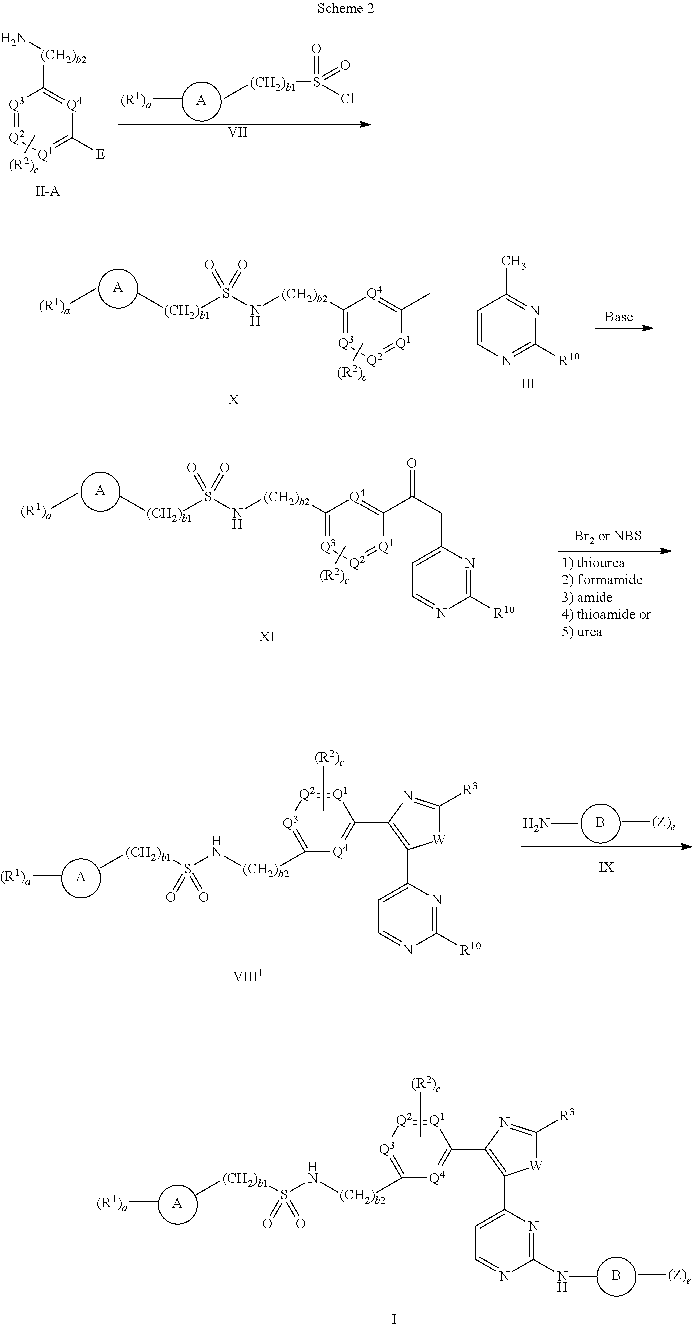

wherein:
b2 is 0 or 1;
R¹⁰ is halo (preferably chloro) or thiomethyl;
E is a suitable carboxylic ester or carboxylic ester equivalent, particularly a methyl ester, ethyl ester, or Weinreb's amide; and
all other variables are as defined above.

The process for preparing the compounds of the invention according to Scheme 2 (all formulas and all variables having been defined above) comprises the steps of:
a) reacting a compound of formula (II-A) with a compound of formula (VII) to prepare a compound of formula (X);
b) condensing the compound of formula (X) with a substituted pyrimidine of formula (III) to prepare a compound of formula (XI);
c) reacting the compound of formula (XI) with a suitable brominating agent, followed by reacting with one of:
   i) a thiourea,
   ii) a formamide,
   iii) an amide,
   iv) a thioamide, or
   v) a urea;
   to prepare a compound of formula (VIII¹);
d) reacting the compound of formula (VIII¹) with an aniline of formula (IX) to prepare a compound of formula (I);
e) optionally converting the compound of formula (I) to a pharmaceutically acceptable salt thereof; and
f) optionally converting the compound of formula (I) or a pharmaceutically acceptable salt thereof to a different compound of formula (I) or a pharmaceutically acceptable salt thereof.

The order of the foregoing steps is not critical to the processes of the present invention and the process may be carried out using any suitable order of steps.

The reaction of the compounds of formula (VIII¹) with the aniline of formula (IX) is carried out in the same manner as described above for the reaction of compounds of formula (VIII) with the aniline of formula (IX).

According to this process, the compound of formula (VIII¹) may be prepared by reacting a compound of formula (XI) with a suitable brominating agent, particularly bromine or NBS, followed by reacting with one of a thiourea, a formamide, an amide, a thioamide, or a urea (including substituted analogs thereof) in the same manner as discussed above for the reaction of a compound of formula (IV).

The compounds of formula (XI) may be prepared by condensing a compound of formula (X) with a compound of formula (III) in the same manner as described above for the condensation of a compound of formula (II) with a compound of formula (III).

A compound of formula (X) may be prepared by reacting the compound of formula (II-A) with a compound of formula (VII) in the same manner as described above for the reaction of a compound of formula (VI) with a compound of formula (VII). Compounds of formula (II-A) may be prepared by reducing a compound of formula (II) using conventional reduction techniques, including those described above for the hydrogenation of a compound of formula (V).

As a further example, compounds of the invention wherein b1 and b2 are 0, may also be prepared according to Scheme 3.

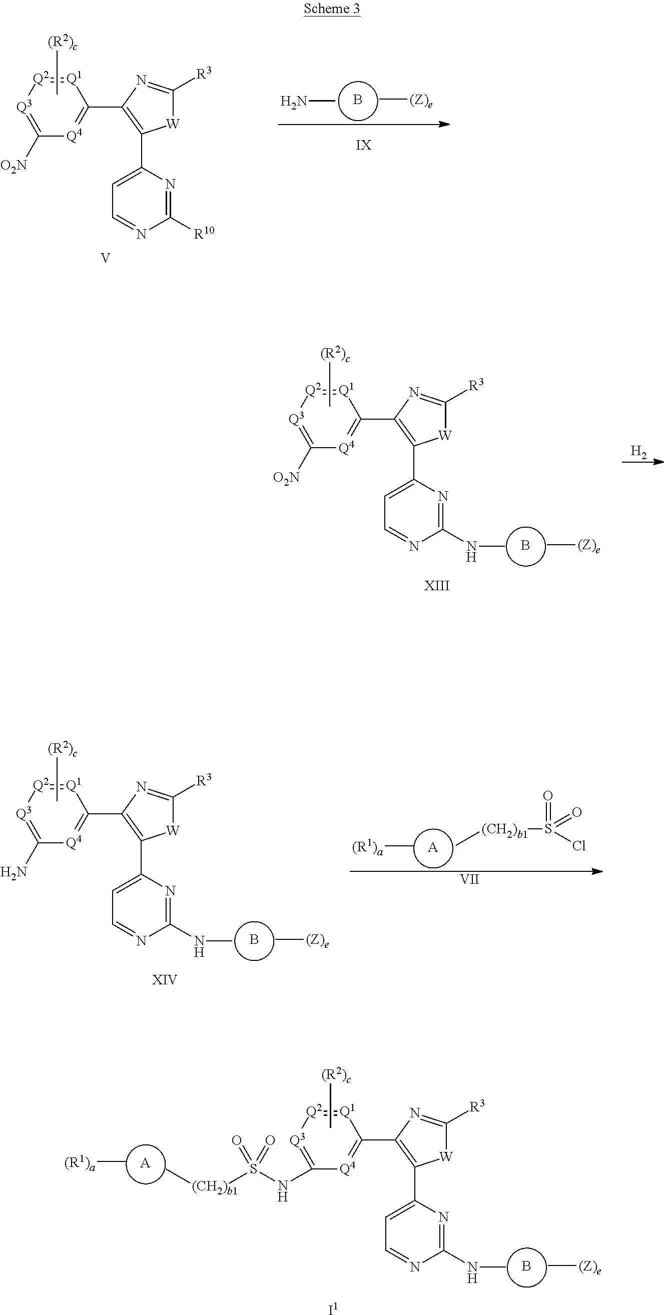

wherein R¹⁰ is halo (preferably chloro) or thiomethyl, b1 and b2 are 0, and all other variables are as defined above.

Generally, the process for preparing the compounds of the invention according to Scheme III (all formulas and all variables having been defined above) comprises the steps of:
a) reacting a compound of formula (V) with an aniline of formula (IX) to prepare a compound of formula (XIII);
b) reducing the compound of formula (XIII) to prepare a compound of formula (XIV);
c) reacting the compound of formula (XIV) with a compound of formula (VII) to prepare a compound of formula (I¹);
d) optionally converting the compound of formula (I¹) to a pharmaceutically acceptable salt thereof; and
e) optionally converting the compound of formula (I¹) or a pharmaceutically acceptable salt thereof to a different compound of formula (I) or a pharmaceutically acceptable salt thereof.

Each of the foregoing steps may be carried out using the techniques described above for analogous reactions with different starting materials. Thus, the reaction of the compounds of formula (V) with the aniline of formula (IX) is carried out in the same manner as discussed above for the reaction of a compound of formula (VIII) with the aniline of formula (IX); the reaction of the compound of formula (XIV) with a compound of formula (VII) is carried out in the same manner as the reaction of a compound of formula (VI) or a compound of formula (II-A) with a compound of formula (VII); and the reducing step may be carried out in the same manner as described for the reduction of a compound of formula (V).

Compounds of the invention, wherein b2 is 0, may also be conveniently prepared by the process outlined in Scheme 4 below.

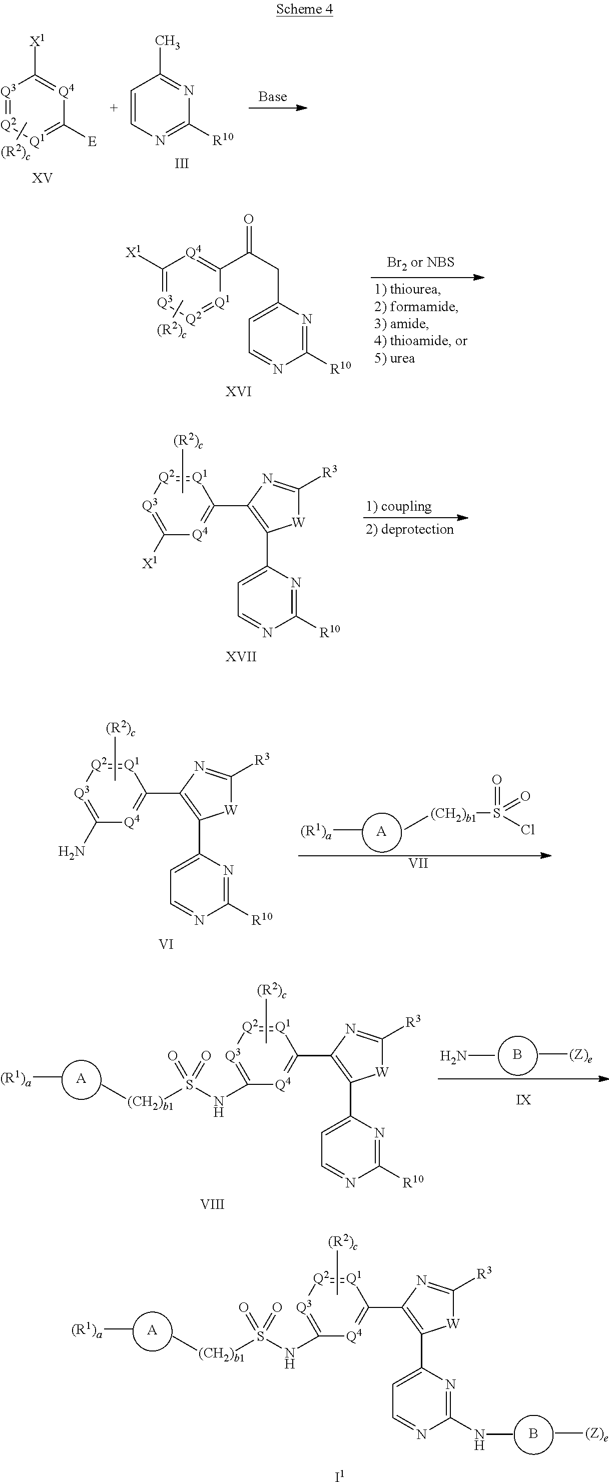

wherein:
R¹⁰ is halo (preferably chloro) or thiomethyl;
E is a suitable carboxylic ester or carboxylic ester equivalent, particularly a methyl ester, ethyl ester, or Weinreb's amide;
X¹ is a halo, preferably a bromo, and
all other variables are as defined above.

More specifically, the process for preparing compounds of the invention according to Scheme 4 comprises the steps of:
a) condensing the compound of formula (XV) with a substituted pyrimidine compound of formula (III) to prepare a compound of formula (XVI);
b) reacting the compound of formula (XVI) with a suitable brominating agent followed by one of:
   i) a thiourea,
   ii) a formamide,
   iii) an amide,
   iv) a thioamide, or
   v) a urea;
   to prepare a compound of formula (XVII);
c) reacting the compound of formula (XVII) with an ammonia equivalent in the presence of an appropriate catalyst, optionally with subsequent deprotection, to prepare a compound of formula (VI);
d) reacting a compound of formula (VI) with a compound of formula (VII) to prepare a compound of formula (VIII);
e) reacting the compound of formula (VIII) with an aniline of formula (IX) to prepare a compound of formula (I¹);
f) optionally converting the compound of formula (I¹) to a pharmaceutically acceptable salt thereof; and
g) optionally converting the compound of formula (I¹) or a pharmaceutically acceptable salt thereof to a different compound of formula (I) or a pharmaceutically acceptable salt thereof.

The order of the foregoing steps is not critical to the process of the present invention, and the process may be carried out using any suitable order of steps.

The reaction of the compounds of formula (VIII) with the aniline of formula (IX) and the reaction of compounds of formula (VI) with compounds of formula (VII) are described above.

Compounds of formula (VI) are prepared by the coupling reaction of a compound of formula (XVII) with an ammonia equivalent in the presence of an appropriate catalyst, optionally with subsequent deprotection. This reaction may be accomplished in several ways. One process will involve the reaction of compound of formula (XVII), wherein X¹ is a halo, preferably a bromide, with an ammonia equivalent, such as benzophenone imine, bis(1,1-dimethylethyl)imidodicarbonate or sodium azide, in the presence of a palladium or copper catalyst capable of inducing such a transformation, for example palladiumdicholorobistriphenyl-phosphine, tris(dibenzylideneacetone)dipalladium, copper acetate, or copper iodide and at a temperature of 25-150° C., preferably 25-60° C. Those skilled in the art will recognize that these coupling reactions will often require the addition of a suitable base, such as aqueous sodium carbonate, cesium carbonate, potassium carbonate, or triethylamine and/or the addition of a suitable ligand for the palladium, for example a trialkylphosphine or a triarylphosphine, for example triphenylphosphine, or a suitable ligand for the copper, for example sodium ascorbate or 2-acetylcyclohexanone.

Those of skill in the art will recognize that the inclusion of an ammonia equivalent, such as benzophenone imine or bis(1,1-dimethylethyl)imidodicarbonate, will require a deprotection to give a compound of formula (VI). The deprotection conditions will be selected from the conventional methods known in the art. For example, when benzophenone imine, bis(1,1-dimethylethyl)imidodicarbonate is used as the ammonia equivalent, deprotection can be accomplished by treatment with an aqueous acid, such as acetic acid or HCl at a temperature of 25° C. to 100° C. When sodium azide is used as the ammonia equivalent, reduction to a compound of formula (VI) can be accomplished by methods well known to those of skill in the art.

Those skilled in the art will recognize that the above reactions may be incompatible with compounds of formula (XVII) when R¹⁰ is a halo, for example, chloride, or may produce mixtures. In such embodiments, those of skill in the art will recognize that the foregoing reactions can be performed with a compound of formula (XVII) in which R¹⁰ is thiomethyl to produce a compound of formula (VI) wherein R¹⁰ is a thiomethyl, and subsequently converting to a compound of formula (VI) in which R¹⁰ is halo using procedures described above. Alternatively, those of skill will recognize that appropriate choice of reaction conditions can minimize the undesired reaction and that any mixtures can be separated using conventional methods.

The compound of formula (XVII) may be prepared by reacting a compound of formula (XVI) with a suitable brominating agent, particularly bromine or NBS, followed by reacting with one of a thiourea, a formamide, an amide, a thioamide, or a urea (including substituted analogs thereof) in the same manner as discussed above for the reaction of a compound of formula (IV).

The compounds of formula (XVI) may be prepared by condensing a compound of formula (XV) with a compound of formula (III) in a the same manner as described above for the condensation of a compound of formula (II) with a compound of formula (III). The compounds of formula (XV) are commercially available or may be prepared using techniques conventional in the art.

Compounds of the invention, wherein b2 is 0, may also be conveniently prepared by the process outlined in Scheme 5.

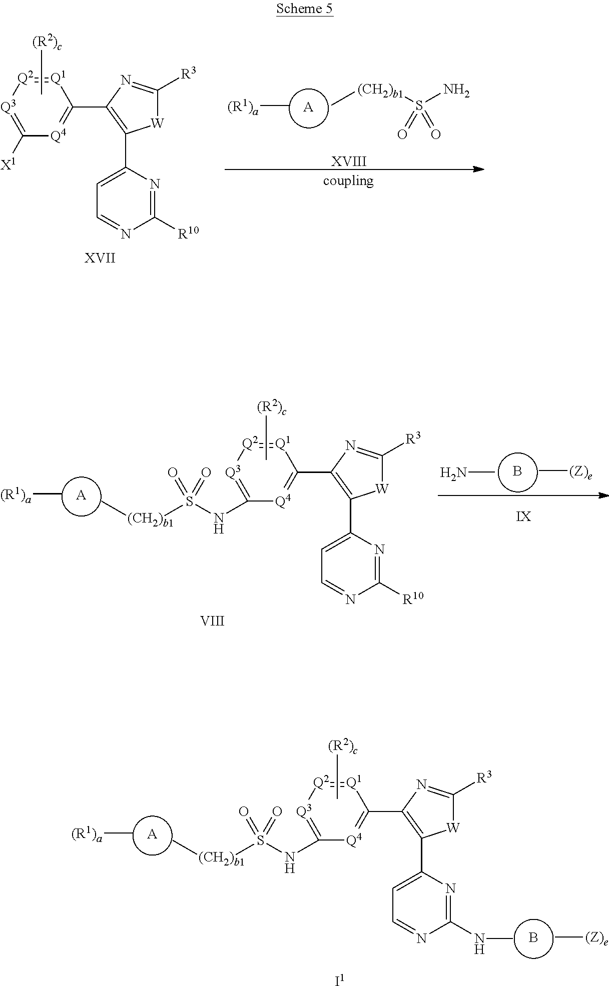

wherein:
R¹⁰ is halo (preferably chloro) or thiomethyl;
X¹ is a halo, preferably a bromo, and
all other variables are as defined above.

More specifically, the process for preparing compounds of the invention comprises the steps of:
a) reacting the compound of formula (XVII) with a compound of formula (XVIII) in the presence of an appropriate catalyst, to prepare a compound of formula (VIII);
b) reacting the compound of formula (VIII) with an aniline of formula (IX) to prepare a compound of formula (I¹);
c) optionally converting the compound of formula (I¹) to a pharmaceutically acceptable salt thereof; and
d) optionally converting the compound of formula (I¹) or a pharmaceutically acceptable salt thereof to a different compound of formula (I) or a pharmaceutically acceptable salt thereof.

The order of the foregoing steps is not critical to the process of the present invention, and the process may be carried out using any suitable order of steps.

The reaction of the compounds of formula (VIII) with the aniline of formula (IX) is described above.

Compounds of formula (VIII) may be prepared by coupling a compound of formula (XVII) with a compound of formula (XVIII). One process will involve the reaction of compound of formula (XVII), wherein X¹ is a halo, preferably a bromide, with a compound of formula (XVIII) in the presence of a palladium or copper catalyst capable of inducing such a transformation, for example palladiumdicholorobistriphenylphosphine, tris(dibenzylideneacetone)dipalladium, copper acetate, or copper iodide and at a temperature of 25-150° C., preferably 25-60° C. Those skilled in the art will recognize that these coupling reactions will often require the addition of a suitable base, such as aqueous sodium carbonate, cesium carbonate, potassium carbonate, or triethylamine and/or the addition of a suitable ligand for the palladium, for example a trialkylphosphine or a triarylphosphine, for example triphenylphosphine, or a suitable ligand for the copper, for example sodium ascorbate or 2-acetylcyclohexanone.

Those skilled in the art will recognize that the above reactions may be incompatible with compounds of formula (XVII) when R¹⁰ is a halo, for example, chloride, or may produce mixtures. In such embodiments, those of skill in the art will recognize that the foregoing reactions can be performed with a compound of formula (XVII) in which R¹⁰ is thiomethyl to produce a compound of formula (VIII) where in R¹⁰ is a thiomethyl, and subsequently converting to a compound of formula (VIII) in which R¹⁰ is halo using procedures described above. Alternatively, those of skill will recognize that appropriate choice of reaction conditions can minimize the undesired reaction and that any mixtures can be separated using conventional methods.

Compounds of formula (XVIII) are commercially available or may be prepared using conventional techniques.

Processes for preparing compounds of formula (XVII) are described above.

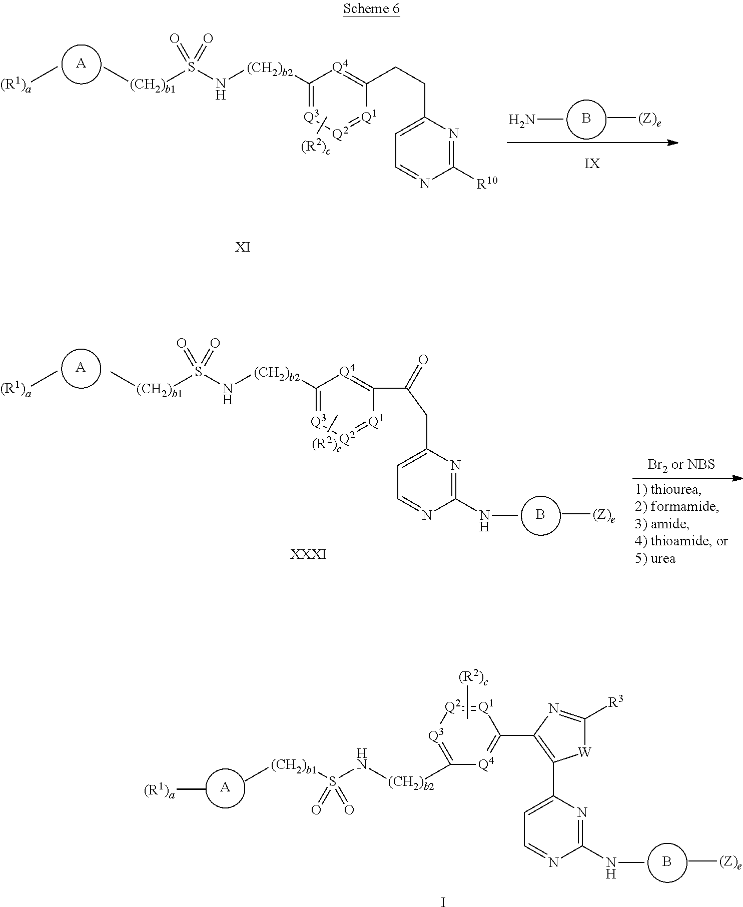

wherein:
R¹⁰ is halo (preferably chloro) or thiomethyl; and
all other variables are as defined above.

In general, the process for preparing compounds of the invention according to Scheme 6 comprises the steps of:

a) reacting the compound of formula (XI) with an aniline of formula (IX) to prepare a compound of formula (XXXI);
b) reacting the compound of formula (XXXI) with a suitable brominating agent, particularly bromine or NBS, followed by reaction with one of:
i) a thiourea,
ii) a formamide,
iii) an amide,
iv) a thioamide, or
v) a urea;
   to prepare a compound of formula (I);
   c) optionally converting the compound of formula (I) to a pharmaceutically acceptable salt thereof; and
   d) optionally converting the compound of formula (I) or a pharmaceutically acceptable salt thereof to a different compound of formula (I) or a pharmaceutically acceptable salt thereof.

As will be apparent to those skilled in the art, the route demonstrates a variation in the order of the steps of a previously described reaction, and thus, the process may be carried out using any suitable order of steps.

The compound of formula (XXXI) is reacted with a suitable brominating agent, particularly bromine or NBS, followed by reaction with one of a thiourea, a formamide, an amide, a thioamide, or a urea (including substituted analogs thereof), to prepare a compound of formula (I) in a manner analogous to the procedure described above for the reaction of the compound of formula (IV) to prepare a compound of formula (V).

The compound of formula (XI) is reacted with an aniline of formula (IX) to prepare the compound of formula (XXXI) in a procedure analogous to the reaction of the compound of formula (VIII) with an aniline of formula (IX) to prepare the compound of formula (I). It will be recognized by those of skill in the art that the aniline of formula (IX) might also condense with the ketone functionality of a compound of formula (XI) to form the corresponding imine or enamine. A compound of formula (XXXI) can be readily produced from the corresponding imine or enamine by reaction with an appropriate acid, such as acetic acid or HCl, optionally in a solvent such as dichloromethane or tetrahydrofuran and at a temperature of about 0° C. to about 50° C., preferably at ambient temperature.

Compounds of formula (XI) may be prepared as described above.

In another embodiment, compounds of the invention may be prepared according to the process outlined in Scheme 7.

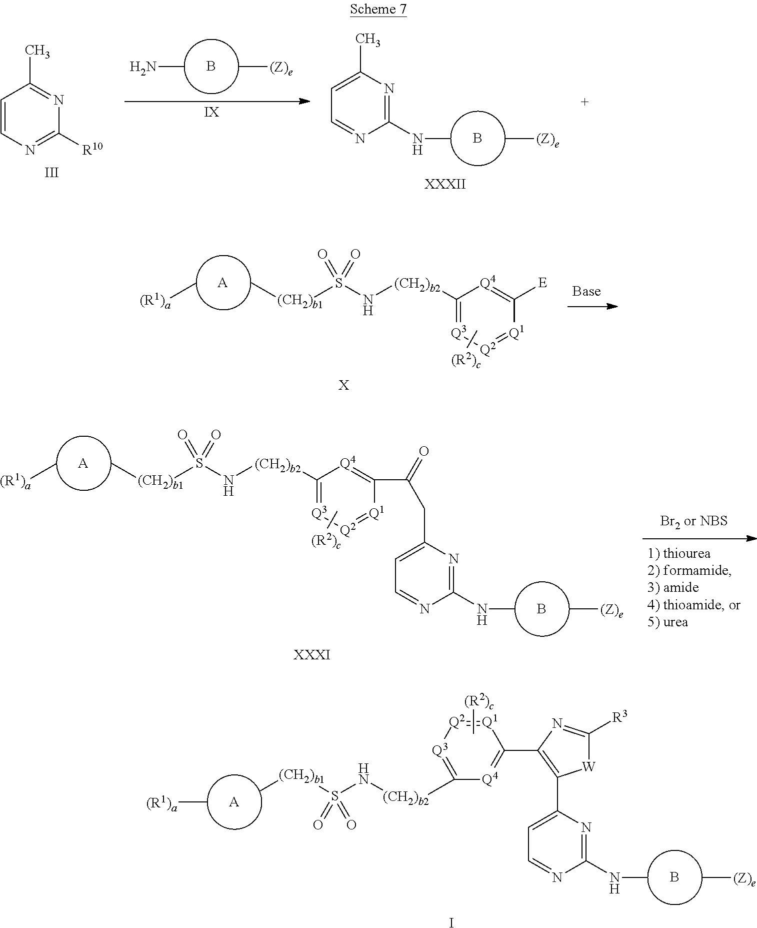

wherein R¹⁰ is halo (preferably chloro) or thiomethyl; and all other variables are as defined above.

In general, the process for preparing compounds of the invention according to Scheme 7 comprises the steps of:

a) reacting the compound of formula (III) with an aniline of formula (IX) to prepare a compound of formula (XXXII);
b) condensing the pyrimidine of formula (XXXII) with a compound of formula (X) to prepare a compound of formula (XXXI);
c) reacting the compound of formula (XXXI) with a suitable brominating agent followed by reaction with one of:
i) a thiourea,
ii) a formamide,
iii) an amide,
iv) a thioamide, or
v) a urea;
   to prepare a compound of formula (I);
   d) optionally converting the compound of formula (I) to a pharmaceutically acceptable salt thereof; and
   e) optionally converting the compound of formula (I) or a pharmaceutically acceptable salt thereof to a different compound of formula (I) or a pharmaceutically acceptable salt thereof.

As will be apparent to those skilled in the art, the order of the foregoing steps is not critical to the process of the present invention, and the process may be carried out using any suitable order of steps.

The compound of formula (XXXI) is reacted with a suitable brominating agent, particularly bromine or NBS, followed by reaction with one of a thiourea, a formamide, an amide, a thioamide, or a urea (including substituted analogs thereof), to prepare a compound of formula (I) in a manner analogous to the procedure described above for the reaction of the compound of formula (IV) to prepare a compound of formula (V).

The compound of formula (XXXI) is prepared by condensing the compound of formula (X) with a substituted pyrimidine of formula (XXXII). This reaction may be carried out in the same manner as described above and as for the condensation of a compound of formula (II) with a substituted pyrimidine of formula (III). Those of skill in the art will recognize that this reaction may require additional base. The preparation of a compound of formula (X) is described above.

The pyrimidine of formula (III) is reacted with an aniline of formula (IX) to prepare the compound of formula (XXXII) in a procedure analogous to the reaction of the compound of formula (VIII) with an aniline of formula (IX) to prepare the compound of formula (I).

It will be appreciated by those skilled in the art that the optimal choice of the reaction sequence employed to prepare a particular compound of the invention may depend upon the specific compound of the invention that is desired as well as the preference and availability of starting materials.

As will be apparent to those skilled in the art, a compound of formula (I) may be converted to another compound of formula (I) using techniques well known in the art. For example, compounds of formula (I) may be modified using conventional techniques to modify or diversify the groups defined by the variable R³ and there by provide different compounds of formula (I). Specifically, a compound of formula (I-1) (wherein R³ is

—NH₂) may be converted to a compound of formula (I-2) by reductive amination of the amine with acetone and sodium cyanoborohydride.

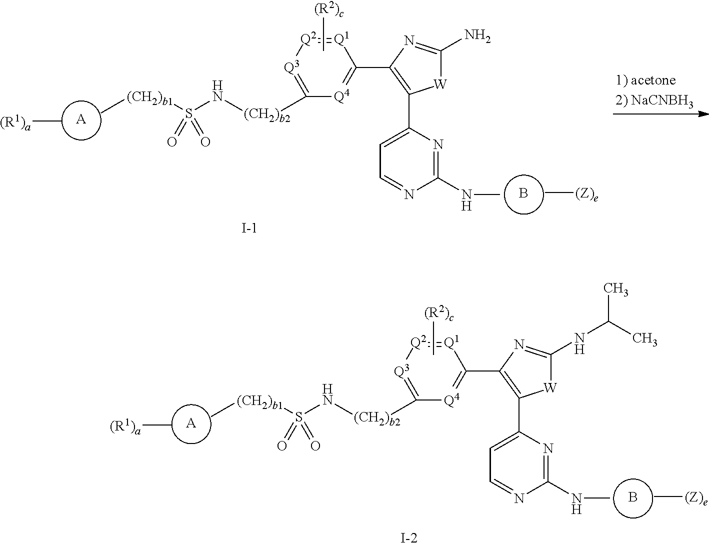

A compound of formula (I-1) may also be converted to a compound of formula (I-3) by reacting with mesyl chloride.

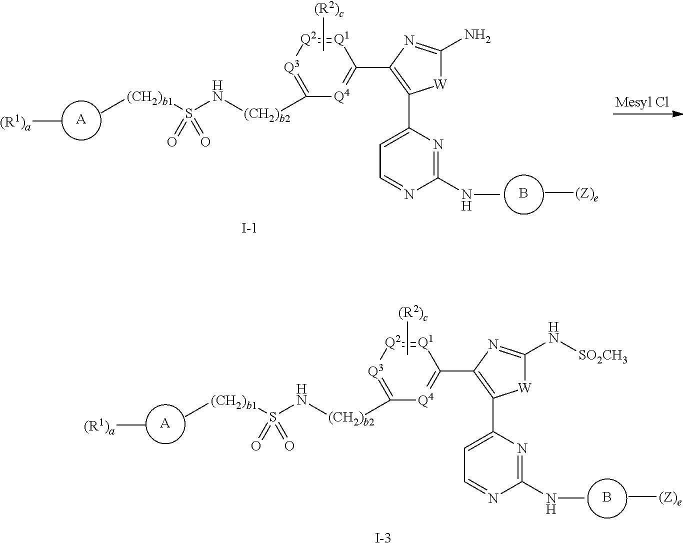

Compounds of formula (I) may also be diversified in the position defined by Z using conventional techniques to convert a compound of formula (I) to a different compound of formula (I). For example, a compound of formula (I-4) may be converted to a compound of formula (I-5) by reacting with morpholine.

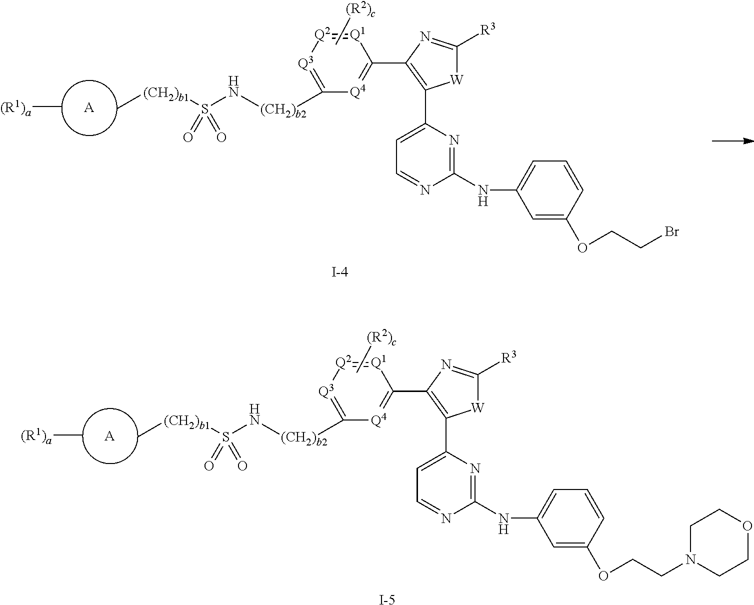

A compound of formula (I-6) may be converted to a compound of formula (I-7) by reacting with acetic anhydride.

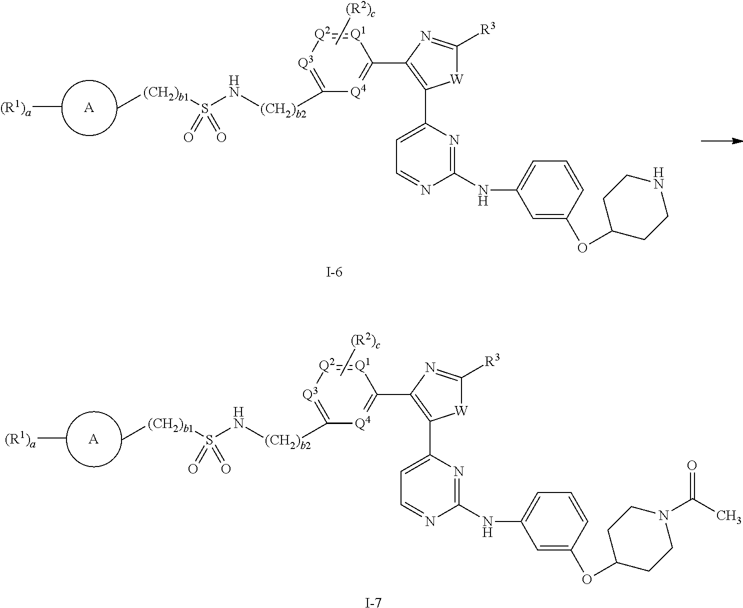

A compound of formula (I) wherein the moiety Ring B-(Z)ₑ is a tetrahydroisoquinoline group wherein the tetrahydroisoquinoline amine is a secondary amine may be converted into another compound of formula (I) wherein the amine is a tertiary amine bearing a methyl group. This transformation may be accomplished through a reductive amination procedure. Procedures for reductive amination are well known in the literature and include, for example, stirring the secondary amine bearing compound in a suitable solvent in the presence of aqueous formaldehyde and sodium triacetoxyborohydride and catalytic acid. Examples of suitable solvents include methylene chloride or N,N-dimethylformamide. An example of a suitable acid is acetic acid.

Based upon this disclosure and the examples contained herein one skilled in the art can readily convert a compound of formula (I) or a pharmaceutically acceptable salt thereof into a different compound of formula (I), or a pharmaceutically acceptable salt thereof.

The present invention also provides radiolabeled compounds of formula (I) and biotinylated compounds of formula (I) and solid-support-bound versions thereof, i.e. a compound of formula (I) having a radiolabel or biotin bound thereto. Radiolabeled compounds of formula (I) and biotinylated compounds of formula (I) can be prepared using conventional techniques. For example, radiolabeled compounds of formula (I) can be prepared by reacting the compound of formula (I) with tritium gas in the presence of an appropriate catalyst to produce radiolabeled compounds of formula (I). In one embodiment, the compounds of formula (I) are tritiated.

The radiolabeled compounds of formula (I) and biotinylated compounds of formula (I) are useful in assays for the identification of compounds which inhibit at least one Raf family kinase and/or at least one ErbB family kinase, for the identification of compounds for the treatment of a condition capable of being treated with a Raf inhibitor or an ErbB inhibitor, e.g., for the treatment of neoplasms susceptible to treatment with a Raf inhibitor or an ErbB inhibitor. The present invention also provides an assay method for identifying such compounds, which method comprises the step of specifically binding a radiolabeled compound of the invention or a biotinylated compound of the invention to the target protein or cellular homogenate. More specifically, suitable assay methods will include competition binding assays. The radiolabeled compounds of the invention and biotinylated compounds of the invention and solid-support-bound versions thereof, can also be employed in assays according to the methods conventional in the art.

The following examples are intended for illustration only and are not intended to limit the scope of the invention in any way. The invention is defined by the claims which follow.

### EXAMPLES

As used herein, the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
atm (atmosphere);
g (grams).
mg (milligrams)
h (hour(s));
min (minutes);
Hz (Hertz);
MHz (megahertz);
i.v. (intravenous);
L (liters);
mL (milliliters);
μl (microliters);
M (molar);
mM (millimolar);
mol (moles);
mmol (millimoles);
mp (melting point)
psi (pounds per square inch);
rt (room temperature);
TLC (thin layer chromatography);
Tᵣ (retention time);
RP (reverse phase;
H₂ (hydrogen);
N₂ (nitrogen);
Ac (acetyl);
ACN (acetonitrile);
Ac₂0 (acetic anhydride);
ATP (adenosine triphosphate);
BOC (tert-butyloxycarbonyl);
BSA (bovine serum albumin)
CHCI₃ (chloroform);
mCPBA (meta-chloroperbenzoic acid);
DCC (dicyclohexylcarbodimiimide);
DCE (dichloroethane);
DCM (CH₂Cl₂; dichloromethane)
DMA (dimethyl acetamide);
DMAP (4-dimethylaminopyridine)
DME (1,2-dimethoxyethane);
DMEM (Dulbecco's modified Eagle medium);
DMF (N;dimethylformamide);
DMSO (dimethylsulfoxide);
EDC (ethylcarbodiimide hydrochloride);
EDTA (ethylenediaminetetraacetic acid);
ET (ethyl;-CH₂CH₃);
EtOH (ethanol);
EtOAc (ethyl acetate);
FMOC (9-fluorenylmethoxycarbonyl);
HCI (hydrochloric acid);
HEPES (4-(2-hydroxyethyl)-1-piperazine ethane sulfonic acid);
HEX (hexanes);
HOAc (acetic acid);
HPLC (high pressure liquid chromatography);
i-PrOH (isopropanol);
K2CO₃ (potassium carbonate);
KOH (potassium hydroxide);
LHMDS (lithium hexamethyldisilazide);
LiOH (lithium hydroxide);
LiOH.H₂O (lithium hydroxide monohydrate);
Me (methyl; -CH₃);
MeOH (methanol);
MgCO₃ (magnesium carbonate);
MgSO₄ (magnesium sulfate);
Na₂CO₃ (sodium carbonate);
NaHCO₃ (sodium bicarbonate);
NaH (sodium hydride);
Na₂SO₄ (sodium sulfate);
NBS is N-bromosuccinamide;
NH₄OH (ammonium hydroxide);
Et (ethyl; -CH2CH3)
EtOH (ethanol);
EtOAc (ethyl acetate);
FMOC (9-fluorenylmethoxycarbonyl);
TEA (triethylamine);
TFA (trifluoroacetic acid);
THF (tetrahydrofuran);
TIPS (triisopropylsilyl);
TMS (trimethyisilyl); and
TMSE (2-(trimethylsilyl)ethyl);

All references to ether are to diethyl ether; brine refers to a saturated aqueous solution of NaCl. Unless otherwise indicated, all temperatures are expressed in ° C. (degrees Centigrade). All reactions are conducted under an inert atmosphere at rt unless otherwise noted.

¹H-NMR spectra were recorded on a Varian VXR-300, a Varian Unity-300, a Varian Unity-400 instrument, a General Electric QE-300, a Bruker 300, or a Bruker 400. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, a SCIEX-APliii, a Finnegan MSQ, Waters SQD, Waters ZQ, or a Finnegan LCQ spectrometer; high resolution MS were obtained using a JOEL SX-102A spectrometer. All mass spectra were taken under electrospray ionization (ESI), chemical ionization (Cl), electron impact (El) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60E-254), visualized with UV light, 5% ethanolic phosphomolybdic acid or p-anisaldehyde solution or mass spectrometry (electrospray or AP). Flash column chromatography was performed on silica gel (230-400 mesh, Merck) or using automated silica gel chromatography (Isco, Inc. Sq 16× or 100 sg Combiflash).

Reported HPLC retention times (RT) were obtained on a Waters 2795 instrument attached to a Waters 996 diode array detector reading 210-500 nm. The column used was a Synergi Max-RP (50×2 mm) model #00B-4337-B0, Solvent gradient was 15% MeOH:water to 100% MeOH (0.1% formic acid) over 6 min. Flow rate was 0.8 mL/min. Injection volume was 3 μL.

### Intermediate 1

### 2-Propen-1-yl {3-[(2-chloro-4-pyrimidinyl)acetyl]phenyl}carbamate

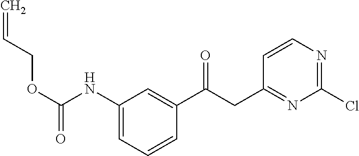

Step A: Ethyl 3-{[(2-propen-1-yloxy)carbonyl]amino}benzoate

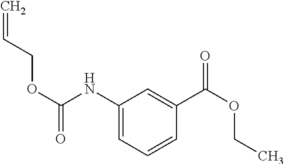

A solution of ethyl-3-aminobenzoate (25.0 g, 151.33 mmol) in DCM (500 mL) was cooled to 0° C. 2,6-Lutidine (19.46 g, 181.60 mmol) was added to the solution followed by addition of allyl chloroformate (20.07 g, 166.46 mmol). Following addition, the reaction was removed from ice bath and stirred at rt for 30 min. The reaction was quenched with saturated NaHCO₃ and the layers were separated. The mixture was extracted with DCM x3, and the combined organics were washed with 10% HCl/H₂O x3, dried over MgSO₄ and the solvent was removed to give ethyl 3-{[(2-propen-1-yloxy)carbonyl]amino}-benzoate (38.80 g, 80% yield). ¹H-NMR (400 MHz, DMSO-d₆) δ 9.96 (s, 1H), 8.15 (s, 1H), 7.66-7.72 (m, 1H), 7.59 (d, J=7.7 Hz, 1H), 7.43 (t, J=7.9 Hz, 1H), 5.94-6.04 (m, 1H), 5.37 (dd, J=17.4 and 1.7 Hz, 1H), 5.24 (dd, J=10.6 and 1.5 Hz, 1H), 4.63 (d, J=5.5 Hz, 2H), 4.31 (q, J=7.3 Hz, 2H), and 1.31 (t, J=7.1 Hz, 3H); ES-LCMS m/z 250 (M+H).

Step B: 2-Propen-1-yl {3-[(2-chloro-4-pyrimidinyl)acetyl]phenyl}carbamate Ethyl 3-{[(2-propen-1-yloxy)carbonyl]amino}benzoate (20.0 g, 80.24 mmol) was dissolved in 1 M LiHMDS in THF (260 mL) and cooled to 0° C. A solution containing 2-chloro-4-methylpyrimidine (10.32 g, 80.24 mmol) in 20 mL dry THF was added to the reaction mixture. The reaction was stirred at 0° C. for 2 h, quenched with MeOH (100 mL), dried directly onto silica, and purified via flash chromatography EtOAc/CH₂Cl₂ 0-100% gradient run over 60 min. The desired fractions were combined and the solvent was removed to give 2-propen-1-yl {3-[(2-chloro-4-pyrimidinyl)acetyl]phenyl}carbamate (13.6 g, 51% yield). ES-LCMS m/z 332 (M+H).

### Intermediate 2

### 3-Fluoro-4-{[2-(1-pyrrolidinyl)ethyl]oxy}phenyl amine hydrochloride

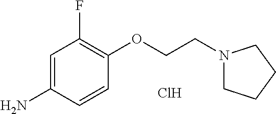

Step A: 1-{2-[(2-Fluoro-4-nitrophenyl)oxy]ethyl}pyrrolidine

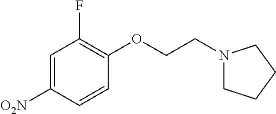

To obtain the target compound, NaH (2.5 g, 63 mmol, 60% dispersion) was placed in a round bottom flask with THF (25 mL) at 0° C. 2-(1-Pyrrolidinyl)ethanol (7.5 mL, 68 mmol) was added portion-wise to the stirring NaH solution over 15 min. This reaction mixture was stirred at 0° C. for 45 min. 3,4-Difluoronitrobenzene (10 g, 63 mmol) was then added portion-wise to the reaction at 0° C. The reaction was allowed to stir and warm to rt over several hours. A few drops of MeOH were added to the reaction. EtOAc and H₂O were then added to the reaction mixture and the desired product was extracted into the organic phase which was then concentrated onto silica gel and purified via column chromatography to yield 8.5 g of the target compound of Step A. (53%) ¹H-NMR (400 MHz, DMSO-d₆) δ ppm 8.06-8.15 (m, 2H), 7.40 (t, J=8.8 Hz, 1H), 4.28 (t, J=5.7 Hz, 2H), 2.82 (t, J=5.7 Hz, 2H), 2.5 (m, 4H), and 1.65 (m, 4H).

Step B: 3-Fluoro-4-{[2-(1-pyrrolidinyl)ethyl]oxy}phenyl amine hydrochloride

To obtain the desired target, 1-{2-[(2-fluoro-4-nitrophenyl)oxy]ethyl}pyrrolidine (8.5 g, 33 mmol) and EtOH (50 mL) were placed in a pressure reaction vessel under N2. Platinum on carbon (500 mg, 5% by wt) was added followed by 40 psi of H2. The reaction was stirred overnight at rt. The reaction was then filtered through celite and the subsequent filtrate was concentrated to dryness. A small amount of EtOAc was added followed by 4 N HCl in dioxane (8.25 mL, 33 mmol). This was then concentrated to dryness to yield a sticky solid which was then sonicated in ether and filtered off as a beige powder to yield 8.0 g of the desired phenyl amine as a hydrochloride salt. ¹H-NMR (400 MHz, DMSO-d₆) δ ppm 6.92 (m, 1H), 6.41 (m, 1 H), 6.31 (m, 1H), 5.28 (s, 2H), 4.20 (m, 2H), 3.54 (s, 2H), 3.47 (m, 2H), 3.06 (s, 2 H), and 1.97 (s, 2H), 1.86 (s, 2H).

### Intermediate 3

### 2-Methylpropanethioamide

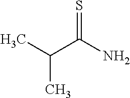

A solution of 2-methylpropanamide (6.53 g, 75.0 mmol) and 2,4-bis(4-methoxyphenyl)-1,3-dithia-2,4-diphosphetane-2,4-disulfide (15.17 g, 37.51 mmol) in THF (100 mL) was heated to reflux for 4 h. The reaction mixture was then cooled to rt and poured into saturated aqueous NaHCO₃ (200 mL). The mixture was extracted with ether (4×100 mL). The organic fractions were combined, dried over Na₂SO₄, filtered, and concentrated. Purification by flash column chromatography (20% EtOAc:hexanes) afforded 4.77 g (62%) of the title compound of Step A. ¹H-NMR (400 MHz, CDCl₃) δ 7.63 (brs, 1H), 6.90 (brs, 1H), 2.88 (m, 1H), and 1.27 (d, 6H, J=6.8 Hz).

### Intermediate 4

### 6-(4-morpholinyl)-3-pyridinamine

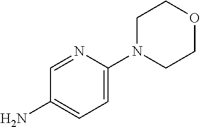

Step A: 4-(5-Nitro-2-pyridinyl)morpholine

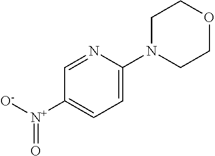

To a solution containing 1.2 mL (13.9 mmol) of morpholine and 20 mL of THF was added 0.66 g (16.4 mmol) of a 60% dispersion of sodium hydride in mineral oil. The reaction was allowed to stir for 15 min and 2.0 g (12.6 mmol) of 2-chloro-5-nitropyridine was added. The reaction mixture was heated at 50° C. overnight, then quenched by the addition of H₂O and extracted with DCM. The combined organic layers were dried over MgSO₄ and the solvents were removed under reduced pressure. The residue was subjected to silica gel chromatography to give 1.65 g (63%) of 4-(5-nitro-2-pyridinyl)morpholine as a yellow solid: ¹H-NMR (400 MHz, DMSO-d₆) δ 8.97 (d, J=2.9 Hz, 1H), 8.25 (dd, J=9.5, 2.9 Hz, 1H), 6.95 (d, J=9.5 Hz, 1H), 3.72-3.75 (m, 4H), and 3.68-3.71 (m, 4H); MS (ESI): 210.28 (M+H⁺).

Step B: 6-(4-Morpholinyl)-3-pyridinamine

A mixture containing 1.65 g (7.8 mmol) of 4-(5-nitro-2-pyridinyl)morpholine, 160 mg of 5% Pt on carbon, and 20 mL of EtOHI was subjected to a 50 psi H2 atmosphere for 5 h. The reaction mixture was filtered through a pad of Celite and the solvents were removed under reduced pressure to give 1.4 g (100%) of 6-(4-morpholinyl)-3-pyridinamine as a purple solid: ¹H-NMR (400 MHz, DMSO-d₆) δ 7.60 (d, J=2.9 Hz, 1 H), 6.92 (dd, J=8.8 and 2.9 Hz, 1H), 6.62 (d, J=8.8 Hz, 1H), 4.59 (brs, 2H), 3.65-3.72 (m, 4H), and 3.17 (dt, J=4.9 and 2.4 Hz, 4H).

### Intermediate 5

### 3-Fluoro-4-{4-[2-(methylsulfonyl)ethyl]-1-piperazinyl}phenyl amine

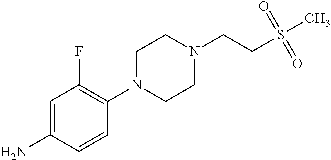

Step A: 1-(2-Fluoro-4-nitrophenyl)-4-[2-(methylsulfonyl)ethyl]piperazine

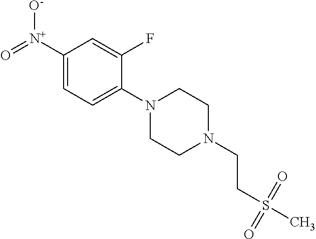

To a solution of 1-(2-fluoro-4-nitrophenyl)piperazine (0.50 g, 2.22 mmol) in i-PrOH (25 mL), methyl vinyl sulfone (0.354 g, 3.33 mmol) was added. The reaction was heated to reflux for 18 h. The reaction was cooled to rt, loaded directly onto silica, and purified via flash chromatography EtOAC/EtOAc:MeOH:NH₄OH (80:19:1) 0-100% gradient over 15 min. The desired fractions were combined and the solvent was removed to give 0.500 g of a yellow solid. ¹H-NMR (400 MHz, DMSO-d₆) δ 7.93-8.00 (m, 2H), 7.13 (td, J=9.1 and 0.7 Hz, 1H), 3.22-3.31 (m, 6H), 3.00 (s, 3H), 2.73 (t, J=6.6 Hz, 2H), and 2.56 (brs, 4H).

Step B: 3-Fluoro-4-{4-[2-(methylsulfonyl)ethyl]-1-piperazinyl}phenyl amine

1-(2-Fluoro-4-nitrophenyl)-4-[2-(methylsulfonyl)ethyl]piperazine (0.500 g, 1.51 mmol) was taken up in EtOH (15 mL) and 10% Palladium/Carbon (0.050 g) was added. The mixture was stirred under H2, 60 psi, for 3 h. The reaction was filtered through a celite plug and the solvent was removed to give 0.450 g, 98% yield, of a yellow solid. ¹H-NMR (400 MHz, DMSO-d₆) δ 6.67-6.74 (m, 1H), 6.21-6.31 (m, 2H), 4.92 (s, 2 H), 3.22-3.28 (m, 3H), 2.97-3.01 (m, 3H), 2.77 (brs, 4H), 2.66-2.72 (m, 2H), and 2.50 (brs, 3H).

### Intermediate 6

### 5-Methyl-2-(methyloxy)-4-{4-[2-(methylsulfonyl)ethyl]-1-piperazinyl}phenyl amine

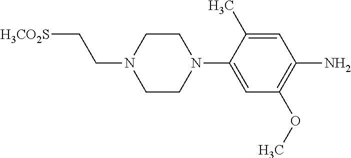

Step A: 1,1-Dimethylethyl 4-[2-(methylsulfonyl)ethyl]-1-piperazinecarboxylate

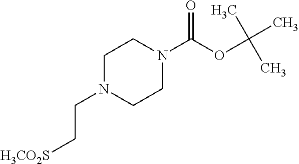

To a solution of 1,1-dimethylethyl 1-piperazinecarboxylate (72.8 g, 390 mmol) in acetonitrile (1.4 L) was added methyl vinyl sulfone (50 g, 470 mmol) and Na₂CO₃ (124 g, 1170 mmol) and the mixture was refluxed overnight. Then the mixture was cooled to rt, and poured into H₂O and extracted with EtOAc. The organic layer was dried over Na₂SO₄ and the solvent was removed under reduced pressure to give the title compound of step A (106 g, 93% yield). ¹H-NMR (400 MHz, CDCl₃) δ ppm 3.35-3.50 (m, 4H), 3.14 (t, J=8.4 Hz, 2H), 3.02 (s, 3H), 2.88 (t, J=8.4 Hz, 2H), 2.39-2.51 (m, 4H), and 1.41-1.57 (m, 9H).

Step B: 5-Fluoro-4-methyl-2-nitrophenol

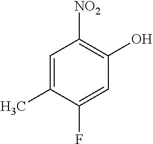

3-Fluoro-4-methylphenol (3.66 g, 29.0 mmol) was dissolved in DCE (32 mL) and tetrabutylammonium bromide (0.935 g, 2.90 mmol) was added. HNO₃ 70% (3.7 mL, 58 mmol) was diluted with H₂O (33 mL) to make a 7% HNO₃ solution. This solution was added to the reaction mixture, which was then stirred at rt for 4 h at which time the reaction was judged complete by TLC. The reaction was poured into H₂O and extracted with DCM (3×). The combined organic layers were dried over MgSO₄, filtered and concentrated in vacuo. The resulting residue was adsorbed onto silica gel and flash chromatographed to give the title compound of step B (2.83 g, 57%). ¹H-NMR (400 MHz, DMSO-d₆) δ ppm 11.10 (s, 1H), 7.89 (d, J=8.1 Hz, 1H), 6.85 (d, J=11.0 Hz, 1H), and 2.13 (s, 3H).

Step C: 5-Fluoro-4-methyl-2-nitrophenyl methyl ether

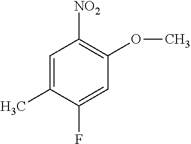

5-Fluoro-4-methyl-2-nitrophenol (2.83 g, 16.5 mmol) was dissolved in DMF (25 mL). K₂CO₃ (3.4 g, 25 mmol) and iodomethane (1.2 mL, 20 mmol) were added and the mixture was stirred at rt overnight. The mixture was then poured into H₂O and stirred until solids crashed out. The solids were filtered and air dried to give the title compound of step C without further purification (2.76 g, 90%). ¹H-NMR (400 MHz, DMSO-d₆) δ ppm 7.92 (d, J=8.1 Hz, 1H), 7.25 (d, J=11.7 Hz, 1H), 3.89 (s, 3H), and 2.19 (d, J=1.5 Hz, 3H).

Step D: 1-[2-(Methylsulfonyl)ethyl]piperazine hydrochloride

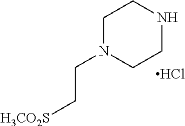

To the solution of 1,1-dimethylethyl 4-[2-(methylsulfonyl)ethyl]-1-piperazinecarboxylate (106 g, 360 mmol) in MeOH (500 mL) was added HCl/MeOH (5M, 1000 mL, 5000 mmol), and the mixture was heated at 50° C. for 1 h before evaporating most of the solvent. The residue was filtered and washed with MeOH to give the desired product as HCl salt (90.3 g, 95% yield). ¹H-NMR (400 MHz, D₂O) δ ppm 3.30-3.72 (m, 12H), and 3.09 (s, 3H).

Step E: 1-[2-Methyl-5-(methyloxy)-4-nitrophenyl]-4-[2-(methylsulfonyl)ethyl]piperazine

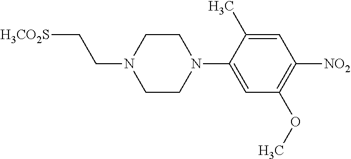

5-Fluoro-4-methyl-2-nitrophenyl methyl ether (5.0 g, 27 mmol), K₂CO₃ (11.2 g, 81 mmol), and 1-[2-(methylsulfonyl)ethyl]piperazine hydrochloride (8.0 g, 35 mmol) were weighed into a 150 mL sealed tube, taken up in 85 mL of DMSO and heated to 100° C. for 24 h. The mixture was poured into 500 mL of H₂O, filtered, washed with H₂O, air dried for 15 min, washed with hexanes (200 mL), washed with 20% Ether/Hexanes (200 mL), and air dried for 2 h to give the title compound of step E (6.6 g, 18.5 mmol, 68%). An additional (540 mg, 5.5%) of product was obtained by extraction of the H₂O layer, followed by flash chromatography. ¹H-NMR (400 MHz, DMSO-d₆) δ ppm 7.75 (s, 1H), 6.70 (s, 1H), 3.90 (s, 3H), 3.31-3.35 (m, 2H), 3.04 (s, 3H), 2.99-3.03 (m, 4H), 2.77 (t, J=6.6 Hz, 2H), 2.56-2.63 (m, 4H), and 2.19 (s, 3H).

Step F: 5-Methyl-2-(methyloxy)-4-{4-[2-(methylsulfonyl)ethyl]-1-piperazinyl}phenyl amine

1-[2-Methyl-5-(methyloxy)-4-nitrophenyl]-4-[2-(methylsulfonyl)ethyl]piperazine from a separate batch (0.257 g, 0.72 mmol) was placed in a 40 mL vial and dissolved in 10 mL of 1:1 EtOAc/MeOH. 5 wt % Platinum(sulfided)/carbon (0.165 g, 0.043 mmol) was added followed quickly by a screw cap septum. The vial was evacuated and filled with N₂ six times to remove any oxygen. The vial was then pressurized with H₂ (balloon). The solution was stirred overnight. The next morning the vessel was evacuated and filled with N₂ six times to remove any H₂. The solution was filtered through celite and evaporated to afford the title compound of step F (0.194 g, 0.59 mmol, 82%). ¹H-NMR (400 MHz, CDCl₃δ ppm 6.54 (s, 2H), 3.80 (s, 3H), 3.30-3.37 (m, 2H), 3.07 (s, 3H), 2.91-2.98 (m, 6H), 2.82-2.90 (m, 4H), and 2.15 (s, 3H).

### Intermediate 7

### 1-Pyrrolidinecarbothioamide

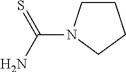

To obtain the title compound, pyrrolidine (1.5 g, 21 mmol) was placed in a round bottom flask under N₂ with stirring. THF (4 mL) was added followed by the drop-wise addition of 4N HCl in dioxane (5.3 mL, 21 mmol). Potassium thiocyanate (2.0 g, 21 mmol) was then added in one portion to the stirring solution of pyrrolidine hydrochloride. This mixture was then stirred at rt for 30 min followed by heating at 100° C. for 2 h. The reaction was then cooled to rt, MeOH (50 mL) was added, and solids that persisted were filtered away. Subsequent concentration of the MeOH/reaction solution yielded 3.0 g of the crude thiourea. ¹H-NMR (400 MHz, DMSO-d₆) δ 8.60 (brs, 2H), 3.07 (m, 4H), and 1.82 (m, 4H).

### Intermediate 8

### 2,2-Dimethylpropanethioamide

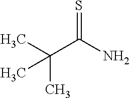

The title compound of Step A was prepared (3.2 g, 36%) from 2,2-dimethylpropanamide (7.59 g, 75.0 mmol) and 2,4-bis(4-methoxyphenyl)-1,3-dithia-2,4-diphosphetane-2,4-disulfide (15.17 g, 37.51 mmol) by a procedure analogous to Intermediate 3. ¹H-NMR (400 MHz, CDCl₃) δ 7.92 (brs, 1H), 7.03 (brs, 1H), and 1.38 (s, 9H).

### Example 1

### N-(3-(2-(Ethylamino)-5-{2-[(3-fluoro-4-[[2-(1-pyrrolidinyl)ethyl]oxy}phenyl)amino]-4-pyrimidinyl}-1,3-thiazol-4-yl)phenyl]benzenesulfonamide

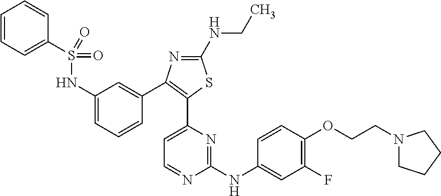

Step A: 2-Propen-1-yl {3-[5-(2-chloro-4-pyrimidinyl)-2-(ethylamino)-1,3-thiazol-4-yl]phenyl}carbamate

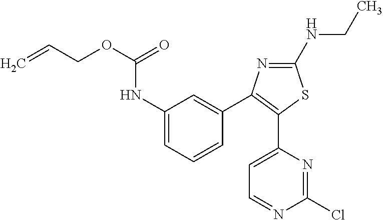

To a suspension of 2-propen-1-yl{3-[(2-chloro-4-pyrimidinyl)acetyl]phenyl}carbamate (2.0 g, 6.0 mmol), prepared in a procedure analogous to Intermediate 1, Step B, in DCM (25 mL) was added NBS (1.1 g, 6.0 mmol). After stirring for 10 min at rt, the solvent was removed on the rotovap. The residue was redissolved in DMF (20 mL), and 1-ethyl-2-thiourea (0.75 g, 7.2 mmol) was added. After 16 hours at rt, the reaction mixture was partitioned between EtOAC and H₂O. The layers were separated, and the aqueous layer was extracted with EtOAc. The combined organic layers were dried over anhydrous MgSO₄, filtered, adsorbed onto silica gel, and purified by column chromatography (eluting with 20-100% EtOAc/hexanes) to generate the desired product in 80% yield (2.0 g, 4.8 mmol). MS (ESI) m/z=416 [M+H]⁺.

Step B: 4-[4-(3-Aminophenyl)-2-(ethylamino)-1,3-thiazol-5-yl]-N-(3-fluoro-4-{[2-(1-pyrrolidinyl)ethyl]oxy}phenyl)-2-pyrimidinamine

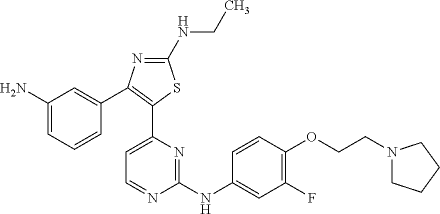

A suspension of 2-propen-1-yl{3-[5-(2-chloro-4-pyrimidinyl)-2-(ethylamino)-1,3-thiazol-4-yl]phenyl}carbamate (250 mg, 0.60 mmol) and (3-fluoro-4-{[2-(1-pyrrolidinyl)ethyl]oxy}phenyl)ammonium chloride, prepared in a procedure analogous to Intermediate 2, Step B (149 mg, 0.57 mmol) in iPrOH (3 mL) was heated for 15 min at 170° C. in a microwave reactor. The reaction mixture was adsorbed onto silica gel and purified by column chromatography (eluting with 0-20% MePH/EtOAc/0.1% NH₄OH). All fractions containing the desired product were combined and partitioned between EtOAc and H₂O (basified to pH>9 with Na₂CO₃). The organic layer was dried over anhydrous Na₂SO₄, filtered, and concentrated to a yellow oil. The crude product was dissolved in THF (3 mL), and a solution of tetra-N-butylammonium fluoride (1.0 M) in THF (1.2 mL, 1.2 mmol) was added. The reaction was heated for 10 min at 170° C. in a microwave reactor. The reaction mixture was diluted with EtOAc, washed with H₂O, dried over anhydrous MgSO₄, filtered, and concentrated to generate the desired product as a brown solid in 83% yield (258 mg, 0.5 mmol). MS (ESI) m/z=520 [M+H]⁺.

Step C: N-[3-(2-(Ethylamino)-5-{2-[(3-fluoro-4-{[2-(1-pyrrolidinyl)ethyl]oxy}phenyl)amino]-4-pyrimidinyl}-1,3-thiazol-4-yl)phenyl]benzenesulfonamide

To a suspension of 1-hydroxy-7-azabenzotriazole (28 mg, 0.21 mmol) and TEA (32 μL, 0.23 mmol) in THF (5 mL) was added benzenesulfonyl chloride (22 μL, 0.17 mmol). The reaction was stirred for 15 min, and then cooled to 0° C. A solution of 4-[4-(3-aminophenyl)-2-(ethylamino)-1,3-thiazol-5-yl]-N-(3-fluoro-4-{[2-(1-pyrrolidinyl)ethyl]oxy}phenyl)-2-pyrimidinamine (100 mg, 0.19 mmol) in THF (5 mL) was added, and the reaction was allowed to warm to rt. After 16 hours, the reaction mixture was filtered, concentrated, redissolved in 1 mL 1:1 MeOH/DMSO, and purified by reverse-phase HPLC (eluting with 10-60% acetonitrile/0.1% trifluoroacetic acid). The product fractions were combined, basified to pH>9 with Na₂CO₃, and extracted with EtOAc. The combined organic layers were dried over anhydrous Na₂SO₄, filtered, and concentrated to generate the desired product in 43% yield (54 mg, 0.08 mmol). ¹H-NMR (400 MHz, d₆-DMSO): δ 10.40 (brs, 1H), 9.49 (s, 1H), 8.24 (t, 1H, J=5.3 Hz), 7.95 (d, 1H, J=5.3 Hz), 7.73-7.80 (m, 3H), 7.50-7.59 (m, 3H), 7.28-7.35 (m, 2H), 7.05-7.20 (m, 4H), 5.97 (d, 1H, J=5.4 Hz), 4.08 (t, 2H, J=5.7 Hz), 3.27 (m, 2H), 2.78 (t, 2H, J=5.8 Hz), 2.52 (brs, 4H), 1.67 (brs, 4H), and 1.17 (t, 3H, J=7.2 Hz); MS (ESI) m/z=660 [M+H]⁺.

### Example 2

### N-{3-[2-Ethyl-5-(2-{[3,4,5-tris(methyloxy)phenyl]amino}-4-pyrimidinyl)-1,3-thiazol-4-yl]phenyl}benzenesulfonamide

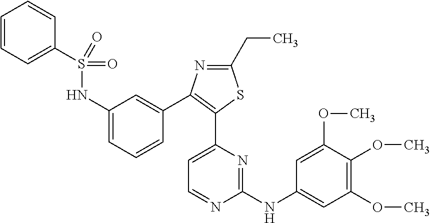

Step A: Ethyl 3-[(phenylsulfonyl)amino]benzoate

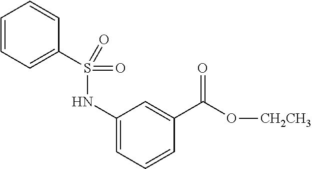

To a solution of ethyl 3-aminobenzoate (4.6 g, 28 mmol) and TEA (4.3 g, 42 mmol) in THF (100 mL), cooled to 0° C., was added benzenesulfonyl chloride (5.0 g, 28 mmol) dropwise over 10 min. The reaction was allowed to warm to rt, and was stirred at rt over the weekend. The reaction mixture was concentrated on the rotovap, and the residue was partitioned between EtOAc and saturated aqueous NaHCO₃. The organic layer was adsorbed onto silica gel and purified by column chromatography (eluting with 0-100% EtOAc/DCM) to generate the desired product in 70% yield (5.96 g, 19.5 mmol). MS (ESI) m/z=304 [M−H]⁺.

Step B: N-{3-[2-(2-chloro-4-pyrimidinyl)acetyl]phenyl}benzenesulfonamide

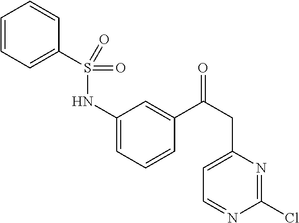

A solution of ethyl 3-[(phenylsulfonyl)amino]benzoate (1.0 g, 3.3 mmol) in THF (20 mL) was cooled to 0° C. A 1.0 M solution of LHMDS in THF (7.5 mL, 7.5 mmol) was added, followed by dropwise addition of a solution containing 2-chloro-4-methylpyrimidine (0.42 g, 3.3 mmol) in THF (10 mL) over 10 min. The reaction mixture was allowed to warm to rt and stirred for 2 h. The solution was concentrated under reduced pressure and partitioned between EtOAc and water. The organic layer was dried over Na2SO4 and filtered. The solvents were removed under reduced pressure and the residue was subjected to silica gel chromatography. Yield: 0.53 g (1.4 mmol, 42% yield). MS (ESI) m/z=388 [M+H]⁺.

Step C: N-{3-[5-(2-Chloro-4-pyrimidinyl)-2-ethyl-1,3-thiazol-4-yl]phenyl}benzenesulfonamide

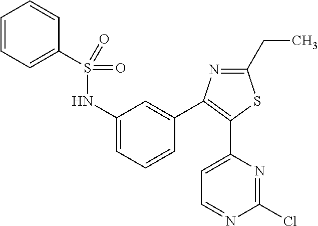

To a suspension of N-{3-[2-(2-chloro-4-pyrimidinyl)acetyl]phenyl}benzenesulfonamide in DCM was added NBS (0.24 g, 1.4 mmol). The reaction mixture was stirred for 20 min and the solvents were removed under reduced pressure. The residue was redissolved in DMF and propanethioamide was added. The reaction mixture was stirred for 1.5 h, then partitioned between EtOAc and water. The organic layer was dried over Na₂SO₄ and filtered. The solvents were removed under reduced pressure and the residue was subjected to silica gel chromatography. Yield: 0.43 g (0.94 mmol, 67% yield). MS (ESI) m/z=457 [M+H]⁺.

Step D: N-{3-[2-Ethyl-5-(2-{[3,4,5-tris(methyloxy)phenyl]amino}-4-pyrimidinyl)-1,3-thiazol-4-yl]phenyl}benzenesulfonamide
General Procedure for the Aniline Displacement To a solution containing 1-4 mL of i-PrOH or trifluoroethanol, optionally with 1,4-dioxane or DMA added as a co-solvent to improve solubility, and 1 equiv of the pyrimidyl chloride is added 1-1.5 equiv of the desired aniline and 1 drop of conc HCl or 4-5 drops of a 4 M solution of HCl in dioxane. The reaction mixture is heated at 70-90° C. for 12-72 h, or heated in a microwave reactor at 150-180° C. for 10-120 min, then allowed to cool to rt. The displacement product is purified by neutralization by the addition of an aqueous solution of NaOH or NaHCO₃, or by the addition of 1-5 equiv of TEA or ammonia and extracted into an organic solvent such as EtOAc or DCM. The residue from this extraction, or directly from evaporation of solvents from the reaction mixture, is then subjected to silica gel chromatography and/or HPLC purification. In certain instances, precipitation from an organic solvent, or treatment of a solution of the compound with MP-isocyante is utilized to remove excess aniline or other lingering impurities.

Specific procedure for N-{3-[2-Ethyl-5-(2-{[3,4,5-tris(methyloxy)phenyl]amino}-4-pyrimidinyl)-1,3-thiazol-4-yl]phenyl}benzenesulfonamide

To a suspension of N-{3-[5-(2-chloro-4-pyrimidinyl)-2-ethyl-1,3-thiazol-4-yl]phenyl}benzenesulfonamide (75 mg, 0.16 mmol) and 3,4,5-tris(methyloxy)phenyl amine (30 mg, 0.16 mmol) in iPrOH (2 mL) was added concentrated HCl (1 drop). The reaction was heated to 170° C. in a microwave reactor for 15 min. The reaction mixture was adsorbed onto silica gel and purified by column chromatography (eluting with 0-100% EtOAc/DCM). The product fractions were not sufficiently pure, so they were concentrated, redissolved in 1 mL 1:1 MeOH/DMSO, and purified by RP-HPLC (eluting with 20-70% acetonitrile/water/0.1% TFA). The product fractions were combined, basified with saturated aqueous NaHCO₃, and extracted with EtOAc. The organic layer was dried over anhydrous NaSO₄, filtered, and concentrated to generate the title compound of Example 2 in 39% yield (38 mg, 0.06 mmol). ¹H-NMR (400 MHz, d₆-DMSO) δ 10.42 (s, 1H), 9.60 (s, 1H), 8.20 (d, 1H, J=5.1 Hz), 7.73 (d, 1H, J=7.0 Hz), 7.51-7.62 (m, 3H), 7.31-7.35 (m, 1H), 7.22 (s, 2H), 7.20 (s, 3H), 6.22 (d, 1H, J=5.1 Hz), 3.79 (s, 6H), 3.61 (s, 3H), 3.01 (q, 2H, J=7.6 Hz), and 1.32 (t, 3H, J=7.6 Hz); MS (ESI) m/z=604 [M+H]⁺.

### Example 3

### N-{3-[5-(2-{[6-(4-Acetyl-1-piperazinyl)-3-pyridinyl]amino}-4-pyrimidinyl)-2-ethyl-1,3-thiazol-4-yl]phenyl}benzenesulfonamide

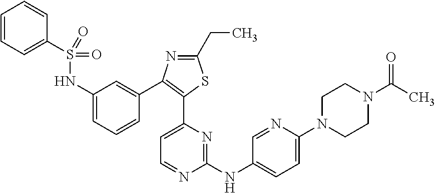

Step A: tent-Butyl 4-(5-nitro-2-pyridinyl)-1-piperazinecarboxylate

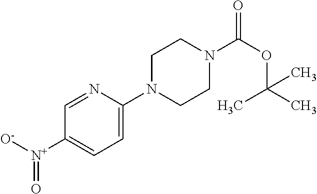

To a solution containing 5.8 g (31.5 mmol) of tert-butyl 1-piperazinecarboxylate and 20 mL of THF at 0° C. was added 1.5 g (37 mmol) of a 60% dispersion of NaH in mineral oil. The reaction mixture was allowed to stir for 20 min and 5.0 g (31.5 mmol) of 2-chloro-5-nitropyridine was added. The reaction mixture was heated at 50° C. overnight, quenched by the addition of H₂O, and extracted with DCM. The combined organic layers were dried over MgSO₄ and the solvent was removed under reduced pressure. The residue was subjected to silica gel chromatography to give 4.89 g (50%) of tert-butyl 4-(5-nitro-2-pyridinyl)-1-piperazinecarboxylate as a yellow solid: ¹H-NMR (400 MHz, DMSO-d₆) δ 8.25 (dd, J=9.5 and 2.9 Hz, 1H), 6.93 (d, J=9.5 Hz, 1H), 3.76-3.78 (m, 4H), 3.41-3.48 (m, 4H), and 1.42 (s, 9H).

Step B: 1-(5-Nitro-2-pyridinyl)piperazine bis trifluoroacetate

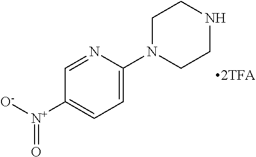

To a solution containing 4.8 g (15.6 mmol) of tert-butyl 4-(5-nitro-2-pyridinyl)-1-piperazinecarboxylate and 50 mL of DCM was added 5 mL of TFA. The reaction mixture was allowed to stir for 3 days and the solvents were removed under reduced pressure to give 6.21 g (91%) of 1-(5-nitro-2-pyridinyl)piperazine bis trifluoroacetate as a yellow solid: ¹H-NMR (400 MHz, DMSO-d₆) δ 9.00 (d, J=2.8 Hz), 8.85 (brs, 3H), 8.33 (dd, J=9.5 and 2.8 Hz, 1H), 7.04 (d, J=9.5 Hz, 1H), 3.94-3.99 (m, 4H), and 3.23 (brs, 4H).

Step C: 1-Acetyl-4-(5-nitro-2-pyridinyl)piperazine

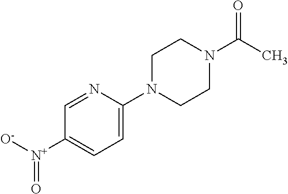

To a solution containing 1.5 g (3.5 mmol) of 1-(5-nitro-2-pyridinyl)piperazine bis trifluoroacetate and 20 mL of THF was added 0.37 mL (3.85 mmol) of acetic anhydride, followed by 1.6 mL (11.6 mmol) of TEA. The reaction mixture was heated at 50° C. overnight and the solvent was removed under reduced pressure. The residue was subjected to silica gel chromatography to give 0.9 g (100%) of 1-acetyl-4-(5-nitro-2-pyridinyl)piperazine as a yellow solid: ¹H-NMR (400 MHz, DMSO-d₆) δ 8.99 (d, J=2.8 Hz), 8.25 (dd, J=9.6 and 2.8 Hz, 1H), 6.95 (d, J=9.5 Hz, 1H), 3.79-3.86 (m, 2H), 3.73-3.79 (m, 2H), 3.57 (dt, J=6.8 and 3.5 Hz, 4H), and 2.05 (s, 3

H).

Step D: 6-(4-Acetyl-1-piperazinyl)-3-pyridinamine

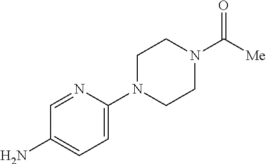

A mixture containing 0.9 g (3.5 mmol) of 1-acetyl-4-(5-nitro-2-pyridinyl)piperazine, 0.09 g of 5% Pt on carbon, and 20 mL of ethanol was subjected to a 60 psi hydrogen atmosphere for 6 h. The reaction mixture was filtered through a pad of Celite, eluting with ethanol and ethyl acetate and the solvent was removed under reduced pressure to give 0.77 g (100%) of 6-(4-acetyl-1-piperazinyl)-3-pyridinamine as a purple solid: ¹H-NMR (400 MHz, DMSO-d₆) δ 7.60 (s, 1H), 6.89 (d, J=9.0 Hz, 1H), 6.66 (d, J=9.0 Hz, 1H), 4.60 (brs, 2H), 3.51 (brs, 4H), 3.24 (brs, 2H), 3.17 (brs, 2H), and 2.03 (brs, 3H).

Step E: N-{3-[5-(2-{[6-(4-Acetyl-1-piperazinyl)-3-pyridinyl]amino}-4-pyrimidinyl)-2-ethyl-1,3-thiazol-4-yl]phenyl}benzenesulfonamide

The title compound of Example 3 was made in a similar procedure to that described in the preparation of Example 2, Step D, replacing 3,4,5-tris(methyloxy)phenyl amine with 6-(4-acetyl-1-piperazinyl)-3-pyridinamine. Yield: 28 mg (0.04 mmol, 27% yield).

¹H-NMR (400 MHz, d₆-DMSO) δ 10.41 (s, 1H), 9.50 (s, 1H), 8.43 (d, 1H, J=3.0 Hz), 8.16 (d, 1H, J=5.5 Hz), 7.85 (dd, 1H, J=2.9 and 9.2 Hz), 7.71-7.74 (m, 2H), 7.51-7.62 (m, 3H), 7.30-7.34 (m, 1H), 7.17-7.22 (m, 3H), 6.83 (d, 1H, J=8.8 Hz), 6.20 (d, 1H, J=5.1 Hz), 3.51-3.57 (m, 4H), 3.43-3.48 (m, 2H), 3.36-3.40 (m, 2H), 3.01 (q, 2H, J=7.5 Hz), 2.04 (s, 3H), and 1.33 (t, 3H, J=7.5 Hz); MS (ESI) m/z=641 [M+H]⁺.

### Example 4

### N-{3-[(2-Ethyl-5-(2-{[3-fluoro-4-(4-morpholinyl)phenyl]amino}-4-pyrimidinyl)-1,3-thiazol-4-yl]phenyl}benzenesulfonamide

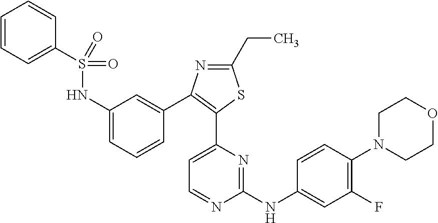

The title compound of Example 4 was made in a similar procedure to that described in the preparation of Example 2, Step D, replacing 3,4,5-tris(methyloxy)phenyl amine with 3-fluoro-4-(4-morpholinyl)phenyl amine. Yield: 30 mg (0.05 mmol, 30% yield). ¹H-NMR (400 MHz, d₆-DMSO) δ 10.42 (s, 1H), 9.76 (s, 1H), 8.22 (d, 1H, J=5.1 Hz), 7.50-7.74 (m, 6H), 7.39 (dd, 1H, J=2.0 and 8.6 Hz), 7.29-7.34 (m, 1H), 7.21 (s, 2H), 7.19 (s, 1H), 6.97 (t, 1H, J=9.5 Hz), 6.26 (d, 1H, J=5.5 Hz), 3.71-3.74 (m, 4H), 3.02 (q, 2H, J=7.5 Hz), 2.93-2.95 (m, 4H), and 1.33 (t, 3H, J=7.6 Hz); MS (ESI) m/z=617 [M+H]⁺.

### Example 5

### N-[3-(2-Ethyl-5-{2-[(4-(methyloxy)-3-{4-[2-(methylsulfonyl)ethyl]-1-piperazinyl}phenyl)amino]-4-pyrimidinyl}-1,3-thiazol-4-yl)phenyl]benzenesulfonamide

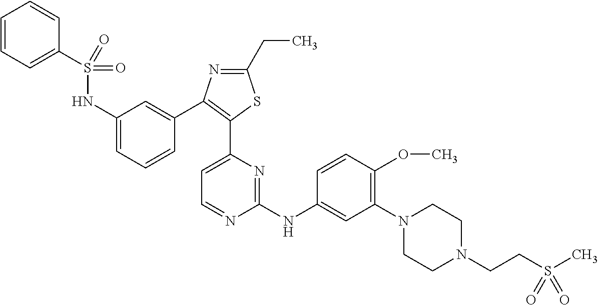

Step A: 2-(Methyloxy)-5-nitrophenyl]piperazine

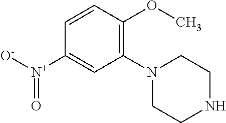

To a suspension of 11 g (57.2 mmol) of 1-[2-(methyloxy)phenyl]piperazine in 10 mL of H₂O at 0° C. was added 60 mL of conc. H₂SO₄ dropwise over 4 h. To this suspension was added 7.2 g (71.2 mmol) of potassium nitrate in portions. The reaction mixture was allowed to stir overnight at rt and quenched by pouring over ice. The mixture was neutralized by the careful addition of aqueous NaOH and extracted with ethyl acetate. The combined organic layers were dried over MgSO₄ and the solvents were removed under reduced pressure. The residue was subjected to silica gel chromatography to give 5.7 g (42%) of 1-[2-(methyloxy)-5-nitrophenyl]piperazine as an orange solid, contaminated with a small amount of a minor regioisomer. The data for the major regioisomer is: ¹H-NMR (400 MHz, DMSO-d₆) δ 7.91 (dd, J=9.0 and 2.8 Hz, 1H), 7.62 (d, J=2.9 Hz, 1H), 7.15 (d, J=9.0 Hz, 1H), 3.92 (s, 3H), 2.91-2.97 (m, 4H), 2.83-2.87 (m, 4H), and 2.67 (brs, 1H); MS (ESI) m/e: 238.1 (M+H⁺).

Step B 1-[2-(Methyloxy)-5-nitrophenyl]-4-[2-(methylsulfonyl)ethyl]piperazine

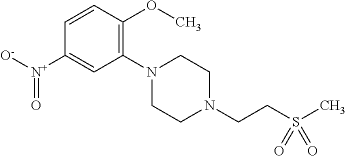

To a mixture of 2-(methyloxy)-5-nitrophenyl]piperazine (1.55 g, 6.53 mmol) and Na₂CO₃ (2 g, 18.87 mmol) stirring in acetonitrile (15 mL) was added methyl vinyl sulfone (1.82 g, 17.13 mmol). The resulting reaction was heated to 80° C. for 2 h. The reaction was incomplete. Methyl vinyl sulfone (1.82 g, 17.13 mmol) was added to the reaction and the reaction was heated for an additional 0.75 h. The reaction was partitioned between EtOAc and H₂O/5:1 (60 mL). The phases were separated and the aqueous phase was further extracted with chloroform: iPrOHI/3:1 (40 mL). The combined organic phase was washed with brine and H₂O, filtered through Whatman 1 PS paper and concentrated in vacuo to the title compound of Step B as an orange-yellow solid (2.24 g; 100%) contaminated with a small amount of excess methyl vinyl sulfone. The reported data represents the major component of the reaction. ¹H-NMR (400 MHz, DMSO-d₆) δ 7.92 (dd, J=9.0, and 2.7 Hz, 1H), 7.64 (d, J=2.9 Hz, 1H), 7.15 (d, J=8.8 Hz, 1H), 3.93 (s, 3H), 3.33 (t, J=6.8 Hz, 2H), 3.05 (s, 4H), 3.03 (s, 3H), 2.77 (t, J=6.8 Hz, 2H), and 2.60 (s, 4H).

Step C: 4-(Methyloxy)-3-{4-[2-(methylsulfonyl)ethyl]-1-piperazinyl}phenyl amine

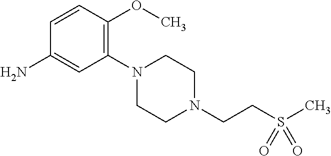

To a stirring mixture of 142-(methyloxy)-5-nitrophenyl]-4-[2-(methylsulfonyl)ethyl]piperazine (2.24 g, 6.52 mmol) in MeOH (30 mL) with nickel (II) chloride hexahydrate (0.8 g; 3.37 mmol) chilled to 0° C. The cautious addition of sodium borohydride (0.55 g; 14.54 mmol) caused a vigorous foaming action and the resulting reaction mixture turned black. The reaction was stirred for 0.5 h before a 2^{nd} equal addition of sodium borohydride. The reaction was stirred for 0.25 h and then the reaction was quenched with 3.0 N sodium hydroxide solution (100 mL).The reaction was partitioned between EtOAc and water/5:1. The reaction was filtered to remove the gross nickel salts. The phases were separated and the organic phase was washed with brine, filtered through Whatman 1 PS paper and concentrated in vacuo to give crude product. Purification by chromatography (0-100%) DCM:MeOH:NH₄OH/84:15:1 to EtOAc gave the desired title compound of Step C as an amber oil (1.65 g, 81%). ¹H-NMR (400 MHz, DMSO-d₆) δ 6.57 (d, 1H), 6.14 (t, J=2.8 Hz, 1H), 6.08 (dd, J=8.4, and 2.7 Hz, 1H), 4.50 (s, 2H), 3.59 (s, 3H), 3.22-3.26 (m, 2H), 3.00 (s, 3H), 2.81-2.89 (m, 4H), 2.70 (t, J=6.7 Hz, 2H), and 2.47-2.54 (m, 4H); MS (ESI) 314 [M+H].

Step D: N-[3-(2-Ethyl-5-{2-[(4-(methyloxy)-3-{4-[2-(methylsulfonyl)ethyl]-1-piperazinyl}phenyl)amino]-4-pyrimidinyl}-1,3-thiazol-4-yl)phenyl]benzenesulfonamide

The title compound of Example 5 was made in a similar procedure to that described in the preparation of Example 2, Step D, replacing 3,4,5-tris(methyloxy)phenyl amine with 4-(methyloxy)-3-{4-[2-(methylsulfonyl)ethyl]-1-piperazinyl}phenyl amine. Yield: 28 mg (0.04 mmol, 24% yield). ¹H-NMR (400 MHz, d₆-DMSO) δ 10.42 (s, 1H), 9.44 (s, 1H), 8.15 (d, 1H, J=5.1 Hz), 7.73 (d, 2H, J=7.4 Hz), 7.51-7.61 (m, 3H), 7.18-7.38 (m, 6H), 6.85 (d, 1H, J=8.8 Hz), 6.17 (d, 1H, J=5.1 Hz), 3.75 (s, 3H), 3.30 (m, 2H), 3.04 (s, 3H), 2.98-3.03 (m, 6H), 2.76 (t, 2H, J=6.4 Hz), 2.58 (brs, 4H), and 1.33 (t, 3H, J=7.5 Hz); MS (ESI) m/z=734 [M+H]⁺.

### Example 6

### N-[3-(2-(1-Methylethyl)-5-{2-[(6-{4-[2-(methyloxy)ethyl]-1-piperazinyl}-3-pyridinyl)amino]-4-pyrimidinyl}-1,3-thiazol-4-yl)phenyl]benzenesulfonamide

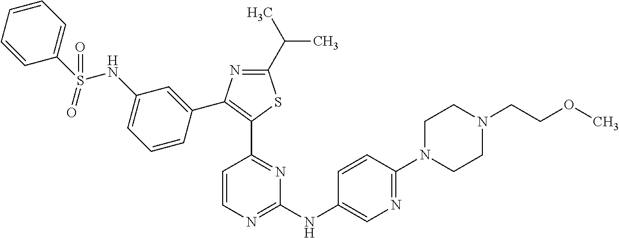

Step A: 1-[2-(Methyloxy)ethyl]-4-(5-nitro-2-pyridinyl)piperazine

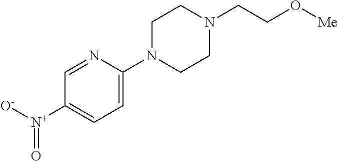

To a solution containing 2.7 g (18.9 mmol) of 1-[2-(methyloxy)ethyl]piperazine and 20 mL of THF at 0° C. was added 0.9 g (23 mmol) of a 60% suspension of sodium hydride in mineral oil. The reaction mixture was allowed to stir for 15 min and 3.0 g (18.9 mmol) of 2-chloro-5-nitropyridine was added. The reaction mixture was heated at 60° C. overnight and then quenched by the addition of H₂O and extracted with EtOAc. The combined organic layers were dried over MgSO₄ and the solvents were removed under reduced pressure. The residue was subjected to silica gel chromatography to give 2.7 g (54%) of 142-(methyloxy)ethyl]-4-(5-nitro-2-pyridinyl)piperazine as a yellow solid: ¹H-NMR (400 MHz, DMSO-d₆) δ 8.95 (d, J=2.8 Hz, 1H), 8.21 (dd, J=9.6 and 2.8 Hz, 1H), 6.94 (d, J=9.7 Hz, 2H), 3.71-3.78 (m, 4H), 3.46 (t, J=5.7 Hz, 2H), 3.24 (s, 3H), and 2.50-2.53 (m, 6H).

Step B: 6-{4-[2-(Methyloxy)ethyl]-1-piperazinyl}-3-pyridinamine hydrochloride

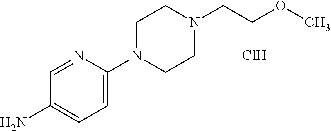

A suspension containing 2.7 g (10.1 mmol) of 142-(methyloxy)ethyl]-4-(5-nitro-2-pyridinyl)piperazine, 0.27 g of 5% Pt on carbon, and 30 mL of EtOH was treated under a 55 psi atmosphere of H₂ for 13 h, then filtered over a pad of Celite. The solvents were removed under reduced pressure to yield 2.4 g (100%) of 6-{-442-(methyloxy)ethyl]-1-piperazinyl}-3-pyridinamine. This material was converted to its corresponding hydrochloride salt by treatment of a EtOAc solution of the freebase with 4.0HCl in ether. Filtration of the resulting mixture gave 6-{4-[2-(methyloxy)ethyl]-1-piperazinyl}-3-pyridinamine hydrochloride as an off white solid: 1H-NMR (400 MHz, CD₃OD) δ 8.05 (brs, 1H), 7.66 (dd, J=9.3 and 2.8 Hz, 1H), 7.13 (d, J=9.2 Hz, 1H), 4.41 (brs, 2H), 3.75-3.80 (m, 2H), 3.66-3.75 (m, 2H), 3.41-3.46 (m, 7H), 3.33-3.41 (m, 2H), and 3.18-3.29 (m, 2H).

Step C: Ethyl 3-[(phenylsulfonyl)amino]benzoate

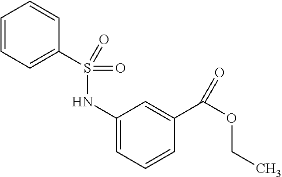

To ethyl 3-aminobenzoate (7.0 g, 42.38 mmol) and pyridine (3.5 g, 44.49 mmol) in THF (500 mL), was added benzenesulfonyl chloride (8.0 g, 44.5 mmol) in THF (250 mL) dropwise. The reaction was stirred at rt overnight. Silica was added and the reaction mixture was concentrated on the rotovap, and the residue was purified by flash column chromatography with an EtOAc hexane mixture to afford 13 g of the title compound of Step C. MS (ESI): 306 [M+H]³⁰.

Step D: N-{3-[2-(2-Chloro-4-pyrimidinyl)acetyl]phenyl}benzenesulfonamide

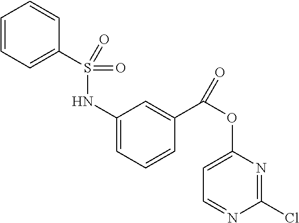

To ethyl 3-[(phenylsulfonyl)amino]benzoate (12 g, 39.75 mmol) and 1N LHMDS in THF (250 mL) was added 2-chloro-4-methylpyrimidine (5.1 g, 39.75 mmol) slowly via addition funnel and the reaction allowed to warm to rt and after 3 h was quenched with 6 N HCl. The layers were separated and the aqueous layer was extracted with EtOAc until the last organic layer showed no spot on TLC. The organic layers were combined, dried over Na₂SO₄ and stripped on the rotovap. The residue was treated with DCM and EtOAc to afford 9.0 g of the title compound of Step D. MS (ESI): 388 [M+H]⁺.

Step E: N-{3-[5-(2-Chloro-4-pyrimidinyl)-2-(1-methylethyl)-1,3-thiazol-4-yl]phenyl}benzenesulfonamide

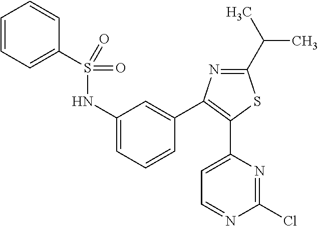

Following a procedure analogous to the procedure described in Example 2, Step C, 2.7 g of the title compound of Step E was prepared using N-{3-[2-(2-chloro-4-pyrimidinyl)acetyl]phenyl}benzenesulfonamide (9.0 g, 23.20 mmol, step B) and 2-methylpropanethioamide (2.87 g, 27.84 mmol), prepared by a procedure analogous to Intermediate 3. ¹H-NMR (400 MHz, DMSO-d₆) δ 10.46 (s, 1H), 8.49 (d, J=5.5 Hz, 1H), 7.75 (d, J=7.3 Hz, 2H), 7.61 (t, J=7.1 Hz, 1H), 7.54 (t, J=7.5 Hz, 2H), 7.35 (t, J=7.9 Hz, 1H), 7.23 (d, J=5.9 Hz, 2H), 7.19 (s, 1H), 6.92 (d, J=5.5 Hz, 1H), 3.23-3.42 (m, 1H), and 1.37 (d, J=7.0 Hz, 6H); MS (ESI): 471 [M+H]⁺.

In an alternative procedure the compound of Step E was prepared by the following procedure.

Step F: 2-Propen-1-yl {3-[5-(2-chloro-4-pyrimidinyl)-2-(1-methylethyl)-1,3-thiazol-4-yl]phenyl}carbamate

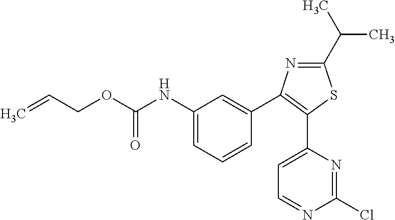

Following a procedure analogous to the procedure described in Example 2, Step C, using 2-propen-1-yl {3-[(2-chloro-4-pyrimidinyl)acetyl]phenyl}carbamate (10.0 g, 30.14 mmol), prepared by a procedure analogous to Intermediate 1, Step B, and 2-methylpropanethioamide (3.73 g, 36.17 mmol), prepared by a procedure analogous to Intermediate 3, 5.74 g of the title compound of Step F was obtained. MS (ESI): 415 [M+H]⁺.

Step G: {3-[5-(2-Chloro-4-pyrimidinyl)-2-(1-methylethyl)-1,3-thiazol-4-yl]phenyl}amine

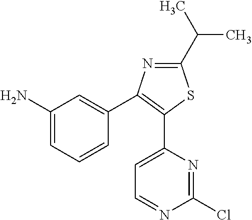

To a solution containing 2-propen-1-yl {3-[5-(2-chloro-4-pyrimidinyl)-2-(1-methylethyl)-1,3-thiazol-4-yl]phenyl}carbamate (5.3 g, 12.77 mmol) and DCM (225 mL) was added tributyltin hydride (5.95 g, 20.43 mmol), followed by and trans-dichlorobis(triphenylphosphine)palladium (II) (0.53 g, 0.64 mmol) and HOAc (1.84 g, 30.65 mmol). At the conclusion of the reaction, silica was added and the volatiles removed under reduced pressure. The residue was purified by flash column chromatography with (84% DCM, 15% MeOH, and 1% NH₄OH): DCM 0% to 100% to afford 3.4 g of the title compound of Step G. ¹H-NMR (400 MHz, DMSO-d₆) δ 8.57 (d, J=5.1 Hz, 1H), 7.16 (d, J=5.1 Hz, 1H), 7.10 (t, J=7.7 Hz, 1H), 6.72-6.75 (m, 1 H), 6.64-6.69 (m, 1H), 6.60-6.63 (m, 1H), 5.28 (s, 2H), 3.27-3.40 (m, 1H), and 1.38 (d, J=7.0 Hz, 6H). MS (ESI): 331 [M+H]⁺.

Step H: N-{3-[5-(2-Chloro-4-pyrimidinyl)-2-(1-methylethyl)-1,3-thiazol-4-yl]phenyl}benzenesulfonamide

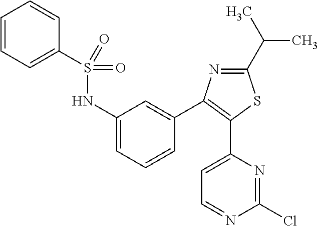

2.61 g of the title compound of Step H was prepared from {3-[5-(2-chloro-4-pyrimidinyl)-2-(1-methylethyl)-1,3-thiazol-4-yl]phenyl}amine (3.16 g, 9.55 mmol, step E) and benzenesulfonyl chloride (1.77 g, 10.0 mmol) following a procedure analogous to Example 1, Step C. MS (ESI): 471 [M+H]⁺.

Step I: N-[3-(2-(1-methylethyl)-5-{2-[(6-{4-[2-(methyloxy)ethyl]-1-piperazinyl}-3-pyridinyl)amino]-4-pyrimidinyl}-1,3-thiazol-4-yl)phenyl]benzenesulfonamide

To a mixture of N-{3-[5-(2-chloro-4-pyrimidinyl)-2-(1-methylethyl)-1,3-thiazol-4-yl]phenyl}benzenesulfonamide (500 mg, 1.06 mmol), 6-{4-[2-(methyloxy)ethyl]-1-piperazinyl}-3-pyridinamine (289.2 mg, 1.06 mmol) and i-PrOH was added 4 N HCl in dioxane (1.06 mL, 4.2 mmol) in a microwave vessel. The mixture was heated at 170° C. for 1 h in a microwave apparatus. The mixture was then allowed to cool to rt and partitioned between 5:1 CHCl₃: i-PrOH and saturated aq. NaHCO₃. The organic layer was separated, dried over Na₂SO₄, filtered, and concentrated to give a dark mixture. The residue was dissolved in CHCl₃, absorbed onto silica gel, and purified by silica gel chromatography (eluting a gradient of 100% A to 70:30 B:A where A=EtOAc, B=5:1:0.1 EtOAc: MeOH: conc NH₄OH) to give, after combining and concentrating relevant fractions, 259.2 mg (36.4% yield) of the title compound of Example 6 as an orange oil. ¹H-NMR (400 mg, DMSO-d₆): δ 10.38 (brs, 1H), 9.42 (s, 1H), 8.34 (d, 1H, J=2.6 Hz), 8.11 (d, 1H, J=5.2 Hz), 7.76 (dd, 1H, J=9.0 and 2.4 Hz), 7.69 (d, 2H, J=7.2 Hz), 7.55 (d, 1H, J=7.2 Hz), 7.49 (m, 2H), 7.26 (d, 1 H, J=7.6 Hz), 7.16 (m, 3H), 6.74 (d, 1H, J=9.2 Hz), 6.16 (d, 1H, J=5.2 Hz), 6.16 (d, 1H, J=5.2 Hz), 3.34 (brs, 4H), 3.27 (s, 6H), 3.20 (s, 3H), and 1.32 (d, 2H, J=6.7 Hz); m/z (ES): 671.4 [M+H]⁺.

### Example 7

### N-[3-(2-(1-methylethyl)-5-{2-[6-{4-[2-(methyloxy)ethyl]-1-piperazinyl}-3-pyridinyl)amino]-4-pyrimidinyl}-1,3-thiazol-4-yl)phenyl]benzenesulfonamide hydrochloride

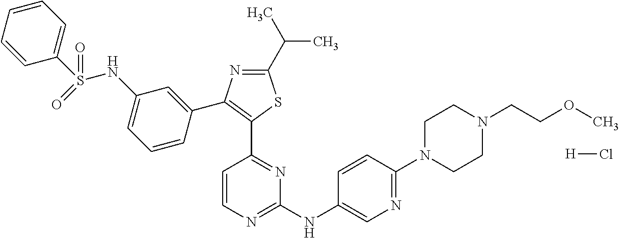

A mixture of N-[3-(2-(1-methylethyl)-5-{2-[(6-{4-[2-(methyloxy)ethyl]-1-piperazinyl}-3-pyridinyl)amino]-4-pyrimidinyl}-1,3-thiazol-4-yl)phenyl]benzenesulfonamide (251.1 mg, 0.374 mmol), prepared in a procedure analogous to Example 6, Step I, in DCM (5 mL) was treated with 2 N HCl in Et₂O (0.187 mL, 0.374 mmol). The cloudy mixture was concentrated with a rotary evaporator and pumped under high vacuum to supply the title compound of Example 7 (249.9 mg, 95% yield) as an orange solid. ¹H-NMR (400 mg, DMSO-d₆) δ 10.41 (s, 1H), 10.30 (brs, 1H), 9.65 (brs, 1H), 8.48 (s, 1H), 8.17 (d, 1H, J=5.2 Hz), 7.92 (d, 1H, J=9.1 Hz), 7.70 (d, 2H, J=7.4 Hz), 7.56 (t, 1 H, J=7.5 Hz), 7.47-7.51 (m, 3H), 7.29 (t, 1H, J=8.1 Hz), 7.15-7.18 (m, 3H), 6.98 (d, 1H, J=8.9 Hz), 6.23 (d, 1H, J=5.1 Hz), 4.26 (d, 2H, J=14 Hz), 3.99 (heptet, 1H, J=7.1 Hz), 3.95 (brs, 2H), 3.69 (t, 2H, J=4.5 Hz), 3.53 (d, 2H, J=11.6 Hz), 3.29 (s, 3H), 3.24-3.32 (m, 6H), 3.09-3.21 (m, 2H), and 1.33 (d, 6H, J=7.0 Hz); m/z (ES): 671.2 [M+H]⁺.

### Example 8

### N-{3-[2-(1-Methylethyl)-5-(2-{[6-(4-morpholinyl)-3-pyridinyl]amino}-4-pyrimidinyl)-1,3-thiazol-4-yl]phenyl}benzenesulfonamide hydrochloride

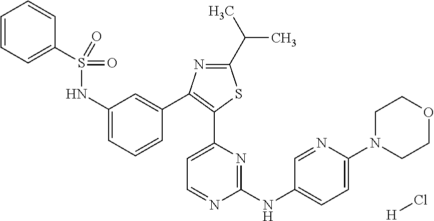

Step A: N-{3-[2-(1-Methylethyl)-5-(2-{[6-(4-morpholinyl)-3-pyridinyl]amino}-4-pyrimidinyl)-1,3-thiazol-4-yl]phenyl}benzenesulfonamide

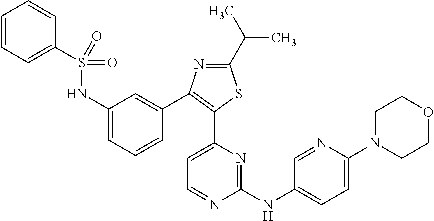

To a suspension of N-{3-[5-(2-chloro-4-pyrimidinyl)-2-(1-methylethyl)-1,3-thiazol-4-yl]phenyl}benzenesulfonamide (282 mg, 0.60 mmol), prepared by a procedure analogous to Example 6, Step E, in a mixture of 4 mL of 1-butanol and 0.4 mL of MeOH was added 6-(4-morpholinyl)-3-pyridinamine (129 mg, 0.72 mmol), prepared by a procedure analogous to Intermediate 4, Step B. The suspension was heated to 170° C. for 80 minutes in a microwave reactor. The crude reaction mixture was concentrated, adsorbed onto silica gel, and purified via flash chromatography with 0-100% EtOAc/DCM to generate 330 mg (90% yield) of the product of Step A as a yellow powder. ES-LCMS m/z 614 (M+H).

Step B: N-{3-[2-(1-Methylethyl)-5-(2-{[6-(4-morpholinyl)-3-pyridinyl]amino}-4-pyrimidinyl)-1,3-thiazol-4-yl]phenyl}benzenesulfonamide hydrochloride

The title compound of Example 8 was synthesized by adding a 4 M solution of HCl in THF (135 μL, 0.54 mmol) to a solution of N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide (330 mg, 0.54 mmol) in DCM (20 mL). After stirring for 30 minutes at room temperature, the solvent was removed, and the residue was dried in vacuo to generate 310 mg (88% yield) of the title compound of Example 8 as a yellow solid. ¹H-NMR (400 MHz, DMSO-d₆) δ 10.42 (s, 1H), 9.76 (brs, 1H), 8.51 (s, 1H), 8.23 (d, J=5.5 Hz, 1H), 8.01 (m, 1H), 7.73 (d, J=7.7 Hz, 2H), 7.60 (t, J=7.7 Hz, 1H), 7.53 (t, J=7.7 Hz, 2H), 7.32 (t, J=7.7 Hz, 1H), 7.18-7.21 (m, 3H), 6.30 (d, J=5.5 Hz, 1H), 6.30 (d, J=5.5 Hz, 1H), 3.48 (m, 4H), 3.31 (m, 1H), and 1.37 (d, J=7.0 Hz, 6H); ES-LCMS m/z 614 (M+H).

### Example 9

### N-{3-[5-(2-{[3-[4-(2-Cyanoethyl)-1-piperazinyl]-4-(methyloxy)phenyl]amino}-4-pyrimidinyl)-2-(1-methylethyl)-1,3-thiazol-4-yl]phenyl}benzenesulfonamide

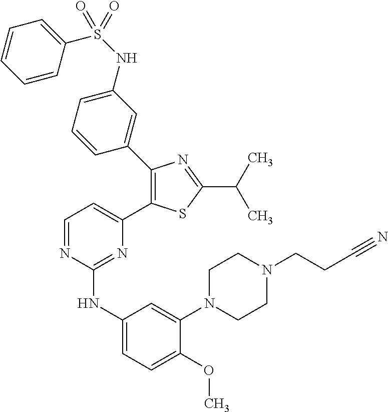

Step A: 1-[2-(Methyloxy)-5-nitrophenyl]piperazine

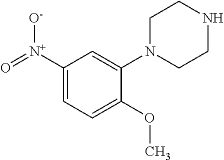

Potassium nitrate (0.63 g, 6.2 mmol) was added portion-wise to a solution of 1-[2-(methyloxy)phenyl]piperazine (1.0 g, 5.2 mmol) in sulfuric acid (5 mL). The resulting brown solution was stirred at rt for 30 min. The reaction solution was slowly added to an ice slurry. The mixture was neutralized with 5 N NaOH and extracted with EtOAc. The organic fraction was washed with brine and dried over magnesium sulfate. The solvent was removed under vacuum and provided 980 mg (66%) of the desired nitration product of Step A. ES-LCMS m/z 238.14 (M+H).

Step B: 3-{4-[2-(Methyloxy)-5-nitrophenyl]-1-piperazinyl}propanenitrile

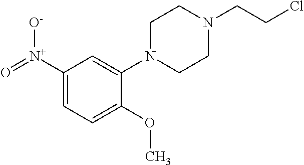

1-[2-(Methyloxy)-5-nitrophenyl]piperazine (0.98 g, 4.1 mmol) was stirred with 3-bromopropanenitrile (0.519 mL, 6.2 mmol), cesium carbonate (2.7 g, 8.3 mmol) and DMF (20 mL). The reaction was stirred at 60° C. for 4 h, but little conversion was observed. The reaction was heated to 100° C. and stirred for 3 days. Solvent was removed under vacuum and the residue was dissolved in EtOAc. The organic layer was washed with brine and dried with magnesium sulfate. The crude was purified by silica gel chromatography 5-100% (90%/9% MeOH/1% NH₄OH)/DCM gradient followed by a second silica gel purification 10-100% EtOAc/Hex gradient to give 0.150 g, 14% yield of the desired nitrile of Step B. ES-LCMS m/z 291.10 (M+H).

Step C: 3-{4-[5-Amino-2-(methyloxy)phenyl]-1-piperazinyl}propanenitrile

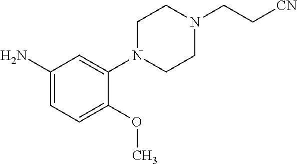

3-{4-[2-(Methyloxy)-5-nitrophenyl]-1-piperazinyl}propanenitrile (0.15 g, 0.52 mmol) was dissolved in EtOH and stirred vigorously with 5% Pd/C (43 mg) under a hydrogen atmosphere at 55 psi. The reaction was stirred at rt for 15 h. The reaction was de-gassed and filtered through Celite. The filtrate was evaporated under vacuum and provided 0.13 g, 97% yield of the desired phenyl amine of Step C as a tan solid. ES-LCMS m/z 261.14 (M+H)

Step D: N-{3-[5-(2-{[3-[4-(2-Cyanoethyl)-1-piperazinyl]-4-(methyloxy)phenyl]amino}-4-pyrimidinyl)-2-(1-methylethyl)-1,3-thiazol-4-yl]phenyl}benzenesulfonamide

N-{3-[5-(2-Chloro-4-pyrimidinyl)-2-(1-methylethyl)-1,3-thiazol-4-yl]phenyl}benzenesulfonamide (0.10 g, 0.21 mmol), prepared by a procedure analogous to Example 6, Step E, 3-{4-[5-amino-2-(methyloxy)phenyl]-1-piperazinyl}propanenitrile (0.050 g, 0.19 mmol), 4 M HCl in dioxane (0.072 mL, 0.29 mmol) and 2,2,2-trifluoroethanol (3 mL) were heated by microwave radiation to 170° C. for 30 min. The reaction was cooled to rt and concentrated to a residue. The crude residue was purified by silica gel chromatography 5-100% (90% DCM:9% MeOH:1% NH₄OH)/DCM gradient and provided 0.053 g, 40% yield of the title compound of Example 9 as a yellow powder. ¹H-NMR (400 MHz, DMSO-d₆) δ 10.43 (s, 1H), 9.48 (s, 1H), 8.17 (d, J=5.1 Hz, 1H), 7.75 (d, J=7.0 Hz, 2H), 7.58-7.66 (m, 1H), 7.47-7.57 (m, 3H), 7.28-7.39 (m, 1H), 7.15-7.26 (m, 4H), 6.86 (d, J=9.2 Hz, 1H), 6.19 (d, J=5.1 Hz, 1H), 3.80 (sept, 1H), 3.76 (s, 3H), 2.97-3.08 (m, 4H), 2.70 (t, J=6.1 Hz, 2H), 2.63-2.66 (m, 2H), 2.54-2.63 (m, J=14.7 Hz, 4H), and 1.38 (d, J=7.0 Hz, 6H); ES-LCMS m/z 695.34 (M+H).

### Example 10

### N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-4-fluorobenzenesulfonamide

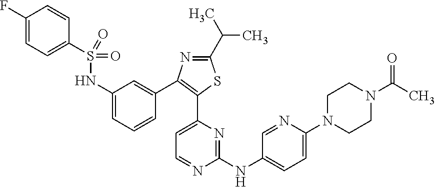

Step A: N-{3-[5-(2-Chloro-4-pyrimidinyl)-2-(1-methylethyl)-1,3-thiazol-4-yl]phenyl}-4-fluorobenzenesulfonamide

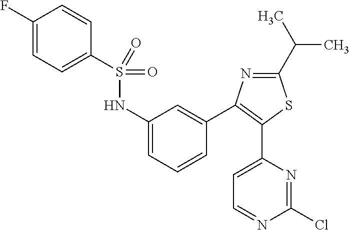

To a solution of 3-[5-(2-chloro-4-pyrimidinyl)-2-(1-methylethyl)-1,3-thiazol-4-yl]phenyl amine (200 mg, 0.61 mmol), prepared by a procedure analogous to Example 6, Step G, and pyridine (54 μL, 0.66 mmol) in DCM (10 mL) was added a solution of 4-fluorobenzenesulfonyl chloride (130 mg, 0.66 mmol) in DCM (5 mL). The reaction was stirred overnight at rt. After 16 h, the reaction was not complete by LC/MS, so more 4-fluorobenzenesulfonyl chloride (33 mg, 0.17 mmol) was added. The reaction was stirred overnight at rt. The reaction mixture was concentrated, adsorbed onto silica gel, and purified via flash chromatography with 0-50% EtOAc/DCM to generate 280 mg (94% yield) of the product of Step A as a white powder. ES-LCMS m/z 489 (M+H).

Step B: N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-4-fluorobenzenesulfonamide

The title compound of Example 148 was synthesized by heating a suspension of N-{3-[5-(2-chloro-4-pyrimidinyl)-2-(1-methylethyl)-1,3-thiazol-4-yl]phenyl}-4-fluorobenzenesulfonamide (100 mg, 0.20 mmol), 6-(4-acetyl-1-piperazinyl)-3-pyridinamine (50 mg, 0.22 mmol), prepared by a procedure analogous to Example 3, Step D, and 4 M HCl in THF (100 μL, 0.40 mmol) in iPrOH (3 mL) overnight at 90° C. The reaction mixture was concentrated, redissolved in 2 mL of MeOH, and purified by reverse-phase HPLC, eluting with 10-60% ACN/0.1% aqueous trifluoroacetic acid. The desired fractions were combined, neutralized with saturated aqueous sodium bicarbonate, and extracted with EtOAc. The organic layer was dried over anhydrous sodium sulfate, filtered, and concentrated to generate 80 mg (60% yield) of the title compound of Example 10 as a yellow solid. ¹H-NMR (400 MHz, DMSO-d₆) δ 10.45 (brs, 1H), 9.51 (s, 1H), 8.42 (d, J=2.5 Hz, 1H), 8.18 (d, J=5.1 Hz, 1H), 7.77-7.85 (m, 4H), 7.32-7.39 (m, 3H), 7.19-7.22 (m, 3H), 6.83 (d, J=9.2 Hz, 1H), 6.23 (d, J=5.1 Hz, 1H), 3.53-3.56 (m, 4H), 3.44-3.46 (m, 2 H), 3.36-3.39 (m, 2H), 3.29 (m, 1H), 2.04 (s, 3H), and 1.36 (d, J=6.6 Hz, 6H); ES-LCMS m/z 673 (M+H).

### Example 11

### N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-3-fluorobenzenesulfonamide

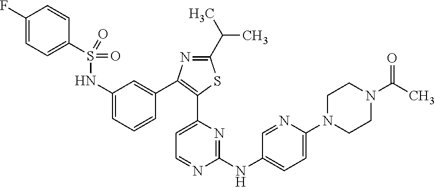

Step A: N-{3-[5-(2-Chloro-4-pyrimidinyl)-2-(1-methylethyl)-1,3-thiazol-4-yl]phenyl}-3-fluorobenzenesulfonamide

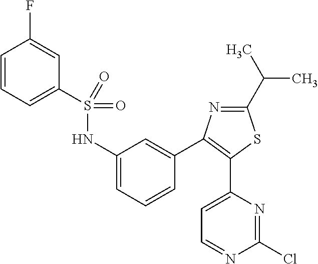

To a solution of 3-[5-(2-chloro-4-pyrimidinyl)-2-(1-methylethyl)-1,3-thiazol-4-yl]phenyl amine (100 mg, 0.30 mmol), prepared by a procedure analogous to Example 6, Step G, and pyridine (27 μL, 0.33 mmol) in DCM (10 mL) was added a solution of 3-fluorobenzenesulfonyl chloride (44 μL, 0.33 mmol) in DCM (5 mL). The reaction was stirred for 72 h at rt and then 24 h at 45° C. The reaction mixture was concentrated, adsorbed onto silica gel, and purified via flash chromatography with 0-100% EtOAc/DCM to generate 114 mg (78% yield) of the product of Step A as a white powder. ES-LCMS m/z 489 (M+H).

Step B: N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-3-fluorobenzenesulfonamide

The title compound of Example 149 was synthesized by heating a suspension of N-{3-[5-(2-chloro-4-pyrimidinyl)-2-(1-methylethyl)-1,3-thiazol-4-yl]phenyl}-3-fluorobenzenesulfonamide (60 mg, 0.12 mmol) and 6-(4-acetyl-1-piperazinyl)-3-pyridinamine (35 mg, 0.13 mmol), prepared by a procedure analogous to Example 3, Step D, in iPrOH (2 mL) at 90° C. for 40 h. The reaction mixture was concentrated, redissolved in 2 mL of MeOH, and purified by reverse-phase HPLC, eluting with 10-60% ACN/0.1% aqueous trifluoroacetic acid. The desired fractions were combined, neutralized with saturated aqueous sodium bicarbonate, and extracted with EtOAc. The organic layer was dried over anhydrous sodium sulfate, filtered, and concentrated to generate 47 mg (58% yield) of the title compound of Example 11 as a yellow solid. ¹H-NMR (400 MHz, DMSO-d₆) δ 10.52 (brs, 1H), 9.50 (s, 1H), 8.40 (s, 1H), 8.16 (d, J=5.0 Hz, 1H), 7.83 (dd, J=9.2 and 2.4 Hz, 1H), 7.47-7.59 (m, 4 H), 7.31-7.36 (m, 1H), 7.18-7.22 (m, 3H), 6.83 (d, J=8.9 Hz, 1H), 6.25 (d, J=5.6 Hz, 1H), 3.53-3.54 (m, 4H), 3.44-3.46 (m, 2H), 3.36-3.39 (m, 2H), 3.29 (m, 1H), 2.04 (s, 3H), and 1.36 (d, J=6.8 Hz, 6H); ES-LCMS m/z 673 (M+H).

### Example 12

### N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-2-fluorobenzenesulfonamide

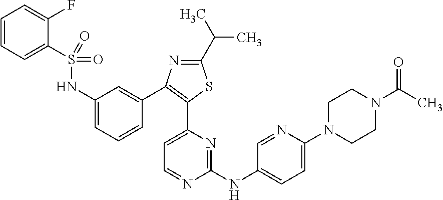

Step A: N-{3-[5-(2-Chloro-4-pyrimidinyl)-2-(1-methylethyl)-1,3-thiazol-4-yl]phenyl}-2-fluorobenzenesulfonamide

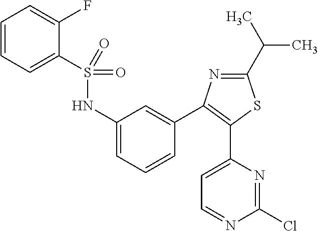

To a solution of 3-[5-(2-chloro-4-pyrimidinyl)-2-(1-methylethyl)-1,3-thiazol-4-yl]phenyl amine (100 mg, 0.30 mmol), prepared by a procedure analogous to Example 6, Step G, and pyridine (27 μL, 0.33 mmol) in DCM (10 mL) was added a solution of 2-fluorobenzenesulfonyl chloride (44 μL, 0.33 mmol) in DCM (5 mL). The reaction was stirred for 72 h at rt and 24 h at 45° C. The reaction mixture was concentrated, adsorbed onto silica gel, and purified via flash chromatography with 0-100% EtOAc/DCM to generate 138 mg (94% yield) of the product of Step A as a white powder. ES-LCMS m/z 489 (M+H).

Step B: N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-2-fluorobenzenesulfonamide

The title compound of Example 150 was synthesized by heating a suspension of N-{3-[5-(2-chloro-4-pyrimidinyl)-2-(1-methylethyl)-1,3-thiazol-4-yl]phenyl}-2-fluorobenzenesulfonamide (100 mg, 0.20 mmol) and 6-(4-acetyl-1-piperazinyl)-3-pyridinamine (58 mg, 0.22 mmol), prepared by a procedure analogous to Example 3, Step D, in iPrOH (2 mL) at 90° C. for 40 h. The reaction mixture was concentrated, redissolved in 2 mL of MeOH, and purified by reverse-phase HPLC, eluting with 10-60% ACN/0.1% aqueous trifluoroacetic acid. The desired fractions were combined, neutralized with saturated aqueous sodium bicarbonate, and extracted with EtOAc. The organic layer was dried over anhydrous sodium sulfate, filtered, and concentrated to generate 47 mg (35% yield) of the title compound of Example 12 as a yellow solid. ¹H-NMR (400 MHz, DMSO-d₆) δ 10.75 (s, 1H), 9.49 (s, 1H), 8.40 (s, 1H), 8.16 (d, J=4.7 Hz, 1H), 7.76-7.85 (m, 2H), 7.63-7.69 (m, 1H), 7.39 (t, J=8.6 Hz, 1H), 7.29-7.31 (m, 2H), 7.17-7.22 (m, 3H), 6.82 (d, J=8.7 Hz, 1H), 6.22 (d, J=5.3 Hz, 1H), 3.52-3.55 (m, 4H), 3.45-3.46 (m, 2H), 3.36-3.39 (m, 2H), 3.28 (m, 1H), 2.04 (s, 3H), and 1.36 (d, J=6.8 Hz, 6H); ES-LCMS m/z 673 (M+H).

### Example 13

### 2,6-Difluoro-N-{3-[5-(2-{[3-fluoro-4-({1-[2-(methylsulfonyl)ethyl]-4-piperidinyl}oxy)phenyl]amino}-4-pyrimidinyl)-2-(1-methylethyl)-1,3-thiazol-4-yl]phenyl}benzenesulfonamide hydrochloride

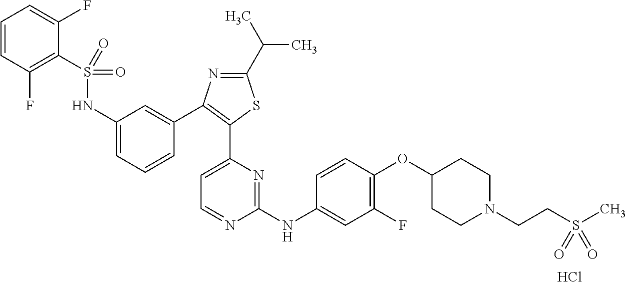

Step A: Phenylmethyl 4-[(2-fluoro-4-nitrophenyl)oxy]-1-piperidinecarboxylate

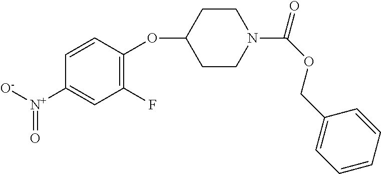

To a chilled (0° C.) stirring solution of 2-fluoro-4-nitrophenol (15 g, mmol), 1-benzyl-4-hydroxy-1-piperidinecarboxylate and PS-triphenylphosphine in DCM was added di-tert-butylazodicarboxylate. The reaction was equilibrated to rt and stirred for 19 h. The reaction was concentrated directly onto silica gel and purified in batches to yield a crude material. Each crude batch was dissolved in ether and washed with 2.0 N sodium hydroxide solution (2×50 mL). All batches were combined to afford the title compound of Step A as an opaque syrup (30 g; 84%). ¹H-NMR (400 MHz, CHLOROFORM-d) δ 7.93-8.13 (m, 2H), 7.30-7.49 (m, 3H), 7.05 (t, J=8.3 Hz, 1H), 6.36 (s, 2H), 5.15 (s, 2H), 4.60-4.77 (m, 1H), 3.66-3.84 (m, 2H), 3.41-3.60 (m, 2H), 1.98 (s, 2H), and 1.88 (s, 2H)

Step B: 4-[(2-Fluoro-4-nitrophenyl)oxy]piperidine hydrobromide

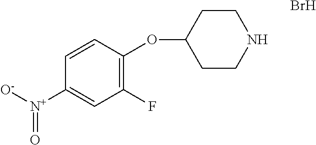

Hydrobromic acid (30 weight % in acetic acid, 26.9 g, 99.73 mmol) was added to phenylmethyl 4-[(2-fluoro-4-nitrophenyl)oxy]-1-piperidinecarboxylate (30 g, 80.13 mmol) and the mixture was stirred at rt for 0.5 h. Ether (400 mL) was slowly added and a white solid was collected by filtration. The solid was washed with ether (100 mL) and hexanes (150 mL) and air-dried to afford the title compound of Step B (12.95 g, 50%): ¹H-NMR (400 MHz, DMSO-d₆) δ 8.19 (dd, J=11.2, and 2.7 Hz, 1H), 8.06-8.14 (m, 1H), 7.47-7.58 (m, 1H), 4.87-4.98 (m, 1H), 3.42 (s, 1H), 3.18-3.30 (m, J=3.7 Hz, 2H), 3.02-3.18 (m, 2H), 2.07-2.21 (m, 2H), and 1.77-1.96 (m, 2H).

Step C: 4-[(2-Fluoro-4-nitrophenyl)oxy]-142-(methylsulfonyl)ethyl]piperidine

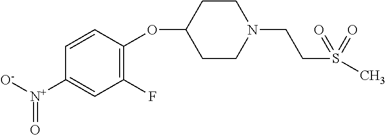

To a mixture of 4-[(2-fluoro-4-nitrophenyl)oxy]piperidine hydrobromide (1.25 g, 3.89 mmol) and sodium carbonate (1 g, 9.43 mmol) stirring in THF (15 mL) was added methyl vinyl sulfone (0.9 g, 8.48 mmol). The resulting reaction was heated to 65° C. for 2-3 h. The reaction was poured into EtOAc (100 mL) and washed with water (20 mL) and brine (20 mL), filtered through Whatman 1 PS paper, and concentrated in vacuo to the title compound of Step C (1.42 g; 100%) contaminated with a small amount of excess methyl vinyl sulfone. The reported data represents the major component of the reaction. ¹H-NMR (400 MHz, DMSO-d₆) δ 8.16 (dd, J=11.2, and 2.7 Hz, 1H), 8.05-8.11 (m, 1H), 7.49 (t, 1H), 4.65-4.75 (m, 1H), 3.26-3.31 (m, 2 H), 3.00-3.05 (m, J=1.5 Hz, 3H), 2.67-2.78 (m, 4H), 2.25-2.41 (m, 2H), 1.91-2.05 (m, 2H), and 1.60-1.76 (m, 2H).

Step D: [3-Fluoro-4-({1-[2-(methylsulfonyl)ethyl]-4-piperidinyl}oxy)phenyl]amine 3-fluoro-4-({1-[2-(methylsulfonyl)ethyl]-4-piperidinyl}oxy)phenyl amine

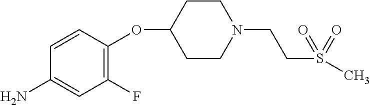

To a stirring mixture of 4-[(2-fluoro-4-nitrophenyl)oxy]-1-[2-(methylsulfonyl)ethyl]piperidine (1.35 g 3.90 mmol) in MeOH (75 mL) was added nickel (II) chloride hexahydrate (0.5 g; 2.1 mmol). The reaction mixture was chilled to 0° C. and sodium borohydride (0.3 g; 7.93 mmol) was added, causing a vigorous foaming action. The resulting reaction mixture turned black. The reaction was stirred for 0.5 h before a 2^{nd} equal addition of sodium borohydride. The reaction was stirred for 0.25 h and then quenched with a 2.0 N sodium hydroxide solution (75 mL). The reaction was partitioned between EtOAc and water/3:1. The mixture was filtered, the phases were separated, and the organic phase was washed with brine. The resulting organic phase was filtered through Whatman 1 PS paper and concentrated in vacuo to the title compound of Step D as an amber oil that was slightly contaminated (1.28 g, 100%). The data represents the title compound of Step D as the major product of the reaction. ¹H-NMR (400 MHz, DMSO-d₆) δ 6.71-6.92 (m, 1 H), 6.36 (dd, J=13.6, and 2.6 Hz, 1H), 6.27 (dd, J=8.1, and 2.2 Hz, 1H), 4.99 (s, 2 H), 3.87-4.01 (m, 1H), 3.26 (t, J=6.6 Hz, 2H), 3.02 (s, 3H), 2.63-2.76 (m, 4H), 2.13-2.27 (m, 2H), 1.77-1.89 (m, 2H), and 1.49-1.64 (m, 2H).

Step E: N-{3-[5-(2-Chloro-4-pyrimidinyl)-2-(1-methylethyl)-1,3-thiazol-4-yl]phenyl}-2,6-difluorobenzenesulfonamide

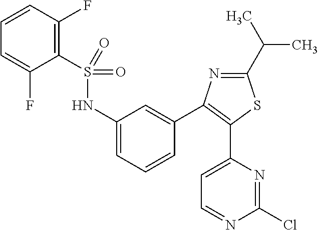

To a solution of 3-[5-(2-chloro-4-pyrimidinyl)-2-(1-methylethyl)-1,3-thiazol-4-yl]phenyl amine (1.0 g, 3.0 mmol), prepared by a procedure analogous to Example 6, Step G, and pyridine (360 μL, 4.5 mmol) in DCM (50 mL) was added a solution of 2,6-difluorobenzenesulfonyl chloride (620 μL, 4.5 mmol) in DCM (25 mL). The reaction was stirred for 48 h at rt. The reaction mixture was concentrated, adsorbed onto silica gel, and purified via flash chromatography with 0-50% EtOAc/DCM to generate 1.39 g (91% yield) of the product of Step E as a white powder. ES-LCMS m/z 507 (M+H).

Step F: 2,6-Difluoro-N-{3-[5-(2-{[3-fluoro-4-({1-[2-(methylsulfonyl)ethyl]-4-piperidinyl}oxy)phenyl]amino}-4-pyrimidinyl)-2-(1-methylethyl)-1,3-thiazol-4-yl]phenyl}benzenesulfonamide hydrochloride

The title compound of Example 13 was synthesized by heating a suspension of N-{3-[5-(2-chloro-4-pyrimidinyl)-2-(1-methylethyl)-1,3-thiazol-4-yl]phenyl}-2,6-difluorobenzenesulfonamide (100 mg, 0.20 mmol) and 3-fluoro-4-({1-[2-(methylsulfonyl)ethyl]-4-piperidinyl}oxy)phenyl amine hydrochloride (71 mg, 0.20 mmol) in iPrOH (2 mL) at 170° C. for 20 min in a microwave reactor. The product was collected by filtration of the reaction and trituration of the crude product with MeOH, generating 106 mg (67% yield) of the title compound of Example 13 as a yellow solid. ¹H-NMR (400 MHz, DMSO-d₆) δ 11.05 (s, 1H), 10.63 (brs, 1H), 9.82 (d, J=5.5 Hz, 1H), 8.24 (d, J=5.1 Hz, 1H), 7.74-7.81 (m, 1H), 7.66-7.71 (m, 1H), 7.35-7.35 (m, 2H), 7.29 (s, 1H), 7.21-7.26 (m, 4H), 7.14-7.19 (m, 1H), 6.32 (d, J=5.5 Hz, 1H), 3.74 (brs, 2H), 3.59 (brs, 2H), 3.46-3.50 (m, 2H), 3.23-3.31 (m, 4H), 3.11 (s, 3H), 2.25 (brs, 1H), 2.07 (brs, 2H), 1.88 (brs, 1H), and 1.38 (d, J=6.9 Hz, 6H); ES-LCMS m/z 787 (M+H).

### Example 14

### 5-({4-[4-(3-{[(2,6-Difluorophenyl)sulfonyl]amino}phenyl)-2-(1-methylethyl)-1,3-thiazol-5-yl]-2-pyrimidinyl}amino)-2-(4-morpholinyl)benzoic acid

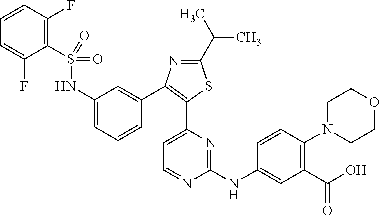

The title compound of Example 14 was synthesized by heating a suspension of N-{3-[5-(2-chloro-4-pyrimidinyl)-2-(1-methylethyl)-1,3-thiazol-4-yl]phenyl}-2,6-difluorobenzenesulfonamide (200 mg, 0.40 mmol), prepared by a procedure analogous to Example 13, Step E, and methyl 5-amino-2-(4-morpholinyl)benzoate (93 mg, 0.40 mmol) in a mixture of 1-butanol (4 mL) and MeOH (0.4 mL) at 170° C. for 80 min in a microwave reactor. The solvent was removed, and the residue was redissolved in 3 mL of water and 1 mL of 1 N NaOH. The solution was heated at 120° C. for 15 min in a microwave reactor. The reaction mixture was acidified with saturated aqueous ammonium chloride and extracted with 10% MeOH/EtOAc. The organic layer was adsorbed onto silica gel and purified via flash chromatography with 0-100% EtOAc/DCM to generate 178 mg (64% yield) of Example 14 as a yellow powder. ¹H-NMR (400 MHz, DMSO-d₆) δ 11.05 (s, 1H), 9.98 (s, 1H), 8.52 (d, J=2.6 Hz, 1H), 8.25 (d, J=5.3 Hz, 1H), 7.91 (dd, J=2.8 and 9.0 Hz, 1H), 7.60-7.71 (m, 2H), 7.37 (t, J=8.0 Hz, 1H), 7.22-7.29 (m, 5H), 6.34 (d, J=5.3 Hz, 1H), 3.80 (s, 4H), 3.27-3.31 (m, 1H), 3.05 (s, 4H), and 1.39 (d, J=6.8 Hz, 6H); ES-LCMS m/z 693 (M+H).

### Example 15

### 2,6-Difluoro-N-{3-[5-[2-(}6-[(9aR)-hexahydropyrazino[2,1-c][1,4]oxazin-8(1H)-yl]-3-pyridinyl}amino)-4-pyrimidinyl]-2-(1-methylethyl)-1,3-thiazol-4-yl]phenyl}benzenesulfonamide

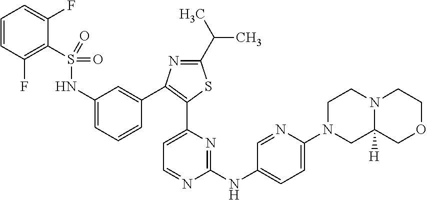

Step A: (9aR)-Octahydropyrazino[2,1-c][1,4]oxazine hydrochloride

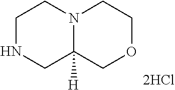

To a solution containing 2.8 g (12.1 mmol) of (9aR)-8-(phenylmethyl)octahydropyrazino[2,1-c][1,4]oxazine and 30 mL of MeOH was added 4.05 mL of 6 N aqueous HCl and 300 mg of 10% palladium on carbon. The reaction mixture was subjected to a hydrogen atmosphere overnight and filtered through Celite, eluting with MeOH. The solvents were removed under reduced pressure to give 1.6 g (62%) of (9aR)-octahydropyrazino[2,1-c][1,4]oxazine hydrochloride as a white foam. NMR (400 MHz, DMSO-d₆) δ 9.99 (brs, 2H), 3.84-4.01 (m, 4H), and 3.00-3.68 (m, 10H).

Step B: (9aR)-8-(5-Nitro-2-pyridinyl)octahydropyrazino[2,1-c][1,4]oxazine

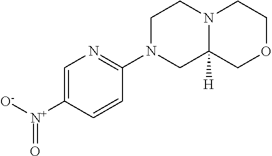

To a solution containing 0.63 g (3.97 mmol) of 2-chloro-5-nitropyridine and 5 mL of acetonitrile was added 0.85 g of (9aR)-octahydropyrazino[2,1-c][1,4]oxazine hydrochloride and 1.64 g (11.9 mmol) of potassium carbonate. The reaction mixture was allowed to stir at rt overnight, then partitioned between EtOAc and water. The water layer was further extracted with EtOAc and the combined organic layers were dried over MgSO₄ and filtered. The solvents were removed under reduced pressure and the residue was subjected to silica gel chromatography to give 0.77 g (73%) of (9aR)-8-(5-Nitro-2-pyridinyl)octahydropyrazino[2,1-c][1,4]oxazine as a yellow oil. ¹H-NMR (400 MHz, DMSO-d₆) δ 8.96 (d, J=2.8 Hz, 1H), 8.23 (dd, J=9.6 and 2.8 Hz, 1H), 6.97 (d, J=9.5 Hz, 1H), 4.49 (d, J=10.4 Hz, 1H), 4.38 (d, J=11.5 Hz, 1H), 3.77 (dt, J=11.1 and 2.7 Hz, 2H), 3.54 (td, J=11.4 and 2.3 Hz, 1H), 3.07-3.19 (m, 2H), 2.84 (ddd, J=11.2, 2.7, and 2.5 Hz, 1H), 2.61-2.71 (m, 2H), and 2.10-2.21 (m, 3H); ESIMS: 265.20 (M+H)⁺.

Step C: 6-[(9aR)-Hexahydropyrazino[2,1-c][1,4]oxazin-8(1H)-yl]-3-pyridinamine

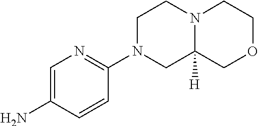

To a solution containing 0.77 g (2.9 mmol) of (9aR)-8-(5-nitro-2-pyridinyl)octahydropyrazino[2,1-c][1,4]oxazine, 5 mL of MeOH, and 10 mL of EtOH was added 70 mg of 5% platinum on carbon. The reaction mixture was subjected to a 40 psi atmosphere of hydrogen for 13 h, and then filtered over Celite. The solvents were removed under reduced pressure to give 0.6 g (88%) of dark solid: ¹H-NMR (400 MHz, DMSO-d₆) δ 7.58 (d, J=2.8 Hz, 1H), 6.90 (dd, J=8.8 and 2.93 Hz, 1H), 6.62 (d, J=8.8 Hz, 1H), 4.57 (s, 2H), 3.83 (d, J=12.3 Hz, 1H), 3.67-3.77 (m, 2 H), 3.48-3.56 (m, 1H), 3.14 (t, J=10.2 Hz, 1H), 2.71-2.79 (m, 1H), 2.61-2.69 (m, 2H), 2.52-2.59 (m, 1H), 2.12-2.22 (m, 3H), and 1.95-2.06 (m, 1H).

Step D: 2,6-Difluoro-N-{3-[5-[2-({6-[(9aR)-hexahydropyrazino[2,1-c][1,4]oxazin-8(1H)-yl]-3-pyridinyl}amino)-4-pyrimidinyl]-2-(1-methylethyl)-1,3-thiazol-4-yl]phenyl}benzenesulfonamide

The title compound of Example 15 was synthesized by heating a suspension of N-{3-[5-(2-chloro-4-pyrimidinyl)-2-(1-methylethyl)-1,3-thiazol-4-yl]phenyl}-2,6-difluorobenzenesulfonamide (100 mg, 0.20 mmol), prepared by a procedure analogous to Example 13, Step E, and 6-[(9aR)-hexahydropyrazino[2,1-c][1,4]oxazin-8(1H)-yl]-3-pyridinamine (51 mg, 0.22 mmol) in a mixture of 1-butanol (2 mL) and MeOH (0.2 mL) at 170° C. for 80 min in a microwave reactor. The reaction mixture was concentrated, redissolved in 2 mL of MeOH, and purified by reverse-phase HPLC, eluting with 10-60% ACN/0.1% aqueous trifluoroacetic acid. The desired fractions were combined, neutralized with saturated aqueous sodium bicarbonate, and extracted with EtOAc. The organic layer was dried over anhydrous sodium sulfate, filtered, and concentrated to generate 54 mg (38% yield) of the title compound of Example 15 as a yellow solid. ¹H-NMR (400 MHz, DMSO-d₆) δ 11.04 (s, 1H), 9.46 (s, 1H), 8.37 (d, J=2.5 Hz, 1H), 8.15 (d, J=5.2 Hz, 1H), 7.79 (d, J=15.3 Hz, 2H), 7.66-7.70 (m, 1H), 7.17-7.37 (m, 6H), 6.78 (d, J=8.6 Hz, 1H), 6.23 (d, J=4.0 Hz, 1H), 4.07 (d, J=10.1 Hz, 1H), 3.98 (d, J=11.9 Hz, 1H), 3.76 (d, J=11.4 Hz, 1H), 3.53 (t, J=10.9 Hz, 1H), 3.28 (m, 1H), 3.16 (t, J=10.5 Hz, 1H), 2.81 (t, J=10.3 Hz, 2H), 2.66 (m, 1H), 2.32 (t, J=11.4 Hz, 2H), 2.15-2.24 (m, 2H), and 1.37 (d, J=6.9 Hz, 6H); ES-LCMS m/z 705 (M+H).

### Example 16

### 2,6-Difluoro-N-{3-[5-{2-[(3-fluoro-4-{4-[2-(methylsulfonyl)ethyl]-1-piperazinyl}phenyl)amino]-4-pyrimidinyl}-2-(1-methylethyl)-1,3-thiazol-4-yl]ohenyl}benzenesulfonamide hydrochloride

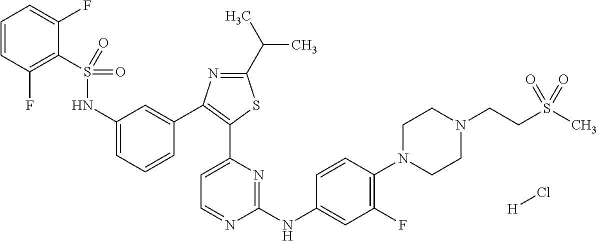

The title compound of Example 16 was synthesized by heating a suspension of N-{3-[5-(2-chloro-4-pyrimidinyl)-2-(1-methylethyl)-1,3-thiazol-4-yl]phenyl}-2,6-difluorobenzenesulfonamide (100 mg, 0.20 mmol), prepared by a procedure analogous to Example 13, Step E, and 3-fluoro-4-{4-[2-(methylsulfonyl)ethyl]-1-piperazinyl}phenyl amine (65 mg, 0.20 mmol), prepared by a procedure analogous to Intermediate 5, Step B, in a mixture of iPrOH (2 mL) and concentrated HCl (1 drop) at 170° C. for 50 min in a microwave reactor. The product was collected by vacuum filtration, washed with iPrOH, and dried in vacuo to generate 52 mg (34% yield) of the title compound of Example 16 as a yellow solid. ¹H-NMR (400 MHz, DMSO-d₆) δ 11.04 (s, 1H), 10.43 (brs, 1H), 9.81 (s, 1H), 8.23 (d, J=5.3 Hz, 1H), 7.66-7.75 (m, 2H), 7.34-7.40 (m, 2H), 7.21-7.29 (m, 5H), 7.04 (t, J=7.3 Hz, 1H), 6.31 (d, J=5.3 Hz, 1H), 3.73 (brs, 2H), 3.63 (brs, 4H), 3.27-3.48 (m, 5H), 3.13 (s, 3H), 3.01-3.07 (m, 2H), and 1.38 (d, J=6.7 Hz, 6H); ES-LCMS m/z 772 (M+H).

### Example 17

### 2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide

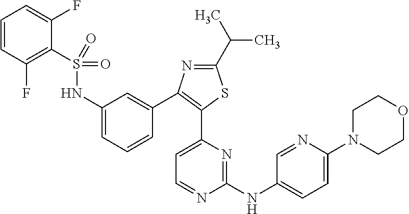

The title compound of Example 17 was synthesized by heating a suspension of N-{3-[5-(2-chloro-4-pyrimidinyl)-2-(1-methylethyl)-1,3-thiazol-4-yl]phenyl}-2,6-difluorobenzenesulfonamide (100 mg, 0.20 mmol), prepared by a procedure analogous to Example 13, Step E, and 6-(4-morpholinyl)-3-pyridinamine (39 mg, 0.22 mmol), prepared by a procedure analogous to Intermediate 4, Step B, in a mixture of 1-butanol (2 mL) and MeOH (0.2 mL) at 170° C. for 80 min in a microwave reactor. The reaction mixture was concentrated, redissolved in 2 mL of 1:1 DMSO/methanol, and purified by reverse-phase HPLC, eluting with 10-60% ACN/0.1% aqueous trifluoroacetic acid. The desired fractions were combined, neutralized with saturated aqueous sodium bicarbonate, and extracted with EtOAc. The organic layer was dried over anhydrous sodium sulfate, filtered, and concentrated to generate 94 mg (72% yield) of the title compound of Example 17 as a yellow solid. ¹H-NMR (400 MHz, DMSO-d₆) δ 11.04 (s, 1H), 9.49 (s, 1H), 8.41 (d, J=2.5 Hz, 1H), 8.15 (d, J=5.1 Hz, 1H), 7.82 (d, J=7.3 Hz, 1H), 7.65-7.70 (m, 1 H), 7.36 (t, J=7.7 Hz, 1H), 7.21-7.28 (m, 5H), 6.79 (d, J=9.1 Hz, 1H), 6.23 (d, J=5.3 Hz, 1H), 3.70 (t, J=,4.8 Hz, 4H), 3.35 (t, J=4.9 Hz, 4H), 3.28-3.30 (m, 1H), and 1.37 (d, J=6.8 Hz, 6H); ES-LCMS m/z 650 (M+H).

### Example 18

### N-{3-[5-(2-{[3-(Dimethylamino)-4-(methyloxy)phenyl]amino}-4-pyrimidinyl)-2-(1-methylethyl)-1,3-thiazol-4-yl]phenyl}-2,6-difluorobenzenesulfonamide trifluoroacetate

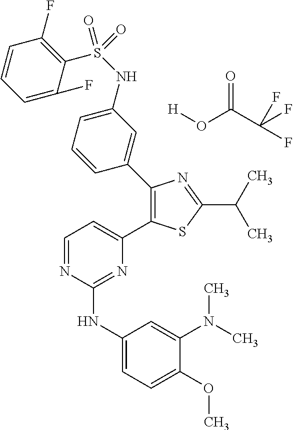

N-{3-[5-(2-Chloro-4-pyrimidinyl)-2-(1-methylethyl)-1,3-thiazol-4-yl]phenyl}-2,6-difluorobenzenesulfonamide (0.150 g, 0.30 mmol), prepared by a procedure analogous to Example 13, Step E, [5-amino-2-(methyloxy)phenyl]dimethylamine (0.045 g, 0.27 mmol), 4 M HCl in dioxane (0.168 mL, 0.67 mmol) and 2,2,2-trifluoroethanol (4 mL) were heated by microwave radiation at 170° C. for 60 min. The reaction was cooled to rt and purified by HPLC (C₁₈ column) 10-100% CH₃CN (0.1% TFA)/H₂0 (0.1% TFA) gradient and provided 0.015 g, 9% yield of the title compound of Example 18 as an orange powder. ¹H-NMR (400 MHz, methanol-d₄) δ 8.20 (d, J=2.2 Hz, 1H), 8.14 (d, J=5.1 Hz, 1H), 7.61-7.67 (m, J=9.2, 2.57 Hz, 2H), 7.35-7.41 (m, 2H), 7.26 (t, J=8.3 Hz, 3H), 7.07 (t, J=8.6 Hz, 1H), 6.37 (d, J=5.1 Hz, 1H), 4.04 (s, 3H), 3.35 (sept, 1H), 3.30 (d, J=1.8 Hz, 6H), and 1.45 (d, J=7.0 Hz, 6 H); ES-LCMS m/z 637.22 (M+H).

### Example 19

### 2,6-Difluoro-N-[3-(2-(1-methylethyl)-5-{2-[(5-methyl-2-(methyloxy)-4-[}4-[2-(methylsulfonyl)ethyl]-1-piperazinyl}phenyl)amino]-4-pyrimidinyl]-1,3-thiazol-4-yl)phenyl]benzenesulfonamide

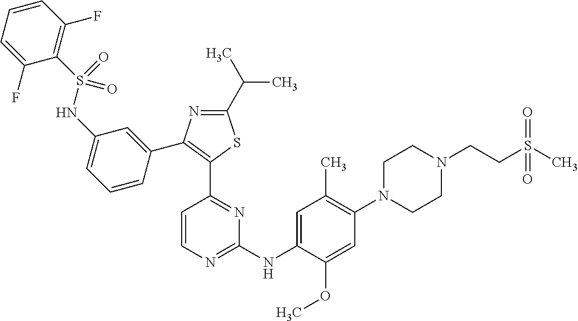

A suspension containing 0.10 g (0.20 mmol) of N-{3-[5-(2-chloro-4-pyrimidinyl)-2-(1-methylethyl)-1,3-thiazol-4-yl]phenyl}-2,6-difluorobenzenesulfonamide, prepared by a procedure analogous to Example 13, Step E, 0.077 g (0.24 mmol) of 5-methyl-2-(methyloxy)-4-{4-[2-(methylsulfonyl)ethyl]-1-piperazinyl}phenyl amine, prepared by a procedure analogous to Intermediate 6, Step F, 2.0 mL of trifluororoethanol, and 0.1 mL of a 4.0 M solution of HCl in dioxane was heated at 170° C. in a microwave reactor for 40 min. The reaction mixture was diluted with EtOAc and ether and filtered. The collected material was subjected to HPLC purification to give 98 mg (62%) of an yellow-orange solid: ¹H-NMR (400 MHz, DMSO-d₆) δ 11.1 (s, 1H), 8.14 (d, J=5.2 Hz, 1H), 8.06 (s, 1H), 7.81 (s, 1H), 7.63-7.74 (m, 1H), 7.37 (t, J=8.0 Hz, 1H), 7.20-7.32 (m, 5H), 6.73 (s, 1H), 6.20 (d, J=5.3 Hz, 1H), 3.83 (s, 3H), 3.31-3.35 (m, 2H), 3.08 (s, 3H), 2.83-2.91 (m, 4H), 2.79 (t, J=7.1 Hz, 2H), 2.61 (brs, 4H), 2.20 (s, 3H), 1.99 (s, 1H), and 1.38 (d, J=6.8 Hz, 6H); HRMS Calcd for C₃₇H₄₂F₂N₇O₆S₃ (M+H)⁺: 798.2372. Found: 798.2378.

### Example 19a

### 2,6-Difluoro-N-[3-(2-(1-methylethyl)-5-(24(5-methyl-2-(methyloxy)-4-[(4-[2-(methylsulfonyl)ethyl]-1-piperazinyl}phenyl)amino]-4-pyrimidinyl}-1,3-thiazol-4-yl)phenyl]benzenesulfonamide hydrochloride

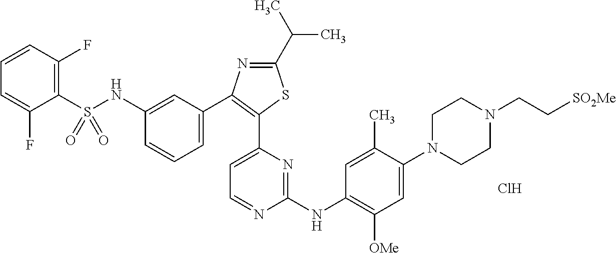

Crude 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide (9.0 g, 11.2 mmol), prepared by a procedure analogous to Example 19, was placed in a fischer porter hydrogenation flask. Trifluoroethanol (150 mL) was added and the reaction mixture was stirred until all was in solution. Platinum on carbon 5% (900 mg) was added and an air purge with vacuum and nitrogen and the reaction was placed under 60 psi hydrogen for 72 h. The reaction mixture was filtered through Celite and concentrated to dryness. A portion of this oil (1 g) was carried forward: EtOH was added and the solids that formed were collected, taken up in DCM, and 2 eq of 4 N HCl in dioxane was added. The solids were filtered and 100 mg was taken up in 10 mL of EtOH and heated to 80° C. Water (0.5 mL) was added dropwise until all went into solution and the reaction mixture was cooled to rt and the solids were filtered off to give a light yellow solid (51 mg). The solid was determined to be 3.6% Cl by weight by ion chromatography corresponding to the mono hydrochloride salt: ¹H-NMR (400 MHz, DMSO-d₆) δ 11.04 (1H, s), 10.78 (1H, s), 8.12 (1H, d, J=4.9 Hz), 8.09 (1H, s), 7.84 (1H, s), 7.62-7.71 (1H, m), 7.34 (1H, t, J=7.8 Hz), 7.18-7.29 (4H, m), 6.69 (1H, s), 6.21 (1H, d, J=4.9 Hz), 3.83 (3H, s), 3.77 (2H, brs), 3.59 (4H, brd, J=9.3 Hz), 3.17-3.34 (5H, m), 3.12 (3H, s), 3.00-3.09 (2H, m), 2.20 (3H, s), and 1.35 (6 H, d, J=6.8 Hz).

### Example 19b

### 2,6-Difluoro-N-(3-(2-(1-methylethyl)-5-(24(5-methyl-2-(methyloxy)-4-(4-[2-(methylsulfonyl)ethyl]-1-piperazinyl}phenyl)amino]-4-pyrimidinyl}-1,3-thiazol-4-yl)phenyl]benzenesulfonamide dihydrochloride

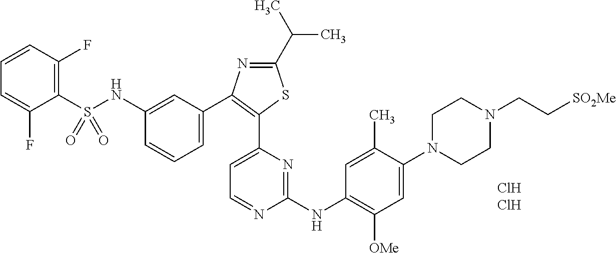

To a solution containing 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl]-1,3-thiazol-4-yl)phenyl]benzenesulfonamide (0.20 g, 0.25 mmol), prepared by a procedure analogous to Example 19 and isopropanol (5 mL) was added HCl (0.125 mL of a 4.0 M solution in dioxane, 0.50 mmol). The reaction mixture was heated at 50° C. overnight, then cooled to rt. The solvents were removed under reduced pressure. The residue was dissolved in hot EtOAc/iPrOH and filtered hot. The resulting solution was allowed to stand and orange crystals were collected by filtration and dried in a vacuum oven over 3 days. The solid, (0.175 g, 0.191 mmol, 76% yield) was determined to be 8.8% Cl by weight by ion chromatography corresponding to the dual HCl salt. ¹H-NMR (400 MHz, DMSO-d₆) δ 11.08 (s, 1H), 11.01 (s, 1H), 8.11-8.21 (m, 2H), 7.86 (s, 1H), 7.63-7.75 (m, 1H), 7.35-7.40 (m, 1H), 7.22-7.32 (m, 4H), 6.72 (s, 1H), 6.24 (d, J=5.1 Hz, 1H), 3.86 (s, 3H), 3.81 (dd, J=8.9 and 6.7 Hz, 2H), 3.57-3.68 (m, 4H), 3.21-3.35 (m, 5H), 3.15 (s, 3H), 3.09 (t, J=12.0 Hz, 2H), 2.23 (s, 3H), and 1.38 (d, J=7.0 Hz, 6H).

### Example 19c

### 2,6-Difluoro-N-[3-(2-(1-methylethyl)-5-{2-[(5-methyl-2-(methyloxy)-4-{4-[2-(methylsulfonynethyl)-1-piperazinyl]phenyl)amino]-4-pyrimidinyl}-1,3-thiazol-4-yl)phenyl]benzenesulfonamide 4-methylbenzenesulfonate

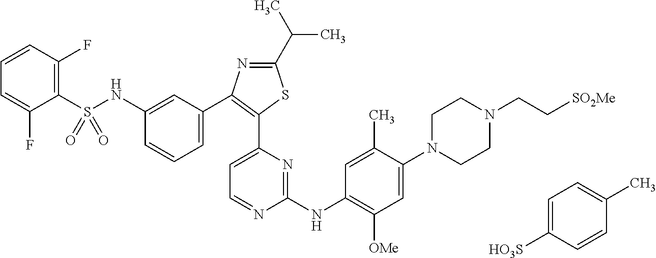

To 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide (0.15 g, 0.19 mmol), prepared by a procedure analogous to Example 19 in isopropanol (4 mL) was added tosic Acid (2.9 M in water) (0.065 mL, 0.19 mmol). The suspension was heated and stirred at 50° C. overnight. The reaction was cooled to rt, filtered, and washed successively with isopropanol and ether to provide the title compound (0.16 g, 0.17 mmol, 88%) as an orange solid. ¹H-NMR (400 MHz, DMSO-d₆) δ 11.07 (s, 1H), 9.61-9.83 (m, 1H), 8.09-8.23 (m, 2H), 7.87 (s, 1H), 7.63-7.78 (m, 1H), 7.48 (d, J=8.0 Hz, 2H), 7.17-7.44 (m, 6H), 7.11 (d, J=7.8 Hz, 2H), 6.73 (s, 1H), 6.25 (d, J=5.1 Hz, 1H), 3.53-3.95 (m, 10H), 3.21-3.41 (m, 4H), 3.15 (s, 3H), 2.88-3.08 (m, 2H), 2.16-2.36 (m, 6H), and 1.38 (d, J=6.9 Hz, 6H).

### Example 19d

### 2,6-Difluoro-N-[3-(2-(1-methylethyl)-5-(24(5-methyl-2-(methyloxy)-4-(4-[2-(methylsulfonynethyl)-1-piperazinyl}phenyl)amino]-4-pyrimidinyl]-1,3-thiazol-4-yl)phenyl]benzenesulfonamide methanesulfonate hydrate

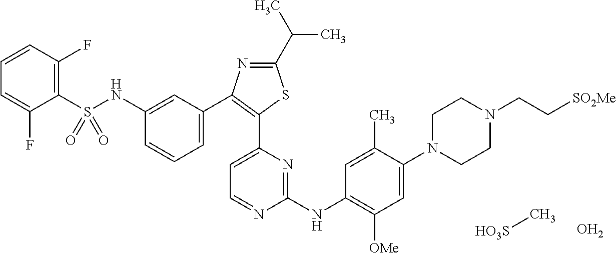

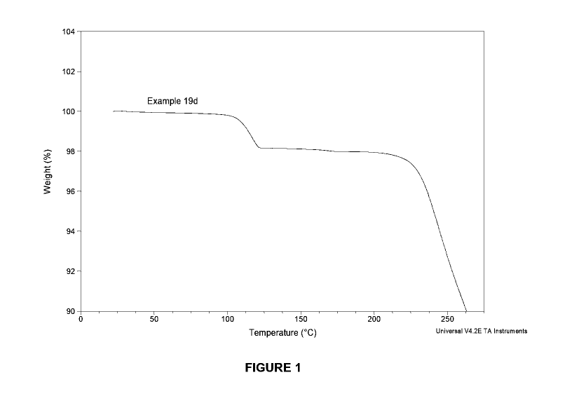

To 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide (0.15 g, 0.19 mmol), prepared by a procedure analogous to Example 19, in isopropanol (5 mL) was added methane sulfonic acid (3.0 M in water) (0.063 mL, 0.19 mmol). The mixture was heated with stirring at 50° C. overnight. (When the acid was added, most of the compound went into solution and crashed out as an orange solid upon heating. Upon further heating, the solid turned bright yellow.) The reaction was cooled and filtered. The solid was washed with isopropanol, followed by ether. The solid was collected and dried overnight in a vacuum pistol at 65° C. to provide the title compound (0.15 g, 0.17 mmol, 89%) as an yellow solid. ¹H-NMR (400 MHz, DMSO-d₆) δ 11.07 (s, 1H), 9.65-9.87 (m, 1H), 8.08-8.22 (m, 2H), 7.87 (s, 1H), 7.62-7.78 (m, 1H), 7.17-7.45 (m, 5H), 6.73 (s, 1H), 6.24 (d, J=5.2 Hz, 1H), 3.86 (s, 3H), 3.57-3.81 (m, 5H), 3.50 (d, J=0.7 Hz, 3H), 3.20-3.39 (m, 4H), 3.15 (s, 3H), 2.88-3.07 (m, 2H), 2.32 (s, 3H), 2.23 (s, 3 H), and 1.38 (d, J=6.9 Hz, 6H). TGA analyis (See FIG. 1) indicates a 1.86% weight loss from 25° C. to 105° C., corresponding to the monohydrate.

### Example 19e

### Mono sodium salt of 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide

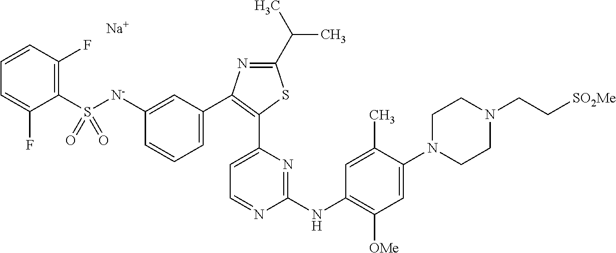

A sodium methoxide solution was prepared by adding sodium hydroxide (7.5 mg, 0.19 mmol) to MeOH (1.5 mL). The resultant solution was added to a stirring suspension of 2,6-difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide (0.15 g, 0.188 mmol), prepared by a procedure analogous to Example 19, in MeOH (3 mL). After stirring for about 1 hr, the solvent was removed under a stream of nitrogen and gentle heat to give a solid. A portion (100 mg) was taken up in about 30 mL of EtOAc, heated, and hot-filtered into a round-bottomed flask. The solvent volume was reduced to about 5-10 mL, seeded (with a previous batch), and allowed to sit overnight covered with a plastic yellow lid. The crystals were filtered to give the title compound (85 mg). The crystals appeared as small rods arranged in a plate-like formation. ¹H-NMR (400 MHz, DMSO-d₆) δ 8.05 (d, J=5.3 Hz, 1H), 7.94 (s, 2H), 7.27 (ddd, J=14.4, 8.2, and 5.9 Hz, 1H), 7.02 (t, J=7.8 Hz, 1H), 6.94 (m, 1H), 6.87 (t, J=8.3 Hz, 3H), 6.73 (s, 1H), 6.60 (m, 1H), 6.38 (d, J=5.3 Hz, 1H), 3.84 (s, 3H), 3.35 (s, 2H), 3.27 (m, 1H), 3.08 (s, 3H), 2.87 (s, 4H), 2.78 (t, J=6.7 Hz, 2H), 2.61 (s, 4H), 2.24 (s, 3H), and 1.37 (d, J=6.9 Hz, 6H).

### Example 20

### 2,6-Difluoro-N-{3-[2-(1-methylethyl)-5-(2-{[2-(methyloxy)-4-(4-{4-[2-(methylsulfonyl) ethyl]-1-piperazinyl}-1-piperidinyl)phenyl]amino}-4-pyrimidinyl)-1,3-thiazol-4-yl]phenyl}benzenesulfonamide

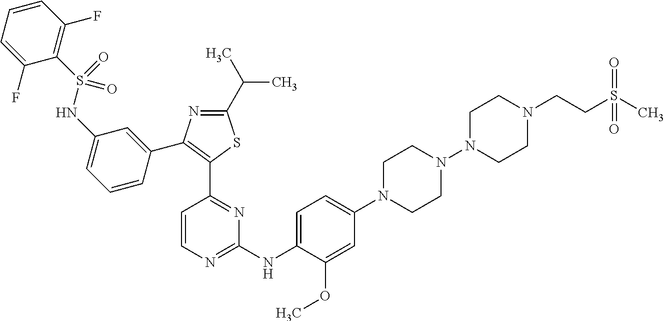

Step A: 5-Fluoro-2-nitrophenyl methyl ether

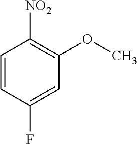

To 2-nitro-5-fluoro-phenol (56.0 g, 357 mmol) and iodomethane (24.5 mL, 393 mmol) in DMF (200 mL) was added K₂CO₃ (54.2 g, 393 mmol). Significant bubbling occurred. The mixture was stirred at rt overnight. The mixture was poured into H₂O (800 mL) and the H₂O washed with ether (3×200 mL). The ether washes were combined and washed with H₂O (2×400 mL). The ether layer was dried (MgSO₄), filtered, and rotovaped down to give the title compound of step A (56.0 g, 327 mmol, 92%) as a yellow solid. ¹H-NMR (400 MHz, CDCl₃) δ 7.96-7.90 (m, 1H), 6.79-6.67 (m, 2H) and, 3.93 (s, 3H).

Step B: 1-[3-(Methyloxy)-4-nitrophenyl]-4-piperidinol

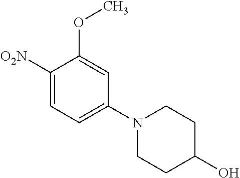

A mixture containing 10 g (58 mmol) of 5-fluoro-2-nitrophenyl methyl ether and 9.7 g (70 mmol) of potassium carbonate in 60 mL of DMSO was stirred at rt overnight. The reaction mixture was filtered and partitioned between DCM and water. The combined organic layers were washed with water and brine and dried over MgSO₄.

The solvent was removed under reduced pressure to give 14.6 g (99%) of 143-(methyloxy)-4-nitrophenyl]-4-piperidinol as a bright yellow solid: ¹H-NMR (400 MHz, d₆-DMSO) δ 7.84 (d, J=9.2 Hz, 1H), 6.54 (d, J=9.6 Hz, 1H), 6.46 (s, 1H), 4.73 (d, J=4.4 Hz, 1H), 3.86 (s, 3H), 3.80-3.69 (m, 3H), 3.18-3.12 (m, 2H), 1.79-1.76 (m, 2H), and 1.42-1.34 (m, 2H).

Step C: 1-[3-(Methyloxy)-4-nitrophenyl]-4-piperidinone

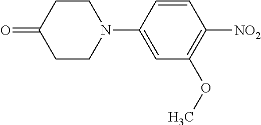

Oxalyl chloride (11.9 mL, 23.78 mmol) was dissolved in DCM (200 mL) and cooled to −78° C. DMSO (3.4 mL, 47.58 mmol) in DCM (15 mL) was added to the solution dropwise via an addition funnel. Upon completion of addition, the reaction was allowed to stir 10 min. 1-[3-(Methyloxy)-4-nitrophenyl]-4-piperidinol (4.04 g, 15.86 mmol) in DMSO (6 mL) and DCM (15 mL) was added dropwise via addition funnel. Upon completion of addition, the reaction was allowed to stir 15 min. TEA (11.0 mL, 79.3 mmol) was then added dropwise via addition funnel. Upon completion of addition, the bath was removed and the reaction was allowed to warm to rt. The reaction was stirred until alcohol was consumed as indicated by TLC. The solvent was removed in vacuo and the residue was taken up in EtOAc. The suspension was washed twice with H₂O, followed by brine. The organic layer was dried with MgSO₄, filtered and concentrated to provide the title compound of step C (3.91 g, 15.59 mmol, 97%) as a bright yellow solid. ¹H-NMR (400 MHz, DMSO-d₆) δ ppm 7.91 (d, J=9.5 Hz, 1H), 6.61 (dd, J=9.5 and 2.6 Hz, 1H), 6.52 (d, J=2.6 Hz, 1H), 3.91 (s, 3 H), 3.82 (t, J=6.2 Hz, 4H), and 2.47-2.51 (m, 4H).

Step D: 1-{1-[3-(Methyloxy)-4-nitrophenyl]-4-piperidinyl}piperazine

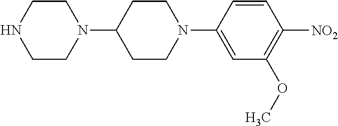

To a solution of 1-[4-amino-3-(methyloxy)phenyl]-4-piperidinone (combined batches) (17.35 g, 69.3 mmol) in toluene (600 mL) was added sequentially TEA (25 mL, 179.4 mmol), 1-Boc-piperazine (25.36 g, 136.2 mmol), and HOAc (6.0 mL, 105.9 mmol). The solution was stirred at rt for 30 min. Sodium triacetoxyborohydride (12.2 g, 57.6 mmol) was added in one portion and stirred for 30 min. A second portion of sodium triacetoxyborohydride (12.2 g, 57.6 mmol) was added and stirring continued for 30 min. A third portion of sodium triacetoxyborohydride (12.2 g, 57.6 mmol) was added. The reaction was stirred (3 h) until ketone was consumed as indicated by TLC. The reaction was quenched with a saturated solution of NaHCO₃ (600 mL) and stirred 2 days. The solution was separated and extracted with DCM (x3), dried with MgSO₄, filtered and concentrated. The resultant solid was dissolved in DCM (600 mL) and cooled to 0° C. TFA (110 mL) was added; the reaction was warmed to rt and stirred overnight. The reaction was cooled to 0° C. and quenched with 6 N NaOH (320 mL) dropwise. The solution was separated and extracted with DCM (x3), dried with MgSO₄, filtered and concentrated. Purification by flash chromatography provided the title compound of step D (18.03 g, 56.10 mmol, 81%) as a yellow solid. ¹H-NMR (400 MHz, DMSO-d₆) δ ppm 7.85 (d, J=9.2 Hz, 1H), 6.56 (dd, J=9.5 and 2.6 Hz, 1H), 6.48 (d, J=2.6 Hz, 1H), 4.03 (d, J=12.8 Hz, 2H), 3.88 (s, 3H), 3.32 (brs, 1H), 2.88-2.98 (m, 2H), 2.67-2.73 (m, 4H), 2.38-2.47 (m, 5H), 1.77-1.84 (m, 2H), and 1.41 (qd, J=12.0 and 3.7 Hz, 2H).

Step E: 1-{1-[3-(Methyloxy)-4-nitrophenyl]-4-piperidinyl}-4-[2-(methylsulfonyl)ethyl]-piperazine

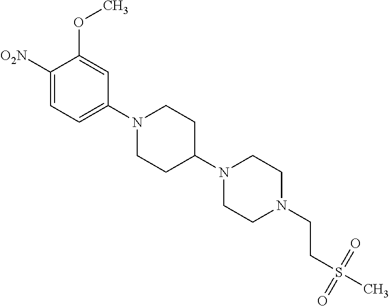

To a suspension of 1-{1-[3-(methyloxy)-4-nitrophenyl]-4-piperidinyl}piperazine (10.0 g, 23.27 mmol) and 1,4-dioxane (400 mL) was added MeOH (˜100 mL) to enhance solubility. Methyl vinyl sulfone (6.1 mL, 69.8 mmol) and Na₂CO₃ (7.4 g, 69.8 mmol) were added and the resultant mixture was heated at 80° C. overnight (−16 h). LCMS indicated that the reaction had gone to completion. The solvent was evaporated and the residue was taken up in DCM (300 mL) and filtered to remove salts. The filtrate was concentrated in vacuo to afford the title compound of step E (9.9 g, >95%) which was carried forward with no further purification. MS (ES+, m/z) 427 (M+1).

Step F: 2-(Methyloxy)-4-(4-{4-[2-(methylsulfonyl)ethyl]-1-piperazinyl}-1-piperidinyl)phenyl amine

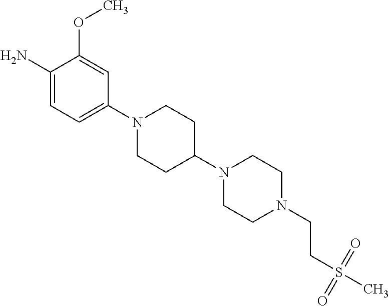

NaBH₄ (2.64 g, 69.8 mmol) was added carefully in portions to a suspension of 1-{1-[3-(methyloxy)-4-nitrophenyl]-4-piperidinyl}-4-[2-(methylsulfonyl)-ethyl]piperazine (9.9 g, 23.3 mmol), nickel(II)chloride hexahydrate (1.66 g, 7 mmol), MeOH (120 mL) and THF (60 mL) at 0° C. The ice bath was removed and the reaction mixture was stirred at rt overnight (˜16 h). The reaction mixture was concentrated onto silica gel and flash chromatography afforded the title compound of step F (6.24 g, 68%) as a brown solid. ¹H-NMR (400 MHz, DMSO-d₆) δ 6.49-6.44 (m, 2H), 6.26 (dd, J=8.4 and 2.6 Hz, 1H), 4.18 (brs, 2H), 3.71 (s, 3H), 3.43-3.36 (m, 2H), 3.25 (t, J=6.8 Hz, 2H), 3.00 (s, 3H), 2.65 (t, J=6.8 Hz, 2H), 2.47-2.36 (m, 4H), 2.23-2.15 (m, 1H), 1.82-1.75 (m, 2H), and 1.54-1.43 (m, 2H).

Step G: 2,6-Difluoro-N-{3-[2-(1-methylethyl)-5-(2-{[2-(methyloxy)-4-(4-{4-[2-(methylsulfonyl)ethyl]-1-piperazinyl}-1-piperidinyl)phenyl]amino}-4-pyrimidinyl)-1,3-thiazol-4-yl]phenyl}benzenesulfonamide

A suspension containing 0.10 g (0.20 mmol) of N-{3-[5-(2-chloro-4-pyrimidinyl)-2-(1-methylethyl)-1,3-thiazol-4-yl]phenyl}-2,6-difluorobenzenesulfonamide, prepared by a procedure analogous to Example 13, Step E, 0.094 g (0.24 mmol) of [2-(methyloxy)-4-(4-{4-[2-(methylsulfonyl)ethyl]-1-piperazinyl}-1-piperidinyl)phenyl]amine, 2.0 mL of trifluoroethanol, and 0.1 mL of a 4.0 M solution of HCl in dioxane was heated at 170° C. in a microwave reactor for 60 min. The reaction mixture was diluted with EtOAc and ether and filtered. The collected material was subjected to HPLC purification to give 92 mg (54%) of an orange solid: ¹H-NMR (400 MHz, DMSO-d₆) δ 11.03 (s, 1H), 8.09 (d, J=5.3 Hz, 1H), 8.03 (s, 1H), 7.62-7.72 (m, 1H), 7.58 (d, J=9.0 Hz, 1H), 7.35 (t, J=8.1 Hz, 1H), 7.15-7.29 (m, 5H), 6.62 (d, J=1.5 Hz, 1H), 6.46 (dd, J=9.0 and 1.8 Hz, 1H), 6.19 (d, J=5.3 Hz, 1H), 3.81 (s, 3H), 3.70 (d, J=11.0 Hz, 2H), 3.27 (t, J=6.3 Hz, 4H), 3.03 (s, 3H), 2.58-2.70 (m, 6H), 2.40-2.47 (m, 6H), 1.79-1.92 (m, 2H), 1.46-1.59 (m, 2H), and 1.37 (d, J=6.8 Hz, 6H); HRMS calcd for C₄₁H₄₉N₈O₅F₂S₃ (M+H⁺): 867.2956. Found: 867.2971.

### Example 21

### 2,6-Difluoro-N-{3-[5-[2-({3-fluoro-4-[4-(2-fluoroethyl)-1-piperazinyl]phenyl}amino)-4-pyrimidinyl]-2-(1-pyrrolidinyl)-1,3-thiazol-4-yl]phenyl}benzenesulfonamide

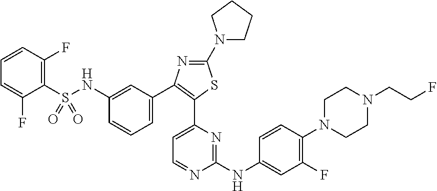

Step A: Ethyl 3-{[(2,6-difluorophenyl)sulfonyl]amino}benzoate

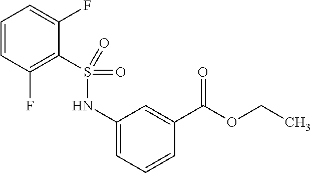

To a solution of ethyl-3-aminobenzoate (50 mL, 333 mmol) and 2,6-difluorobenzenesulfonyl chloride (44.2 mL, 333 mmol) in DCM (DCM) (300 mL) at 0° C. was added pyridine (32.2 mL, 400 mmol). The reaction mixture was warmed to rt, stirred for 36 h, and quenched with 2 mL NH₃ (7 M in MeOH). The suspension washed with 10% NaHSO₄ and the organic extracts combined and passed through a short column of silica gel. Residual material was flushed from the column with 10% MeOH/EtOAc. The organic extracts were combined and the solvent removed under reduced pressure to provide 107.9 g (95%) of the title compound of Step A. ¹H-NMR (400 MHz, DMSO-d6) δ ppm 11.20 (s, 1H), 7.77 (s, 1H), 7.71 (t, J=7.4 Hz, 1H), 7.63 (d, J=7.3 Hz, 1H), 7.35-7.49 (m, 2H), 7.29 (t, J=9.3 Hz, 2H), 4.28 (q, J=7.1 Hz, 2H), and 1.29 (t, J=7.1 Hz, 3H).

Step B: N-{3-[(2)-2-(2-Chloro-4-pyrimidinyl)-1-hydroxyethenyl]phenyl}-2,6-difluorobenzenesulfonamide

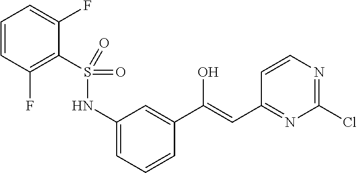

To a stirring solution of ethyl 3-{[(2,6-difluorophenyl)sulfonyl]amino}benzoate (47.9 g, 140 mmol) in 100 mL anhydrous THF at 0° C. was added 1M LiHMDS in THF (421 mL, 421 mmol). A solution of 2-chloro-4-methylpyrimidine (19.9 g, 154 mmol) in 100 mL of anhydrous THF was added to the reaction mixture over 30 min and warmed to rt. The reaction mixture was quenched with 50 mL of MeOH and concentrated to a black solid under vacuum. The residue was partitioned between DCM and 10% NaHSO₄. The aqueous and suspended solids were extracted 2× with DCM and the combined organic extracts were filtered through a pad of Celite, concentrated, and passed through a short silica gel column (elution with THF) to provide 57 g (96%) of the title compound of Step B. ¹H-NMR (400 MHz, DMSO-d6) δ ppm 11.03-11.34 (m, 1H), 8.49-8.91 (m, 1H), 7.79 (d, J=7.4 Hz, 1H), 7.65-7.76 (m, 2H), 7.55-7.63 (m, 1H), 7.50 (t, J=7.7 Hz, 1H), 7.35-7.47 (m, 1H), 7.22-7.34 (m, 2H), 6.43 (s, 1 H), and 4.60 (s, 1H); ES-LCMS m/z 423.93 (M+H).

Step C: N-{3-[5-(2-Chloro-4-pyrimidinyl)-2-(1-pyrrolidinyl)-1,3-thiazol-4-yl]phenyl}-2,6-difluorobenzenesulfonamide

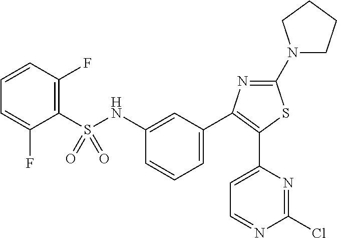

To a stirring suspension of N-{3-[(2-chloro-4-pyrimidinyl)acetyl]phenyl}-2,6-difluorobenzenesulfonamide (1.0 g, 2.36 mmol, 1.0 eq) in DCM (˜5 mL) was added NBS (0.44 g, 2.48 mmol, 1.05 eq). Upon formation of a red solution (˜10 min) the reaction mixture was concentrated to a solid and taken up in dioxane (10 mL). To this solution was added Mg₂CO₃ (0.38 g) followed by the 1-pyrrolidinecarbothioamide (0.384 g, 2.95 mmol, 1.25 eq), prepared by a procedure analogous to Intermediate 7. After stirring 3 h, the mixture was quenched with water (50 mL) and 1N HCl (10 mL) and stirred 0.25 h. The mixture was filtered and the resultant solid triturated with EtOAc/Hex to give 0.52 g (41%) of the title compound of Step C. ¹H-NMR (400 MHz, DMSO-d₆) δ ppm 11.11 (s, 1H), 8.14 (d, J=5.7 Hz, 1H), 7.65-7.74 (m, 1H), 7.41 (t, J=7.7 Hz, 1H), 7.18-7.29 (m, 5H), 6.44 (d, J=5.5 Hz, 1H), 3.45-3.52 (m, 4 H), and 1.98-2.05 (m, 4H).

Step D: 1-(2-Fluoroethyl)-4-(2-fluoro-4-nitrophenyl)piperazine

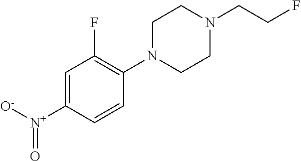

To a solution of 1-(2-fluoro-4-nitrophenyl)piperazine (0.50 g, 2.22 mmol) in DMF (25 mL), 1-bromo-2-fluoroethane (0.42 g, 3.3 mmol) and sodium carbonate (0.71 g, 6.7 mmol) were added and heated to 70° C. for 18 h. The reaction mixture was cooled to rt, diluted with EtOAc, and washed with NaHCO₃ x2 and brine. The organic layer was dried over MgSO₄, loaded onto silica, and purified via flash chromatography using EtOAc/Hex gradient 0-100%. The desired fractions were combined and the solvent was removed 0.51 g, 85% yield, of the title compound of Step D. ¹H-NMR (400 MHz, DMSO-d₆) δ ppm 7.96-8.06 (m, 2H), 7.17 (t, J=8.9 Hz, 1H), 4.63 (t, J=4.9 Hz, 1H), 4.51 (t, J=4.9 Hz, 1H), 3.27-3.31 (m, 4H), 2.72 (t, J=4.9 Hz, 1H), and 2.59-2.66 (m, 5H).

Step E: 3-Fluoro-4-[4-(2-fluoroethyl)-1-piperazinyl]phenyl amine

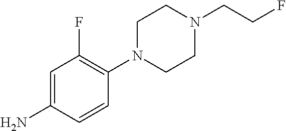

1-(2-Fluoroethyl)-4-(2-fluoro-4-nitrophenyl)piperazine was taken up in methanol (25 mL) and 10% Palladium/Carbon (0.050 g) was added. The mixture was stirred under H₂, 60 psi, for 3 h and filtered through a celite plug. The solvent was removed to give 0.47 g, 91% yield, of a 3-fluoro-4-[4-(2-fluoroethyl)-1-piperazinyl]phenyl amine: ES-LCMS m/z 242 (M+H).

Step F: 2,6-Difluoro-N-{3-[5-[2-({3-fluoro-4-[4-(2-fluoroethyl)-1-piperazinyl]phenyl}amino)-4-pyrimidinyl]-2-(1-pyrrolidinyl)-1,3-thiazol-4-yl]phenyl}benzenesulfonamide (Title Compound)

To a stirring solution of N-{3-[5-(2-chloro-4-pyrimidinyl)-2-(1-pyrrolidinyl)-1,3-thiazol-4-yl]phenyl}-2,6-difluorobenzenesulfonamide (0.074 g, 1.0 eq) in 2,2,2-trifluoroethanol (2 mL) was added 3-fluoro-4-[4-(2-fluoroethyl)-1-piperazinyl]phenyl amine (0.038 g, 1.0 eq), and PTSA (0.063 g, 2.4 eq). The reaction mixture was heated overnight at 100° C. in a sealed vial. The reaction mixture was quenched with 2 M NH₃ in MeOH (3 mL), concentrated onto silica gel, and purified by column chromatography (gradient elution: 0 to 50% DCM/10% MeOH in DCM) to provide 56 mg (55%) of the title compound. ¹H-NMR (400 MHz, DMSO-d₆) δ ppm 11.05 (s, 1 H), 9.53 (s, 1H), 7.94 (d, J=5.5 Hz, 1H), 7.77 (d, J=17.4 Hz, 1H), 7.64-7.73 (m, 1H), 7.38 (t, J=8.3 Hz, 2H), 7.22-7.29 (m, 4H), 7.17 (d, J=7.7 Hz, 1H), 6.97 (t, J=9.3 Hz, 1H), 5.91 (d, J=5.3 Hz, 1H), 4.63 (t, J=4.7 Hz, 1H), 4.51 (t, J=4.6 Hz, 1H), 3.40-3.49 (m, 4H), 2.92-3.01 (m, 4H), 2.68-2.74 (m, 1H), 2.58-2.68 (m, 5 H), and 1.97-2.06 (m, 4H).

### Example 22

### 2-Fluoro-N-{2-fluoro-5-[5-{2-[(3-fluoro-4-}4-[2-(methylsulfonyl)ethyl]-1-piperazinyl}phenyl)amino]-4-pyrimidinyl}-2-(1-methylethyl)-1,3-thiazol-4-yl]phenyl}benzenesulfonamide hydrochloride

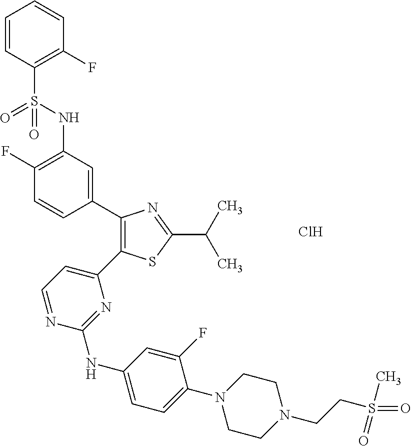

Step A: Ethyl 4-fluoro-3-{[(2-propen-1-yloxy)carbonyl]amino}benzoate

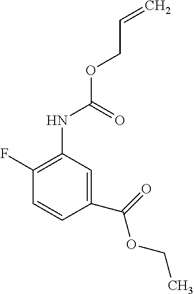

To a solution containing 4-fluoro-3-aminobenzoate (6.78 g, 37 mmol), pyridine (3.6 mL, 44 mmol), and THF (35 mL) at 0° C. was added dropwise allyl chloroformate (4.4 mL, 41 mmol). The reaction mixture was allowed to warm to rt and stir until TLC indicated a complete reaction. The reaction mixture was partitioned between EtOAc and water. The organic laters were washed with aqueous NH₄Cl, aqueous NaHCO₃, and brine, then dried over Na₂SO₄ and the solvent was removed under reduced pressure to give a white powder. 9.37 g (96%). ESIMS m/z: 267.99 (M+H)⁺.

Step B: 2-Propen-1-yl{5-[(2-chloro-4-pyrimidinypacetyl]-2-fluorophenyl}carbamate

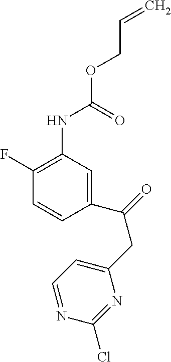

In a procedure analogous to Intermediate 1, Step B, 12 g (98%) of crude 2-propen-1-yl{5-[(2-chloro-4-pyrimidinypacetyl]-2-fluorophenyl}carbamate was prepared from ethyl 4-fluoro-3-{[(2-propen-1-yloxy)carbonyl]amino}benzoate (9.37 g, 35 mmol), 4-methyl-2-chloropyrimidine (4.94 g, 38 mmol) and LHMDS (112.2 mL of a 1M solution in THF, 112 mmol). ESIMS m/z: 349.98 (M+H)⁺.

Step C: 2-Propen-1-yl{5-[5-(2-chloro-4-pyrimidinyl)-2-(1-methylethyl)-1,3-thiazol-4-yl]-2-fluorophenyl}carbamate

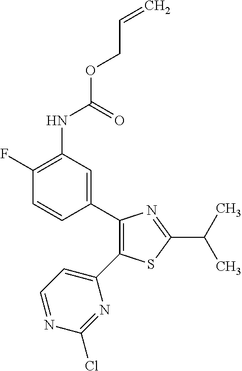

In a procedure analogous to Example 2, Step C, 1.5 g (43%) of 2-propen-1-yl{5-[5-[(2-chloro-4-pyrimidinyl)-2-(1-methylethyl)-1,3-thiazol-4-yl]-2-fluorophenyl}carbamate was prepared from 2-propen-1-yl{5-[(2-chloro-4-pyrimidinypacetyl]-2-fluorophenyl}carbamate (2.8 g, 8 mmol). ESIMS m/z: 432.82 (M+H)⁺.

Step D: {5-[5-(2-Chloro-4-pyrimidinyl)-2-(1-methylethyl)-1,3-thiazol-4-yl]-2-fluorophenyl}amine

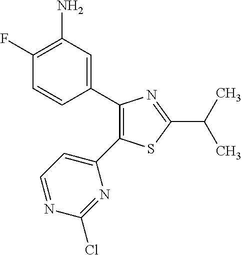

In a procedure analogous to Example 6, Step G, 1.1 g (92%) of {5-[5-(2-chloro-4-pyrimidinyl)-2-(1-methylethyl)-1,3-thiazol-4-yl]-2-fluorophenyl}amine was prepared from 2-propen-1-yl{5-[5-(2-chloro-4-pyrimidinyl)-2-(1-methylethyl)-1,3-thiazol-4-yl]-2-fluorophenyl}carbamate (1.5 g, 3.5 mmol). ESIMS m/z: 349.27 (M+H)⁺).

Step E: N-{5-[5-(2-Chloro-4-pyrimidinyl)-2-(1-methylethyl)-1,3-thiazol-4-yl]-2-fluorophenyl}-2-fluorobenzenesulfonamide

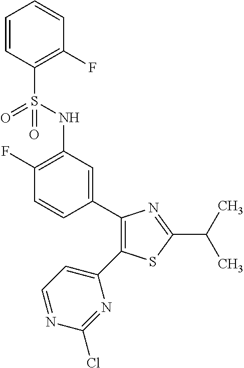

5-[5-(2-Chloro-4-pyrimidinyl)-2-(1-methylethyl)-1,3-thiazol-4-yl]-2-fluorophenyl amine (1 g, 2.87 mmol), and 2-fluorobenzenesulfonyl chloride (0.669 g, 3.44 mmol) were combined in a round bottom flask with DCM (28 mL). Pyridine (0.3 mL, 3.73 mmol) was then added via syringe and the resulting mixture was allowed to stir 4 days at rt. EtOAc and water were then added to the crude reaction and the desired was extracted into the organic layer. The organic EtOAc layer was then washed with brine, treated with sodium sulfate, filtered and concentrated to dryness to yield a beige powder 1.15 g (75% Yield). ¹H-NMR (400 MHz, DMSO-d₆) 5 ppm 10.67 (1H, s), 8.58 (1H, d, J=5.3 Hz), 7.71 (2H, t, J=7.0 Hz), 7.36-7.51 (3H, m), 7.20-7.36 (2H, m), 7.08 (1H, d, J=5.1 Hz), 3.34 (1H, m), and 1.38 (6H, d, J=6.9 Hz).

Step F: 2-Fluoro-N-{2-fluoro-545-{2-[(3-fluoro-4-{4-[2-(methylsulfonyl)ethyl]-1-piperazinyl}phenyl)amino]-4-pyrimidinyl}-2-(1-methylethyl)-1,3-thiazol-4-yl]phenyl}benzenesulfonamide hydrochloride

N-{5-[5-(2-Chloro-4-pyrimidinyl)-2-(1-methylethyl)-1,3-thiazol-4-yl]-2-fluorophenyl}-2-fluorobenzenesulfonamide from two combined batches (1.5 g, 2.96 mmol), and 3-fluoro-4-{4-[2-(methylsulfonyl)ethyl]-1-piperazinyl}phenyl amine (0.89 g, 2.96 mmol), prepared by a procedure analogous to Intermediate 5, Step B, were placed into a microwave reaction vessel. Trifluoroethanol (10 mL) and conc. HCl (15 drops) were then added and the resulting mixture was heated in the microwave for 30 min at 170° C. The reaction is complete by LCMS. TEA (2 mL) was added to the reaction mixture which was then concentrated to dryness. The resulting crude mixture was dissolved in a small amount of DCM and loaded onto the top of a 40 g ISCO silica gel column. Chromatography with EtOAc and hexanes resulted in fractions containing the desired product which were concentrated to dryness. The resulting solid was sonicated in ether and filtered off to yield 1.58 g of a yellow powder. This powder was then taken back up in ether and treated with 4 N HCl in dioxane (0.5 mL). Solids changed color and were filtered off to yield 1.65 g of the target compound. (72% Yield). ¹H-NMR (400 MHz, DMSO-d₆) δ ppm 11.06 (1H, brs), 10.63 (1H, s), 9.85 (1 H, s), 8.33 (1H, d, J=5.1 Hz), 7.72-7.79 (1H, m), 7.64-7.71 (2H, m), 7.37-7.47 (4H, m), 7.24-7.32 (2H, m), 7.06 (1H, t, J=9.4 Hz), 6.46 (1H, d, J=5.2 Hz), 3.7-3.85 (2H, brs), 3.2-3.7 (9H, m), 3.05-3.20 (5H, m), and 1.39 (6H, d, J=6.9 Hz); MS (ESI) m/z 772.24 (M+H)⁺.

### Example 23

### 2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide

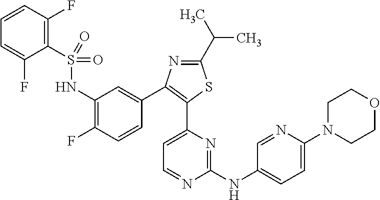

Step A: Ethyl 3-{[(2,6-difluorophenyl)sulfonyl]amino}-4-fluorobenzoate

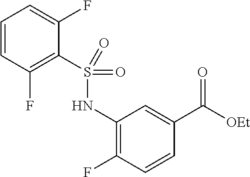

To a solution of ethyl 3-amino-4-fluorobenzoate (5.47 g, 30 mmol) and pyridine (2.55 mL, 33 mmol) in DCM (150 mL) was added 2,6-difluorobenzenesulfonyl chloride (4.45 mL, 33 mmol). The reaction was stirred overnight at rt. After 16 h, the reaction mixture was concentrated, triturated with ether, and dried in vacuo to generate 7.87 g (66% yield) of the product of Step A as a white powder. ES-LCMS m/z 360 (M+H).

Step B: N-{5-[(2-Chloro-4-pyrimidinypacetyl]-2-fluorophenyl}-2,6-difluorobenzenesulfonamide

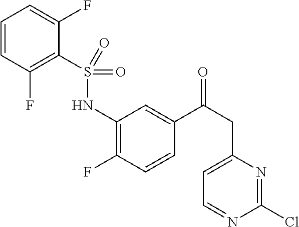

To a solution of ethyl 3-{[(2,6-difluorophenyl)sulfonyl]amino}-4-fluorobenzoate (5.0 g, 13.9 mmol) in THF (100 mL) was added 1.0 M LHMDS in THF (34.8 mL, 34.8 mmol). A solution of 2-chloro-4-methylpyrimidine (2.7 g, 20.9 mmol) in THF (100 mL) was added dropwise over 30 min, and the reaction was stirred overnight at rt. The reaction was quenched with 10 mL of MeOH and concentrated, and the residue was partitioned between EtOAc and saturated aqueous sodium bicarbonate. The aqueous layer was extracted with 2×50 mL EtOAc, and the combined organic layers were passed through a pad of silica gel, concentrated, and adsorbed onto silica gel. The crude product was purified via flash chromatography with 0-100% EtOAc/DCM to generate 3.07 g (50% yield) of the product of Step B as a white powder. ES-LCMS m/z 443 (M+H).

Step C: N-{5-[5-(2-Chloro-4-pyrimidinyl)-2-(1-methylethyl)-1,3-thiazol-4-yl]-2-fluorophenyl}-2,6-difluorobenzenesulfonamide

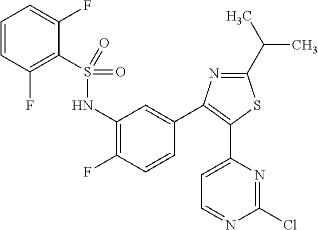

To a solution of N-{5-[(2-chloro-4-pyrimidinypacetyl]-2-fluorophenyl}-2,6-difluorobenzenesulfonamide (1.0 g, 2.3 mmol) in DMF (10 mL) was added NBS (0.49 g, 2.8 mmol). After stirring for 45 min at rt, 2-methylpropanethioamide (0.35 g, 3.4 mmol), prepared by a procedure analogous to Intermediate 3, was added, and the reaction was stirred at rt. After 4 h, the reaction mixture was partitioned between ether and saturated aqueous sodium bicarbonate. The organic layer was washed with brine, dried over anhydrous sodium sulfate, filtered, and concentrated to generate 0.49 g (41% yield) of the product of Step C as a yellow powder. ES-LCMS m/z 525 (M+H).

Step D: 2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide

The title compound of Example 161 was synthesized by heating a suspension of N-{5-[5-(2-chloro-4-pyrimidinyl)-2-(1-methylethyl)-1,3-thiazol-4-yl]-2-fluorophenyl}-2,6-difluorobenzenesulfonamide (75 mg, 0.14 mmol) and 6-(4-morpholinyl)-3-pyridinamine (28 mg, 0.16 mmol), prepared by a procedure analogous to Intermediate 4, Step B, in a mixture of 1-butanol (2 mL) and MeOH (0.2 mL) at 170° C. for 60 min in a microwave reactor. The reaction mixture was concentrated, redissolved in 2 mL of MeOH, and purified by reverse-phase HPLC, eluting with 10-60% ACN/0.1% aqueous trifluoroacetic acid. The desired fractions were combined, neutralized with saturated aqueous sodium bicarbonate, and extracted with EtOAc. The organic layer was dried over anhydrous sodium sulfate, filtered, and concentrated to generate 41 mg (44% yield) of the title compound of Example 23 as a yellow solid. ¹H-NMR (400 MHz, DMSO-d₆) δ 10.95 (s, 1H), 9.49 (s, 1H), 8.42 (d, J=2.6 Hz, 1H), 8.18 (d, J=4.8 Hz, 1H), 7.87 (dd, J=9.1 and 2.4 Hz, 1H), 7.61 (brs, 1H), 7.41 (d, J=7.7 Hz, 1H), 7.15-7.25 (m, 4H), 6.81 (d, J=9.2 Hz, 1H), 6.35 (d, J=4.9 Hz, 1H), 3.70 (t, J=4.8 Hz, 4H), 3.35 (t, J=4.9 Hz, 4H), 3.28-3.30 (m, 1H), and 1.36 (d, J=7.0 Hz, 6H); ES-LCMS m/z 668 (M+H).

### Example 24

### N-{5-[5-(2-{[6-(4-Cyano-1-piperidinyl)-3-pyridinyl]amino}-4-pyrimidinyl)-2-(1-methylethyl)-1,3-thiazol-4-yl]-2-fluorophenyl}-2,6-difluorobenzenesulfonamide

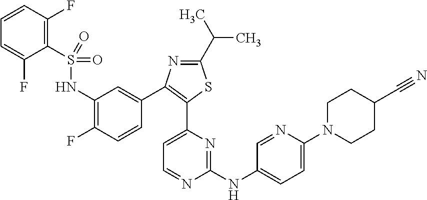

### Step A: 4-Piperidinecarbonitrile

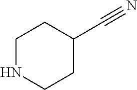

4-Piperidinecarboxamide (10 g, 78 mmol) was refluxed in thionyl chloride (35 mL) for 4 h until the suspension became homogeneous. The reaction was cooled to rt and poured onto 300 g of crushed ice. The ice slurry melted and the solution was treated with potassium hydroxide pellets until pH=9. The reaction was concentrated to a residue which was partitioned between water and chloroform. The aqueous layer was extracted with four portions of chloroform and the organic fractions were combined and concentrated. The reaction yielded 3.1 g (36%) of the 4-piperidinecarbonitrile as a brown oil. ¹H-NMR (400 MHz, DMSO-d6) δ ppm 2.84-2.98 (m, 1H), 2.79 (ddd, J=12.3, 6.0, and 3.7 Hz, 2H), 2.44-2.66 (m, 2H), 2.24 (brs, 1H), 1.66-1.84 (m, 2H), and 1.44-1.64 (m, 2H).

Step B: 1-(5-Nitro-2-pyridinyl)-4-piperidinecarbonitrile

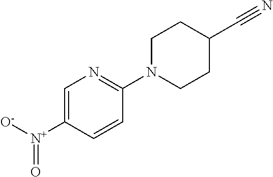

4-Piperidinecarbonitrile (3.1 g, 28.1 mmol), 2-chloro-5-nitropyridine (4.0 g, 25.6 mmol) and potassium carbonate (5.3 g, 38.3 mmol) were stirred for 15 h at rt in acetonitrile (20 mL). The solvent was removed under vacuum and the residue was partitioned between DCM and water. The aqueous layer was discarded and the organic suspension was washed a second time with water. The organic suspension was separated and concentrated under vacuum. The reaction and work-up yielded 5.85 g (99%) of the 1-(5-nitro-2-pyridinyl)-4-piperidinecarbonitrile as a yellow powder. ¹H-NMR (400 MHz, DMSO-d6) δ ppm 8.95 (d, J=2.8 Hz, 1H), 8.21 (dd, J=9.6 and 2.8 Hz, 1H), 6.97 (d, J=9.6 Hz, 1H), 4.05 (ddd, J=13.7, 6.0, and 3.8 Hz, 2H), 3.57 (ddd, J=13.8, 9.1, and 3.3 Hz, 2H), 3.13-3.24 (m, 1H), 1.95 (dddd, J=10.0, 6.6, 3.4, and 3.2 Hz, 2H), and 1.66-1.81 (m, 2H).

Step C: 1-(5-Amino-2-pyridinyl)-4-piperidinecarbonitrile

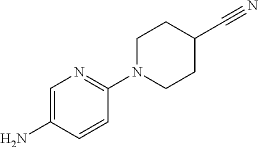

The 1-(5-nitro-2-pyridinyl)-4-piperidinecarbonitrile (5.85 g, 25.2 mmol) was combined with 5% Palladium on carbon (1.0 g) in a 10:1 mixture of DCM/MeOH (30 mL). The reaction was stirred vigorously for 15 h under a 55 psi H₂ atmosphere. The reaction mixture was filtered through Celite and the filtrate was concentrated under vacuum. The reaction crude was purified in three steps: 1) silica gel chromatography (gradient: 40-100% EtOAc/hexanes), 2) silica gel chromatography (gradient: 0-30% MeOH/EtOAc) and 3) silica gel chromatography (gradient: 10-100% [90 DCM: 9 MeOH: 1NH₄OH]/DCM). Purification yielded 1.5 g (29%) of 1-(5-amino-2-pyridinyl)-4-piperidinecarbonitrile as a brown powder. ¹H-NMR (400 MHz, DMSO-d6) δ ppm 7.59 (d, J=2.8 Hz, 1H), 6.91 (dd, J=8.8 and 2.8 Hz, 1H), 6.65 (d, J=8.8 Hz, 1H), 4.58 (s, 2H), 3.50 (ddd, J=12.9, 6.6, and 3.5 Hz, 2H), 3.06-3.19 (m, 2H), 3.01 (dt, J=8.4 and 4.0 Hz, 1H), 1.90 (ddd, J=9.7, 6.5, and 3.2 Hz, 2H), and 1.61-1.82 (m, 2H).

Step D: N-{5-[5-(2-{[6-(4-cyano-1-piperidinyl)-3-pyridinyl]amino}-4-pyrimidinyl)-2-(1-methylethyl)-1,3-thiazol-4-yl]-2-fluorophenyl}-2,6-difluorobenzenesulfonamide N-{5-[5-(2-Chloro-4-pyrimidinyl)-2-(1-methylethyl)-1,3-thiazol-4-yl]-2-fluorophenyl}-2,6-difluorobenzenesulfonamide (150 mg, 0.29 mmol), prepared by a procedure analogous to Example 23, Step C, 1-(5-amino-2-pyridinyl)-4-piperidinecarbonitrile (53 mg, 0.26 mmol) and 4 M HCl in 1,4-dioxane (97 uL, 0.39 mmol) were combined with 3,3,3-trifluoroethanol (3 mL) in a sealed microwave vial. The reaction was heated for 30 min. at 170° C. The reaction was cooled and concentrated to a residue and purified directly by silica gel chromatography (gradient: 0-100% (90DCM:9MeOH:1NH₄OH)/DCM). Purification yielded 50 mg (28%) of N-{5-[5-(2-{[6-(4-cyano-1-piperidinyl)-3-pyridinyl]amino}-4-pyrimidinyl)-2-(1-methylethyl)-1,3-thiazol-4-yl]-2-fluorophenyl}-2,6-difluorobenzenesulfonamide as a brown powder. ¹H-NMR (400 MHz, DMSO-d6) δ ppm 10.72-11.03 (m, 1H), 9.49 (brs, 1H), 8.40 (d, J=2.6 Hz, 1H), 8.22 (d, J=5.1 Hz, 1H), 7.84 (dd, J=9.1 and 2.5 Hz, 1H), 7.57-7.78 (m, 1H), 7.36-7.54 (m, 2H), 7.14-7.36 (m, 3H), 6.84 (d, J=9.2 Hz, 1H), 6.38 (d, J=5.0 Hz, 1H), 3.72 (ddd, J=13.1, 6.1, and 3.5 Hz, 2H), 3.22-3.40 (m, 3H), 2.99-3.22 (m, 1H), 1.93 (ddd, J=9.6, 6.3, and 3.2 Hz, 2H), 1.58-1.84 (m, 2H), and 1.28-1.45 (m, 6H); ES-LCMS m/z 691.20 (M+H).

### Example 25

### 2,6-Difluoro-N-{2-fluoro-5-[5-[2-({3-fluoro-4-[(4-(2-fluoroethyl)-1-piperazinyl]phenyl}amino)-4-pyrimidinyl]-2-(1-methylethyl)-1,3-thiazol-4-yl]phenyl}benzenesulfonamide

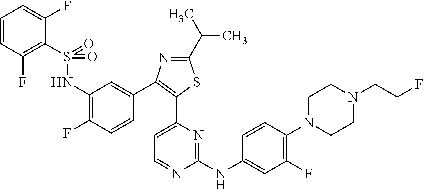

The title compound of Example 25 was synthesized by heating a suspension of N-{5-[5-(2-chloro-4-pyrimidinyl)-2-(1-methylethyl)-1,3-thiazol-4-yl]-2-fluorophenyl}-2,6-difluorobenzenesulfonamide (75 mg, 0.14 mmol), prepared by a procedure analogous to Example 23, Step C, and {3-fluoro-4-[4-(2-fluoroethyl)-1-piperazinyl]phenyl}ammonium chloride (prepared by a procedure analogous to Example 21, Step E, (44 mg, 0.16 mmol) in 2-propanol (2 mL) at 170° C. for 20 min in a microwave reactor. The reaction mixture was concentrated, redissolved in 2 mL MeOH, and purified by reverse-phase HPLC, eluting with 10-80% ACN/0.1% aqueous trifluoroacetic acid. The desired fractions were combined, neutralized with saturated aqueous sodium bicarbonate, and extracted with EtOAc. The organic layer was dried over anhydrous sodium sulfate, filtered, and concentrated to generate 51 mg (50% yield) of the title compound as a yellow solid. ¹H-NMR (400 MHz, DMSO-d₆) δ 9.71 (s, 1H), 8.17 (d, J=5.1 Hz, 1H), 7.70 (dd, J=15.4 and 2.2 Hz, 1H), 7.49 (m, 1H), 7.37 (d, J=8.1 Hz, 2H), 7.04-7.16 (m, 4H), 6.97 (t, J=9.4 Hz, 1H), 6.37 (d, J=5.2 Hz, 1H), 4.62 (t, J=4.8 Hz, 1H), 4.50 (t, J=4.9 Hz, 1H), 3.27-3.30 (m, 1H), 2.96 (s, 4H), 2.71 (t, J=4.8 Hz, 1H), 2.60-2.65 (m, 5H), and 1.37 (d, J=6.8 Hz, 6H); ES-LCMS m/z 730 (M+H).

### Example 26

### 2,6-Difluoro-N-{2-fluoro-5-[5-(2-{(6-(4-morpholinyl)-3-pyridinyl]amino}-4-pyrimidinyl)-2-(1-pyrrolidinyl)-1,3-thiazol-4-yl]phenyl}benzenesulfonamide

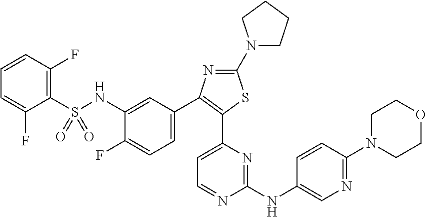

Step A: N-{5-[5-(2-Chloro-4-pyrimidinyl)-2-(1-pyrrolidinyl)-1,3-thiazol-4-yl]-2-fluorophenyl}-2,6-difluorobenzenesulfonamide

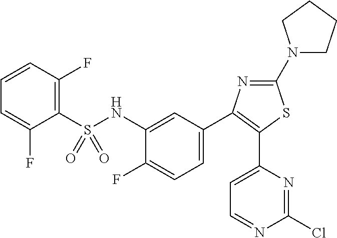

To a stirring suspension of N-{5-[(2-chloro-4-pyrimidinyl)acetyl]-2-fluorophenyl}-2,6-difluorobenzenesulfonamide (7.3 g, 16.5 mmol, 1.0 eq), prepared by a procedure analogous to Example 23, Step B, in DCM (˜35 mL) was added NBS (3.1 g, 17.4 mmol, 1.05 eq). Upon formation of a red solution (˜10 min) the reaction mixture was concentrated to a solid and taken up in dioxane (70 mL). To this solution was added Mg₂CO₃ (3.2 g) followed by 1-pyrrolidinecarbothioamide (3.2 g, 24.7 mmol, 1.5 eq), prepared by a procedure analogous to Intermediate 7. After stirring 3 h, the mixture was quenched with water (350 mL) and 1N HCl (70 mL) and stirred 0.25 h. The liquid was decanted and the remaining solids dissolved in EtOAc and precipitated with hexanes to give a dark brown solid which was about 75% pure. This solid was dissolved in EtOAc (125 mL) and precipitated with hexanes (100 mL) to give 6.1 g (67%) of the title compound of Step A. ¹H-NMR (400 MHz, DMSO-d₆) δ ppm 11.01 (s, 1H), 8.23 (d, J=5.5 Hz, 1H), 7.68-7.77 (m, 1H), 7.38-7.44 (m, 2H), 7.31-7.38 (m, 1H), 7.28 (t, J=9.3 Hz, 2H), 6.68 (d, J=5.7 Hz, 1H), 3.43-3.51 (m, 4H), and 2.02 (s, 4H).

Step B: 2,6-Difluoro-N-{2-fluoro-5-[5-(2-{[6-(4-morpholinyl)-3-pyridinyl]amino}-4-pyrimidinyl)-2-(1-pyrrolidinyl)-1,3-thiazol-4-yl]phenyl}benzenesulfonamide (Title Compound)

To a stirring solution of N-{5-[5-(2-chloro-4-pyrimidinyl)-2-(1-pyrrolidinyl)-1,3-thiazol-4-yl]-2-fluorophenyl}-2,6-difluorobenzenesulfonamide (0.10 g, 1.0 eq) in 2,2,2-trifluoroethanol (2 mL) was added 6-(4-morpholinyl)-3-pyridinamine (0.03 g, 1.0 eq), prepared by a procedure analogous to Intermediate 4, Step B, and 4 N HCl in dioxane (0.16 mL, 4 eq). The reaction was heated in the microwave at 175° C. for 50 min. The reaction mixture was quenched with 2 M NH₃ in MeOH (3 mL), concentrated onto silica gel, and purified by column chromatography (gradient elution: 0 to 50% DCM/10% MeOH in DCM). The cleanest fractions were combined, concentrated to a solid, and triturated with EtOAc/Hex to provide 28 mg (22%) of the title compound. ¹H-NMR (400 MHz, DMSO-d₆) δ ppm 10.94 (s, 1H), 9.30 (s, 1H), 8.44 (s, 1H), 7.99 (d, J=5.1 Hz, 1H), 7.94 (d, J=9.0 Hz, 1H), 7.70 (s, 1H), 7.39 (d, J=7.1 Hz, 2H), 7.23-7.34 (m, 3H), 6.83 (d, J=9.0 Hz, 1H), 6.07 (d, J=5.3 Hz, 1H), 3.68-3.74 (m, 4H), 3.41-3.48 (m, 4H), 3.34-3.38 (m, J=4.2 Hz, 4H), and 1.97-2.05 (m, 4H).

### Example 27

### N-{5-[2-(Dimethylamino)-5-(2-{[6-(4-morpholinyl)-3-pyridinyl]amino}-4-pyrimidinyl)-1,3-thiazol-4-yl]-2-fluorophenyl}-2,6-difluorobenzenesulfonamide

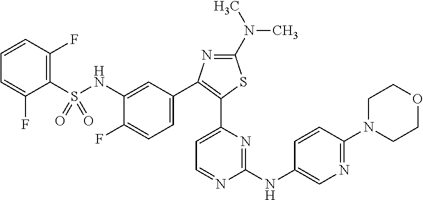

Step A: N-{5-[5-(2-Chloro-4-pyrimidinyl)-2-(dimethylamino)-1,3-thiazol-4-yl]-2-fluorophenyl}-2,6-difluorobenzenesulfonamide

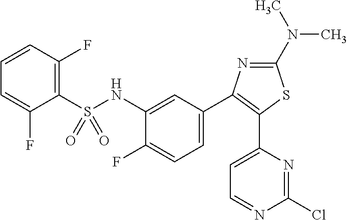

To a stirring suspension of N-{5-[(2-chloro-4-pyrimidinyl)acetyl]-2-fluorophenyl}-2,6-difluorobenzenesulfonamide (1.0 g, 2.26 mmol, 1.0 eq), prepared by a procedure analogous to Example 23, Step B, in DCM (˜5 mL) was added NBS (0.42 g, 2.38 mmol, 1.05 eq). Upon formation of a red solution (˜10 min) the reaction mixture was concentrated to a solid and taken up in dioxane (10 mL). To this solution was added Mg₂CO₃ (0.35 g) followed by N,N-dimethylthiourea (0.35 g, 3.39 mmol, 1.5 eq). After stirring 3 h, the mixture was concentrated onto silica gel and purified by column chromatography (gradient elution: 0 to 50% EtOAc/Hex to provide 167 mg (14%) of the title compound of Step A. ¹H-NMR (400 MHz, DMSO-d₆) δ ppm 11.01 (s, 1H), 8.24 (d, J=5.5 Hz, 1H), 7.68-7.76 (m, 1H), 7.34-7.44 (m, J=7.3 Hz, 2H), 7.24-7.34 (m, 3H), 6.71 (d, J=5.5 Hz, 1H), and 3.15 (s, 6H).

Step B: N-{5-[2-(dimethylamino)-5-(2-{[6-(4-morpholinyl)-3-pyridinyl]amino}-4-pyrimidinyl)-1,3-thiazol-4-yl]-2-fluorophenyl}-2,6-difluorobenzenesulfonamide (Title Compound)

To a stirring solution of N-{5-[5-(2-chloro-4-pyrimidinyl)-2-(dimethylamino)-1,3-thiazol-4-yl]-2-fluorophenyl}-2,6-difluorobenzenesulfonamide (0.10 g, 1.0 eq) in 2,2,2-trifluoroethanol (2 mL) was added 6-(4-morpholinyl)-3-pyridinamine (0.041 g, 1.0 eq), prepared by a procedure analogous to Intermediate 4, Step B, and 4 N HCl in dioxane (0.21 mL, 4 eq). The reaction was heated in the microwave at 175° C. for 20 min. The reaction mixture was quenched with 2 M NH₃ in MeOH (3 mL), concentrated onto silica gel, and purified by column chromatography (gradient elution: 0 to 50% DCM/10% MeOH in DCM) to provide 58 mg (41%) of the title compound. ¹H-NMR (400 MHz, DMSO-d₆) δ ppm 10.93 (brs, 1H), 9.32 (s, 1H), 8.43 (s, 1H), 8.01 (d, J=5.1 Hz, 1H), 7.94 (d, J=8.8 Hz, 1H), 7.71 (dt, J=14.0 and 7.1 Hz, 1H), 7.40 (d, J=7.3 Hz, 2H), 7.24-7.33 (m, 3H), 6.83 (d, J=9.2 Hz, 1H), 6.10 (d, J=5.1 Hz, 1H), 3.71 (t, 4H), 3.35 (t, 4H), and 3.12 (s, 6H).

### Example 28

### N-{5-[2-(1,1-Dimethylethyl)-5-(2-{[[6-(4-morpholinyl)-3-pyridinyl]amino}-4-pyrimidinyl)-1,3-thiazol-4-yl]-2-fluorophenyl]-2,6-difluorobenzenesulfonamide hydrochloride

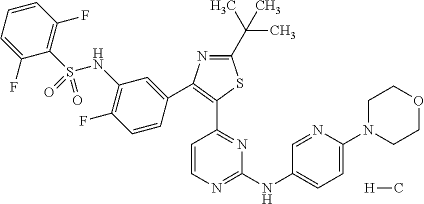

Step A: N-{5-[5-(2-Chloro-4-pyrimidinyl)-2-(1,1-dimethylethyl)-1,3-thiazol-4-yl]-2-fluorophenyl}-2,6-difluorobenzenesulfonamide

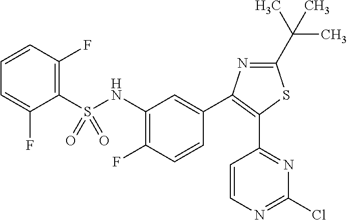

To a solution of N-{5-[(2-chloro-4-pyrimidinypacetyl]-2-fluorophenyl}-2,6-difluorobenzenesulfonamide (10.00 g, 22.63 mmol) prepared by a procedure analogous to Example 23, Step B in DMF (90 mL) was added NBS (4.23 g, 23.77 mmol). The reaction stirred 30 min at rt and then 2,2-diethylpropanethioamide (4.64 g, 39.6 mmol) prepared by a procedure analogous to Intermediate 8 was added. The reaction stirred 16 h at rt. The reaction mixture was then diluted with EtOAc (700 mL) and washed with water (2×500 mL). The organic fraction was dried over sodium sulfate, filtered, and concentrated onto silica gel. Purification by ISCO chromatography (20 to 100% EtOAc:hexanes) afforded N-{5-[5-(2-chloro-4-pyrimidinyl)-2-(1,1-dimethylethyl)-1,3-thiazol-4-yl]-2-fluorophenyl}-2,6-difluorobenzenesulfonamide (4.12 g, 7.26 mmol, 32.1% yield) as an off-white solid. ¹H-NMR (400 MHz, DMSO-d₆): δ 11.01 (s, 1H), 8.55 (d, J=5.3 Hz, 1H), 7.61-7.90 (m, 1H), 7.39-7.54 (m, 2H), 7.19-7.39 (m, 3H), 7.12 (d, J=5.3 Hz, 1H), and 1.43 (s, 9H); MS (ESI): 538.99 [M+H]⁺.

Step B: N-{5-[2-(1,1-Dimethylethyl)-5-(2-{[6-(4-morpholinyl)-3-pyridinyl]amino}-4-pyrimidinyl)-1,3-thiazol-4-yl]-2-fluorophenyl}-2,6-difluorobenzenesulfonamide hydrochloride (Title Compound)

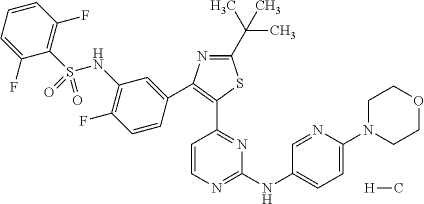

A suspension of N-{5-[5-(2-chloro-4-pyrimidinyl)-2-(1,1-dimethylethyl)-1,3-thiazol-4-yl]-2-fluorophenyl}-2,6-difluorobenzenesulfonamide (3.00 g, 5.57 mmol) prepared by a procedure analogous to Example 28, Step A and 6-(4-morpholinyl)-3-pyridinamine (0.998 g, 5.57 mmol) prepared by a procedure analogous to Intermediate 4, Step B in 2,2,2-trifluoroethanol (28 mL) with 90 drops of concentrated hydrochloric acid was divided into three portions which were sequentially heated at 170° C. in a microwave for 90 min, then recombined and concentrated onto silica gel. Purification by ISCO chromatography (0 to 100% (15% MeOH:1% ammonium hydroxide:DCM):EtOAc) afforded a solid which was suspended in EtOAc (25 mL) and treated with hydrochloric acid (4 N in 1,4-dioxane, 760 μl, 3.04 mmol). The mixture stirred 20 min at rt. The resultant solid was then collected by vacuum filtration and dried overnight in a vacuum oven to afford N-{5-[2-(1,1-dimethylethyl)-5-(2-{[6-(4-morpholinyl)-3-pyridinyl]amino}-4-pyrimidinyl)-1,3-thiazol-4-yl]-2-fluorophenyl}-2,6-difluorobenzenesulfonamide hydrochloride (1.77 g, 2.341 mmol, 42.1% yield) as a dark tan solid. ¹H-NMR (400 MHz, DMSO-d₆) δ 10.98 (s, 1H), 10.01 (brs, 1H), 8.65 (brs, 1H), 8.33 (d, J=5.1 Hz, 1H), 8.15 (d, J=9.3 Hz, 1H), 7.59-7.85 (m, 1H), 7.45 (d, J=7.9 Hz, 2H), 7.17-7.41 (m, 4H), 6.53 (d, J=5.1 Hz, 1H), 3.70-3.88 (m, 4H), 3.57-3.70 (m, 4H), and 1.45 (s, 9H); MS (ESI): 682.16 [M+H]⁺.

### Example 29

### 2,6-Difluoro-N-{2-fluoro-3-[5-[2-({3-fluoro-4-[4-(2-fluoroethyl)-1-piperazinyl]phenyl}amino)-4-pyrimidinyl]-2-(1-methylethyl)-1,3-thiazol-4-yl]phenyl}benzenesulfonamide

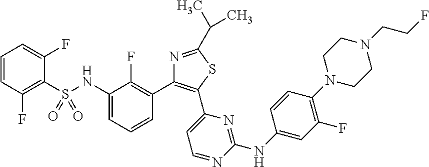

Step A: Methyl 3-bromo-2-fluorobenzoate

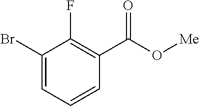

To a 100 mL round bottom flask was added 3-bromo-2-fluorobenzoic acid (10.4 g, 47.5 mmol), MeOH (100 mL, 2472 mmol) and sulfuric acid (6 mL, 113 mmol). The reaction mixture was refluxed for 1 hr. After cooling to rt, the MeOH was removed under reduced pressure and the acidic residue was poured into cold water and EtOAc, the layers were separated and the aqueous layer was extracted with EtOAc. The organic layers were combined, washed with brine, dried over sodium sulfate and concentrated under reduced pressure to afford 10.02 g of methyl 3-bromo-2-fluorobenzoate. ¹H-NMR (400 MHz, DMSO-d₆) δ 7.95 (ddd, J=8.1, 6.4, and 1.7 Hz, 1H), 7.82-7.87 (m, 1H), 7.26 (t, J=7.9 Hz, 1H), and 3.86 (s, 3H).

Step B: Methyl 3-amino-2-fluorobenzoate

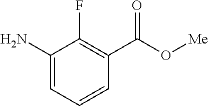

In a 500 mL flask was placed 1,1-dimethylethyl carbamate (6.03 g, 51.5 mmol), methyl 3-bromo-2-fluorobenzoate (10 g, 42.9 mmol), Pd₂(dba)₃.CHCl₃ (0.89 g, 0.86 mmol), xantphos (1.49 g, 2.57 mmol) and cesium carbonate (16.8 g, 51.5 mmol). The flask was sealed with a rubber septum, placed under high vac, and toluene (200 mL) was added. Three cycles of high vac/N₂ were performed and the reaction mixture was stirred at 90° C. overnight. The reaction was filtered through a pad of celite with EtOAc washing and concentrated. To the residue was added DCM (200 mL) followed by TFA (50 mL, 649 mmol), and the mixture was stirred at rt for 1 h. The volatiles were removed under reduced pressure and the residue was taken up in EtOAc and washed with saturated NaHCO₃ and brine. The organic layer was dried over sodium sulfate and stripped onto silica and column chromatographed on silica with 5% to 50% EtOAc:Hexane to give 5.53 g (76%) of the title compound of Step B. ¹H-NMR (400 MHz, DMSO-d₆) δ 6.92-7.01 (m, 3H), 5.37 (s, 2H), and 3.81 (s, 3H). MS (ESI): 170 [M+H]⁺.

Step C: Methyl 3-{[(2,6-difluorophenyl)sulfonyl]amino}-2-fluorobenzoate

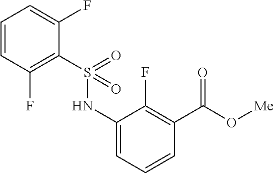

In a 500 mL flask was placed methyl 3-amino-2-fluorobenzoate (5.5 g, 32.5 mmol) and DCM (100 mL) and pyridine (2.9 mL, 35.8 mmol) was added. 2,6-Difluorobenzenesulfonyl chloride (7.6 g, 35.8 mmol) in DCM (50 mL) was added dropwise via addition funnel and the reaction mixture was allowed to stir at rt overnight. The reaction mixture was stripped onto silica and column chromatographed on silica with 5% to 100% EtOAc:Hexane to give 9.75 g (87%) of the title compound of Step C. ¹H-NMR (400 MHz, DMSO-d₆) δ 10.98 (s, 1H), 7.64-7.82 (m, 3H), 7.46-7.61 (m, 1H), 7.29 (t, J=8.8 Hz, 2H), and 3.81 (s, 3H). MS (ESI): 346 [M+H]⁺.

Step D: N-{3-[(2-Chloro-4-pyrimidinyl)acetyl]-2-fluorophenyl}-2,6-difluorobenzenesulfonamide

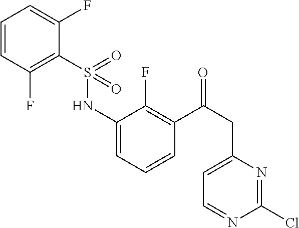

In a 1000 mL flask was placed methyl 3-{[(2,6-difluorophenyl)sulfonyl]amino}-2-fluorobenzoate (9.64 g, 27.9 mmol) and THF (200 mL) was added. The flask was placed in an ice/water bath and LHMDS (90 mL, 90 mmol) was added. 2-Chloro-4-methylpyrimidine (4.5 g, 35.0 mmol) in THF (60 mL) was added dropwise via addition funnel. After the addition was complete, the reaction was allowed to warm to 20° C. over 1 h. The THF volume was reduced to half under reduced pressure and then treated with 6 N HCl. EtOAc was added and the layers were separated. The aqueous layer was extracted twice with EtOAc and the combined organic layer was washed once with brine, dried over sodium sulfate, and concentrated. The residue was triturated with EtOAc/ether to afford 8.71 g (71%) of the title compound of Step D. MS (ESI): 442 [M+H]+.

Step E: N-{3-[5-(2-Chloro-4-pyrimidinyl)-2-(1-methylethyl)-1,3-thiazol-4-yl]-2-fluorophenyl}-2,6-difluorobenzenesulfonamide

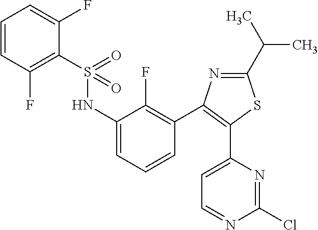

In a 250 mL flask was placed N-{3-[(2-chloro-4-pyrimidinyl)acetyl]-2-fluorophenyl}-2,6-difluorobenzenesulfonamide (4 g, 9.05 mmol) and DMF (60 mL) was added. NBS (1.62 g, 9.10 mmol) was added and, after stirring at rt for 40 min, 2-methylpropanethioamide (1.4 g, 13.6 mmol), prepared by a procedure analogous to Intermediate 3, was added. After 4 h at rt, the reaction mixture was poured into 800 mL of EtOAc and washed 4 times with 250 mL of H₂O, washed once with 200 mL of brine, and dried over sodium sulfate. Silica gel was added and the volatiles were removed under reduced pressure. Column chromatography with 10% to 60% EtOAc:Hexane gave 2.15 g (45%) of the title compound of Step E. ¹H-NMR (400 MHz, DMSO-d₆) δ 11.43 (s, 1H), 9.06 (d, J=5.3 Hz, 1H), 8.12-8.30 (m, 1H), 7.93-8.06 (m, 2H), 7.84 (t, J=7.9 Hz, 1H), 7.75 (t, J=9.2 Hz, 2H), 7.37 (d, J=5.3 Hz, 1H), 3.77-3.93 (m, 1H), and 1.89 (d, J=6.8 Hz, 6H). MS (ESI): 524 [m]⁺.

Step F: 2,6-Difluoro-N-{2-fluoro-3-[5-[2-({3-fluoro-4-[4-(2-fluoroethyl)-1-piperazinyl]phenyl}amino)-4-pyrimidinyl]-2-(1-methylethyl)-1,3-thiazol-4-yl]phenyl}benzenesulfonamide

The title compound of Example 29 (0.053 g, 48%) was made following the general procedure of Example 2, Step D from N-{3-[5-(2-chloro-4-pyrimidinyl)-2-(1-methylethyl)-1,3-thiazol-4-yl]-2-fluorophenyl}-2,6-difluorobenzenesulfonamide (0.08 g, 0.15 mmol) and 3-fluoro-4-[4-(2-fluoroethyl)-1-piperazinyl]phenyl amine (0.054 g, 0.22 mmol), prepared by a procedure analogous to Example 21, Step E. ¹H-NMR (400 MHz, DMSO-d₆) δ 10.84 (brs, 1H), 9.74 (s, 1H), 8.24 (d, J=5.1 Hz, 1H), 7.60-7.72 (m, 2H), 7.41-7.48 (m, 1H), 7.32-7.41 (m, 2H), 7.29 (t, J=7.9 Hz, 1H), 7.22 (t, J=9.0 Hz, 2H), 6.93-7.02 (m, 1H), 6.14 (d, J=5.1 Hz, 1H), 4.60-4.70 (m, 1H), 4.48-4.57 (m, 1H), 3.26-3.42 (m, 1H), 2.93-3.03 (m, 4H), 2.76 (t, J=4.8 Hz, 1H), 2.60-2.73 (m, 5H), and 1.38 (d, J=7.0 Hz, 6H). MS (ESI): 730 [M+H]⁺.

### Example 30

### 2,6-Difluoro-N-{2-fluoro-3-[5-(2-{[5-fluoro-6-(4-morpholinyl)-3-pyridinyl]amino}-4-pyrimidinyl)-2-(1-methylethyl)-1,3-thiazol-4-yl]phenyl}benzenesulfonamide

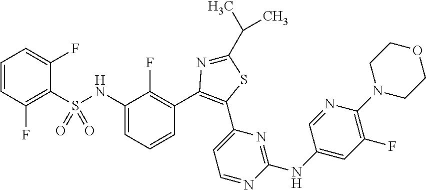

Step A: 5-Fluoro-6-(4-morpholinyl)-3-pyridinamine

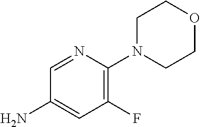

4-(3-Fluoro-2-pyridinyl)morpholine (0.50 g, 2.74 mmol) in sulfuric acid (0.47 mL) was treated portion-wise with potassium nitrate (0.33 g, 3.26 mmol). After stirring at rt for 1 h, the reaction mixture was quenched with 6 N sodium hydroxide and saturated sodium bicarbonate. The product was extracted with EtOAc, dried over sodium sulfate, and the volatiles were removed under reduced pressure. The residue was taken up in EtOH and acetic acid, 10% palladium on carbon was added, and the reaction mixture was stirred overnight under hydrogen at 60 psi. The reaction mixture was filtered through a pad of celite with MeOH washing to afford 0.25 g (47%) the title compound of Step A. ¹H-NMR (400 MHz, DMSO-d₆) δ 7.46 (d, J=0.9 Hz, 1H), 6.80 (dd, J=14.3 and 2.2 Hz, 1H), 5.09 (s, 2H), 3.61-3.77 (m, 4H), and 3.02-3.14 (m, 4H); MS (ESI): 198 [M+H]⁺.

Step B: 2,6-Difluoro-N-{2-fluoro-3-[5-(2-{[5-fluoro-6-(4-morpholinyl)-3-pyridinyl]amino}-4-pyrimidinyl)-2-(1-methylethyl)-1,3-thiazol-4-yl]phenyl}benzenesulfonamide

The title compound (0.044 g, 34%) was made following the general procedure of Example 2, Step D from N-{3-[5-(2-chloro-4-pyrimidinyl)-2-(1-methylethyl)-1,3-thiazol-4-yl]-2-fluorophenyl}-2,6-difluorobenzenesulfonamide (0.10 g, 0.19 mmol), prepared by a procedure analogous to Example 29, Step E, and 5-fluoro-6-(4-morpholinyl)-3-pyridinamine (0.045 g, 0.23 mmol). ¹H-NMR (400 MHz, DMSO-d₆) δ 10.90 (s, 1H), 9.87 (s, 1H), 8.32 (d, J=1.5 Hz, 1H), 8.27 (d, J=5.5 Hz, 1H), 8.01 (dd, J=15.2 and 2.0 Hz, 1H), 7.63-7.73 (m, 1H), 7.39-7.48 (m, 2H), 7.31 (t, J=7.9 Hz, 1H), 7.24 (t, J=9.0 Hz, 2H), 6.18 (d, J=5.1 Hz, 1H), 3.69-3.79 (m, 4H), 3.30-3.41 (m, 1H), 3.22-3.31 (m, 4H), and 1.38 (d, J=7.0 Hz, 6H); MS (ESI): 686 [M+H]⁺.

### Example 31

### N-{2,3-Difluoro-5-[5-(2-{[5-fluoro-6-(4-morpholinyl)-3-pyridinyl]amino}-4-pyrimidinyl)-2-(1-methylethyl)-1,3-thiazol-4-yl]phenyl}-3-fluorobenzenesulfonamide

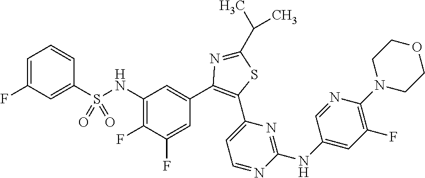

Step A: Methyl 3-bromo-4,5-difluorobenzoate

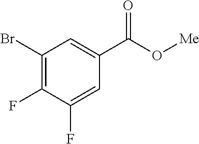

The title compound (4.49 g, 58%) of Step A was made from 3-bromo-4,5-difluorobenzoic acid (5.2 g, 21.9 mmol), MeOH (40 mL), and sulfuric acid (3 mL) in a procedure analogous to the procedure described in Example 29, Step A. ¹H-NMR (400 MHz, CDCl₃) δ 7.73 (dd, J=10.6, 8.2 Hz, 1H), 7.51 (dd, J=9.5 and 7.1 Hz, 1H), and 3.93 (s, 3H).

Step B: Methyl 3-amino-4,5-difluorobenzoate

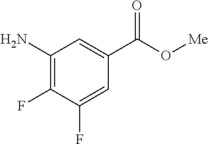

The title compound (1.66 g, 50%) of Step B was made in a procedure analogous to the procedure described in Example 29, Step B from methyl 2-bromo-4,5-difluorobenzoate. ¹H-NMR (400 MHz, DMSO-d₆) δ 8.13 (brs, 2H), 7.27 (d, J=7.3 Hz, 1H), 6.99 (ddd, J=10.6, 7.0, and 2.2 Hz, 1H), and 3.80 (s, 3H). MS (ESI), 188 [M+H]⁺.

Step C: Methyl 3,4-difluoro-5-{[(3-fluorophenyl)sulfonyl]amino}benzoate

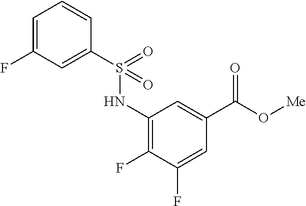

The title compound (1.09 g, 66%) of Step C was made in a procedure analogous to the procedure described in Example 29, Step C from methyl 3-amino-4,5-difluorobenzoate (0.090 g, 4.81 mmol) and 3-fluorobenzenesulfonyl chloride (0.38 g, 5.05 mmol). MS (ESI): 344 [M−H]⁺.

Step D: N-{5-[(2-Chloro-4-pyrimidinypacetyl]-2,3-difluorophenyl}-3-fluorobenzenesulfonamide

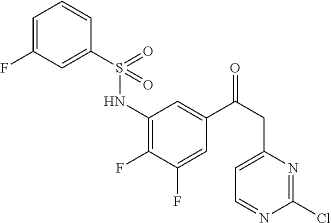

The title compound (0.90 g, 64%) of Step D was made in a procedure analogous to the procedure described in Example 29, Step D from methyl 3,4-difluoro-5-{[(3-fluorophenyl)sulfonyl]amino}benzoate (1.1 g, 3.2 mmol) and 2-chloro-4-methylpyrimidine (0.45 g, 3.50 mmol). MS (ESI): 442 [M+H]⁺.

Step E: N-{5-[5-(2-chloro-4-pyrimidinyl)-2-(1-methylethyl)-1,3-thiazol-4-yl]-2,3-difluorophenyl}-3-fluorobenzenesulfonamide

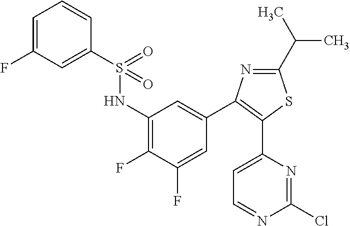

The title compound (0.40 g, 37%) of Step E was made in a procedure analogous to the procedure described in Example 29, Step E from N-{5-[(2-chloro-4-pyrimidinypacetyl]-2,3-difluorophenyl}-3-fluorobenzenesulfonamide (0.90 g, 2.04 mmol) and 2-methylpropanethioamide (0.32 g, 3.06 mmol), prepared by a procedure analogous to Intermediate 3. ¹H-NMR (400 MHz, DMSO-d₆) δ 10.81 (s, 1H), 8.58 (d, J=5.5 Hz, 1H), 7.53-7.65 (m, 5H), 7.22 (d, J=5.5 Hz, 1H), 7.15 (dt, J=6.5 and 1.7 Hz, 1H), 3.28-3.39 (m, 1H), and 1.38 (d, J=7.00 Hz, 6H); MS (ESI): 525 [M+H]⁺.

Step F: N-{2,3-Difluoro-5-[5-(2-{[5-fluoro-6-(4-morpholinyl)-3-pyridinyl]amino}-4-pyrimidinyl)-2-(1-methylethyl)-1,3-thiazol-4-yl]phenyl}-3-fluorobenzenesulfonamide The title compound (0.023 g, 17%) was made following the general procedure of Example 2, Step D from N-{5-[5-(2-chloro-4-pyrimidinyl)-2-(1-methylethyl)-1,3-thiazol-4-yl]-2,3-difluorophenyl}-3-fluorobenzenesulfonamide (0.105 g, 0.20 mmol) and 5-fluoro-6-(4-morpholinyl)-3-pyridinamine (0.047 g, 0.24 mmol), prepared by a procedure analogous to Example 30, Step A. ¹H-NMR (400 MHz, DMSO-d₆) δ 10.78 (s, 1H), 9.87 (s, 1H), 8.38 (d, J=4.9 Hz, 1H), 8.25 (d, J=0.7 Hz, 1H), 7.88 (s, 1 H), 7.58 (d, J=5.1 Hz, 1H), 7.48-7.55 (m, 4H), 7.14 (s, 1H), 6.60 (d, J=5.1 Hz, 1H), 3.69-3.75 (m, 4H), 3.28-3.37 (m, 1H), 3.20-3.28 (m, 4H), and 1.38 (d, J=6.8 Hz, 6H); MS (ESI): 686 [M+H]⁺.

### Example 32

### 2,6-Difluoro-N-{4-[2-(1-methylethyl)-5-(2-{[6-(4-morpholinyl}-3-pyridinyl]amino}-4-pyrimidinyl)-1,3-thiazol-4-yl]-2-pyridinyl}benzenesulfonamide

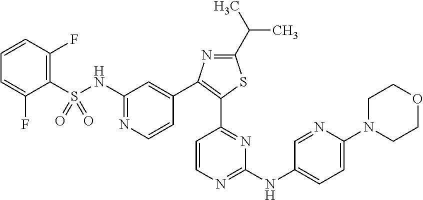

Step A: Methyl 2-{[(2,6-difluorophenyl)sulfonyl]amino}-4-pyridinecarboxylate

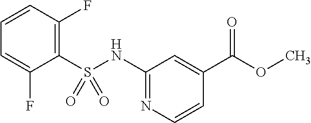

2,6-Difluorobenzenesulfonylchloride (1.52 g; 7.15 mmol) was added to a solution of methyl-2-aminopyridine-4-carboxylate (1.0 g; 6.57 mmol) dissolved in excess anhydrous pyridine (5 mL) in a sealed tube. The tube was heated between 80-90° C. for 16 h. A resulting mushy residue was added to water (30 mL) and stirred for 1 h and a tacky camel tan solid was collected by filtration as the crude product (1.25 g). The solid was triturated with MeOH, collected by filtration to give the desired title compound of Step A (1.02 g, 47%). ¹H-NMR (400 MHz, DMSO-d₆) δ 8.20 (d, J=4.7 Hz, 1H), 7.56-7.74 (m, 2H), 7.30 (d, J=4.0 Hz, 1H), 7.24 (t, J=9.2 Hz, 2H), and 3.88 (s, 3H).

Step B: N-{4-[(2-Chloro-4-pyrimidinyl)acetyl]-2-pyridinyl}-2,6-difluorobenzenesulfonamide

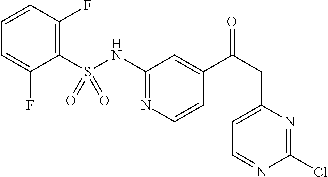

Methyl 2-{[(2,6-difluorophenyl)sulfonyl]amino}-4-pyridinecarboxylate (1.2 g, 3.66 mmol) was dissolved in THF (8 mL) and cooled to 0° C. in an oven-dried flask, 1.0 M LiHMDS in hexanes (9 mL, 9 mmol) was then added dropwise. A solution of the 2-chloro-4-methylpyrimidine (0.4 g; 3.11 mmol) in THF (4 mL) was slowly added via dropping funnel. The reaction darkened and was stirred at 0° C., then warmed to rt. The reaction was quenched with MeOH while chilled and concentrated in vacuo to a tacky orange residue. This residue was dissolved in EtOAc (150 mL) and washed with water (2×25 mL), filtered through Whatman 1 PS paper, and concentrated in vacuo to a crude orange solid residue (1.28 g). It was later discovered that a major portion of the desired product was water-soluble and concentration in vacuo led to a 2^{nd} crude crop of material (1.39 g). Purification of the 1^{st} sample by chromatography led to no pure material. The 2nd residue was initially triturated with acetone: ether: MeOH (1:10:1). When the volume was reduced to ˜75%, three independent crops of camel tan solid were collected by filtration and air-dried to give the titled compound of Step B (0.21 g; 0.30 g, 0.16 g; total=0.65 g, 76%). ¹H-NMR (400 MHz, DMSO-d₆) δ ppm 13.29 (brs, 1H), 8.54 (brs, 1H), 8.03 (s, 1H), 7.55-7.72 (m, 4H), 7.21 (t, J=9.0 Hz, 4H), and 6.48 (brs, 1H);

Step C: N-{4-[5-(2-Chloro-4-pyrimidinyl)-2-(1-methylethyl)-1,3-thiazol-4-yl]-2-pyridinyl}-2,6-difluorobenzenesulfonamide

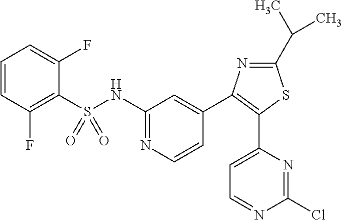

Re-crystallized NBS (0.2 g, 0.56 mmol) was added to a solution of N-{4-[(2-chloro-4-pyrimidinyl)acetyl]-2-pyridinyl}-2,6-difluorobenzenesulfonamide (0.21 g; 0.49 mmol) in DMF (4 mL) in an ice bath, the reaction was immediately removed and the reaction allowed to warm to rt. To this mixture was added the 2-methylpropanethioamide (prepared by a procedure analogous to Intermediate 3, 0.2 g, 1.9 mmol) in DMF (1 mL) at 0° C. and the reaction was stirred for 1 h at rt. The reaction was concentrated to a syrup, but a tacky golden solid was precipitated when the syrup was diluted with H₂O, Similarly, other batches of the starting N-{4-[(2-chloro-4-pyrimidinyl)acetyl]-2-pyridinyl}-2,6-difluorobenzenesulfonamide (0.3 g; 0.71 mmol and 0.16 g; 0.38 mmol) were reacted under identical conditions to yield other samples of crude material. A TLC comparison of these materials led to them being combined into one crude sample for purification. An initial re-crystallization from DCM with hexanes and isopropanol lead to no pure material. Purification by chromatography (0-100%) EtOAc: hexanes gave the desired title compound of Step C as a solid (0.142 g, 17%). ¹H-NMR (400 MHz, DMSO-d₆, variable temperature experiment at 80° C.) 6 8.62 (d, J=5.2 Hz, 1H), 7.95-8.08 (m, 1H), 7.51-7.65 (m, 1H), 7.40 (d, J=4.9 Hz, 2H), 7.08-7.23 (m, 2H), 7.02-7.08 (m, 1H), 3.33-3.43 (m, 1H), and 1.41 (d, J=6.9 Hz, 6H).

Step D: 2,6-Difluoro-N-{4-[2-(1-methylethyl)-5-(2-{[6-(4-morpholinyl)-3-pyridinyl]amino}-4-pyrimidinyl)-1,3-thiazol-4-yl]-2-pyridinyl}benzenesulfonamide

The general procedure (170° C., 1.75 h; microwave, 2,2,2-trifluoroethanol (5 mL) with p-toluenesulfonic acidic monohydrate (0.045 g, 0.2 mmol)) of Example 2, Step D was used for the reaction of N-{4-[5-(2-chloro-4-pyrimidinyl)-2-(1-methylethyl)-1,3-thiazol-4-yl]-2-pyridinyl}-2,6-difluorobenzenesulfonamide (0.07 g, 0.14 mmol) and 6-(4-morpholinyl)-3-pyridinamine (prepared by a procedure analogous to Intermediate 4, Step B, 0.046 g, 0.26 mmol) to give a crude product. Purification by chromatography (0-100% DCM:MeOH: ammonium hydroxide/84:15:1) to EtOAc gave desired title compound. This crude product was triturated with MeOH, collected by filtration, washed with hexanes, and air-dried to give an orange solid (0.050 g, 55%). ¹H-NMR (400 MHz, DMSO-d₆, variable temperature experiment at 80° C.) δ 9.25 (s, 1H), 8.28-8.38 (m, 2H), 7.95 (brs, 1H), 7.77 (dd, J=8.9 and 2.3 Hz, 1H), 7.57 (t, J=6.9 Hz, 1H), 7.47-7.52 (m, 1H), 7.11 (t, J=9.2 Hz, 2H), 6.98 (dd, J=7.3 and 2.6 Hz, 1H), 6.72 (d, J=9.0 Hz, 1H), 6.66 (d, J=5.1 Hz, 1H), 3.72 (t, J=4.7 Hz, 4H), 3.35-3.43 (m, 5H), and 1.41 (d, J=6.9 Hz, 6H).

### Example 33

### 2,6-Difluoro-N-{4-[5-(2-{[5-fluoro-6-(4-morpholinyl-3-pyridinyl]amino}-4-pyrimidinyl)-2-(1-methylethyl)-1,3-thiazol-4-yl]-2-pyridinyl}benzenesulfonamide

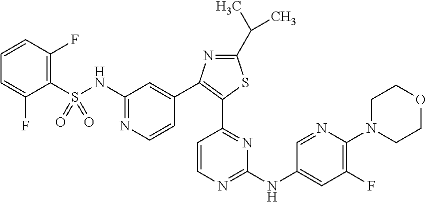

The general procedure (175° C.; 0.5 h; 2,2,2-trifluoroethanol (4 mL) in a microwave reactor, with 7 drops of conc. HCl) was used for the reaction of N-{4-[5-(2-chloro-4-pyrimidinyl)-2-(1-methylethyl)-1,3-thiazol-4-yl]-2-pyridinyl}-2,6-difluorobenzenesulfonamide, 0.075 g, 0.15 mmol), prepared by a procedure analogous to Example 32, Step C, and 3-fluoro-4-[4-(2-fluoroethyl)-1-piperazinyl]phenyl amine (0.04 g, 0.17 mmol), prepared by a procedure analogous to Example 30, Step A, to give a crude product. Purification by chromatography (0-100%) DCM:MeOH: ammonium hydroxide/84:15:1) to EtOAc gave desired title compound. This crude product was triturated with MeOH, collected by filtration, washed with hexanes and air-dried to give a brownish-yellow solid (0.050 g, 50%). ¹H-NMR (400 MHz, DMSO-d₆) δ 9.88 (s, 1H), 8.39 (d, J=5.1 Hz, 1H), 8.26 (brs, 1 H), 7.83-8.04 (m, 2H), 7.49-7.66 (m, 2H), 7.11 (ddd, J=2.6, 1.6, and 1.4 Hz, 2 H), 6.96 (brs, 1H), 6.78 (brs, 1H), 3.63-3.81 (m, 5H), 3.20-3.27 (m, 4H), and 1.39 (d, J=6.9 Hz, 6H).

### Examples 34-177

The following examples 34-177, were prepared by procedures outlined above and similar to Examples 1-33.

| Example | Structure | Characterization |
|---|---|---|
| 34 | 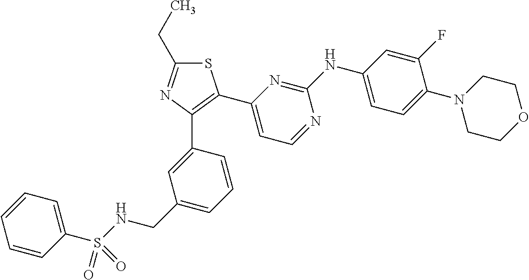 | MS (ESI) 631.19 [M + H]+ |
| 35 | 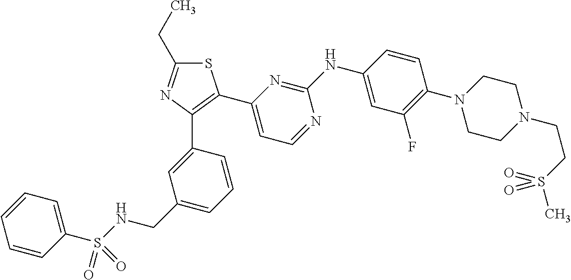 | MS (ESI) 736.12 [M + H]+ |
| 36 | 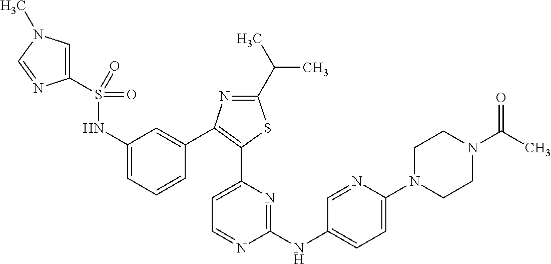 | MS (ESI) 659 [M + H]+ |
| 37 | 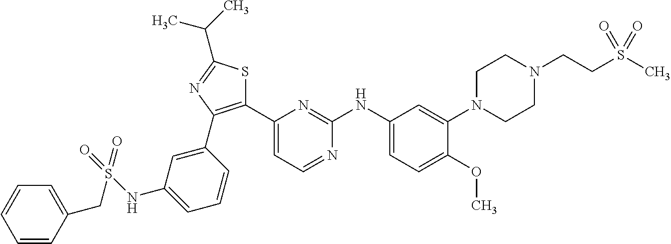 | MS (ESI) 760.58 [M − H] |
| 38 | 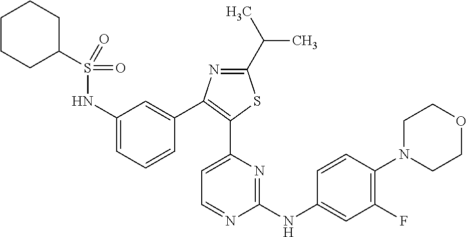 | MS (ESI) 637 [M + H]+ |
| 39 | 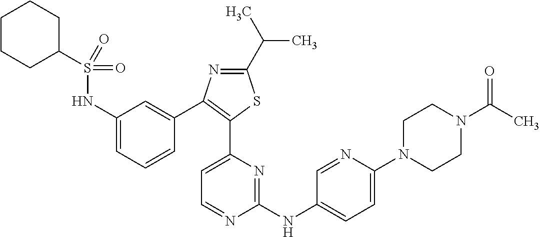 | MS (ESI) 661 [M + H]+ |
| 40 | 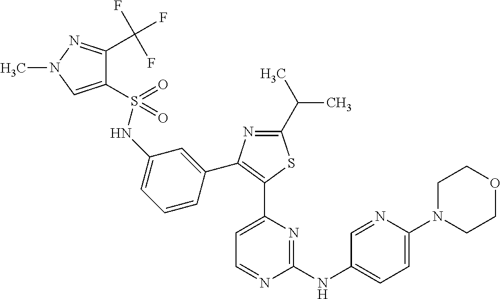 | MS (ESI) 686.22 [M + H]+ |
| 41 | 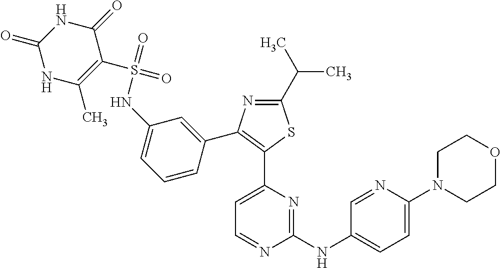 | MS (ESI) 662.17 [M + H]+ |
| 42 | 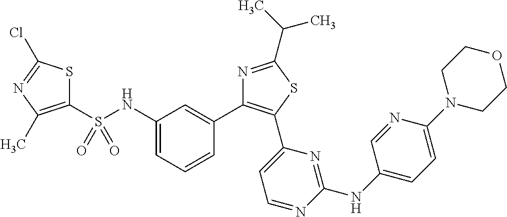 | MS (ESI) 669.30 [M + H]+ |
| 43 | 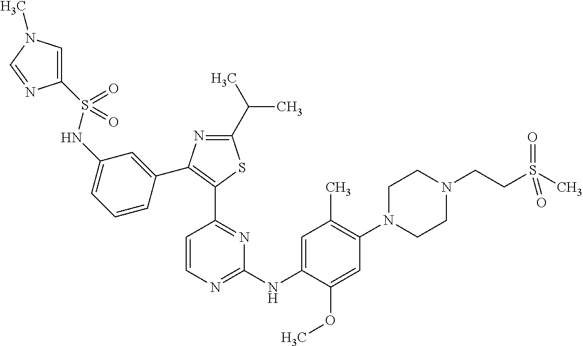 | MS (ESI) 766.41 [M + H]+ |
| 44 | 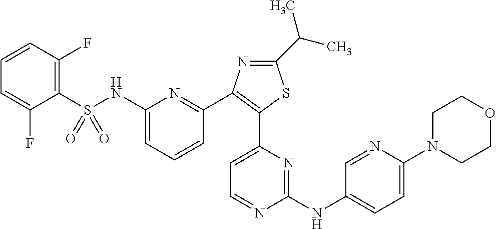 | MS (ESI) 651.13 [M + H]+ |
| 45 | 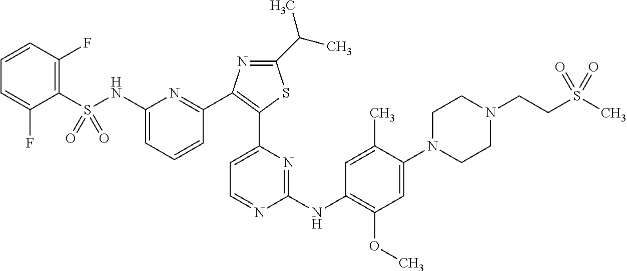 | MS (ESI) 799.29 [M + H]+ |
| 46 | 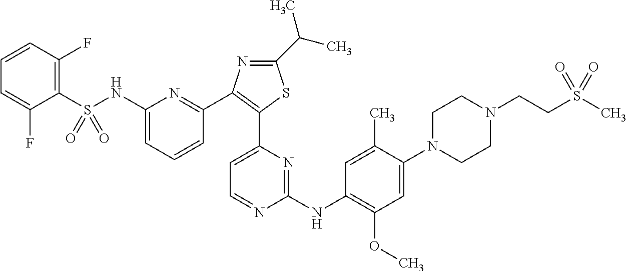 | MS (ESI) 678.17 [M + H]+ |
| 47 | 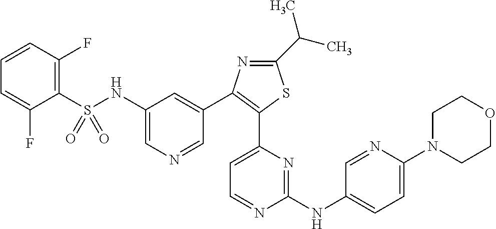 | MS (ESI) 651.12 [M + H]+ |
| 48 | 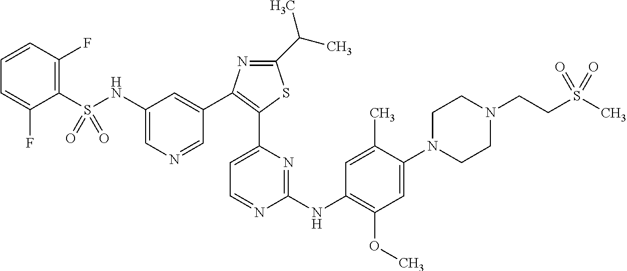 | MS (ESI) 799.28 [M + H]+ |
| 49 | 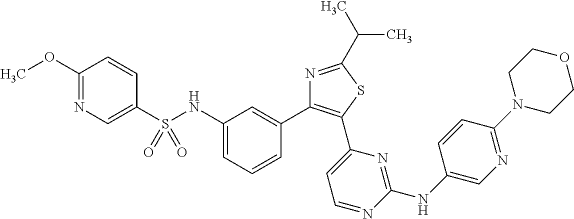 | MS (ESI) 645.21 [M + H]+ |
| 50 | 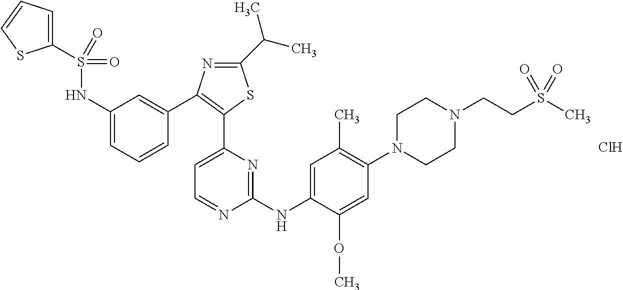 | MS (ESI) 768 [M + H]+ |
| 51 | 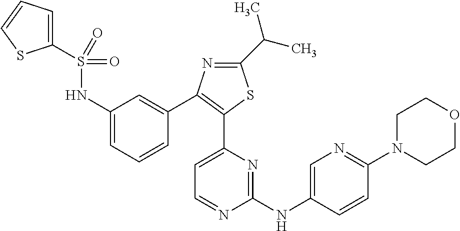 | MS (ESI) 620 [M + H]+ |
| 52 | 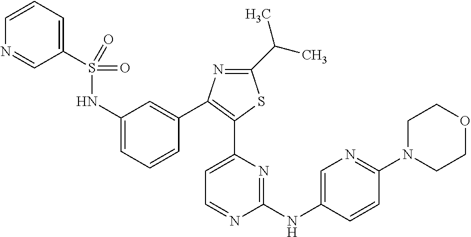 | MS (ESI) 615 [M + H]+ |
| 53 | 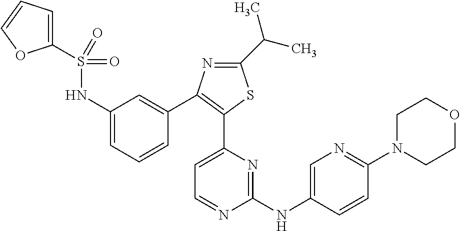 | MS (ESI) 604 [M + H]+ |
| 54 | 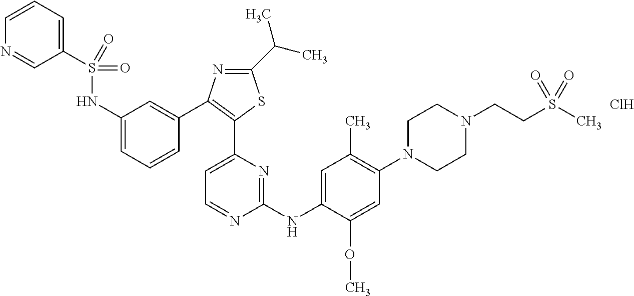 | MS (ESI) 763 [M + H]+ |
| 55 | 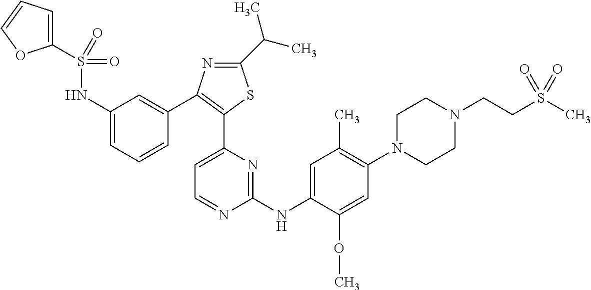 | MS (ESI) 752 [M + H]+ |
| 56 | 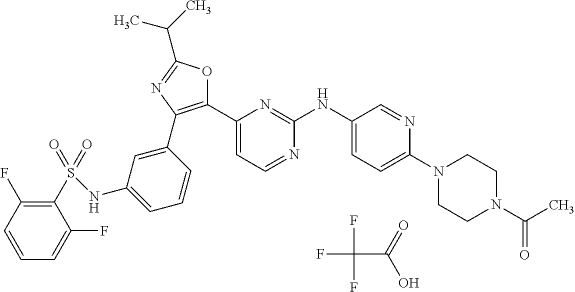 | MS (ESI) 675.24 [M + H]+ (free base) |
| 57 | 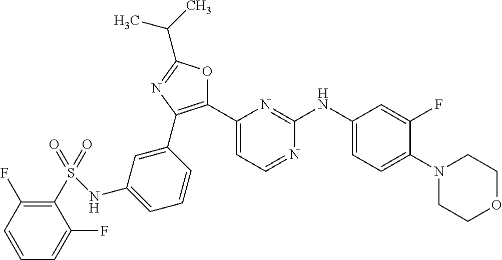 | MS (ESI) 651.26 [M + H]+ |
| 58 | 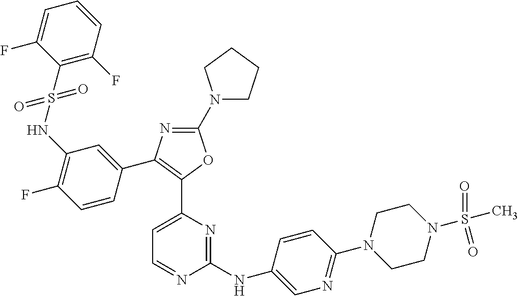 | MS (ESI) 689.07 [M + H]+ |
| 59 | 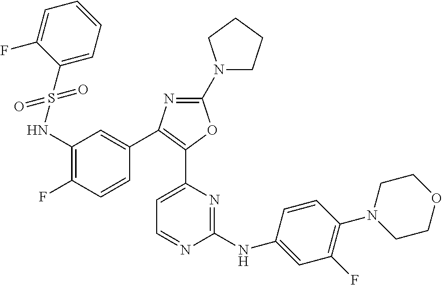 | MS (ESI) 678.19 [M + H]+ |
| 60 | 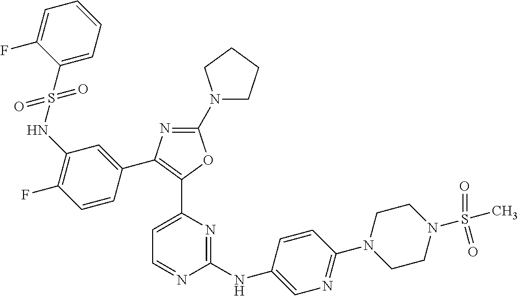 | MS (ESI) 738.19 [M + H]+ |
| 61 | 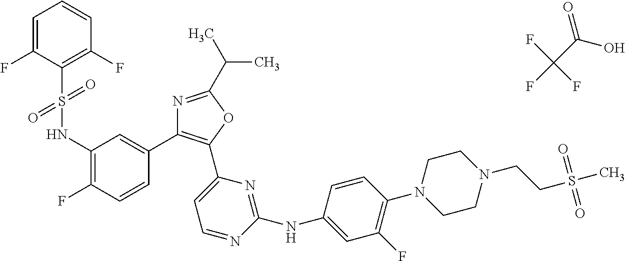 | MS (ESI) 774.21 [M + H]+ |
| 62 | 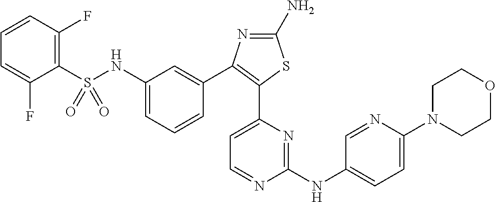 | MS (ESI) 622.36 [M + H] |
| 63 | 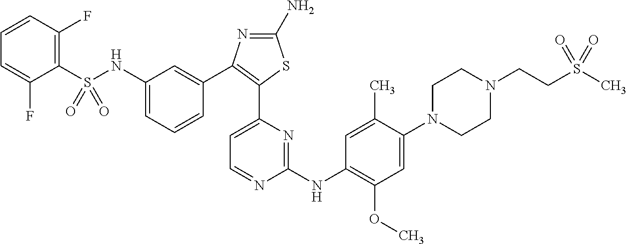 | MS (ESI) 770.88 [M + H] |
| 64 | 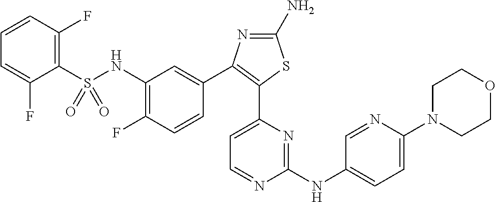 | MS (ESI) 641.11 [M + H]+ |
| 65 | 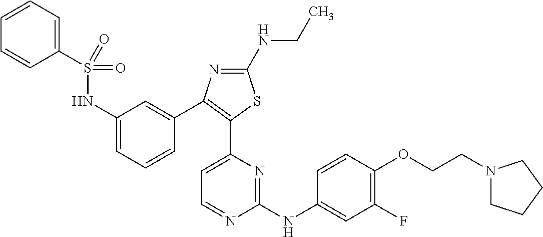 | MS (ESI) 660.80 [M + H]+ |
| 66 | 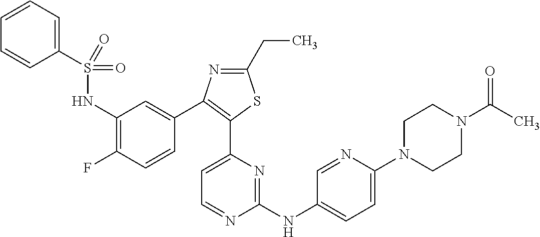 | MS (ESI) 659.44 [M + H]+ |
| 67 | 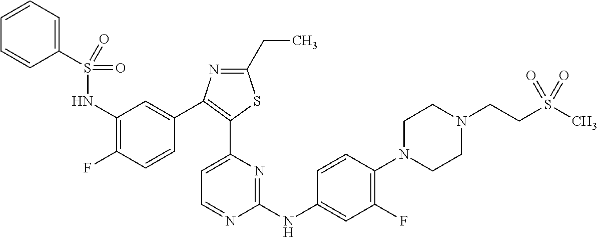 | MS (ESI) 740.41 [M + H]+ |
| 68 | 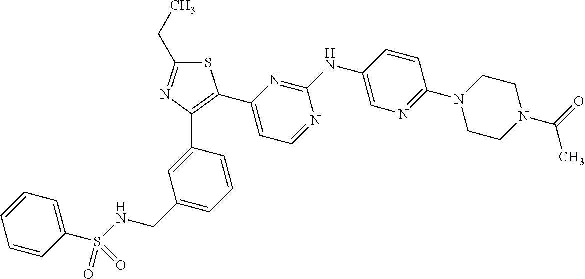 | MS (ESI) 655.10 [M + H]+ |
| 69 | 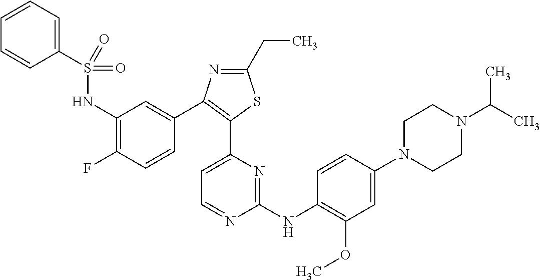 | MS (ESI) 688.15 [M + H]+ |
| 70 | 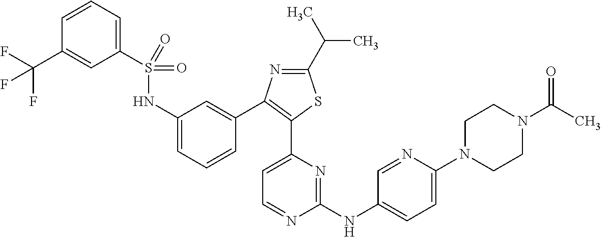 | MS (ESI) 723.15 [M + H]+ |
| 71 | 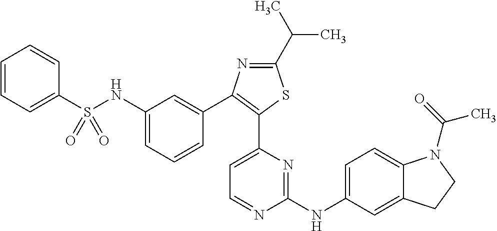 | MS (ESI) 611.10 [M + H]+ |
| 72 | 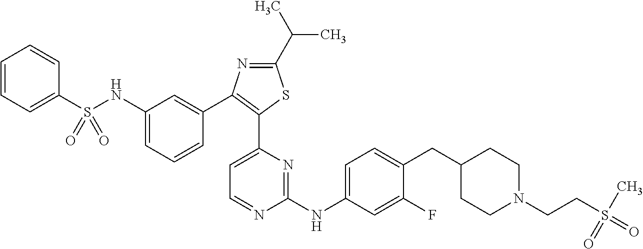 | MS (ESI) 749.11 [M + H]+ |
| 73 | 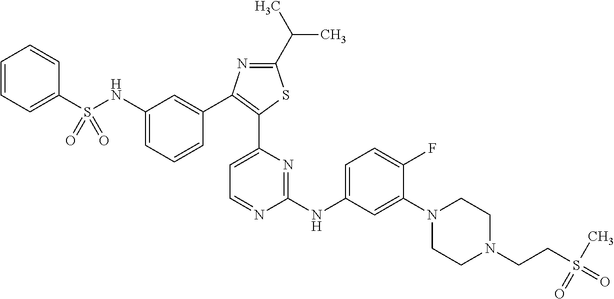 | MS (ESI) 736.14 [M + H]+ |
| 74 | 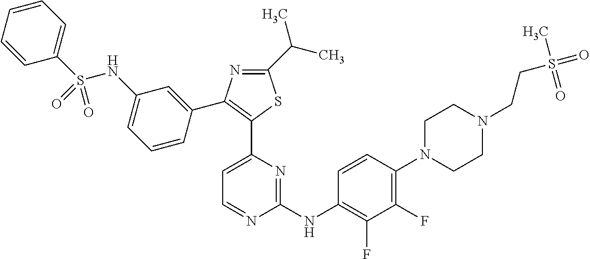 | MS (ESI) 754.31 [M + H]+ |
| 75 | 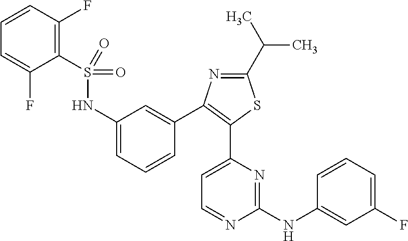 | MS (ESI) 652.01 [M + H]+ |
| 76 | 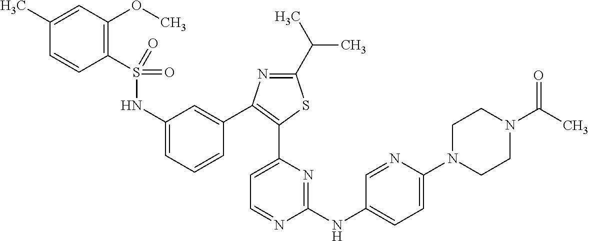 | MS (ESI) 699.32 [M + H]+ |
| 77 | 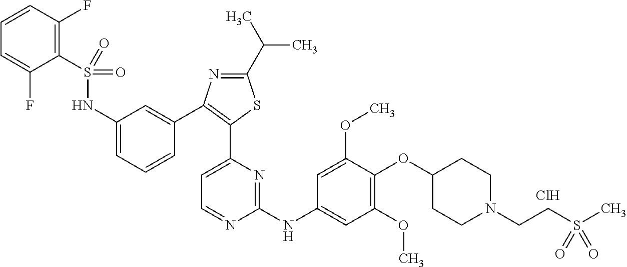 | MS (ESI) 629.48 [M + H]+ |
| 78 | 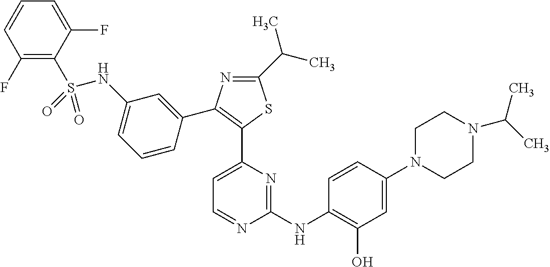 | MS (ESI) 706.43 [M + H]+ |
| 79 | 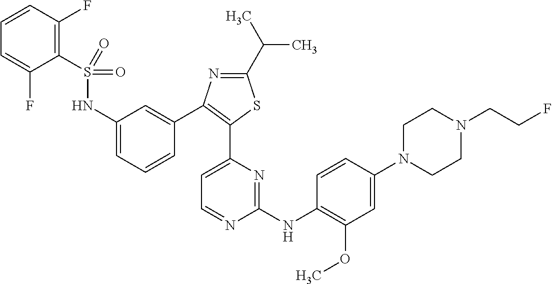 | MS (ESI) 724.35 [M + H]+ |
| 80 | 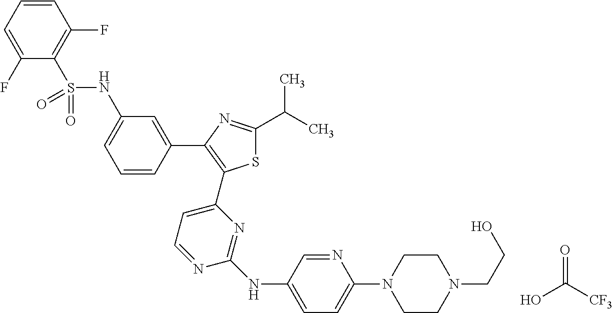 | MS (ESI) 693.31 [M + H]+ |
| 81 | 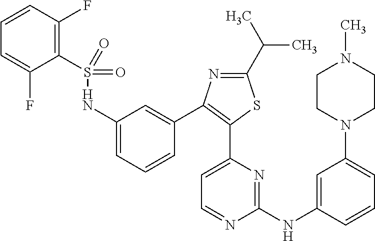 | MS (ESI) 662.26 [M + H]+ |
| 82 | 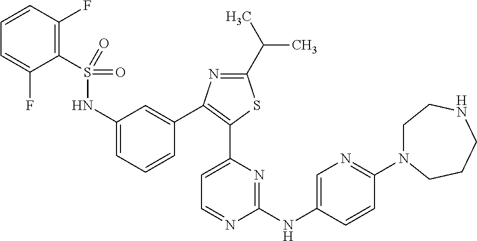 | MS (ESI) 663.32 [M + H]+ |
| 83 | 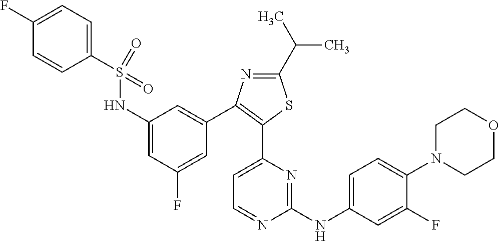 | MS (ESI) 667.26 [M + H]+ |
| 84 | 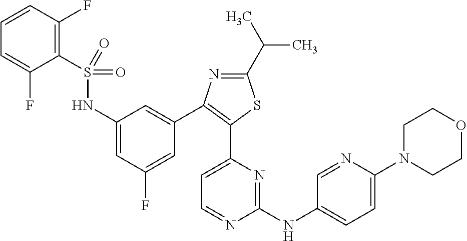 | MS (ESI) 668.26 [M + H]+ |
| 85 | 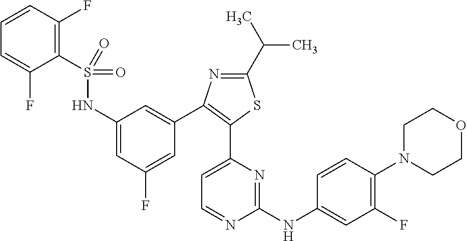 | MS (ESI) 685.21 [M + H]+ |
| 86 | 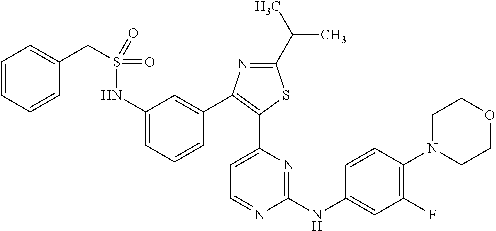 | MS (ESI) 644.26 [M + H]+ |
| 87 | 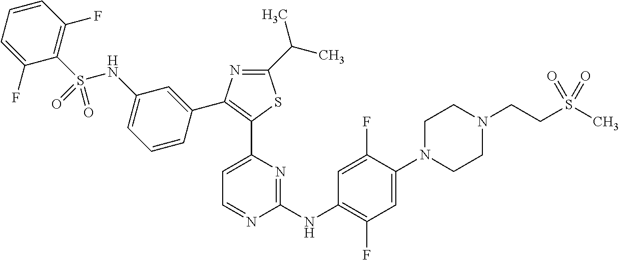 | MS (ESI) 790.28 [M + H]+ |
| 88 | 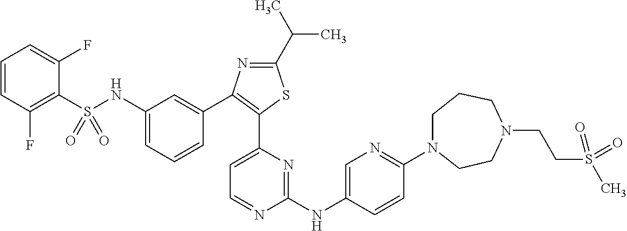 | MS (ESI) 769.31 [M + H]+ |
| 89 | 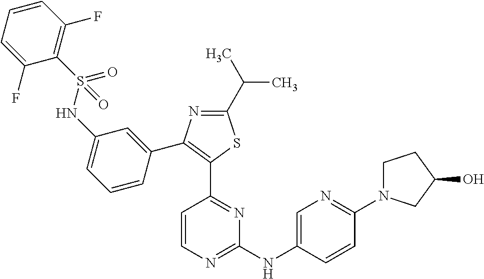 | MS (ESI) 650.16 [M + H]+ |
| 90 | 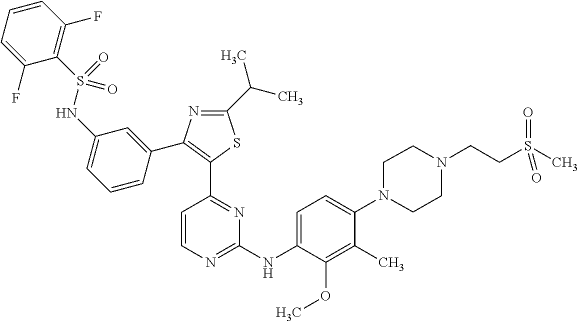 | MS (ESI) 797.67 [M + H]+ |
| 91 | 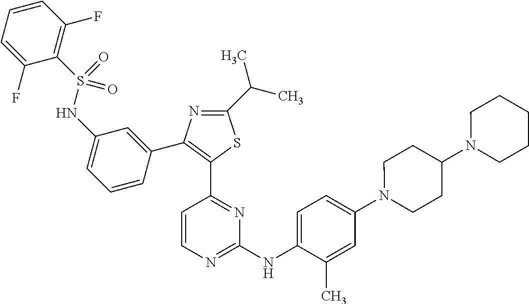 | MS (ESI) 744.25 [M + H]+ |
| 92 | 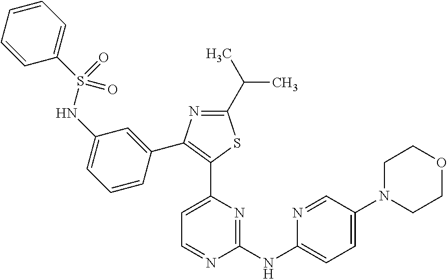 | MS (ESI) 614.25 [M + H]+ |
| 93 | 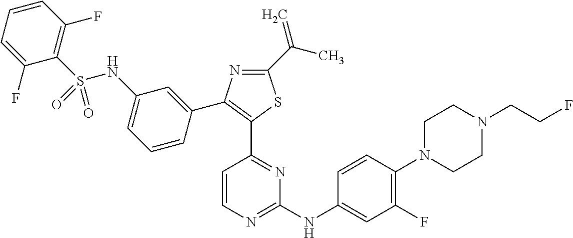 | MS (ESI) 710.21 [M + H]+ |
| 94 | 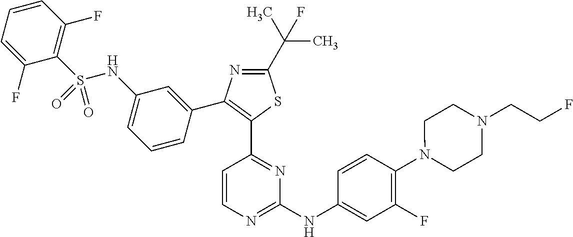 | MS (ESI) 730.20 [M + H]+ |
| 95 | 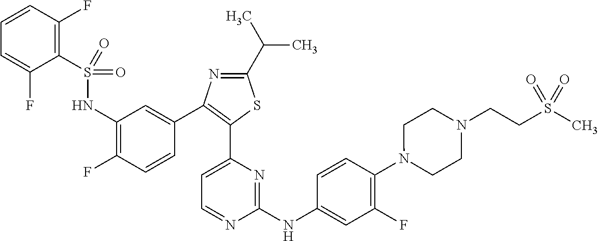 | MS (ESI) 790.18 [M + H]+ |
| 96 | 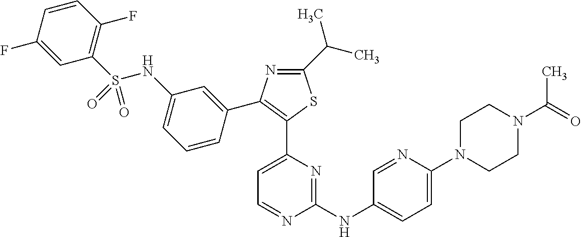 | MS (ESI) 691.15 [M + H]+ |
| 97 | 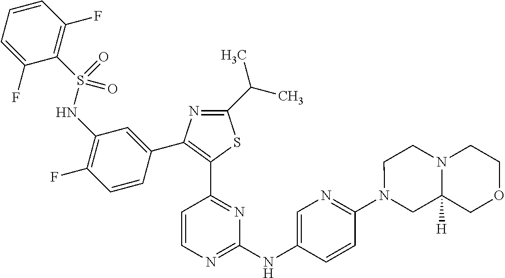 | MS (ESI) 723.20 [M + H]+ |
| 98 | 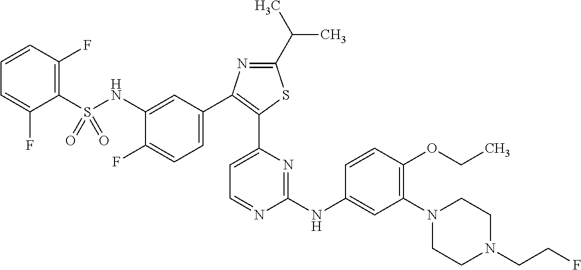 | MS (ESI) 756.20 [M + H]+ |
| 99 | 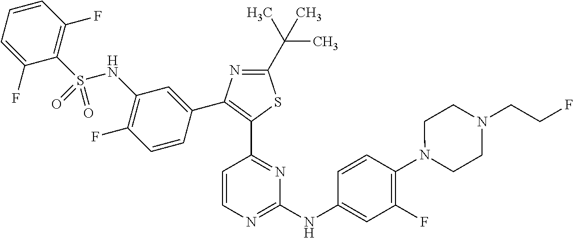 | MS (ESI) 744.56 [M + H]+ |
| 100 | 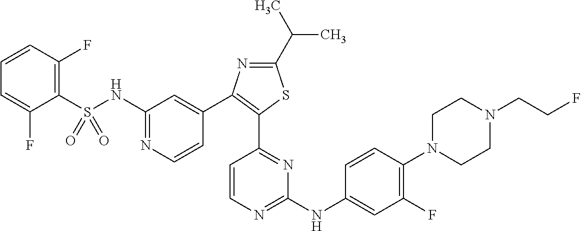 | MS (ESI) 713.18 [M + H]+ |
| 101 | 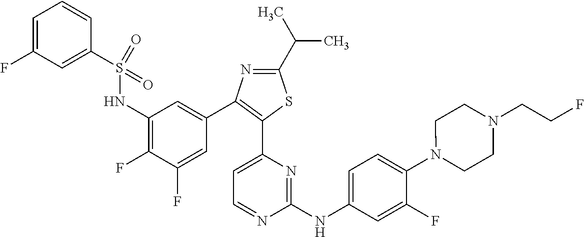 | MS (ESI) 730.50 [M + H]+ |
| 102 | 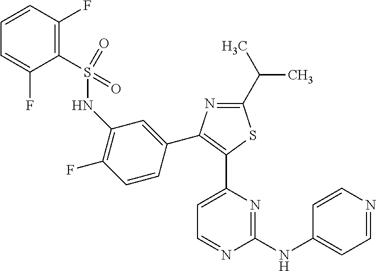 | MS (ESI) 583.05 [M + H]+ |
| 103 | 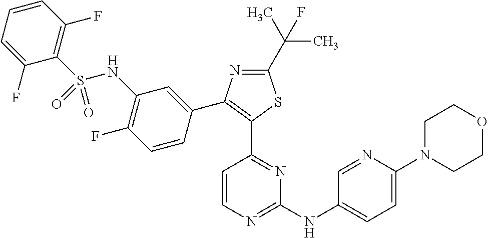 | MS (ESI) 686.48 [M + H]+ |
| 104 | 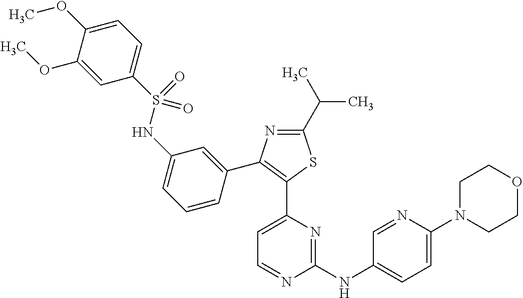 | MS (ESI) 674.50 [M + H]+ |
| 105 | 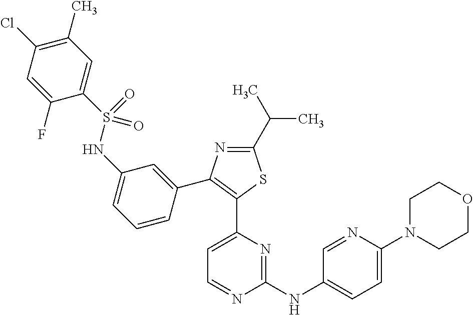 | MS (ESI) 681.40 [M + H]+ |
| 106 | 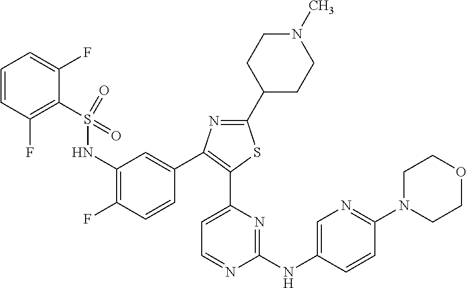 | MS (ESI) 723.59 [M + H]+ |
| 107 | 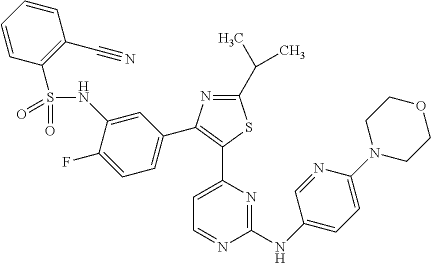 | MS (ESI) 657.15 [M + H]+ |
| 108 | 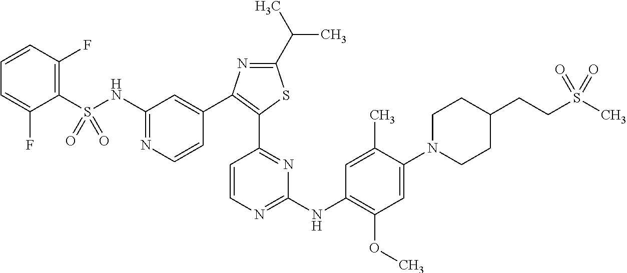 | MS (ESI) 798.31 [M + H]+ |
| 109 | 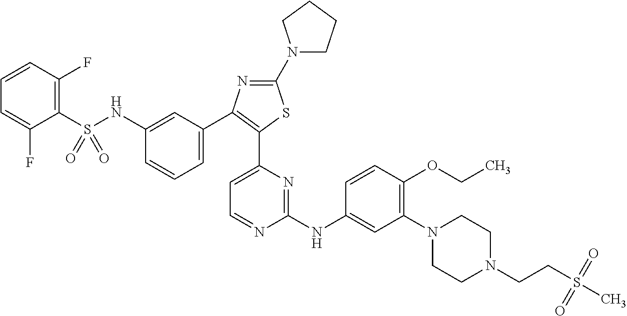 | MS (ESI) 825.35 [M + H]+ |
| 110 | 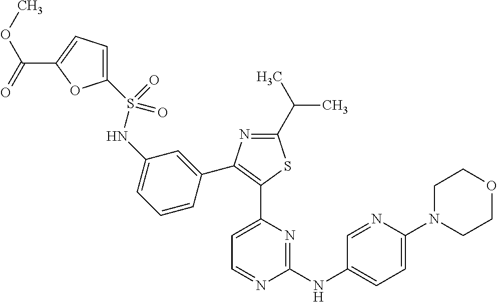 | MS (ESI) 662.5 [M + H]+ |
| 111 | 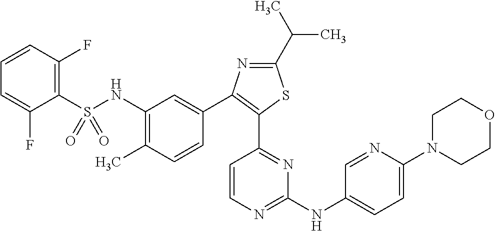 | MS (ESI) 664.18 [M + H]+ |
| 112 | 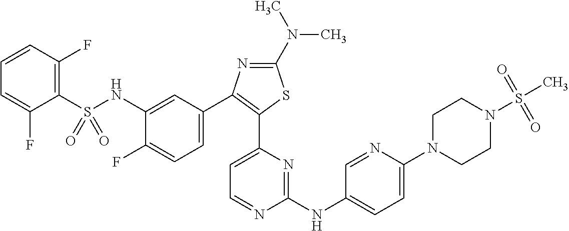 | MS (ESI) 746.13 [M + H]+ |
| 113 | 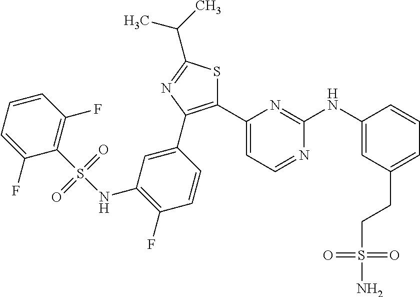 | MS (ESI) 689.07 [M + H]+ |
| 114 | 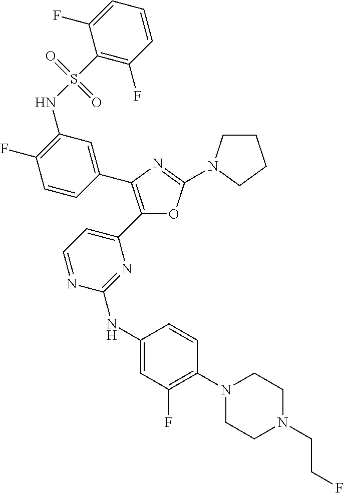 | MS (ESI) 741.27 [M + H]+ |
| 115 | 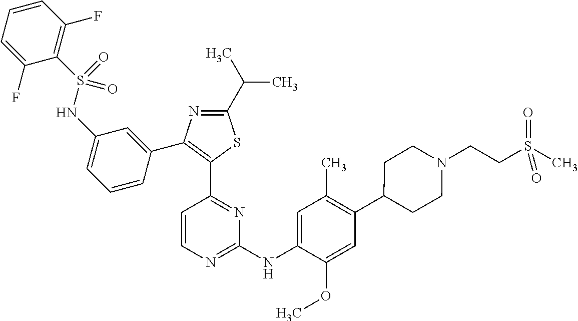 | MS (ESI) 796.48 [M]+ |
| 116 | 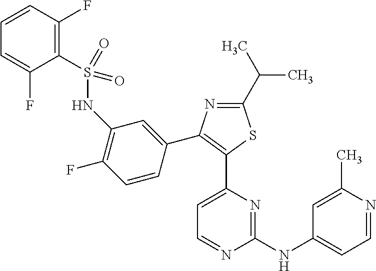 | MS (ESI) 597.12 [M + H]+ |
| 117 | 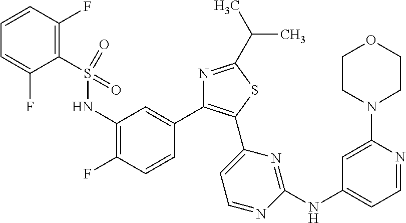 | MS (ESI) 668.20 [M + H]+ |
| 118 | 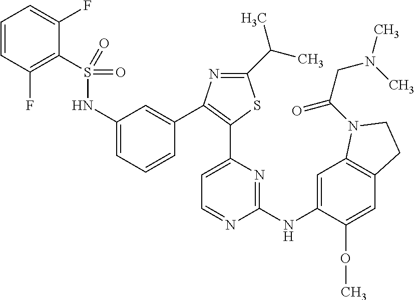 | MS (ESI) 718.32 [M − H]− |
| 119 | 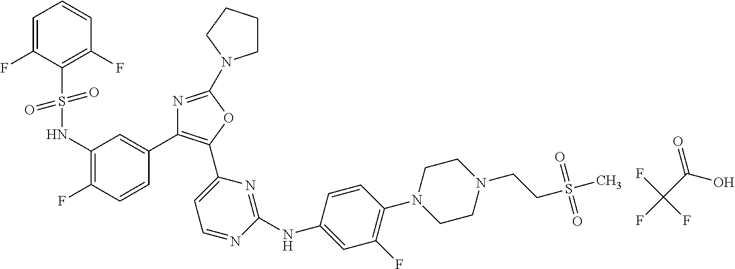 | MS (ESI) 801.31 [M + H]+ |
| 120 | 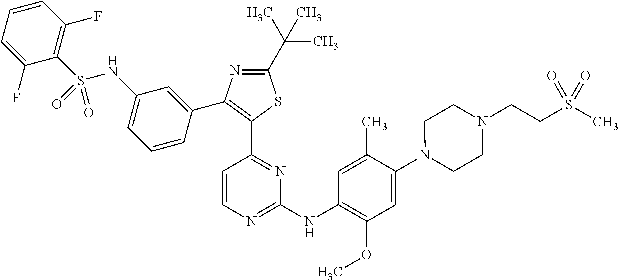 | MS (ESI) 812.32 [M + H]+ |
| 121 | 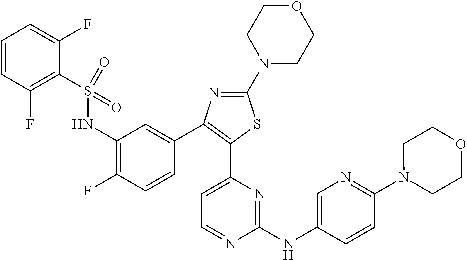 | MS (ESI) 711.16 [M + H]+ |
| 122 | 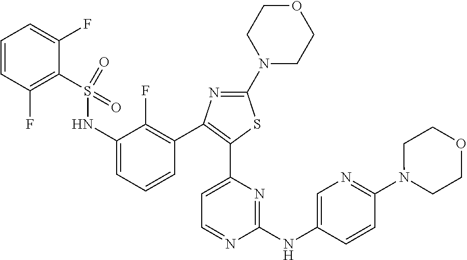 | MS (ESI) 711.13 [M + H]+ |
| 123 | 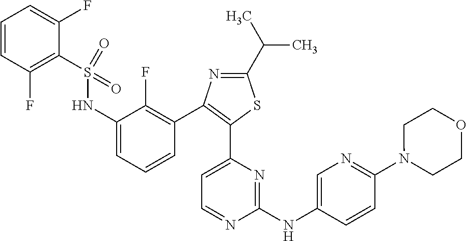 | MS (ESI) 668.08 [M + H]+ |
| 124 | 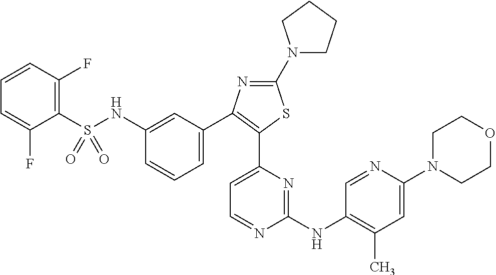 | MS (ESI) 691.14 [M + H]+ |
| 125 | 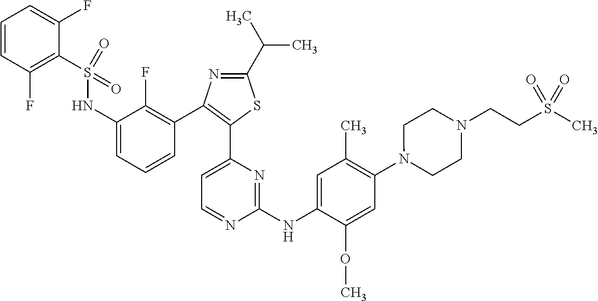 | MS (ESI) 816.24 [M + H]+ |
| 126 | 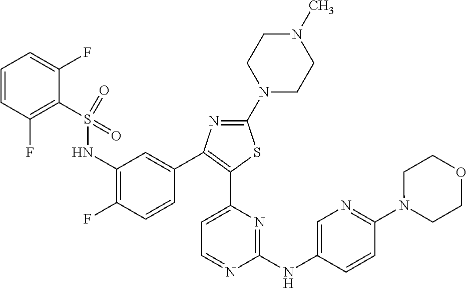 | MS (ESI) 724.20 [M + H]+ |
| 127 | 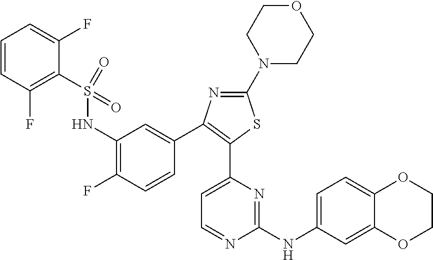 | MS (ESI) 683.22 [M + H]+ |
| 128 | 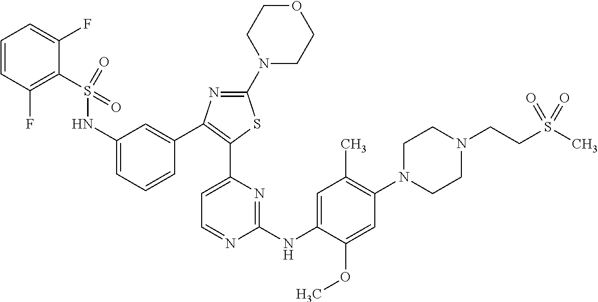 | MS (ESI) 841.23 [M + H]+ |
| 129 | 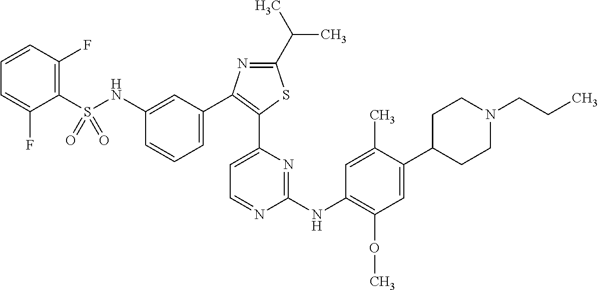 | MS (ESI) 830.28 [M + H]+ |
| 130 | 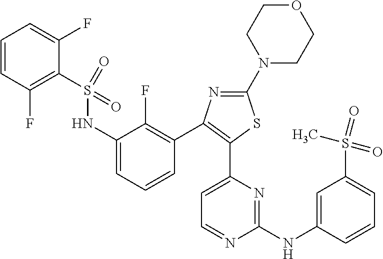 | MS (ESI) 703.18 [M + H]+ |
| 131 | 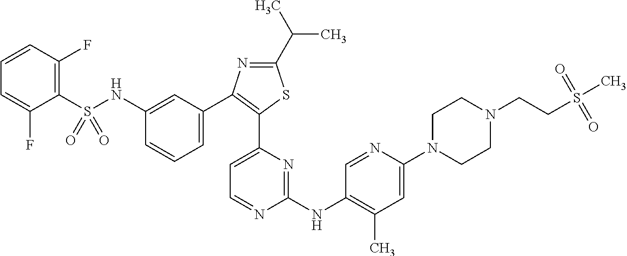 | MS (ESI) 769.32 [M + H]+ |
| 132 | 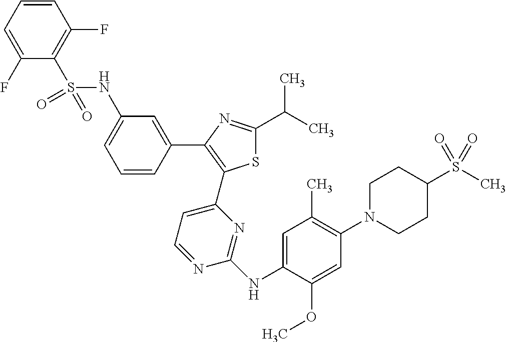 | MS (ESI) 810.44 [M + H]+ |
| 133 | 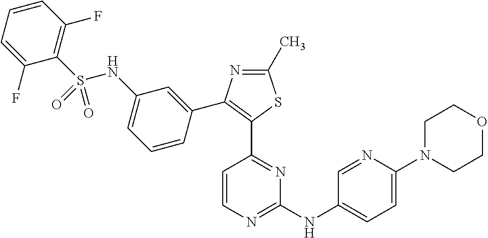 | MS (ESI) 622.11 [M + H]+ |
| 134 | 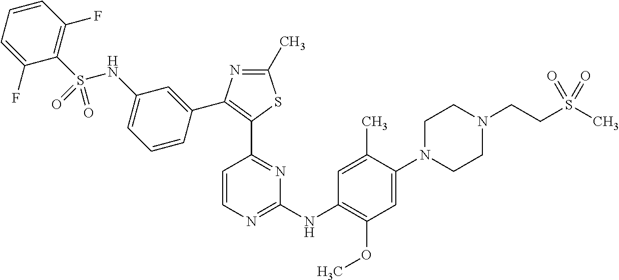 | MS (ESI) 770.27 [M + H]+ |
| 135 | 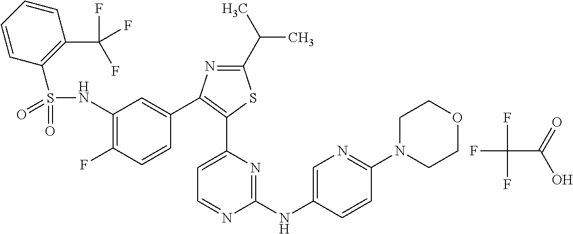 | MS (ESI) 700.21 [M + H]+ |
| 136 | 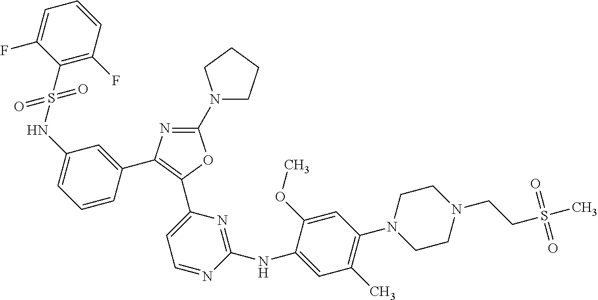 | MS (ESI) 809.35 [M + H]+ |
| 137 | 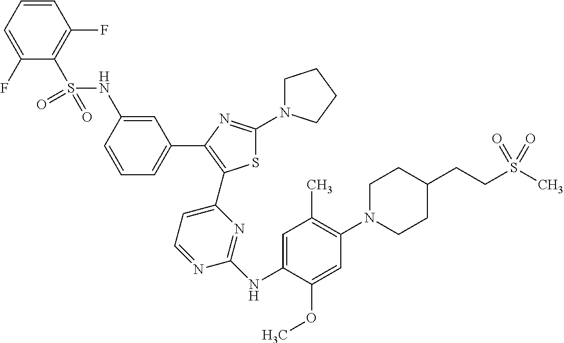 | MS (ESI) 824.37 [M + H]+ |
| 138 | 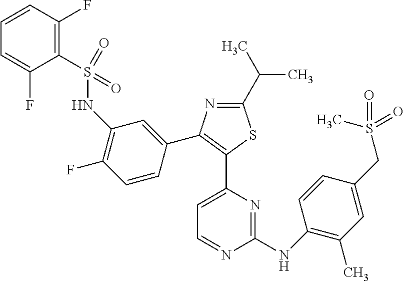 | MS (ESI) 688.24 [M + H]+ |
| 139 | 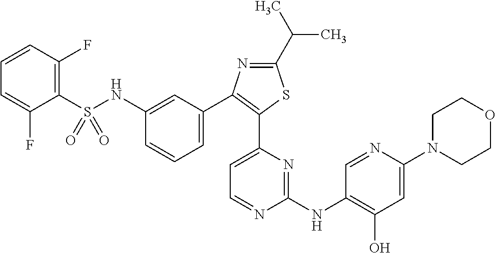 | MS (ESI) 666.31 [M + H]+ |
| 140 | 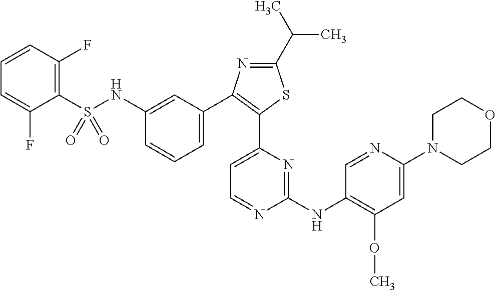 | MS (ESI) 672.79 [M + H]+ |
| 141 | 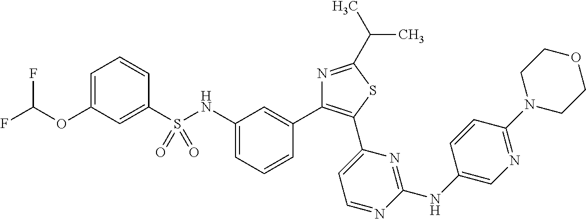 | MS (ESI) 681.21 [M + H]+ |
| 142 | 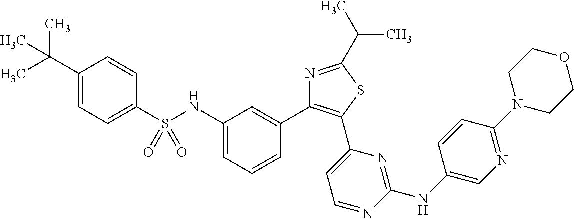 | MS (ESI) 670.27 [M + H]+ |
| 143 | 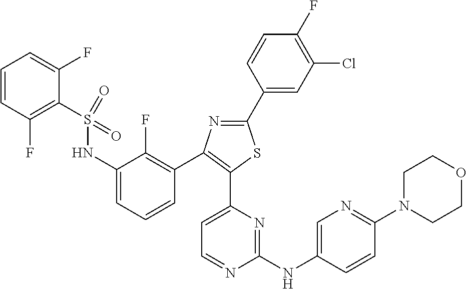 | MS (ESI) 754.29 [M]+ |
| 144 | 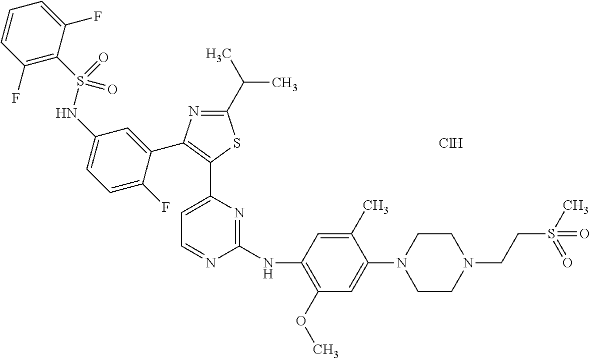 | MS (ESI) 816.28 [M + H]+ |
| 145 | 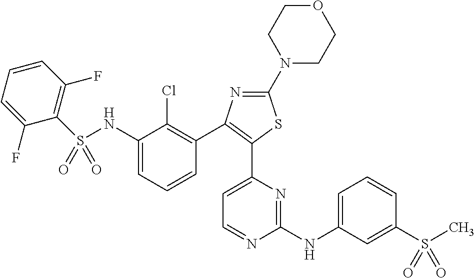 | MS (ESI) 719.15 [M + H]+ |
| 146 | 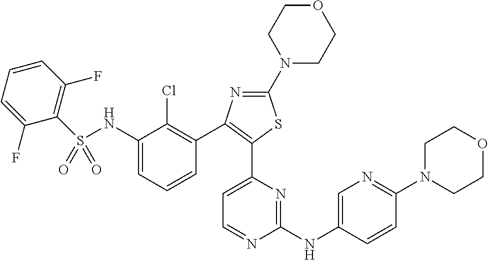 | MS (ESI) 727.26 [M + H]+ |
| 147 | 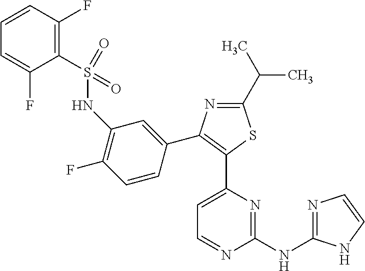 | MS (ESI) 572.18 [M + H]+ |
| 148 | 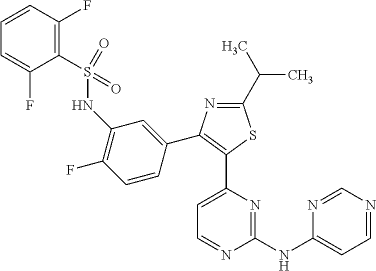 | MS (ESI) 584.17 [M + H]+ |
| 149 | 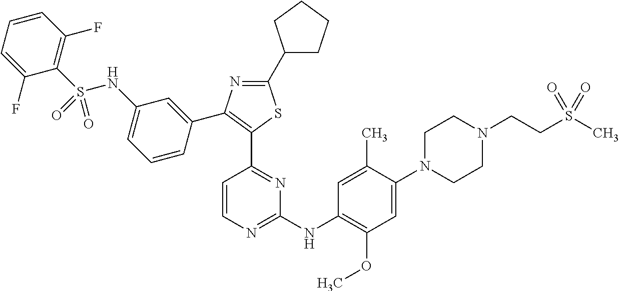 | MS (ESI) 824.41 [M]+ |
| 150 | 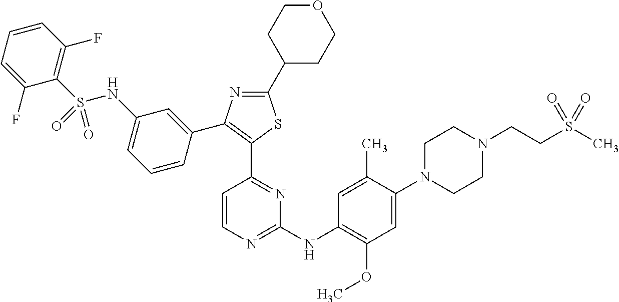 | MS (ESI) 840.39 [M]+ |
| 151 | 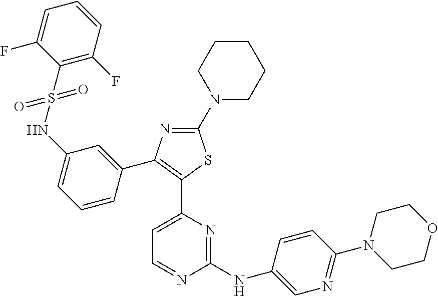 | MS (ESI) 691.21 [M]+ |
| 152 | 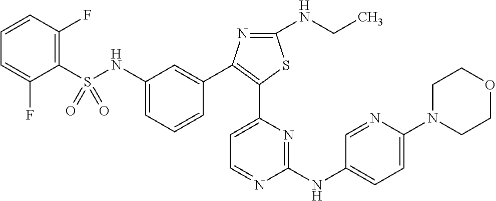 | MS (ESI) 650.90 [M + H]+ |
| 153 | 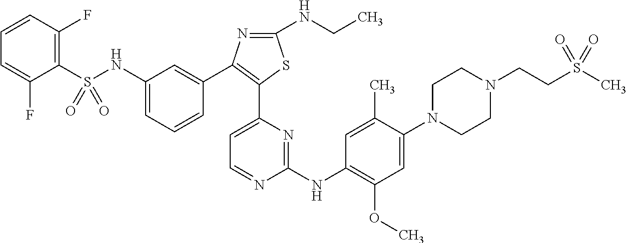 | MS (ESI) 799.00 [M + H]+ |
| 154 | 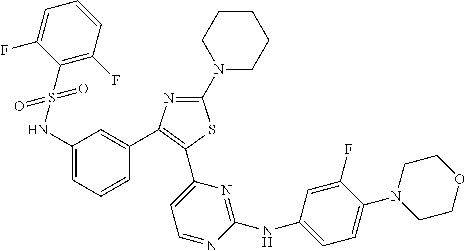 | MS (ESI) 708.27 [M + H]+ |
| 155 | 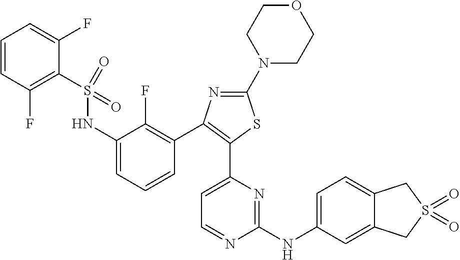 | MS (ESI) 715.16 [M + H]+ |
| 156 | 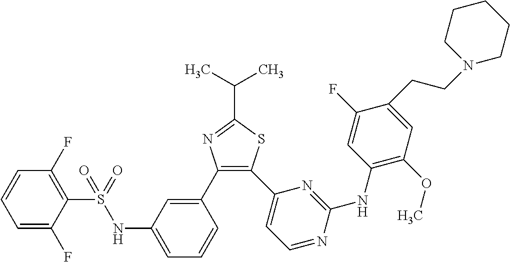 | MS (ESI) 723.31 [M + H]+ |
| 157 | 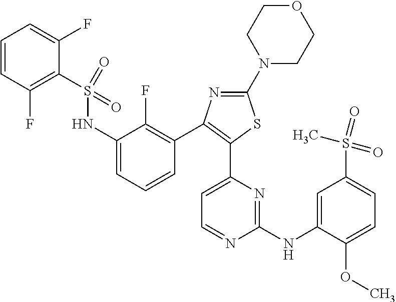 | MS (ESI) 733.17 [M + H]+ |
| 158 | 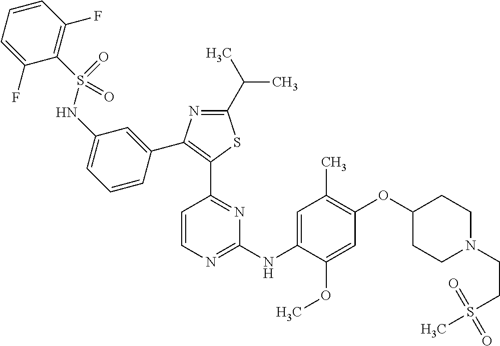 | MS (ESI) 813.39 [M + H]+ |
| 159 | 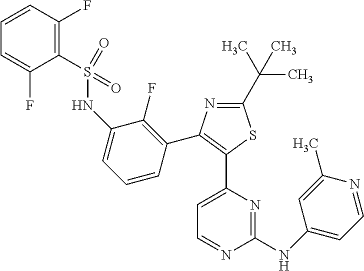 | MS (ESI) 611.28 [M + H]+ |
| 160 | 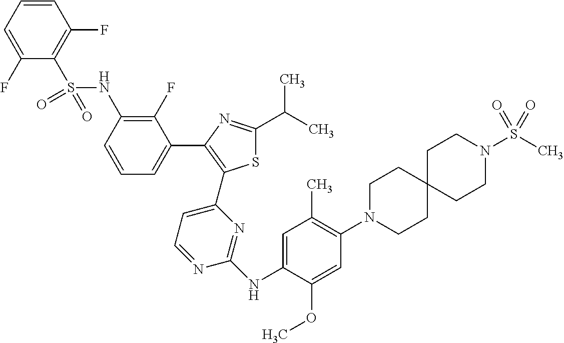 | MS (ESI) 856.41 [M + H]+ |
| 161 | 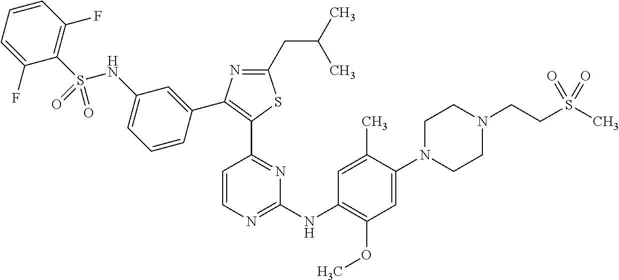 | MS (ESI) 812.32 [M + H]+ |
| 162 | 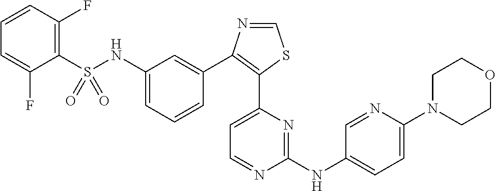 | MS (ESI) 608.07 [M + H]+ |
| 163 | 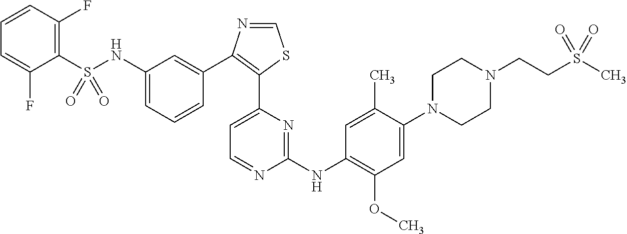 | MS (ESI) 756.32 [M + H]+ |
| 164 | 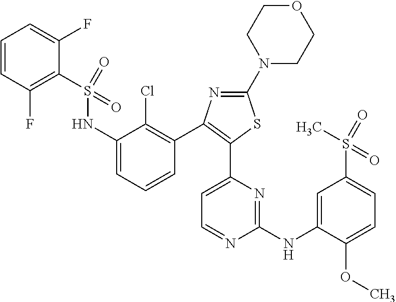 | MS (ESI) 749.19 [M + H]+ |
| 165 | 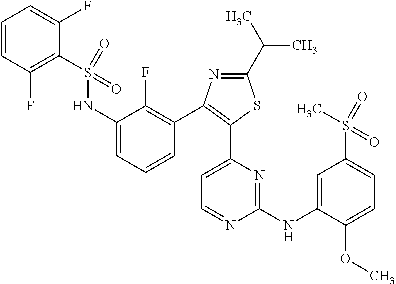 | MS (ESI) 690.18 [M + H]+ |
| 166 | 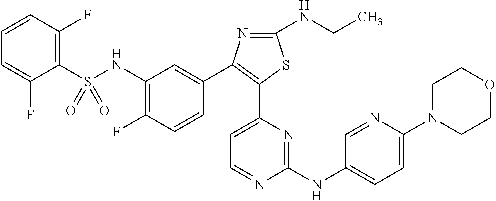 | MS (ESI) 669.11 [M + H]+ |
| 167 | 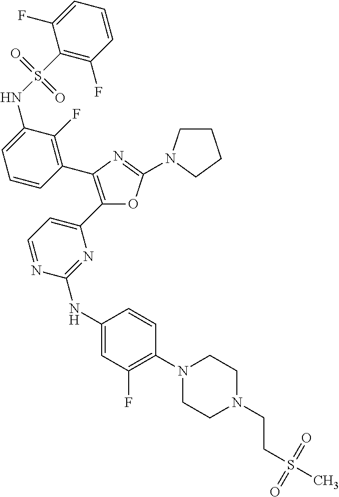 | MS (ESI) 801.19 [M + H]+ |
| 168 | 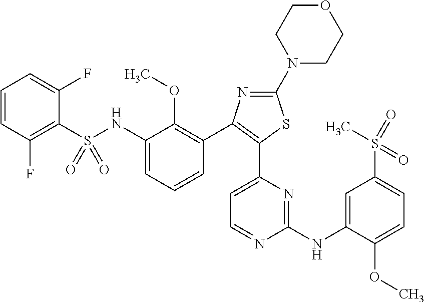 | MS (ESI) 745.47 [M + H]+ |
| 169 | 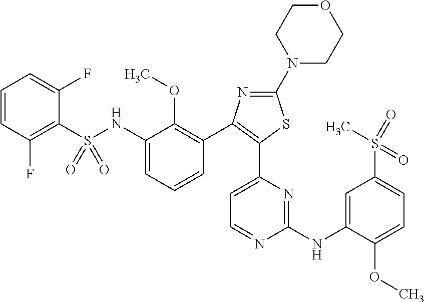 | MS (ESI) 715.59 [M + H]+ |
| 170 | 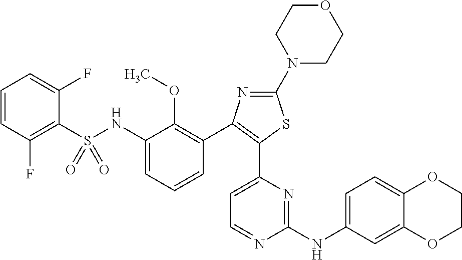 | MS (ESI) 695.27 [M + H]+ |
| 171 | 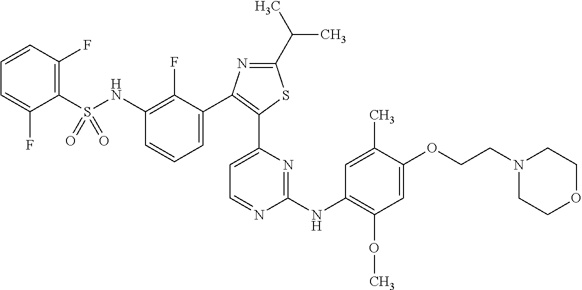 | MS (ESI) 755.15 [M + H]+ |
| 172 | 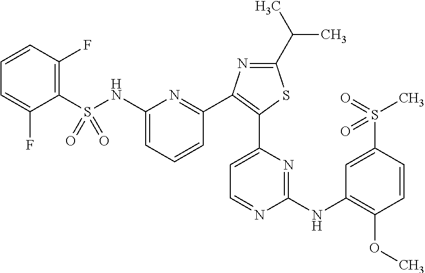 | MS (ESI) 673.11 [M + H]+ |
| 173 | 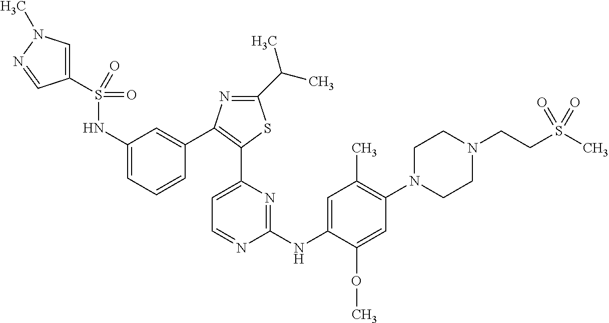 | MS (ESI) 766.25 [M + H]+ |
| 174 | 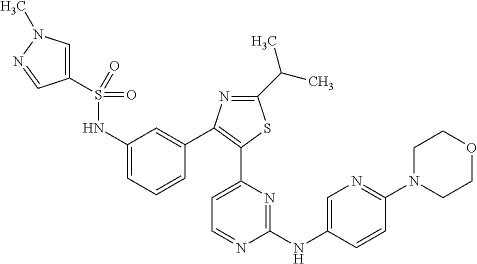 | MS (ESI) 618.17 [M + H]+ |
| 175 | 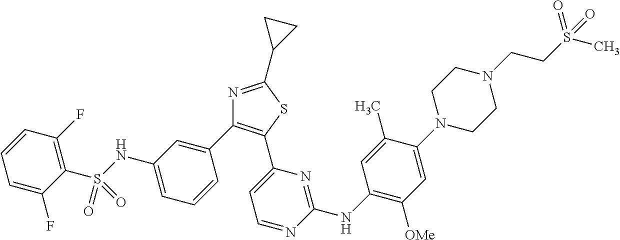 | MS (ESI) 796.38 [M + H]+ |
| 176 | 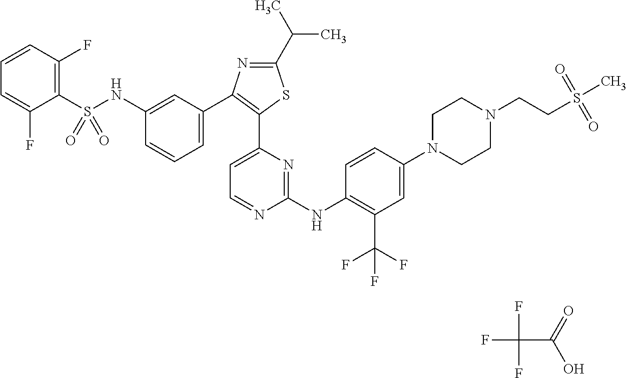 | MS (ESI) 822.49 [M + H]+ |
| 177 | 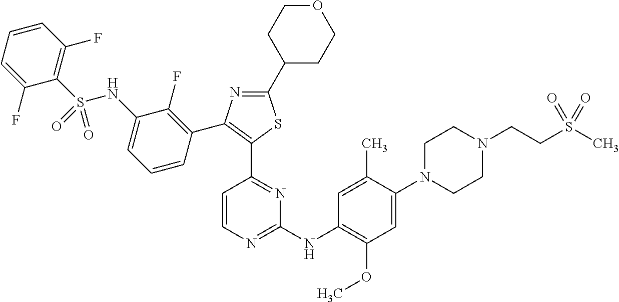 | MS (ESI) 858.58 [M + H]+ |

### Biological Examples

Compounds of the present invention were tested for ErbB family and B-Raf protein kinase inhibitory activity in substrate phosphorylation assays and cell proliferation assays.

### A. Enzyme Assays—EGFR and ErbB-2 Assays:

Compounds of the present invention were tested for EGFR and ErbB-2 protein tyrosine kinase inhibitory activity in substrate phosphorylation assays using enzymes purified from a baculovirus expression system. Reagent production was conducted essentially as described in Brignola, P. S., et al, (2002) J. Biol. Chem. 277(2):1576-1585.

The method measures the ability of the isolated enzyme to catalyse the transfer of the gamma-phosphate from ATP onto the tyrosine residue of a biotinylated synthetic peptide referenced “Peptide C” in Brignola, P. S., et al, (2002) J. Biol. Chem. 277(2):1576-1585. The extent of tyrosine phosphorylation was measured using an anti-phosphotyrosine antibody, and quantified by homogenous time-resolved fluorescence (HTRF).

Reactions were performed in black 384-well polystyrene flat-bottom plates in a final volume of 20 μL. Assays were performed by adding 10 μL of each of the following solutions, Substrate Mix and Enzyme Mix: The Substrate Mix contained 100 mM 3-[N-morpholino]propanesulfonic acid (MOPS) (pH 7.5), 2 mM MnCl₂, 20 μM ATP, 0.01% Tween-20, 0.1 mg/mL (BSA), 0.8 uM peptide substrate, and 1 mM dithiothreitol. The Enzyme Mix contained 100 mM MOPS (pH7.5); 0.01% Tween-20; 0.1 mg/mL BSA, and either 0.8 nM EGFR, 10 nM ErbB2, or 1 nM ErbB4. The Enzyme Mix was added to the compound plates and the plates were incubated at 20° C. for 1 hr. The reactions were then started by adding the Substrate Mix.

After initiating the reaction with the method described above, the reactions were allowed to proceed for 90 minutes at 20° C. The reactions were then terminated by the addition of 20 μL 100 mM EDTA to each well. 40 μL/well of HTRF detection mix were added to the assay plates. The final concentrations of the detection reagents were: 100 mM HEPES (pH7.5), 0.1 mg/mL BSA, 15 nM streptavidin-labeled allophycocyanin (PerkinElmer), and 1 nM europium-labeled anti-phosphotyrosine antibody (Perkin Elmer). Assay plates were left unsealed and were counted in a Wallac Multilabel Counter 1420 (PerkinElmer).

Compounds under analysis were dissolved in dimethylsulfoxide (DMSO) to 1.0 mM and serially diluted 1 to 3 with DMSO through twelve dilutions. 1 μL of each concentration was transferred to the corresponding well of an assay plate. This creates a final compound concentration range from 0.00027 to 47.6 μM.

The data for dose responses were plotted as % Inhibition calculated with the data reduction formula 100*(1-(U1−C2)/(C1−C2)) versus concentration of compound where U1 is the unknown value, C1 is the average control value obtained for 4.76% DMSO, and C2 is the average control value obtained for 0.035 M EDTA. Data were fitted with a curve described by:

y=((Vmax*x)/(K+x))+Y2

where Vmax is the upper asymptote, Y2 is the Y intercept, and K is the IC50. The results for each compound were recorded as pIC50s, calculated as follows:

pIC50=−Log 10(K)

Many of the exemplified compounds Examples 1-177 were run in the recited assay and the results are reported in the following Table 1. In the following table:
“+” indicates no pIC50 measurement greater than 6 against EGFR,
“++” indicates at least one pIC50 measurement greater than 6 against EGFR but no measurement greater than pIC50 of 7; and
“+++” indicates at least one pIC50 measurement of greater than 7 against EGFR.

TABLE 1
EGFR Activity

| Example | Activity |
|---|---|
| 1 | +++ |
| 2 | + |
| 3 | + |
| 4 | + |
| 5 | ++ |
| 6 | + |
| 8 | ++ |
| 9 | +++ |
| 10 | + |
| 11 | ++ |
| 12 | +++ |
| 13 | ++ |
| 14 | ++ |
| 15 | ++ |
| 16 | ++ |
| 17 | ++ |
| 18 | +++ |
| 19 | +++ |
| 20 | +++ |
| 21 | +++ |
| 22 | ++ |
| 23 | ++ |
| 24 | + |
| 25 | ++ |
| 26 | ++ |
| 27 | ++ |
| 28 | + |
| 29 | + |
| 30 | + |
| 31 | + |
| 32 | + |
| 33 | + |
| 34 | + |
| 35 | ++ |
| 36 | +++ |
| 37 | +++ |
| 38 | + |
| 39 | ++ |
| 40 | + |
| 41 | + |
| 42 | + |
| 43 | + |
| 44 | + |
| 45 | +++ |
| 46 | + |
| 47 | + |
| 48 | + |
| 49 | + |
| 50 | +++ |
| 51 | + |
| 52 | ++ |
| 53 | + |
| 54 | ++ |
| 55 | +++ |
| 56 | + |
| 57 | ++ |
| 58 | + |
| 59 | + |
| 60 | + |
| 61 | + |
| 62 | ++ |
| 63 | ++ |
| 64 | ++ |
| 65 | +++ |
| 66 | + |
| 67 | + |
| 68 | ++ |
| 69 | ++ |
| 70 | + |
| 71 | ++ |
| 72 | ++ |
| 73 | ++ |
| 74 | + |
| 75 | + |
| 76 | + |
| 77 | +++ |
| 78 | ++ |
| 79 | ++ |
| 80 | +++ |
| 81 | +++ |
| 82 | +++ |
| 83 | + |
| 84 | ++ |
| 85 | + |
| 86 | + |
| 87 | + |
| 88 | ++ |
| 89 | ++ |
| 90 | + |
| 91 | +++ |
| 92 | + |
| 93 | ++ |
| 94 | + |
| 95 | ++ |
| 96 | ++ |
| 97 | ++ |
| 98 | +++ |
| 99 | + |
| 100 | + |
| 101 | + |
| 102 | + |
| 103 | + |
| 104 | + |
| 105 | + |
| 106 | ++ |
| 107 | ++ |
| 108 | + |
| 109 | +++ |
| 110 | + |
| 111 | ++ |
| 112 | ++ |
| 113 | ++ |
| 114 | + |
| 115 | + |
| 116 | ++ |
| 117 | ++ |
| 118 | ++ |
| 119 | + |
| 120 | + |
| 121 | ++ |
| 122 | ++ |
| 123 | + |
| 124 | + |
| 125 | +++ |
| 126 | ++ |
| 127 | ++ |
| 128 | +++ |
| 129 | + |
| 130 | + |
| 131 | + |
| 132 | + |
| 133 | + |
| 134 | +++ |
| 135 | + |
| 136 | ++ |
| 138 | + |
| 139 | + |
| 140 | + |
| 141 | + |
| 142 | + |
| 143 | + |
| 144 | ++ |
| 145 | + |
| 146 | + |
| 147 | + |
| 148 | + |
| 149 | + |
| 150 | +++ |
| 151 | + |
| 152 | ++ |
| 153 | +++ |
| 154 | + |
| 155 | ++ |
| 156 | ++ |
| 157 | + |
| 158 | +++ |
| 159 | + |
| 160 | ++ |
| 161 | +++ |
| 162 | ++ |
| 163 | +++ |
| 164 | + |
| 165 | ++ |
| 166 | ++ |
| 167 | ++ |
| 168 | + |
| 169 | + |
| 170 | + |
| 171 | ++ |
| 172 | ++ |
| 173 | + |
| 174 | + |
| 175 | ++ |
| 176 | + |
| 177 | +++ |

Many of the exemplified compounds Examples 1-177 were run in the recited assay and the results are reported in the following Table 2. In the following table:
“+” indicates no pIC50 measurement greater than 6 against ErbB2
“++” indicates at least one pIC50 measurement greater than 6 against ErbB2 but no measurement greater than pIC50 of 7; and
“+++” indicates at least one pIC50 measurement of greater than 7 against ErbB2.

TABLE 2
ErbB2 Activity

| Example | Activity |
|---|---|
| 1 | ++ |
| 2 | + |
| 3 | + |
| 4 | + |
| 5 | ++ |
| 6 | ++ |
| 8 | + |
| 9 | +++ |
| 10 | + |
| 11 | ++ |
| 12 | ++ |
| 13 | + |
| 14 | + |
| 15 | ++ |
| 16 | ++ |
| 17 | + |
| 18 | ++ |
| 19 | +++ |
| 20 | +++ |
| 21 | +++ |
| 22 | +++ |
| 23 | ++ |
| 24 | ++ |
| 25 | +++ |
| 26 | ++ |
| 27 | ++ |
| 28 | ++ |
| 29 | ++ |
| 30 | + |
| 31 | + |
| 32 | + |
| 33 | + |
| 34 | ++ |
| 35 | ++ |
| 36 | + |
| 37 | +++ |
| 38 | ++ |
| 39 | + |
| 40 | + |
| 41 | + |
| 42 | + |
| 43 | ++ |
| 44 | + |
| 45 | +++ |
| 46 | + |
| 48 | +++ |
| 49 | + |
| 50 | +++ |
| 51 | ++ |
| 52 | ++ |
| 53 | + |
| 54 | +++ |
| 55 | +++ |
| 56 | + |
| 57 | + |
| 58 | + |
| 59 | + |
| 60 | + |
| 61 | ++ |
| 62 | ++ |
| 63 | +++ |
| 64 | +++ |
| 65 | +++ |
| 66 | + |
| 67 | ++ |
| 68 | ++ |
| 69 | ++ |
| 70 | + |
| 71 | +++ |
| 72 | + |
| 73 | ++ |
| 74 | + |
| 75 | + |
| 76 | + |
| 77 | +++ |
| 78 | ++ |
| 79 | ++ |
| 80 | + |
| 81 | ++ |
| 82 | ++ |
| 83 | + |
| 84 | + |
| 85 | ++ |
| 86 | + |
| 87 | ++ |
| 88 | ++ |
| 89 | ++ |
| 90 | + |
| 91 | +++ |
| 92 | + |
| 93 | ++ |
| 94 | + |
| 95 | ++ |
| 96 | + |
| 97 | ++ |
| 98 | +++ |
| 99 | +++ |
| 100 | + |
| 101 | ++ |
| 102 | + |
| 103 | + |
| 104 | + |
| 105 | ++ |
| 106 | ++ |
| 107 | ++ |
| 108 | + |
| 109 | +++ |
| 110 | + |
| 111 | ++ |
| 112 | ++ |
| 113 | +++ |
| 114 | ++ |
| 115 | + |
| 116 | ++ |
| 117 | +++ |
| 118 | ++ |
| 119 | ++ |
| 120 | +++ |
| 121 | ++ |
| 122 | ++ |
| 123 | + |
| 124 | ++ |
| 125 | +++ |
| 126 | ++ |
| 127 | ++ |
| 128 | +++ |
| 129 | +++ |
| 130 | ++ |
| 131 | ++ |
| 132 | +++ |
| 133 | + |
| 134 | ++ |
| 135 | ++ |
| 136 | ++ |
| 137 | + |
| 138 | + |
| 139 | + |
| 140 | ++ |
| 141 | ++ |
| 142 | ++ |
| 143 | + |
| 144 | +++ |
| 145 | + |
| 146 | + |
| 147 | + |
| 148 | + |
| 149 | ++ |
| 150 | +++ |
| 151 | + |
| 152 | ++ |
| 153 | +++ |
| 154 | + |
| 155 | ++ |
| 156 | ++ |
| 157 | + |
| 158 | +++ |
| 159 | ++ |
| 160 | ++ |
| 161 | +++ |
| 162 | + |
| 163 | +++ |
| 164 | + |
| 165 | ++ |
| 166 | +++ |
| 167 | +++ |
| 168 | + |
| 169 | + |
| 170 | + |
| 171 | ++ |
| 172 | +++ |
| 173 | ++ |
| 174 | + |
| 175 | +++ |
| 176 | + |
| 177 | +++ |

### B. Enzyme Assays—B-Raf Assays:

Compounds of the present invention were tested for B-Raf protein serine kinase inhibitory activity in a B-Raf Accelerated MEK ATPase assay (BRAMA). The BRAMA assay is a high sensitivity assay which measures an intrinsic MEK-mediated ATP hydrolysis uncoupled from downstream ERK phosphorylation by coupling the formation of ADP to NADH oxidation through the enzymes pyruvate kinase and lactate dehydrogenase. When ADP production is initiated by addition of catalytic amounts of an activated Raf enzyme and non-phosphorylated MEK, one observes robust ADP production concomitant with Raf-mediated phosphorylation of MEK.

Method A: Specific details of the assay are disclosed in: C. Rominger, M. Schaber, E. May. Assay for B-Raf Activity Based on Intrinsic MEK ATPase Activity. Statutory Invention Registration 11/084,993 (March, 2005).
Method B: The method is the same as method A, but includes the following changes: 1) the assay was performed with a final MEK concentration of 150 nM and 2) the assay was read as single end point instead of a kinetic read.

Many of the exemplified compounds Examples 1-177 were run in the recited assay and the results are reported in the following Table 3. In the following table:
“+” indicates no pIC50 measurement greater than 6 against B-Raf
“++” indicates at least one pIC50 measurement greater than 6 against B-Raf but no measurement greater than pIC50 of 7; and
“+++” indicates at least one pIC50 measurement of greater than 7 against B-Raf.

TABLE 3
B-Raf Activity

| | Activity |
|---|---|
| Example | (Method) |
| 1 | ++ (A) |
| 3 | +++ (B) |
| 4 | ++ (A) |
| 5 | +++ (A) |
| 6 | +++ (A) |
| 8 | +++ (B) |
| 9 | +++ (B) |
| 10 | +++ (B) |
| 11 | +++ (B) |
| 12 | +++ (B) |
| 13 | +++ (B) |
| 14 | +++ (B) |
| 15 | +++ (B) |
| 16 | +++ (B) |
| 17 | +++ (B) |
| 18 | +++ (B) |
| 19 | +++ (B) |
| 20 | +++ (B) |
| 21 | +++ (B) |
| 22 | +++ (B) |
| 23 | +++ (B) |
| 24 | +++ (B) |
| 25 | +++ (B) |
| 26 | +++ (B) |
| 27 | +++ (B) |
| 28 | +++ (B) |
| 29 | +++ (B) |
| 30 | +++ (B) |
| 31 | +++ (B) |
| 32 | +++ (B) |
| 33 | +++ (B) |
| 34 | +++ (B) |
| 35 | ++ (B) |
| 36 | +++ (B) |
| 37 | ++ (B) |
| 38 | ++ (B) |
| 39 | +++ (B) |
| 40 | ++ (B) |
| 41 | ++ (B) |
| 42 | +++ (B) |
| 43 | ++ (B) |
| 44 | +++ (B) |
| 45 | ++ (B) |
| 46 | ++ (B) |
| 47 | +++ (B) |
| 48 | ++ (B) |
| 49 | +++ (B) |
| 50 | +++ (B) |
| 51 | +++ (B) |
| 52 | +++ (B) |
| 53 | +++ (B) |
| 54 | +++ (B) |
| 55 | +++ (B) |
| 56 | +++ (B) |
| 57 | +++ (B) |
| 58 | ++ (B) |
| 59 | +++ (B) |
| 60 | +++ (B) |
| 61 | +++ (B) |
| 62 | +++ (B) |
| 63 | ++ (B) |
| 64 | +++ (B) |
| 65 | +++ (A) |
| 66 | +++ (B) |
| 67 | ++ (B) |
| 68 | ++ (B) |
| 69 | ++ (B) |
| 70 | ++ (B) |
| 71 | +++ (B) |
| 72 | +++ (B) |
| 73 | +++ (B) |
| 74 | +++ (B) |
| 75 | +++ (B) |
| 76 | ++ (B) |
| 77 | +++ (B) |
| 78 | +++ (B) |
| 79 | +++ (B) |
| 80 | +++ (B) |
| 81 | +++ (B) |
| 82 | +++ (B) |
| 83 | +++ (B) |
| 84 | +++ (B) |
| 85 | +++ (B) |
| 86 | ++ (B) |
| 87 | +++ (B) |
| 88 | +++ (B) |
| 89 | +++ (B) |
| 90 | +++ (B) |
| 91 | +++ (B) |
| 92 | +++ (B) |
| 93 | +++ (B) |
| 94 | ++ (B) |
| 95 | +++ (B) |
| 96 | +++ (B) |
| 97 | +++ (B) |
| 98 | +++ (B) |
| 99 | +++ (B) |
| 100 | +++ (B) |
| 101 | +++ (B) |
| 102 | +++ (B) |
| 103 | ++ (B) |
| 104 | ++ (B) |
| 105 | ++ (B) |
| 106 | +++ (B) |
| 107 | +++ (B) |
| 108 | +++ (B) |
| 109 | +++ (B) |
| 110 | +++ (B) |
| 111 | ++ (B) |
| 112 | +++ (B) |
| 113 | +++ (B) |
| 114 | ++ (B) |
| 115 | +++ (B) |
| 116 | +++ (B) |
| 117 | +++ (B) |
| 118 | ++ (B) |
| 119 | +++ (B) |
| 120 | +++ (B) |
| 121 | +++ (B) |
| 122 | +++ (B) |
| 123 | +++ (B) |
| 124 | +++ (B) |
| 125 | +++ (B) |
| 126 | +++ (B) |
| 127 | ++ (B) |
| 128 | ++ (B) |
| 129 | +++ (B) |
| 130 | +++ (B) |
| 131 | +++ (B) |
| 132 | ++ (B) |
| 133 | +++ (B) |
| 134 | ++ (B) |
| 135 | +++ (B) |
| 136 | ++ (B) |
| 137 | ++ (B) |
| 138 | +++ (B) |
| 139 | +++ (B) |
| 140 | +++ (B) |
| 141 | ++ (B) |
| 142 | +++ (B) |
| 143 | +++ (B) |
| 144 | ++ (B) |
| 145 | +++ (B) |
| 146 | +++ (B) |
| 147 | ++ (B) |
| 148 | ++ (B) |
| 149 | ++ (B) |
| 150 | +++ (B) |
| 151 | +++ (B) |
| 152 | +++ (B) |
| 153 | +++ (B) |
| 154 | +++ (B) |
| 155 | +++ (B) |
| 156 | +++ (B) |
| 157 | +++ (B) |
| 158 | +++ (B) |
| 159 | +++ (B) |
| 160 | +++ (B) |
| 161 | ++ (B) |
| 162 | +++ (B) |
| 163 | ++ (B) |
| 164 | +++ (B) |
| 165 | +++ (B) |
| 166 | +++ (B) |
| 167 | +++ (B) |
| 168 | +++ (B) |
| 169 | +++ (B) |
| 170 | +++ (B) |
| 171 | +++ (B) |
| 172 | +++ (B) |
| 173 | ++ (B) |
| 174 | +++ (B) |
| 175 | ++ (B) |
| 176 | +++ (B) |
| 177 | +++ (B) |

### C. Cellular Assays—Cell Growth Inhibition Assay

Human breast tumor cells (BT474) were cultured in RPMI (Mediatech 50-011-PB), containing 10% FBS (JRH Biosciences 12176) and 1% penicillin-streptomycin (Invitrogen 15140). Human head and neck tumor cells (HN5) were cultured in high glucose DMEM (Mediatech 50-013-PB) containing 10% FBS and 1% penicillin-streptomycin. Human colon tumor cells (Colo205) were cultured in RPMI (Mediatech 50-020-PB) containing 10% FBS and 1% penicillin-streptomycin. Human melanoma cancer cells (SK-MEL-28) were cultured in EMEM with nonessential amino acids (Mediatech 50-011-PB) containing 10% FBS, 1% sodium pyruvate (JT Baker 3354-04), and 1% penicillin-streptomycin. Human melanoma cancer cells (A375P F11s) were cultured in RPMI (Mediatech 50-020-PB) containing 10% FBS, 1% sodium pyruvate, and 1% penicillin-streptomycin. Human colon cancer cells (HT-29) were cultured in high glucose DMEM (Mediatech 50-013-PB) containing 10% FBS and 1% penicillin-streptomycin. Human melanoma cancer cells (SK-MEL-3) were cultured in McCoy's 5A (50-025-PB) containing 15% FBS, and 1% penicillin-streptomycin. All cell lines were maintained at 37° C. in a humidified 5% CO₂, 95% air incubator. Cells were harvested using trypsin/EDTA (Invitrogen 25200), counted using a haemocytometer, and plated. For 96-well assays (using white full-area NUNC plates cat. #136102), cells were plated in 105 μL at the following densities (cells/well): HNS, 500; Colo 205, 500; SK-MEL-28, 500; A375P F11s, 500; HT-29, 500; SK-MEL-3, 500; and BT474, 3000. For 384-well assays (white full-area NUNC plates, cat. #781080), cells were plated in 48 μL at the following densities (cells/well): HNS, 500; Colo205, 500; SK-MEL-28, 500; A375P F11s, 500; HT-29, 500; SK-MEL-3, 500; and BT474, 2000.

The next day, compounds were diluted as follow: For 96-well assays, 13.5 μL of compound in DMSO were diluted using nine (9) serial 1:3 dilutions of 4.5 μL in 9 of DMSO. Medium (270 μL/well of RPMI with 10% FBS and 1% penicillin-streptomycin) was added to the plates. Aliquots (7 μL) were added to cells in the final assay giving a final DMSO concentration of 0.2%. For 384-well assays, 15 μL of compound in DMSO were diluted using nine (9) serial 1:3 dilutions of 5 μL in 10 μL of DMSO, followed by a further dilution of 5 μL of compound with 95 μL of medium, of which 2 μL were added to cells in the final assay giving a final DMSO concentration of 0.2%. Cells were incubated at 37° C., 5% CO₂ for 3 days.

Total ATP was measured (as a surrogate estimate of cell number) using CellTiter-Glo® reagent (Promega G7571). Briefly, plates were removed from the incubator and allowed to equilibrate to room temperature for 30 minutes. CellTiter-Glo® (25 μL or 55 μL for 384-well or 96-well assays, respectively) reagent was added to each well and plates were shaken on an orbital plate shaker for 2 minutes. Plates were incubated without shaking for a further 30 minutes and read on an LJL Analyst GT reader in luminometer mode with an integration time of 0.5 seconds per well. Percent inhibition of cell growth was calculated relative to DMSO vehicle-treated control wells. Concentration of compound required to give 50% inhibition of vehicle-treated control cell growth (IC₅₀) was interpolated using a 4-parameter fit for determining IC₅₀ using the following equation: Y=A +((B−A)/(1+((C/X)̂D))) where X=IC₅₀.

Many of the compounds of Examples 1-177 were run in the recited assays and the results are reported in the following Table 4. In the following table:
“+” indicates that the compound showed activity of >1 μM in HN5 tumor cells;
“++” indicates that the compound showed activity of between 100 nM and 1 μM in HN5 tumor cells; and
“+++” indicates that the compound showed activity of less than 100 nM in HN5 tumor cells.

TABLE 4
Activity in HN5 Tumor Cells

| Example | Activity |
|---|---|
| 1 | ++ |
| 2 | ++ |
| 3 | + |
| 4 | ++ |
| 5 | ++ |
| 6 | ++ |
| 8 | + |
| 9 | ++ |
| 10 | + |
| 11 | + |
| 12 | ++ |
| 13 | ++ |
| 14 | + |
| 15 | ++ |
| 16 | ++ |
| 17 | ++ |
| 18 | ++ |
| 19 | + |
| 20 | + |
| 21 | ++ |
| 22 | + |
| 23 | + |
| 24 | + |
| 25 | + |
| 26 | + |
| 27 | + |
| 28 | + |
| 29 | ++ |
| 30 | +++ |
| 31 | + |
| 32 | + |
| 33 | + |
| 34 | + |
| 36 | + |
| 38 | + |
| 39 | + |
| 42 | +++ |
| 44 | + |
| 47 | + |
| 49 | + |
| 51 | ++ |
| 52 | ++ |
| 53 | +++ |
| 56 | + |
| 57 | ++ |
| 58 | + |
| 62 | ++ |
| 65 | ++ |
| 66 | + |
| 70 | + |
| 71 | ++ |
| 72 | ++ |
| 73 | ++ |
| 74 | + |
| 75 | + |
| 77 | +++ |
| 78 | + |
| 79 | + |
| 80 | ++ |
| 81 | ++ |
| 82 | ++ |
| 83 | ++ |
| 84 | ++ |
| 85 | ++ |
| 87 | + |
| 88 | ++ |
| 89 | ++ |
| 91 | + |
| 92 | ++ |
| 93 | + |
| 94 | + |
| 95 | ++ |
| 96 | ++ |
| 97 | + |
| 98 | + |
| 99 | + |
| 100 | +++ |
| 101 | ++ |
| 102 | + |
| 106 | ++ |
| 107 | + |
| 109 | + |
| 112 | + |
| 113 | + |
| 114 | + |
| 116 | + |
| 117 | ++ |
| 119 | + |
| 120 | + |
| 121 | ++ |
| 122 | ++ |
| 123 | ++ |
| 124 | + |
| 126 | ++ |
| 127 | + |
| 128 | ++ |
| 129 | + |
| 130 | ++ |
| 131 | + |
| 133 | ++ |
| 140 | + |
| 145 | + |
| 146 | +++ |
| 151 | ++ |
| 152 | ++ |
| 155 | ++ |
| 157 | + |
| 159 | +++ |
| 162 | ++ |
| 166 | ++ |
| 167 | ++ |
| 169 | + |

Many of the compounds of Examples 1-177 were run in the recited assay and the results are reported in the following Table 5. In the following table:
“+” indicates that the compound showed activity of >1 μM in BT474 tumor cells;
“++” indicates that the compound showed activity of between 100 nM and 1 μM in BT474 tumor cells; and
“+++” indicates that the compound showed activity of less than 100 nM in BT474 tumor cells.

TABLE 5
Activity in BT474 Tumor Cells

| Example | Activity |
|---|---|
| 1 | ++ |
| 2 | + |
| 3 | + |
| 4 | + |
| 5 | ++ |
| 6 | + |
| 7 | + |
| 8 | + |
| 9 | + |
| 10 | + |
| 11 | + |
| 12 | + |
| 13 | + |
| 14 | + |
| 15 | + |
| 16 | + |
| 17 | + |
| 18 | + |
| 19 | + |
| 20 | + |
| 21 | + |
| 22 | + |
| 23 | + |
| 24 | + |
| 25 | + |
| 26 | + |
| 27 | + |
| 28 | + |
| 29 | + |
| 30 | + |
| 31 | + |
| 32 | + |
| 33 | + |
| 34 | + |
| 36 | + |
| 38 | + |
| 39 | + |
| 42 | + |
| 44 | + |
| 47 | + |
| 49 | + |
| 51 | + |
| 52 | + |
| 53 | + |
| 56 | + |
| 57 | + |
| 58 | + |
| 62 | + |
| 65 | + |
| 66 | + |
| 70 | + |
| 71 | + |
| 72 | + |
| 73 | + |
| 74 | + |
| 75 | + |
| 77 | + |
| 78 | + |
| 79 | + |
| 80 | + |
| 81 | + |
| 82 | + |
| 83 | + |
| 84 | + |
| 87 | + |
| 88 | + |
| 89 | + |
| 91 | + |
| 92 | + |
| 93 | + |
| 94 | + |
| 95 | + |
| 96 | + |
| 97 | + |
| 98 | + |
| 99 | + |
| 100 | + |
| 101 | + |
| 102 | + |
| 106 | + |
| 107 | + |
| 109 | ++ |
| 112 | + |
| 113 | ++ |
| 114 | + |
| 116 | + |
| 117 | + |
| 119 | + |
| 120 | + |
| 121 | + |
| 122 | + |
| 123 | + |
| 124 | + |
| 126 | + |
| 127 | + |
| 128 | ++ |
| 129 | + |
| 130 | + |
| 131 | + |
| 133 | + |
| 140 | + |
| 145 | + |
| 146 | + |
| 151 | + |
| 152 | ++ |
| 155 | + |
| 157 | + |
| 159 | + |
| 162 | + |
| 166 | + |
| 167 | + |
| 169 | + |

Many of the compounds of Examples 1-177 were run in the recited assay and the results are reported in the following Table 6. In the following table:
“+” indicates that the compound showed activity of >1 μM in Colo205 tumor cells;
“++” indicates that the compound showed activity of between 100 nM and 1 μM in Colo205 tumor cells; and
“+++” indicates that the compound showed activity of less than 100 nM in Colo205 tumor cells.

TABLE 6
Activity in Colo205 Tumor Cells

| Example | Activity |
|---|---|
| 1 | ++ |
| 2 | +++ |
| 3 | +++ |
| 4 | ++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 24 | +++ |
| 25 | +++ |
| 26 | +++ |
| 27 | ++ |
| 28 | +++ |
| 29 | +++ |
| 30 | +++ |
| 31 | ++ |
| 32 | +++ |
| 33 | +++ |
| 34 | ++ |
| 35 | + |
| 36 | ++ |
| 37 | + |
| 38 | ++ |
| 39 | ++ |
| 40 | + |
| 41 | + |
| 42 | +++ |
| 43 | + |
| 44 | +++ |
| 45 | ++ |
| 46 | + |
| 47 | +++ |
| 48 | +++ |
| 49 | +++ |
| 50 | +++ |
| 51 | +++ |
| 52 | +++ |
| 53 | +++ |
| 54 | +++ |
| 55 | +++ |
| 56 | +++ |
| 57 | +++ |
| 58 | ++ |
| 59 | ++ |
| 60 | ++ |
| 61 | ++ |
| 62 | +++ |
| 63 | ++ |
| 64 | + |
| 65 | ++ |
| 66 | ++ |
| 67 | ++ |
| 68 | + |
| 69 | + |
| 70 | ++ |
| 71 | +++ |
| 72 | ++ |
| 73 | +++ |
| 74 | ++ |
| 75 | ++ |
| 76 | + |
| 77 | +++ |
| 78 | +++ |
| 79 | ++ |
| 80 | +++ |
| 81 | +++ |
| 82 | +++ |
| 83 | ++ |
| 84 | +++ |
| 85 | +++ |
| 86 | + |
| 87 | +++ |
| 88 | +++ |
| 89 | +++ |
| 90 | ++ |
| 91 | +++ |
| 92 | ++ |
| 93 | +++ |
| 94 | +++ |
| 95 | +++ |
| 96 | +++ |
| 97 | +++ |
| 98 | ++ |
| 99 | +++ |
| 100 | +++ |
| 101 | ++ |
| 102 | ++ |
| 103 | ++ |
| 104 | ++ |
| 105 | ++ |
| 106 | ++ |
| 107 | ++ |
| 108 | ++ |
| 109 | +++ |
| 110 | ++ |
| 111 | + |
| 112 | ++ |
| 113 | ++ |
| 114 | ++ |
| 115 | ++ |
| 116 | +++ |
| 117 | +++ |
| 118 | + |
| 119 | ++ |
| 120 | +++ |
| 121 | +++ |
| 122 | +++ |
| 123 | +++ |
| 124 | ++ |
| 125 | ++ |
| 126 | +++ |
| 127 | ++ |
| 128 | +++ |
| 129 | ++ |
| 130 | +++ |
| 131 | +++ |
| 132 | ++ |
| 133 | +++ |
| 134 | ++ |
| 135 | ++ |
| 136 | ++ |
| 137 | +++ |
| 138 | ++ |
| 139 | ++ |
| 140 | +++ |
| 141 | ++ |
| 142 | + |
| 143 | ++ |
| 144 | ++ |
| 145 | +++ |
| 146 | +++ |
| 147 | ++ |
| 148 | ++ |
| 149 | +++ |
| 150 | +++ |
| 151 | +++ |
| 152 | +++ |
| 153 | +++ |
| 154 | +++ |
| 155 | +++ |
| 156 | ++ |
| 157 | +++ |
| 158 | +++ |
| 159 | +++ |
| 160 | +++ |
| 161 | +++ |
| 162 | +++ |
| 163 | ++ |
| 164 | +++ |
| 165 | +++ |
| 166 | ++ |
| 167 | +++ |
| 169 | +++ |
| 170 | +++ |
| 171 | +++ |
| 172 | + |
| 173 | ++ |
| 174 | +++ |
| 175 | +++ |
| 176 | +++ |
| 177 | +++ |

Many of the compounds of Examples 1-177 were run in the recited assay and the results are reported in the following Table 7. In the following table:
“+” indicates that the compound showed activity of >1 μM in SK-MEL-28 tumor cells;
“++” indicates that the compound showed activity of between 100 nM and 1 μM in SK-MEL-28 tumor cells; and
“+++” indicates that the compound showed activity of less than 100 nM in SK-MEL-28 tumor cells.

TABLE 7
Activity in SK-MEL-28 Tumor Cells

| Example | Activity |
|---|---|
| 1 | ++ |
| 2 | +++ |
| 3 | +++ |
| 4 | ++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | ++ |
| 20 | +++ |
| 21 | +++ |
| 22 | +++ |
| 23 | +++ |
| 24 | +++ |
| 25 | ++ |
| 26 | +++ |
| 27 | ++ |
| 28 | +++ |
| 29 | +++ |
| 30 | +++ |
| 31 | ++ |
| 32 | +++ |
| 33 | +++ |
| 34 | ++ |
| 35 | + |
| 36 | ++ |
| 37 | + |
| 38 | ++ |
| 39 | ++ |
| 40 | + |
| 41 | + |
| 42 | +++ |
| 43 | + |
| 44 | +++ |
| 45 | ++ |
| 46 | + |
| 47 | +++ |
| 48 | ++ |
| 49 | +++ |
| 50 | ++ |
| 51 | +++ |
| 52 | +++ |
| 53 | +++ |
| 54 | +++ |
| 55 | ++ |
| 56 | +++ |
| 57 | +++ |
| 58 | ++ |
| 59 | ++ |
| 60 | + |
| 61 | ++ |
| 62 | +++ |
| 63 | + |
| 64 | + |
| 65 | ++ |
| 66 | ++ |
| 67 | ++ |
| 68 | + |
| 69 | + |
| 70 | ++ |
| 71 | +++ |
| 72 | ++ |
| 73 | +++ |
| 74 | ++ |
| 75 | ++ |
| 76 | + |
| 77 | +++ |
| 78 | +++ |
| 79 | ++ |
| 80 | +++ |
| 81 | +++ |
| 82 | +++ |
| 83 | ++ |
| 84 | +++ |
| 85 | +++ |
| 86 | + |
| 87 | +++ |
| 88 | +++ |
| 89 | +++ |
| 90 | ++ |
| 91 | +++ |
| 92 | ++ |
| 93 | ++ |
| 94 | +++ |
| 95 | +++ |
| 96 | +++ |
| 97 | +++ |
| 98 | ++ |
| 99 | ++ |
| 100 | +++ |
| 101 | ++ |
| 102 | ++ |
| 103 | ++ |
| 104 | + |
| 105 | + |
| 106 | ++ |
| 107 | ++ |
| 108 | + |
| 109 | +++ |
| 110 | + |
| 111 | + |
| 112 | ++ |
| 113 | ++ |
| 114 | ++ |
| 115 | +++ |
| 116 | +++ |
| 117 | +++ |
| 118 | + |
| 119 | ++ |
| 120 | ++ |
| 121 | ++ |
| 122 | +++ |
| 123 | +++ |
| 124 | ++ |
| 125 | +++ |
| 126 | +++ |
| 127 | ++ |
| 128 | ++ |
| 129 | ++ |
| 130 | +++ |
| 131 | +++ |
| 132 | ++ |
| 133 | +++ |
| 134 | ++ |
| 135 | ++ |
| 136 | ++ |
| 137 | ++ |
| 138 | +++ |
| 139 | ++ |
| 140 | +++ |
| 141 | ++ |
| 142 | + |
| 143 | +++ |
| 144 | ++ |
| 145 | +++ |
| 146 | +++ |
| 147 | ++ |
| 148 | ++ |
| 149 | ++ |
| 150 | +++ |
| 151 | +++ |
| 152 | +++ |
| 153 | ++ |
| 154 | +++ |
| 155 | +++ |
| 156 | ++ |
| 157 | +++ |
| 158 | +++ |
| 159 | +++ |
| 160 | ++ |
| 161 | ++ |
| 162 | +++ |
| 163 | ++ |
| 164 | +++ |
| 165 | +++ |
| 166 | +++ |
| 167 | +++ |
| 168 | + |
| 169 | +++ |
| 170 | ++ |
| 171 | +++ |
| 172 | + |
| 173 | ++ |
| 174 | +++ |
| 175 | ++ |
| 176 | +++ |
| 177 | +++ |

Many of the compounds of Examples 1-177 were run in the recited assay and the results are reported in the following Table 8. In the following table:
“+” indicates that the compound showed activity of >1 μM in A375P F11s tumor cells;
“++” indicates that the compound showed activity of between 100 nM and 1 μM in A375P F11s tumor cells; and
“+++” indicates that the compound showed activity of less than 100 nM in A375P F11s tumor cells.

TABLE 8
Activity in A375P F11s Tumor Cells

| Example | Activity |
|---|---|
| 1 | ++ |
| 2 | +++ |
| 3 | +++ |
| 4 | ++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | ++ |
| 20 | ++ |
| 21 | +++ |
| 22 | +++ |
| 23 | +++ |
| 24 | +++ |
| 25 | ++ |
| 26 | +++ |
| 27 | +++ |
| 28 | +++ |
| 29 | +++ |
| 30 | +++ |
| 31 | ++ |
| 32 | +++ |
| 33 | +++ |
| 34 | ++ |
| 36 | ++ |
| 38 | ++ |
| 39 | ++ |
| 42 | ++ |
| 44 | ++ |
| 47 | +++ |
| 49 | +++ |
| 51 | +++ |
| 52 | +++ |
| 53 | +++ |
| 56 | ++ |
| 57 | ++ |
| 58 | ++ |
| 62 | +++ |
| 65 | ++ |
| 70 | ++ |
| 71 | +++ |
| 72 | ++ |
| 73 | +++ |
| 74 | ++ |
| 75 | ++ |
| 77 | +++ |
| 78 | +++ |
| 79 | ++ |
| 80 | +++ |
| 81 | +++ |
| 82 | +++ |
| 83 | ++ |
| 84 | +++ |
| 85 | +++ |
| 87 | +++ |
| 88 | +++ |
| 89 | +++ |
| 91 | ++ |
| 92 | ++ |
| 93 | ++ |
| 95 | ++ |
| 96 | +++ |
| 97 | +++ |
| 98 | ++ |
| 99 | ++ |
| 100 | +++ |
| 101 | ++ |
| 102 | ++ |
| 106 | +++ |
| 107 | ++ |
| 109 | +++ |
| 112 | ++ |
| 113 | ++ |
| 114 | ++ |
| 115 | ++ |
| 116 | ++ |
| 117 | ++ |
| 119 | ++ |
| 120 | ++ |
| 121 | ++ |
| 122 | +++ |
| 123 | +++ |
| 124 | + |
| 126 | ++ |
| 127 | + |
| 128 | ++ |
| 129 | ++ |
| 130 | +++ |
| 131 | +++ |
| 133 | +++ |
| 140 | +++ |
| 145 | +++ |
| 146 | +++ |
| 151 | +++ |
| 152 | +++ |
| 155 | +++ |
| 157 | +++ |
| 159 | +++ |
| 162 | +++ |
| 166 | ++ |
| 167 | +++ |
| 170 | ++ |

Many of the compounds of Examples 1-177 were run in the recited assay and the results are reported in the following Table 9. In the following table:
“+” indicates that the compound showed activity of >1 μM in HT-29 tumor cells;
“++” indicates that the compound showed activity of between 100 nM and 1 μM in HT-29 tumor cells; and
“+++” indicates that the compound showed activity of less than 100 nM in HT-29 tumor cells.

TABLE 9
Activity in HT-29 Tumor Cells

| Example | Activity |
|---|---|
| 1 | ++ |
| 2 | ++ |
| 3 | ++ |
| 4 | ++ |
| 5 | +++ |
| 6 | +++ |
| 7 | +++ |
| 8 | ++ |
| 9 | ++ |
| 10 | ++ |
| 11 | +++ |
| 12 | +++ |
| 13 | ++ |
| 14 | ++ |
| 15 | +++ |
| 16 | +++ |
| 17 | +++ |
| 18 | +++ |
| 19 | + |
| 20 | + |
| 21 | +++ |
| 22 | ++ |
| 23 | +++ |
| 24 | +++ |
| 25 | ++ |
| 26 | + |
| 27 | ++ |
| 28 | ++ |
| 29 | ++ |
| 30 | ++ |
| 31 | +++ |
| 32 | + |
| 33 | ++ |
| 34 | + |
| 36 | + |
| 38 | + |
| 39 | + |
| 42 | ++ |
| 44 | ++ |
| 47 | ++ |
| 49 | +++ |
| 51 | +++ |
| 52 | +++ |
| 53 | +++ |
| 56 | ++ |
| 57 | ++ |
| 58 | + |
| 62 | ++ |
| 65 | ++ |
| 66 | + |
| 70 | + |
| 71 | +++ |
| 72 | ++ |
| 73 | ++ |
| 74 | + |
| 75 | + |
| 77 | +++ |
| 78 | + |
| 79 | ++ |
| 80 | ++ |
| 81 | ++ |
| 82 | ++ |
| 83 | + |
| 84 | +++ |
| 85 | ++ |
| 87 | ++ |
| 88 | +++ |
| 89 | + |
| 91 | + |
| 92 | + |
| 93 | + |
| 94 | +++ |
| 95 | + |
| 96 | +++ |
| 97 | + |
| 98 | + |
| 99 | + |
| 100 | ++ |
| 101 | ++ |
| 102 | ++ |
| 106 | ++ |
| 107 | ++ |
| 109 | ++ |
| 112 | ++ |
| 113 | + |
| 114 | + |
| 115 | ++ |
| 116 | ++ |
| 117 | ++ |
| 119 | ++ |
| 120 | ++ |
| 121 | ++ |
| 122 | +++ |
| 123 | +++ |
| 124 | + |
| 126 | ++ |
| 127 | + |
| 128 | +++ |
| 129 | ++ |
| 130 | +++ |
| 131 | +++ |
| 133 | +++ |
| 140 | +++ |
| 145 | +++ |
| 146 | +++ |
| 151 | +++ |
| 152 | +++ |
| 155 | +++ |
| 157 | +++ |
| 159 | +++ |
| 162 | ++ |
| 166 | ++ |
| 167 | +++ |
| 170 | ++ |

Many of the compounds of Examples 1-177 were run in the recited assay and the results are reported in the following Table 10. In the following table:
“+” indicates that the compound showed activity of >1 μM in SK-MEL-3 tumor cells;
“++” indicates that the compound showed activity of between 100 nM and 1 μM in SK-MEL-3 tumor cells; and
“+++” indicates that the compound showed activity of less than 100 nM in SK-MEL-3 tumor cells.

TABLE 10
Activity in SK-MEL-3 Tumor Cells

| Example | Activity |
|---|---|
| 1 | ++ |
| 2 | +++ |
| 3 | +++ |
| 4 | ++ |
| 5 | +++ |
| 6 | +++ |
| 7 | +++ |
| 8 | +++ |
| 9 | +++ |
| 10 | ++ |
| 11 | +++ |
| 12 | +++ |
| 13 | +++ |
| 14 | +++ |
| 15 | +++ |
| 16 | +++ |
| 17 | +++ |
| 18 | +++ |
| 19 | ++ |
| 20 | ++ |
| 21 | +++ |
| 22 | ++ |
| 23 | +++ |
| 24 | ++ |
| 25 | ++ |
| 26 | ++ |
| 27 | +++ |
| 28 | +++ |
| 29 | +++ |
| 30 | +++ |
| 31 | ++ |
| 32 | +++ |
| 33 | +++ |
| 34 | + |
| 36 | ++ |
| 38 | + |
| 39 | ++ |
| 42 | +++ |
| 44 | ++ |
| 47 | +++ |
| 49 | +++ |
| 51 | +++ |
| 52 | +++ |
| 53 | +++ |
| 56 | +++ |
| 57 | ++ |
| 58 | + |
| 62 | +++ |
| 65 | ++ |
| 70 | ++ |
| 71 | +++ |
| 72 | +++ |
| 73 | +++ |
| 74 | ++ |
| 75 | ++ |
| 77 | +++ |
| 78 | +++ |
| 79 | ++ |
| 80 | +++ |
| 81 | +++ |
| 82 | +++ |
| 83 | ++ |
| 84 | +++ |
| 85 | +++ |
| 87 | +++ |
| 88 | +++ |
| 89 | +++ |
| 91 | ++ |
| 92 | ++ |
| 93 | ++ |
| 95 | ++ |
| 96 | +++ |
| 97 | +++ |
| 98 | ++ |
| 99 | ++ |
| 100 | +++ |
| 101 | ++ |
| 102 | ++ |
| 106 | +++ |
| 107 | ++ |
| 109 | +++ |
| 112 | ++ |
| 113 | ++ |
| 114 | + |
| 115 | ++ |
| 116 | ++ |
| 117 | ++ |
| 120 | +++ |
| 121 | ++ |
| 122 | +++ |
| 123 | +++ |
| 124 | + |
| 126 | ++ |
| 127 | + |
| 128 | +++ |
| 129 | ++ |
| 130 | +++ |
| 131 | +++ |
| 133 | +++ |
| 140 | +++ |
| 145 | +++ |
| 146 | +++ |
| 151 | +++ |
| 152 | +++ |
| 155 | +++ |
| 157 | +++ |
| 159 | +++ |
| 162 | +++ |
| 166 | ++ |
| 167 | +++ |
| 170 | ++ |

D. In vivo Experiments

A375P F11s and Colo205 cells were cultured in RPMI 1640 medium supplemented with 10% fetal bovine serum and 1% sodium pyruvate or 10% fetal bovine serum and without sodium pyruvate, respectively. Tumor cells (1.75×10⁶ A375P F11 s or 5×10⁶ Colo205) were implanted subcutaneously into the right flank of athymic mice on Day 1. To facilitate their growth, A375P F11s cells were suspended in Matrigel diluted 1:1 in phosphate-buffered saline before implantation. When tumors had reached approximately 200 mm³ in volume (Day 19-22), tumor-bearing mice were randomized into study groups (n=7 or 8). Animals were dosed orally once or twice daily for a 14-day period. The compound of Example 19b was dosed in a 0.5% HPMC/0.2% Tween 80 pH 7-8 vehicle. Tumor growth was measured twice a week using calipers for the duration of the study. Tumor volumes were calculated as a product of (length×width×width)/2 and median values were used to compare groups. Complete regressions (CR) were defined as three consecutive tumor measurements of ≦13.5 mm³. Partial regressions were defined as three consecutive measurements of ≦50% of starting tumor volume. Tumor growth delay was defined as the difference in time taken for treated and control groups to reach 1000 mm³ (T-C1000).

In the following table:
“−” indicates no response
“+” indicates growth delay (1-2×doubling)
“++” indicates growth delay (>2×doubling)
“+++” indicates stable disease
“++++” indicates partial regression
“+++++” indicates complete regression

TABLE 11
In vivo Evaluation

| Tumor Line | Dosage | | Response |
|---|---|---|---|
| A375P F11s | 300 | mg/kg qd | ++++(+) |
| A375P F11s | 100 | mg/kg qd | ++++(+) |
| A375P F11s | 30 | mg/kg qd | +++ (during dosing)* |
| A375P F11s | 100 | mg/kg bid | ++++(+) |
| A375P F11s | 30 | mg/kg bid | ++++ |
| A375P F11s | 30 | mg/kg bid | ++++(+) |
| A375P F11s | 10 | mg/kg bid | ++ |
| A375P F11s | 3 | mg/kg bid | + |
| A375P F11s | 1 | mg/kg bid | − |
| Colo205 | 300 | mg/kg qd | ++++ |
| Colo205 | 100 | mg/kg qd | ++(++) |
| Colo205 | 30 | mg/kg qd | − |
| Colo205 | 100 | mg/kg bid | ++++ |
| Colo205 | 30 | mg/kg bid | ++ |

*Median tumor volume during dosing indicated stable disease; however, following the cessation of dosing, tumor volumes quickly rebounded and resulted in no tumor growth delay.

### Pharmaceutical Formulation Example

### Preparation of Capsules Containing a Compound of the Invention (Freebase)

Contents in each capsule:
   =60 mg Active Pharmaceutical ingredient (API)+60 mg Avicel+13 mg SSG.
   133 mg total powder in a size 0 hard gelatin capsule. The Avicel/SSG weight may be reasonably approximate.

### Procedure:

1. Separate the halves of hard-gelatin capsule and mark/identify each as appropriate/needed.
2. Place the bottom capsule half in capsule filler with the filling funnel on top.
3. Weigh the components (Avicel, Sodium Starch Glycolate (SSG), API) onto a single weigh paper (tared on an analytical balance between each weighing).
4. Record weights of each component.
5. Carefully and thoroughly mix the dry powders on the weigh paper with a small spatula.
6. Carefully transfer the mixed powders into the capsule through the funnel.
7. Place the top half onto the capsule and close until secure, shake capsule to mix/distribute contents.
8. IF powder begins to near top of capsule, gently tap capsule and powder should settle.
9. Place the capsule into a small appropriately labeled bottle (but large enough to easily remove it).

### Pharmaceutical Formulation Example

### Preparation of Tablets Containing a Compound of the Invention (Freebase)

| | Quantity | |
|---|---|---|
| Component | (mg/tablet) | % w/w |
| Core Tablet | | |
| API | 405.0 | 71.6 |
| Lactose monohydrate | 59.0 | 10.4 |
| Polysorbate 80 | 1.0 | 0.2 |
| Povidone | 40.0 | 7.1 |
| Colloidal Silicon Dioxide | 5.5 | 1.0 |
| Crospovidone | 51.0 | 9.0 |
| Magnesium Stearate | 4.5 | 0.8 |
| Purified Water | qs | |
| Film Coating | | |
| Opadry ® Orange, YS-1-13065-A | 17.0 | 3.0 |
| Purified water | qs | |

### Procedure:

1. Sieve Lactose, Silicon dioxide, Crospovidone and half Povidone.

### 2. Add API.

3. Granulate in High Shear Granulator with granulating solution containing dissolved Polysorbate 80 and other half of Povidone in Purified water.
4. Mill using Comil 197, 0.375″ screen.
5. Dry using Fluid Bed Dryer
6. Mill using Comil 197, 0.075″ screen
7. Add Crospofidone, magnesium stearate.
8. Blend 5 minute
9. Compress tablet
10. Aqueous film coat tablet

What is claimed is:

1. A compound of formula (I): 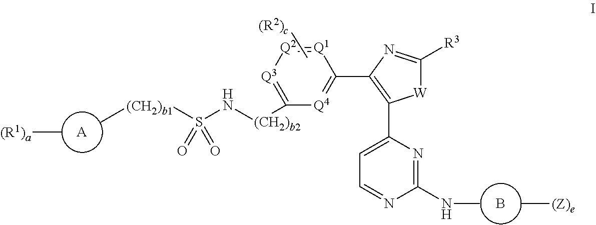
wherein:
a is 0, 1, 2 or 3;
each R¹ is the same or different and is independently selected from halo, alkyl, haloalkyl, —OR⁶, —CO₂H, —CO₂R⁶, and —CN;
Ring A is C₃₋₆cycloalkyl, phenyl, and 5-6 membered heterocycle or heteroaryl having 1 or 2 heteroatoms selected from N, O and S;
one of b1 and b2 is 0 and the other is 0 or 1;
c is 0, 1 or 2;
each R² is the same or different and is independently selected from halo, alkyl, haloalkyl, —OR⁶, and —CN;
each of Q¹, Q², Q³, and Q⁴ is CH or C—R² or one of Q¹, Q², Q³, and Q⁴ is N and the others are CH or C—R²;
W is —O— or —S—;
R³ is selected from H, alkyl, haloalkyl, alkenyl, C₃₋₆cycloalkyl, phenyl, Het, —CH₂-Het,
—NR⁶R⁷, —N(R⁶)—C₃₋₆cycloalkyl, —N(R⁶)Het, —N(R⁶)R⁵—Het, —N(R⁶)—R⁵—OR⁷, —N(R⁶)—R⁵—NR⁶R⁷, —N(H)C(O)R⁶, —N(R⁶)—C(O)—NR⁶R⁷, —N(H)SO₂R⁶, —N(R⁶)—R⁵—S(O)₂R⁷, and —N(R⁶)—S(O)₂—NR⁶R⁷,
wherein each of said cycloalkyl is optionally substituted with 1 or 2 substituents which are the same or different and are independently selected from halo, C₁₋₃alkyl, haloC₁₋₃alkyl, OH, O—C₁₋₃alkyl, oxo, S(C₁₋₃alkyl), SO₂, NH₂,
N(H)C₁₋₃alkyl and N(C₁₋₃alkyl)₂, and
wherein said phenyl is optionally substituted with 1, 2 or 3 substituents which are the same or different and are each independently selected from halo,
C₁₋₃alkyl, haloC₁₋₃alkyl, O—C₁₋₃alkyl, C₁₋₃alkylene-O—C₁₋₃alkyl, OH,
C₁₋₃alkylene-OH, NH₂, N(H)C₁₋₃alkyl, N(C₁₋₃alkyl)₂, CN and NO₂;
each Het is the same or different and is independently a 4-6 membered heterocycle having 1 or 2 heteroatoms selected from N, O and S and optionally substituted with 1 or 2 substituents which are the same or different and are each independently selected from halo,
C₁₋₃alkyl, O—C₁₋₃alkyl, C₁₋₃alkylene-O—C₁₋₃alkyl, OH,
C₁₋₃alkylene-OH, oxo, SO₂(C₁₋₃alkyl), C₁₋₃alkylene-SO₂(C₁₋₃alkyl), NH₂, N(H)C₁₋₃alkyl, N(C₁₋₃alkyl)₂, CN, and —CH₂CN;
each R⁵ is the same or different and is independently C₁₋₄alkylene;
Ring B is selected from phenyl, 9-10 membered aryl, 5-6 membered heteroaryl and 9-10 membered heteroaryl, each heteroaryl having 1, 2, or 3 heteroatoms selected from N, O and S;
wherein when Ring B is selected from phenyl and 5-6 membered heteroaryl, then
e is 0, 1, 2 or 3; and
each Z is the same or different and is independently selected from:
halo, alkyl, haloalkyl, alkenyl,
Het², —R⁵Het², Het³-Het²,
oxo, —OR⁶, —R⁵—OR⁶, —O—R⁵—OR⁶, —OHet², —O—R⁵—Het², —O—R⁵—NR⁶R⁷, —O—R⁵—S(O)₂R⁶, —C(O)R⁶, —C(O)CH₂NR⁶R⁷, —CO₂R⁶, —R⁵—CO₂R⁶, —S(O)_{f}R⁶
—R⁵—S(O)₂R⁶, —S(O)₂NR⁶R⁷, —R⁵—S(O)₂NR⁶R⁷, —S(O)₂—R⁵—NR⁶R⁷, —NR⁶R⁷, —R⁵—NR⁶R⁷, —N(R⁶)Het², —N(R⁶)—R⁵—OR⁷, —N(R⁶)—R⁵—S(O)_{f}R⁷, —N(R⁶)—R⁵—CN, —N(R⁶)—R⁵—NR⁶R⁷, —N(H)S(O)₂R⁶, —N(R⁶)—C(O)—NR⁶R⁷, —N(R⁶)—S(O)₂—NR⁶R⁷, —CN, and —R⁵—ON; and
wherein when Ring B is selected from 9-10 membered aryl and 9-10 membered heteroaryl, then:
e is 0, 1 or 2; and
each Z is the same or different and is independently selected from:
halo, alkyl, oxo, —C(O)R⁶, —C(O)CH₂NR⁶R⁷, —OR⁶, and —NR⁶R⁷;
each Het² is the same or different and is independently a heterocycle optionally substituted with 1 or 2 substituents which are the same or different and are each independently selected from halo, C₁₋₃alkyl, haloC₁₋₃alkyl,
O—C₁₋₃alkyl, C₁₋₃alkylene-O—C₁₋₃alkyl, OH, C₁₋₃alkylene-OH, oxo, C(O)(C₁₋₃alkyl), SO₂(C₁₋₃alkyl), C₁₋₃alkylene-SO₂(C₁₋₃alkyl), NH₂, N(H)C₁₋₃alkyl, N(C₁₋₃alkyl)₂, CN and C₁₋₃alkylene-CN;
Het³ is a 5-6 membered heterocycle having 1 or 2 heteroatoms selected from N, O and S and optionally substituted with 1 or 2 substituents which are the same or different and are each independently selected from halo,
C₁₋₃alkyl, haloC₁₋₃alkyl, and O—C₁₋₃alkyl;
f is 0, 1 or 2; and
each R⁶ and each R⁷ is the same or different and is independently H, alkyl or haloalkyl;
or a pharmaceutically acceptable salt thereof.

2. The compound according to claim 1, wherein a is 0, 1 or 2.

3. The compound according to claim 1, wherein each R¹ is the same or different and is independently selected from halo, alkyl, haloalkyl, and —OR⁶.

4. The compound according to claim 1, wherein Ring A is phenyl or 5-6 membered heteroaryl.

5-6. (canceled)

7. The compound according to claim 1, wherein each of Q¹, Q², Q³, and Q⁴ is CH.

8. The compound according to claim 1, wherein Q³ is N, and Q¹, Q², and Q⁴ are all CH.

9-15. (canceled)

16. The compound according to claim 1, wherein W is S.

17-20. (canceled)

21. The compound according to claim 1, wherein Ring B is phenyl or 5-6 membered heteroaryl having 1 or 2 heteroatoms selected from N, O and S.

22. The compound according to claim 1, wherein Ring B is phenyl.

23-28. (canceled)

29. A compound selected from:
N-[3-(2-(1-Methylethyl)-5-{2-[(6-{4-[2-(methyloxy)ethyl]-1-piperazinyl}-3-pyridinyl)amino]-4-pyrimidinyl}-1,3-thiazol-4yl)phenyl]benzenesulfonamide;
N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-4-fluorobenzenesulfonamide;
N-{3-[5-(2-{[6-(4-Acetyl-1-piperazinyl)-3-pyridinyl]amino}-4-pyrimidinyl)-2-(1-methylethyl)-1,3-thiazol-4-yl]phenyl}-2-fluorobenzenesulfonamide;
2,6-Difluoro-N-{3-[5-(2-{[3-fluoro-4-({1-[2-(methylsulfonyl)ethyl]-4-piperidinyl}oxy)phenyl]amino}-4-pyrimidinyl)-2-(1-methylethyl)-1,3-thiazol-4-yl]phenyl}benzenesulfonamide hydrochloride;
5-({4-[4-(3-{[(2,6-Difluorophenyl)sulfonyl]amino}phenyl)-2-(1-methylethyl)-1,3-thiazol-5-yl]-2-pyrimidinyl}amino)-2-(4-morpholinyl)benzoic acid;
2,6-Difluoro-N-{3-[5-[2-({6-[(9aR)-hexahydropyrazino[2,1-c][1,4]oxazin-8(1H)-yl]-3-pyridiinyl}amino)-4-pyridinyl]-2-(1-methylethyl)-1,3-thiazol-4-yl]phenyl}benzenesulfonamide;
2,6-Difluoro-N-{3-[5-{2-[(3-fluoro-4-{4-[2-(methylsulfonyl)ethyl]-1-piperazinyl}phenyl)amino]-4-pyrimidinyl}-2-(1-methylethyl)-1,3-thiazol-4-yl]phenyl}benzenesulfonamide hydrochloride;
2,6-Difluoro-N-{3-[2-(1-methylethyl)-5-(2-{[6-(4-morpholinyl)-3-pyridinyl]amino}-4-pyrimidinyl)-1,3-thiazol-4-yl]phenyl}benzenesulfonamide;
N-{3-[5-(2-{[3-(Dimethylamino)-4-(methyloxy)phenyl]amino}-4-pyrimidinyl)-2-(1-methylethyl)-1,3-thiazol-4-yl]phenyl}-2,6-difluorobenzenesulfonamide trifluoroacetate;
2,6-Difluoro-N-[3-(2-(1-methylethyl)-5-{2-[(5-methyl-2-(methyloxy)-4-{4-[2-(methylsulfonyl)ethyl]-1-piperazinyl}phenyl)amino]-4-pyrimidinyl}-1,3-thiazol-4-yl)phenyl]benzenesulfonamide;
2,6-Difluoro-N-{3-[2-(1-methylethyl)-5-(2-{[2-(methyloxy)-4-(4-{4-[2-(methylsulfonyl)ethyl]-1-piperazinyl}-1-piperidinyl)phenyl]amino}-4-pyrimidinyl)-1,3-thiazol-4-yl]phenyl}benzenesulfonamide;
2,6-Difluoro-N-{3-[5-[2-({3-fluoro-4-[4-(2-fluoroethyl)-1-piperazinyl]phenyl}amino)-4-pyrimidinyl]-2-(1-pyrrolidinyl)-1,3-thiazol-4-yl]phenyl}benzenesulfonamide;
2-Fluoro-N-{2-fluoro-5-[5-{2-[(3-fluoro-4-{4-[2-(methylsulfonyl)ethyl]-1-piperazinyl}phenyl)amino]-4-pyrimidinyl}-2-(1-methylethyl)-1,3-thiazol-4-yl]phenyl}benzenesulfonamide hydrochloride;
2,6-Difluoro-N-{2-fluoro-5-[2-(1-methylethyl)-5-(2-{[6-(4-morpholinyl)-3-pyridinyl]amino}-4-pyrimidinyl)-1,3-thiazol-4-yl]phenyl}benzenesulfonamide;
N-{5-[5-(2-{[6-(4-Cyano-1-piperidinyl)-3-pyridinyl]amino}-4-pyrimidinyl)-2-(1-methylethyl)-1,3-thiazol-4-yl]-2-fluorophenyl}-2,6-difluorobenzenesulfonamide;
2,6-Difluoro-N-{2-fluoro-5-[5-[2-({3-fluoro-4-[4-(2-fluoroethyl)-1-piperazinyl]phenyl}amino)-4-pyrimidinyl]-2-(1-methylethyl)-1,3-thiazol-4-yl]phenyl}benzenesulfonamide;
2,6-Difluoro-N-{2-fluoro-5-[5-(2-{[6-(4-morpholinyl)-3-pyridinyl]amino}-4-pyrimidinyl)-2-(1-pyrrolidinyl)-1,3-thiazol-4-yl]phenyl}benzenesulfonamide;
N-{5-[2-(1,1-Dimethylethyl)-5-(2-{[6-(4-morpholinyl)-3-pyridinyl]amino}-4-pyrimidinyl)-1,3-thiazol-4-yl]-2-fluorophenyl}-2,6-difluorobenzenesulfonamide hydrochloride;
2,6-Difluoro-N-{2-fluoro-3-[5-(2-{[5-fluoro-6-(4-morpholinyl)-3-pyridinyl]amino}-4-pyrimidinyl)-2-(1-methylethyl)-1,3-thiazol-4-yl]phenyl}benzenesulfonamide; and
2,6-Difluoro-N-{4-[2-(1-methylethyl)-5-(2-{[6-(4-morpholinyl)-3-pyridinyl]amino}-4-pyrimidinyl)-1,3-thiazol-4-yl]-2-pyridinyl}benzenesulfonamide;
and free base or pharmaceutically acceptable salts thereof.

30-36. (canceled)

37. A pharmaceutical composition comprising a compound according to claim 1 further comprising a pharmaceutically acceptable carrier, diluent or excipient.

38. The pharmaceutical composition according to claim 37 further comprising a chemotherapeutic agent.

39-40. (canceled)

41. A method for treating melanoma in a mammal in need thereof, said method comprising administering to the mammal a therapeutically effective amount of a compound according to claim 1.

42-61. (canceled)
